SEQUENCE LISTING (331 sequences)

SEQ ID NO: 1 - protein, 196 aa, synthetic construct
MAAVSNRGRAPRQNNQRWKMQPDDFKSNYLPSQHPKVVYLMYAIKWRRGTIWRGWCSNNPTQHAEVNFLENCFKPLSSASCSITWVLSTTPCGKCSKRILEFLRIHPNVTLKIFAAKIFKHLDIRNRQGLRNLTEKGVIIRIMSPADYSSSWKRFVAYQHGEEDYWPWNFILHILLNCIELHRIILGLPPVLNLEY

SEQ ID NO: 2 - protein, 255 aa, synthetic construct
MFLLSGIETEREKHTPVKQDTAWEAVVKAMATGWNRDHESRGITKGGKISQETFIDTYDPSVLRREQYMLYEIKWSNNKRPRQSCCHSTQTEHAEIYFLEDVFQKQRSDPSAHCFITWFVSWSPCGDCCKQIRDFLKEQPNVKLFIYVARIYWHKREINRQGLRNLMNIGVSIRVMYLPVYRYCWRTFVDKEDKYEDYWPRHFAPWIMLYSLELQSILQNIPSCLKISSFKNQSPVFSLCVDEKQKRALALANPY

SEQ ID NO: 3 - protein, 194 aa, synthetic construct
HYPEIQPKEFKDVYDPSVLPKVTYLLYEIKWGNSTKIWRNWCRNTSTQHAEINFLENAFRERSFNPLTHCSITWFLSWSPCWKCSQSVVEFLKTYPKVNLEMYVARLFWHEDRSNRLGLRDLVMNGVTIRVMDLSAYNYCWRTFVSHQNVDDYWPWHLTPWIMFFSFELQLILQVSRSLGGKDSIQKLSRDMYR

SEQ ID NO: 4 - protein, 239 aa, synthetic construct
CTGHKGPKARWQIQPKEFRAIFDPSAFPKVTYLLYEIKWGSSTKFWRKWCLNTPTQHAEINCLENDFKAIHHGPSVHCSIIWFLSWSPCGLCCRLIIHFLRAHPKVTLQIYIGRLFRHRDERNRQGLRDLVSSGVNVYIMSLPAYDYCWRTFVDHRVREDDIYWPCYFTPWMIFYMLEFQCILQGLPPCLQIWLKNYTGSPVFQLVLQESHRKILWQSLHTANSPQQRLIFRPRPSTEH

SEQ ID NO: 5 - protein, 258 aa, synthetic construct
HTADQGRRIKSWEFTSFFDPSQLRKETSLLSEVWWGSSSRLWRSCSQNSTHHVEQSFLDHFQAQRHLGPSTTCTITWFLSWSPCWECARAIRTFLDKYPTVKLTIYVARLYWHMDPQNRQGLRNLVKGGVDVRIMGPSEHYYCWRNFVDYSRGEEEEWPRYHRVPIHLFDLELHCICLDLPACLTISPVKHQQLLSFSLTLHTCHYQKLPPALLLLLGLVRPPPPQTSLSVAHRGRGPGTVNGGEWDPNNWGWFWVLR

SEQ ID NO: 6 - protein, 197 aa, synthetic construct
RSSKMPEELFYKYFYPKKDRTGLTTTYLCYELRTQVDATTYEVEKGWIEDEDGVHAYLLFLEKFSALNLDPDKKYDVTLYASFSPCYECAKALAEFLKAHPNVSITIKVANLYNCEPGSPNKEGLRLLKEAGVTISIMDEADYKYVWNTFVDNKGKEYVPPEGLKENSEELQKKLEEILTPPEVEEARRRALREAGL

SEQ ID NO: 7 - DNA, 516 bp, synthetic construct
  source 1..516: mol_type = other DNA
atgggccccctcctggacgggattgctgctcggctggaggccgtgagagccgccctgctgggggagggcgggggcgccggggacgacgagccccccgccgtgccctgggacagagtggagagactgagaagagagctgccccctcccgtggtgcccaacaccgggcaaaagacccacggcagatggatcggccccgacggccaagctagacccatcgtgagcggccgggacgacaagagcgtgctggtgaaccccctgctgagaggcaagggcgcccccggccccacaagaagagacagcgacgtggagatgaagctggccgcccacatggccgctagaggcatcagacacgccaccgtggtgatcaacaacaccccctgcagaggccccctgagatgcgacaccctggtgcccatcctgctgcccgagggcagcaccctgaccgtgcacggcatcaacgagaacggcacaagaacaagaatcagatacaccggcggcgctagaccctggtggagc

SEQ ID NO: 8 - DNA, 10696 bp, synthetic construct
  source 1..10696: mol_type = other DNA
gctagcgccaccatgaaacggacagccgacggaagcgagttcgagtcaccaaagaagaagcggaaagtcatggccgccgtgagcaacagaggcagagcccctagacagaacaatcagagatggaagatgcagcccgacgacttcaagagcaactacctgcctagccaacaccccaaggtggtgtacctgatgtacgccatcaagtggagaagaggcaccatctggagaggctggtgcagcaacaaccccacacagcacgccgaggtgaacttcctggagaactgcttcaagcccctgagcagcgcctcctgcagcatcacctgggtgctgagcaccaccccctgcggcaagtgcagcaagagaatcctggagttcctgagaatccaccccaacgtgaccctgaagatcttcgccgccaagatcttcaagcacctggacatcagaaacagacaaggcctgagaaacctgaccgagaagggcgtgatcatcagaatcatgagccccgccgactacagcagcagctggaagagattcgtggcctatcagcacggcgaggaggactactggccctggaacttcatcctgcacatcctgctgaactgcatcgagctgcacagaatcatcctgggcctgccccccgtgctgaacctggagtactctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 9 - DNA, 10873 bp, synthetic construct
  source 1..10873: mol_type = other DNA
gctagcgccaccatgaaacggacagccgacggaagcgagttcgagtcaccaaagaagaagcggaaagtcatgttcctgctgagcggcatcgaaaccgagagagagaagcacacccccgtgaagcaagacaccgcctgggaggccgtggtgaaggccatggccaccggctggaaccgggaccacgagagcagaggcatcaccaagggcggcaagatcagccaagaaaccttcatcgacacctacgaccctagcgtgctgagaagagagcagtacatgctgtacgagatcaagtggagcaacaacaaaagacctagacagagctgctgccacagcacacagaccgagcacgccgagatctacttcctggaggacgtgtttcagaagcagagaagcgaccctagcgcccactgcttcatcacctggttcgtgagctggagcccctgcggcgactgctgcaagcagatcagagacttcctgaaggagcagcccaacgtgaagctgttcatctacgtggctagaatctactggcacaagagagagatcaacagacaaggcctgagaaacctgatgaacatcggcgtgagcatcagagtgatgtacctgcccgtgtacagatactgctggagaaccttcgtggacaaggaggacaagtacgaggactactggcctagacacttcgccccctggatcatgctgtacagcctggagctgcagagcatcctgcagaacatccctagctgcctgaagatcagcagcttcaagaatcagagccccgtgttcagcctgtgcgtggacgagaagcagaagagagccctggccctggccaacccctactctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 10 - DNA, 10690 bp, synthetic construct
  source 1..10690: mol_type = other DNA
gctagcgccaccatgaaacggacagccgacggaagcgagttcgagtcaccaaagaagaagcggaaagtccactaccccgagattcagcccaaggagttcaaggacgtgtacgaccctagcgtgctgcccaaggtgacctacctgctgtacgagatcaagtggggcaacagcaccaagatctggagaaactggtgcagaaacacaagcacacagcacgccgagatcaacttcctggagaacgccttcagagagcggagcttcaaccccctgacccactgcagcatcacctggttcctgagctggagcccctgctggaagtgcagccaaagcgtggtggagttcctcaagacctaccccaaggtgaacctggagatgtacgtggctagactgttctggcacgaggacagaagcaacagactgggcctgcgggacctggtgatgaacggcgtgaccatcagagtgatggacctgagcgcctacaactactgctggagaaccttcgtgagccatcagaacgtggacgactactggccctggcacctgaccccctggatcatgttcttcagcttcgagctgcagctgatcctgcaagtgagcagaagcctgggcggcaaggacagcattcagaagctgagcagagacatgtacagatctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 11 - DNA, 10825 bp, synthetic construct
  source 1..10825: mol_type = other DNA
gctagcgccaccatgaaacggacagccgacggaagcgagttcgagtcaccaaagaagaagcggaaagtctgcaccggccacaagggccccaaggctagatggcagattcagcccaaggagttcagagccatcttcgaccctagcgccttccccaaggtgacctacctgctgtacgagatcaagtggggcagcagcaccaagttctggagaaagtggtgcctgaacacccccacacagcacgccgagatcaactgcctggagaacgacttcaaggccatccaccacggccctagcgtgcactgcagcatcatctggttcctgagctggagcccctgcggcctgtgctgcagactgatcatccacttcctgagagcccaccccaaggtcaccctgcagatctacatcggcagactgttcagacaccgggacgagagaaacagacaaggcctgcgggacctggtgagcagcggcgtgaacgtgtacatcatgagcctgcccgcctacgactactgctggagaaccttcgtggaccacagagtgagagaggacgacatctactggccctgctacttcaccccctggatgatcttctacatgctggagtttcagtgcatcctgcaaggcctgcccccctgcctgcagatctggctgaagaactacaccggcagccccgtgtttcagctggtgctgcaagagagccacagaaagatcctgtggcagagcctgcacaccgccaacagccctcagcagagactgatcttcagacctagacctagcaccgagcactctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 12 - DNA, 10882 bp, synthetic construct
  source 1..10882: mol_type = other DNA
gctagcgccaccatgaaacggacagccgacggaagcgagttcgagtcaccaaagaagaagcggaaagtccacaccgccgaccaaggcagaagaatcaagagctgggagttcacatccttcttcgaccctagccaactgagaaaggagacaagcctgctgagcgaggtgtggtggggcagcagcagcagactgtggagaagctgcagccaaaacagcacccaccacgtggagcagagcttcctggaccacttccaagctcagagacacctgggccctagcaccacctgcaccatcacctggttcctgagctggagcccctgctgggagtgcgctagagccatcagaaccttcctggacaagtaccccaccgtgaagctgaccatctacgtggctagactgtactggcacatggaccctcagaacagacaaggcctgagaaacctggtgaagggcggcgtggacgtgagaatcatgggccctagcgagcactactactgctggagaaacttcgtggactacagcagaggcgaggaagaggagtggcctagataccacagagtgcccatccacctgttcgacctggagctgcactgcatctgcctggacctgcccgcctgcctgaccatcagccccgtgaagcatcagcagctgctgagcttcagcctgaccctgcacacctgtcactatcagaaactgccccccgctctgctcctgctcctcggcctggtcagacccccccctccccaaacaagcctctccgtcgcccacagagggagaggccccggcaccgtcaacggcggcgagtgggaccccaacaactggggctggttctgggtgctgagatctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 13 - DNA, 10699 bp, synthetic construct
  source 1..10699: mol_type = other DNA
gctagcgccaccatgaaacggacagccgacggaagcgagttcgagtcaccaaagaagaagcggaaagtcagaagcagcaagatgcccgaggagctgttctacaagtacttctaccccaagaaggacagaaccggcctgaccacaacctacctgtgctacgagctgagaacccaagtggacgccaccacctacgaggtggagaagggctggatcgaggacgaggacggcgtgcacgcctacctgctgttcctggagaagttcagcgccctgaacctggaccccgacaagaagtacgacgtgaccctgtacgcctccttcagcccctgctacgagtgcgccaaggccctggccgagttcctgaaggcccaccccaacgtgagcatcaccatcaaggtggccaacctgtacaactgcgagcccggcagccccaacaaggagggcctgagactgctgaaggaggccggcgtgaccatcagcatcatggacgaggccgactacaagtacgtgtggaacaccttcgtggacaacaagggcaaggagtacgtgccccccgagggcctgaaggagaacagcgaggagctgcagaagaagctggaggagatcctgaccccccccgaggtggaggaggctagacggagagccctgagagaggccggcctgtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 14 - protein, 11 aa, synthetic construct
SPKKKRKVEAS

SEQ ID NO: 15 - protein, 11 aa, synthetic construct
GPKKKRKVAAA

SEQ ID NO: 16 - protein, 7 aa, synthetic construct
PKKKRKV

SEQ ID NO: 17 - protein, 16 aa, synthetic construct
KRPAATKKAGQAKKKK

SEQ ID NO: 18 - protein, 9 aa, synthetic construct
PAAKRVKLD

SEQ ID NO: 19 - protein, 11 aa, synthetic construct
RQRRNELKRSP

SEQ ID NO: 20 - protein, 38 aa, synthetic construct
NQSSNFGPMKGGNFGGRSSGPYGGGGQYFAKPRNQGGY

SEQ ID NO: 21 - protein, 42 aa, synthetic construct
RMRIZFKNKGKDTAELRRRRVEVSVELRKAKKDEQILKRRNV

SEQ ID NO: 22 - protein, 8 aa, synthetic construct
VSRKRPRP

SEQ ID NO: 23 - protein, 8 aa, synthetic construct
PPKKARED

SEQ ID NO: 24 - protein, 8 aa, synthetic construct
POPKKKPL

SEQ ID NO: 25 - protein, 12 aa, synthetic construct
SALIKKKKKMAP

SEQ ID NO: 26 - protein, 5 aa, synthetic construct
DRLRR

SEQ ID NO: 27 - protein, 7 aa, synthetic construct
PKQKKRK

SEQ ID NO: 28 - protein, 10 aa, synthetic construct
RKLKKKIKKL

SEQ ID NO: 29 - protein, 10 aa, synthetic construct
REKKKFLKRR

SEQ ID NO: 30 - protein, 20 aa, synthetic construct
KRKGDEVDGVDEVAKKKSKK

SEQ ID NO: 31 - protein, 17 aa, synthetic construct
RKCLQAGMNLEARKTKK

SEQ ID NO: 32 - protein, 9 aa, synthetic construct
PAAKKKKLD

SEQ ID NO: 33 - protein, 32 aa, synthetic construct
SGGSSGGSSGSETPGTSESATPESSGGSSGGS

SEQ ID NO: 34 - protein, 10 aa, synthetic construct
SGGSGGSGGS

SEQ ID NO: 35 - protein, 5 aa, synthetic construct
GGGGS

SEQ ID NO: 36 - protein, 5 aa, synthetic construct
EAAAK

SEQ ID NO: 37 - protein, 12 aa, synthetic construct
MKRTADGSEFES

SEQ ID NO: 38 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 39 - protein, 228 aa, synthetic construct
AAPVLPPPLPPNLPLKIDPETFKKYFDPKVQSSETLLLYEIDFGTENGVIRGSLFSGDKHVILQFIERLESEFGLTPDTKAEITLYLSHSPCGACCDALIAFLKRYPNIKVNIYVSKLYRHENPENRAGIRRLIAAGVTVKIMDDEDYKYIWKNFVNVKPSELNNYPKYDPFYINCYRYELACIKLGLPPALKVTLSPDPNNVTFSLELTEEHYEKLPKELIEATGVK

SEQ ID NO: 40 - protein, 228 aa, synthetic construct
AAPVLPPPPDPDLPRKIEPELFDRYFDPRVQLEETLLCYEIDFGKPDSVERDTLFSGDEHVIVKFIKRYESEYNLTPDTKANITLYLSHSPCGECCAAIIAFLKKYPNIKLNIVVARLRNPADPANRAGIRALIAAGVEVRVMTDEDYRYLWQNFVRRKPEDLDKYPQYDPRYINYYRLELECIRLGLPPALVVELSPDPNDVTFTLELRPEHYERLPPELLAATGLE

SEQ ID NO: 41 - protein, 228 aa, synthetic construct
GSLLLPPPPDPNLPKKIAPETFKYYFDPKVQSKETLLLYEIDFGKEDSLIRDSLFSGDEHVILQFIKKLESEFGLTPDTKANITLYLSHSPCGACCAALIAFKAKYPNIKINIYVAQLRNHTDPANRAGIRALIAAGVTVKVMDDEIYKYIWENFVDVKPEDRDKYPKYDPKYINYYRYELECIKLGLEPALEVTLSPDPNNVTFTLNLTPEHYEKVPKELLESLGVK

SEQ ID NO: 42 - protein, 228 aa, synthetic construct
GVVVPPPPVDPDADKKIAPEDFEKYFDPRLLTEETLLLYEIDFGGPDSIYRDTLFSGDKHVELIFIERLESEPGLSPDTKADITLYLSHSPCGECCDAIVEFLKRYPNIKLNIYVARLNNPADPKNRAGIRKLIEAGVKIEVMDDEIYKYCWDNFVNRPPSQANDYPKFDPFYINLYRYELECIKLGLPPAVVIEKSPNPEDVKFSVKLTPEHYEKLPKELIDSLGLK

SEQ ID NO: 43 - protein, 228 aa, synthetic construct
GLIIPPKPRNPDEDKKIEKETFEKYFDPRNFTKERLLLYEIDYGGPNSIERDSLFSGKEHVETIFIKEYETTPGLSPDTKAHITLYLSWSPCGACCAEIIAFLKKYPNIKLTIYVSKLYNHQDPANRAGIRALIAAGVEIKIMDDEDYKYVWKNFVRVKPEDKNKYPVFDPFYINLYKLELECIKLGLPPALVVEIKPDPKDVKFSIKLQPEHYEKLPKELLESLGLK

SEQ ID NO: 44 - protein, 228 aa, synthetic construct
GIVVLPKPVDPNESKKIDKETFEKYFDPRNFTKKTLLLYSIDFGGPDSIVLDTLFSGDKHVIDIFIEKLESTPGLSPDTKATITLYLSHSPCGECCDRIVAFLKRYPNIKLNIVVSRLYNHADPKNRAGIRRLIAAGVTIRIMDQEDYEEIWKKFVKVKPEDKDKYPVFDEKYINYYKLELECIKLGLPPALVVEIKPNPEDVKFSIKLTPEHYEKLPKELLESLGLK

SEQ ID NO: 45 - protein, 228 aa, synthetic construct
ALLLVPPPEPKEKENLIKPEEFKKYFDPRLLLKETLLLYEIEYGGPGTVERSVLFSGDEHVILQFIKRFEASPGLSPDTKAHITLYLSWSPCGECCKALVAFKKKYPNIKITIYVSRLRNHTNPENREGIRLLIEAGIEIKVMTHEDLKYVWENFVDKPESEKNSYPIYDEKYITLYEYELECIKLGLEPALIVEVSPDPNNVTFSLNLKPEHYEKLPKELLESLGVK

SEQ ID NO: 46 - protein, 228 aa, synthetic construct
ALLLVPPPPDPALANLVPPEEFKKYFNPLNLLKRTLLLYEIKYGGPGTEERGVLFSGDEHVIDIFIKRFTARPDLTKDTKAKITLYLSHSPCGACCDRIVAFLKEYPNIELDIVVSKLRNHADPENRAGIRRLIAAGVTVRIATQEDYEYMWKNFVDVKPEDKNKYPVYPEEYLTYYRFELECIKLGLPPALVVKKKPDPNDVEFTLELTPEHYEKLPPELLASTGVK

SEQ ID NO: 47 - protein, 228 aa, synthetic construct
ALLLLPPPLPPSLSNLVPPELFKRYFDPRILSKETLLLYEIDYGGPNTIERKTLFSGDKHVIDIFIEELENRPGLSKDTKMNITLYLSWSPCGKCCDRIVEFKKKYPNIKINIVVSRLRNHTDPENRAGIRKLVDAGVKVRIATQDDLKYMWKNFVNVPPSELNSYPVYDPFYITLYEFELECIKLGLPPALVITQSPDPNNVTFSINLEPIHYEKLPKEMLEALGVE

SEQ ID NO: 48 - protein, 228 aa, synthetic construct
GGLLPPPPEDPNLPKKIPPEEFERYFDPRRLLRETLLCYEIDYGGDEPPELDTLFSGEEHVILQFIKRFEADPRFSKDTKATITLYLSHSPCGECCAALIAFKKKYPNVKINIYVARLRDHADPANRAGIRALVAAGVTIRIMDDEILKYVWKKFVNRKPEDLDKYPVYDPKYLTYYRVELECILLGLPPCLEVVRTPDPDDLVLRLRLQPEHYEKLPKELLDSLGIK

SEQ ID NO: 49 - protein, 228 aa, synthetic construct
GGLLLPPPLDPNLSRKIKPEEFEEYFDPRRESKKRLLCVEIDRGGDEPPERFTMFEGDEHVEIQFIKEFTADPRFSPDDKVRIRLYLSHSPCGECCAAIIAFKAAYPNVEIEIVVARLYNPEDPANRAGIRALVAAGVTIRIADQEDYKYVWKNFVLKKPEDKDKYPVFDPRYLRLYEVELECILLGLPPCVRRILTPDPDNLVFELLLTPEHYARLPRELLEALGVA

SEQ ID NO: 50 - protein, 228 aa, synthetic construct
AGPLPPPPPDPDEPLKIPYEEFEYYFNPTILTKKTLLLVEIERGGPIPPIRFSLFSGDQHVIVQFIERFEADPSVSPDDKATITLYLSHSPCGECCDALVAFLARHPNVTINIVVARLRNHRDPANRAGLRRLRDAGVTIRVMDQEDYEEIYKNFVNRKPEDRDKYPIYDKKYITYYEVELECIFLGLPPCVEEIITPDPNNLIFKIKLTPEHYEKLPKELLDSLGIK

SEQ ID NO: 51 - protein, 228 aa, synthetic construct
SGGLLPPPVPPNNKNLIPPELFEEYFDPTNLSKRTLLLYEIEFEGTNKIERGVLFSGDEHVILQFIKEFESKYKIDPNQKIKITLYLSHSPCGACCDALVAFLKKHPNIEIEIYVARLRNHQLPENRAGIRRLVDAGVKVKIMDQEIYKYCWDNFVDKSPENKDNYPVYDPRYLAYYRLELECILLGLPPCLEVTVDPEPGKVTFTLNLTPEHYEKIPPELLAATGIK

SEQ ID NO: 52 - protein, 228 aa, synthetic construct
GGGLLPPIPDPNTKNLIKPEDFEKYFNPKNLSKKTLLLYEIKFEGTDNIEKKVLFSGKKHVILIFIEEFEKNHKIDKNQKINITLYLSHSPCGECCDAIVKFLKKYPNIKLNIYVAKLYNHSDPKNRAGIRKLIEAGVNIKIMDQNDYKYIWENFVDKPESEKNDYPVYDPKYLNYYKLELECIKLGLPPCLTVTVTPEPGKVTFTLNLQPEHYEKLPQELIDSTGLK

SEQ ID NO: 53 - protein, 228 aa, synthetic construct
GGLLLPPPRNPNDKNLIKPEEFKEAFDPKNLSKRTLLLYEIKVEGTDKVERGVLYSGDKHVYLQFIEEFESKHKLDPNQKIKITLYLSWSPCGECCDAIVAFLKKHPNISLEIVVAKLRNHEDPKNRAGIRRLIAAGVTIRIMDQEDYKYIWDNFVDKSPENKNDYPVYDEKYLNYYKLELECIKLGLPPCLKVTVDPTPGKVTFELILEEEHYKKIPKELLESTGIK

SEQ ID NO: 54 - protein, 238 aa, synthetic construct
MGEHWQYAGSGEYIPQDQFEENFDPSVLLSETHLLSELTWGGRPYKHWYENTEHCHAEIHFLENFSSKNGSCIITWYLSWSPCAECSARIADFMKENTNVKLNIHVARLYLHDDKHTRQGLRYLMKMKRVTIQIMTIPDYKYCWNTFLEDDGEDESDDYGGYAGVHEDEDESDDDDYLPTHFAPWIMLYSLELSCILQDFAPCLKIIQGNHMSPTFQLHVQDQEQKRLLEPANPWGAD

SEQ ID NO: 55 - protein, 182 aa, synthetic construct
KAAILLSNLFFRWQMEPEAFQRNFDPREFPECTLLLYEIHWDNNTSRNWCTNKPGLHAEENFLQIFNEKIDIKQDTPCSITWFLSWSPCYPCSQAIIKFLEAHPNVSLEIKAARLYMHQIDCNKEGLRNLGRNRVSIMNLPDYRHCWTTFVVPRGANEDYWPQDFLPAITNYSRELDSILQD

SEQ ID NO: 56 - protein, 216 aa, synthetic construct
RRIEPWEFEDFFDPRQFRPETCLLYEVRWGSSRNAWRSTARNTTRHAEVNFLERFAAERHFDKPVSCSITWFLSWSPCWECSQAIGAFLSQHPQVTLAIHVTRLFHHEDEQNRQGLRDLLARGVTLQVMGDSEYAHCWRTFVNSPPGAEGHYPRYPSDFTRLYALELHCIILGLPPCLEILRRYQNQFTLFRLVPQNCHYQMIPHLNFFVVRHYFF

SEQ ID NO: 57 - protein, 262 aa, synthetic construct
MHHSARLPPNCIVSRYANAPWTVLPLPLPPTEAPATGDDTLRRRIEPWEFEAFFNPQELRREACLLYQITWSSHKVWRETAKNTVDSHVEVNFIQNLTAGRYCRPSTRCSILWFLSWSPCSSCSKAIRLFLSQHPGVSLVIYVARLFQHMDPQNRQGLRELIHSGVTIQVMRPQEYDYCWKNFVNYPPGQEEHWPRYPVQCMTLYNLELYCIIHNLPPCVRISKQRQSQLAFFSLGLENVHYQRIPPPLLLLTGLVFVFPWK

SEQ ID NO: 58 - protein, 235 aa, synthetic construct
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 59 - protein, 236 aa, synthetic construct
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 60 - protein, 267 aa, synthetic construct
MGTPKDTKSRHLAGRCHELHNSRGRSPESDTMASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSTAIREFLSQHPGVTLVIFVARLFQHMDRRNRQGLKDLVTSGVTVQVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 61 - protein, 197 aa, synthetic construct
KPEKMKEEDFYYYFYNAEDRTGRTTNYLCYVLKKKLSATTTETESGWLKDENGVHVYDKFLEYFEKLNLDPDLEYDITLYASFSPCYECAKRIAEWLAKHPNVKLTIKASNLYNCGPDSPNPEGLRLLKEAGVTISIMDYEDYKYVWETFVDNKGKEYVPFEGFEENSKKLQAKLESILTPPEEAAALAELLAALGL

SEQ ID NO: 62 - protein, 197 aa, synthetic construct
KPEKMDEATFRYYFYNAADNTGRTKRYLCYELRKQLSATETEVESGWLEDEDGVCVYDKFLEYLEELDLDPDLEYDVTLYASWSPCYECAARIAAFLAAHPNVRLTIKAARLYNCDPDSPNPAGLRLLAAAGVTISIMTEADYEYVWNTFVDNNGKAYVAPAGLAENSARLQAQLDAILTPPEAAAAAAAALAAAGL

SEQ ID NO: 63 - protein, 197 aa, synthetic construct
KPEKMPEALFYYYFNNAEDVTGRTTRYLCYVLETQLSATTTEVQKGWLRDEDGVHVYDKFLAYLEKLNLDPDKTYNVTLYASFSPCYECAKRIAAWLKAHPNVSLTIKTARLYDCDENSPNPEGLRLLKEAGVKISIMTEADYKYCWDTFVDNNGKPYVPYPGLEENSKKLQEKLEKILTPPEEEAAAAALAAALGL

SEQ ID NO: 64 - protein, 197 aa, synthetic construct
KSEKMPEEEFYYYFYNAKNNEGRTVRYLCYELYEEKGPEKVLLDKGWLKSENGEHVINLLLKKLEKLKLDPKKKYHLKLYMSFSPCYECCKKLAEWKKKHPNVSITIKMANIYVNPEDPRDPEGLRLLKEAGVEISIMTLADYEYVWNTFVDNNGKPYVPFPGLEENSKALQAELDAILTPPEAAAAAAAAAAAAGL

SEQ ID NO: 65 - protein, 197 aa, synthetic construct
KSPKMPEEEFYYYFYNAADTTGRTVRYLCYELYVKEGEKEVLLDKGWLKSENGVHVELLLLKYLSKLDLDPEKEYHLVLYMSFSPCYECCKALAEWLKKHPNLKVTIKMARIYVDPEDPRQPEGLRLLAEAGVTISIMTEADYEYVWNTFVENNGKPYVPYPGQAENSARLAAELAAILTPPAAAAAAAAAAAAAGL

SEQ ID NO: 66 - protein, 197 aa, synthetic construct
KSKKIDEKTFYYYFKPERNTTGRTKRLLCYELYKEEGPEKVLLDSGWLESENGKHVIDLLLEKLSKLNLDPNKKYHLILYLSFSPCYECAKRLAEWKKEHPNISITIKMARIYRNPEDPRDPEGLRLLKEAGVKISIMTKEDFEYVWNTFVDNNGKEYKPFEGFEENSKELQKELDAILTPPELAAAAAALAALLGL

SEQ ID NO: 67 - protein, 197 aa, synthetic construct
KSPKMKKDDFEFYFKNVKDATGRTKEYLCYEVVKQLSETEEEVEKGYLVSENGVHVYDLFLEKLEKLNLDPNKKYNVTLYASFSPCYECAKRLAAFLAKHPNVSLKIKTARLYNVEPGSPNAEGLRLLKAAGVEISIMTEADFEYVWNTYVDNNGEAYVPWEGQEENSKELQAKLEEILTPPEERAAAAALAAALGL

SEQ ID NO: 68 - protein, 197 aa, synthetic construct
KSPKLPEDEFKFYFRNEEDPTGRTKRYLLYELTTQLSETTEEVEKGYLVSEDGVHVINKFLEYLSKKNLDPNKKYNVTLYLSHSPCYECAKALAEFLKKNPNVSLKIVCSRLYNVEPGTPNAEGLRLLKEAGVEISIATKADYEYAWNTFVDNNGKPYVPWEGEEENSKKLQKKLEEILTPEEEKKKEEEERKKKGL

SEQ ID NO: 69 - protein, 197 aa, synthetic construct
KSEKLDEKTFYFEFKPAENREGRNTRYLCYEVKTKLSETEEEVEKGYLKSENGVHVYDLFLEKLEKKNLDPDKEYDVTLYSSFSPCYECCKRLAEFLAKHPNVKLTIVTSNLYNVEPGSPNPEGLRLLAAAGVTIRIATRADAKHVWETFVDNNGKPFVPWEGFEENSARLAARLAAILTPPEAAAAAAAAAAAAGR

SEQ ID NO: 70 - protein, 197 aa, synthetic construct
PSPKMPEAVFYREFAPVADPTGRTTTYLCYVLTLKDPEKDVVLDKGWLRDKDGVHVELLLLEKLSKLPLDPNKKYHLELYLSHSPCYECAKAIAEWLKKNPNISLTIHMARIYVDPNDPRWAEGLRLLAEAGVKISIMTRADFEYVWNTFVDNNGRPYVPFPGLEENSARLAAELAAILTPPEAAAAAAAAAAAAGL

SEQ ID NO: 71 - protein, 197 aa, synthetic construct
KSPKLPEATFYYYFNPVENPTGRTERYLLYELTLLNPDKNVVLDKGWLKDENGKHVYDILLEKLSKLNLDPSKKYKLTLYTSFSPCYECAKRLAEWLKKHPNVSVEIKMSRIYIDPNDPRWKEGLRLLKEAGVKISIMTEEDYKYAWETFVDNKGKKYVPFKGFEENSKKLQKELDEILKPEEEKKKEEEEKKKLGL

SEQ ID NO: 72 - protein, 197 aa, synthetic construct
ESPLMPKETFYYYFDPVANPEGRTETYLCYELTELNPEKDVVLESGWLKDENGKHVYLLLLEKLSKLPLDPDKRYHLRLYASHSPCYECAEAIAAWLAAHPNVSLEIAMARIYVDPNDPRWAEGLRLLAEAGVEIRVMTAADAEYVWNTFVDNNGEPYVAPPGFEENAARLQAELDEILTPPEEKAAAAAAAAAAGL

SEQ ID NO: 73 - protein, 197 aa, synthetic construct
KSPLIDEKTFYYYFKPEENAEGRTTTYLCYELKTKVDENTYEIESGWLEDENGKCVYLLFLEKFEKLNLDPDKEYNITLYSSFSPCYECAEAIAAWLKKHPNVKLTIKTSRLYNCEPGSPNKEGLRLLKEAGVEISIMTYEDAKYVWNTYVKNNGKEYVPFEGFEERSKKLQAKLEEILTPPEEKAAAAALAAALGL

SEQ ID NO: 74 - protein, 197 aa, synthetic construct
KSPKIDEETFYRYFNNEEDRKGLNKTYLCYEIKTKVDENTYEVEKGWLEDEDGKHVVDLFLEYLSKLNLDPDKEYDVTLYMSFSPCYECAKKLAEFLKKNPNVKITIKVANLYNCEPGSPNKEGIRLLKEAGVKVSIMTEEDYKYLWNTFVDNNGKEYVPFEGFEERSKKLQAELEKILTPPEEKKKLEELLKELGL

SEQ ID NO: 75 - protein, 212 aa, synthetic construct
EALAALAALLAALTQPGPAAPVPPAPPPAPGACPPVPLKPDGSVDLAALNAELAARGVTGLDGVDPTPVPGFDPAAFHPACSAGLAPRQPGQPTTGKLFLPDGRRLNSTPLTGTGGKSAWDLSCIKPEYRDLAPARGDLAGNVAAYARRTDIKELVLYMNAAPSTLNGKGSLYRLHDFLLPGYTLYVWWQDAQGNWQLTVYKGTGEAYVPGV

SEQ ID NO: 76 - protein, 212 aa, synthetic construct
AAAAAAAAAAAAAAAPGPGAPVPPAPAPAPGAAAPPPRRPDGSVDRAALAARFAARGLTGLDGVDPTPVPNFDPNKYNPALSEGLEPRKPGEPVRGKLYLPDGKRLNKTPLTGTGGTSAWDLAEIRPEYRDLAPARGDLEGNVAALMRDLDIKEAVLVMNAAPSDLNGRGSLHRLKDFLRPGYTLHVWYRDENGEWKLKVFEGTGEAFVPGV

SEQ ID NO: 77 - protein, 212 aa, synthetic construct
ALLAPLAAALAALTAPGPAAPVPPPPPPVPGACTPVPLRPDGTVDLAALKAELAARGLTGLDGRDPTPVPGFDPNKFHPACVEGLEPQKPGEPTRGFLFLPDGRRLNSTPLTGTGGKSCWDLSCIKPEFRDLAPAQGDLEGCVAAWARRTGLKELVLYMNRAPSKLNGKGSLYTLKDFLLPGYTLHVWYQDEEGNWKLKVFKGTGAAYVPGV

SEQ ID NO: 78 - protein, 212 aa, synthetic construct
EKERLAEILAKLLNPSPPPAPAPPKPPPPPPPPRPLPLKPDGTVDLEALRALLKALGVKGLEGLDLTPIPNFDPNKYNPAAKEGLPPRKPGEPTLGNLFTPDGVKVNTKPLAASDGKSAWDLSLIKPEYRDLAPARGDLEGNVAALMYKFNIKEAVLYMNAAPSKLNGKGSLYTLNNFLPKGYTLYVWYQDENGNWKLKTYTGTGEAFIPGA

SEQ ID NO: 79 - protein, 212 aa, synthetic construct
ERERLARRLRRLRRPRPPPAPRPPRPRRRPLPPRPLPRRPDGKVDLRALRALLRALGVEGLEGLDWTPVPGFDPNKYNPACSAGLPAQKPGEPVQGRLFRPDGVRVNAEPLTSSDGVSAFDLSLIKPEFRDLAPARGDLAGNVAAYAWENNIKELVLYMNRAPSKLNGKGSLYTLKNFLPKGYTLYVWYQDANGNWKLKKYEGTGEAFIPGA

SEQ ID NO: 80 - protein, 212 aa, synthetic construct
KKEELAKKLEKLLNPKKPPKPKPKKPKRKPIKPRPPPLDEKGKIDFEALRKLLIELGVKGLEGLDKTPIPNFDPTKYNPKASEGLAPQKPGEPTQGRLFSPNGVRLNKKPLTSSDGVSAWDLSKIKPEYRDKDPAKGDLVGNVAAYAYRNNIKELVLYMNRAPSTNNGKGSLYTAKDFLKKGYTLYVWYRDANGNWKLKVYKGTGKAFIPGA

SEQ ID NO: 81 - protein, 172 aa, synthetic construct
APAEAAAAAAAAAAALAAALGLGGPPAGAAPPALPEEEVDRALAQLPAPVVPGTGQKVHGLWKGPDGEWKPITEGENEYSKLVNPILAAKGAPGPTPLADLVEMQLAAYMAANGIRHATVVMNALPETGPGKADELVPIILPPGSTLTVHGIDADGKRSRVTFHGGAKPPWQ

SEQ ID NO: 82 - protein, 172 aa, synthetic construct
APAAAAAAAAAAAAALAAALGVGGPPAGPEPPPLPEEEIAAALATLPPPVVPGTGQRVHGLWRGPDGRWRPITEGEDERSALVNPLLAARGAPGPTPLATDVALKLAAYMAAHGIRAATVVVNAEMLRGPGRADELVPVILPPGAVLTVHGVDAAGRRSRRVFRGGATPPWA

SEQ ID NO: 83 - protein, 172 aa, synthetic construct
VPAAALAAAAAAAAALAAALGLGGPAAGAAPPALPEAEIDALLAQLPAPVVPGTGQRVHGLYRDASGEWRPITEGEDELSKLVNPLLAALGAPGPTPLATLVEMKLAAYMAANGIKHATVVINALMERGPGKADELVPIILPEGATLTVHGIDADGRRSREVFRGGAKPPWA

SEQ ID NO: 84 - protein, 172 aa, synthetic construct
AAAAAAAAAAAALAALAAALGVGAPPAGAEPPVVPQAEIDAMLAQLPAPVVPGTGQKVHGLVKGADGQWRPIVSGENELSKLVNPLLASLGAPGPTPNSTAVELQTAAYMAANGIKHATVVVNALPAKGPGKPDELVPIILPEGSTLTVYGIDANGNRSKVTFKGGKKPAWQ

SEQ ID NO: 85 - protein, 172 aa, synthetic construct
AAAAAAAAAAAAAAAAAAALGVGAPPAGAEPPELPEAEVEALLAQLPPPVRPGTGQRVHGLWRGADGEWRPITSGEDELSRLVNPLLAARGAPGPTPRATDVELKLAAYMAAEGIRHATVVLNALPARGPGKPDELVPILLPEGATLTVHGVDAAGRRSRVTYRGGATPPWA

SEQ ID NO: 86 - protein, 172 aa, synthetic construct
AAAAAAAAAAAAAAAAAAALGVGAPPAGLEPPRVPAAEVERALAQLPPPVVPGTGQLVHGLWRGPDGRWRPITSGENELSKLVNPILAAKGAPGDTPLSTLVELQLAAYMAAEGIRHATVVVNALPPTGPGKPDELVPIILPEGASLTVYGVDAAGNRSEVTFRGGATPAWA

SEQ ID NO: 87 - protein, 212 aa, synthetic construct
SLLAPLLAALAPLAAAPPAPAPPVPPAPPPPPPSVLPLNPDGTLDLEALKALFKAKGLTGLDGFSETPVPNFDPDKFNPACFEGLAPQKPGEPVRGRLFLPDGKRLNSEPLTSTNGTSIFDLSLIKPEYRDLAPARGDLAGAVAAYAIYLDIKELVLAMNRAPSKLNGKGSYYKLKDFLKEGYTLYVWYRDANGNWKLKKFEGTGKAFIPGA

SEQ ID NO: 88 - protein, 212 aa, synthetic construct
AALAALAAAAAALAAAPPAPAPPRPPKPPAPPPSVLPLRPDGTLDLEALRAEYRAKGLTGLDGVSTTPVPGFDPTKYNPACFEGLEPQKPGEPVKGRLFRPDGTRVNSTPLTDTGGTSLWDLSRIRPEYRDLAPARGDLAGNVAALMRDLGIREAVLAMNAAPSDLNGRGSLHTLHDFLLPGYTLYVWYQDENGNWKLKTYRGTGAAFRPGE

SEQ ID NO: 89 - protein, 212 aa, synthetic construct
AAAAAAAEAAAKAAAAPPAPAPPVPPAPPPPPPSVLPLKPDGTLDLAALKALFAAKGLTGLDGFSETPVPDFDPDKYNPACFEGLEPRKPGEPTRGNLFLPDGKRVNSEPLTSTGGTSLWDLSKIKPEYRDLAPARGDLLGCVAALMTYLDWKEAVLCMNRAPSKLNGKGSLYTANKFLKKGYTLYVWYRDANGNWKLKKFEGTGEAYIPGA

SEQ ID NO: 90 - protein, 226 aa, synthetic construct
LDAALAAARAALAAAKAQLAAHAAAAAGSTDPAPAAELAALRAQLAALEAQLAALEAQRKALAAAAEALAAAPAGPLKPPPAPPPPPPPPPFDPTKADPAKAALIPPKVGDAPRVAIVFDENGNEVMGLTADGATGPAATAPWKAPYASYTALRADVLAHAAALAYQKGLKTVTVYTNAAPDTSATGAAANLADVLPKGATLYIHLVHADGSTTLTTVKGTGAAWA

SEQ ID NO: 91 - protein, 226 aa, synthetic construct
LEAALAAAEAELARLEAELAAHRAAAKGSTDPAPAAELAALEARIAEARRRLAALAEEKKKAEEEKLKKELKPEKKEKKPPKPEPPPPPPAPDPAVTDPAWAALIPPAVGDDPETAIVRDENGNEVLGLTTAGATGPAATAPWLAPYSTFARLRASVFAHAAALMYQKGLKTATVYTNKPPDTAPDGAAKNLADVLPKGATLYVHLVHADGSTTLYVYKGTGKAWA

SEQ ID NO: 92 - protein, 226 aa, synthetic construct
LEERLEAALAALAAAEAELAALRAASAGSADPAPAARLAALEAEIARLREEIARLEAELEERRREEERLRSEPEEPEEPPPLPAPPPPPPPFDPEKPNPELAAKIPPAVGDDARTAVVYDENGNEVLGLTTDGATGPAATAPWKAPYASYTELRADVLAHAAALAYQRGLKTITVYINKAPSTSATGAAKNLAAVLPKGATMYIHLVHADGTTTLYVVKGTGSAWA

SEQ ID NO: 93 - protein, 168 aa, synthetic construct
PAAAPAPPAPAPGGAPLPPPPPSPAPRPLPDAEVEALAAQLPAPVVPGSGQLVHGLYVAEDGEVHSITSGESALSERVIALLAAKGWPRRPRNAGDVVLQLAAYMVAHGIKHVTLVINAAPSTGTPDSADTLVPVLLPEGATLEVHGRTADGAPVRRVFRGGAAPPWA

SEQ ID NO: 94 - protein, 168 aa, synthetic construct
PAAAPAAPAPAPGGAPLPLPPPSPAPKPLPEEEIERLAAQLPPPVVPGTGQLVHGLYVGEDGEVKEITSGESELAELVREILAAKGWPHAPSNAGGVELQLAAYMVANGIKHVTLVINAAPSTGTPDSADTLVPIILPKGATLTVYGRTADGKPVRVTYEGGATPPWA

SEQ ID NO: 95 - protein, 168 aa, synthetic construct
SAPAPAPPAPAPGGAPAPLPPPTPAPKVLPEEEIAALAATLPAPVVPGSGQRVHGRWVGEDGEVRSLTSGEDALAERVRALLAAQGIPEAPENAGGVELKLAAEMVARGIKHATLVINAAPSTGTPYSADTLVPVILPPGATLTVYGRTAAGEPVVVTYEGGATPAWA

SEQ ID NO: 96 - protein, 168 aa, synthetic construct
PPAPPVPEEVPPGPGPLPVPEAGPEPRPLPEAEIEALMAQLPPPVVPGTGQRTHGLYVGEDGEVRSITSGWDALADAVRALLAALGWPRAPRNAHGVELKLAAYMVAHGIRHATLVLNAAPSRGRPDSADALVPVLLPEGAVLEVYGVTAEGRRVRVVYRGGARPAWA

SEQ ID NO: 97 - protein, 168 aa, synthetic construct
PPPPPRPEVLPEGAAPLPVPEGAPLPRPLPAAEVERLASQLPPPVVPGSGQKVHGLYVDASGAVRSITSGDDADAAAVRALLAALGWPEEPTRADGVELKLAARMVAEGVRHVTLVMNAAPSTGGPDSADALVPVLLPEGAELLVYGRTADGRPVRVLFRGGARPAWA

SEQ ID NO: 98 - protein, 168 aa, synthetic construct
PPEPVKPRELPAGAGAVPVPAAGPAPRPLPEERVEELFARLPPPVVPGSGQRVHGLYVDESGEVREIVSGDDALAALVRERLAALGWPEEPRRADDVELKLAARMVAEGIRHVTLVVNAAPSRGEPDSADTLVPILLPEGAVLRVYGRTAAGERVVVEYRGGAKPPWS

SEQ ID NO: 99 - protein, 167 aa, synthetic construct
AAIVTAPVVPLVWVGDTPVVVPDGTPPAYILEALAELAGEPRETGLLFDKNGNRIGPRITAGDEDPELRAGVVELLRADPWFRAHHPPDTVLPVARRVEAKAAVWAVRNDIKEAILVINSDSVGPPAPDPGLGPETAVPHILPEGYTLTVWTSDKTSVTLVGKRPRP

SEQ ID NO: 100 - protein, 167 aa, synthetic construct
AAVVTAPVPVVVWVGDVPVLVPTAKPPAYVLAALAELAGKKRETGLIFDRNGNRIGPEITAGEEDPELRAGVVELLRADPWFRAHHPPDTVLRAALDVACKAAVWAVRHNIREAILVTNRDTVGPPAPDPGLDARTAVPHILPEGWTLTVWRKDKTSETLVGRRPRP

SEQ ID NO: 101 - protein, 167 aa, synthetic construct
PLVVLAPVPPLVWVGDVPVVVPTGEPPAFVREALAELEGEERETGLLFDKNGNRIGPRIVAGREDPELRADVVALLRADPWFRAHHPPDTVLPVAEDVEAKAAAWMVKNNIKEAILVINRDTVGPPAPAPGLGPEVAVPHILPEGYTLTVYRRDGTSVTLEGKRPKP

SEQ ID NO: 102 - protein, 167 aa, synthetic construct
APLPLGVPPVLVPAPPLPLPPPDAPTPAWVKEALAELAGSERETGLIFDKNGNRIGPRITAGDEDPELRAGVQELLAADPYFRAHFPPDRRFRVARLVEAKAAVWAVKNNIKEAVLVTNAPVVGPASGDPHDTPLVAVPLILPEGWTLTVYRSDETSVVLKGTRPLP

SEQ ID NO: 103 - protein, 167 aa, synthetic construct
APAPAGTPVVAVPAPPLPLPAPAEPTPEWVKKALEELKGSKKETGLIFDENGNRIGPRITAGDEDPELRALVQELLRADPYFREHFPPDRQFRVARGVTAKAAAWCVKNNIKNAVLVINSDVVGPETGDPYDTDRVAVPLILPEGWTLTVYRSDETSVVLKGKRPLP

SEQ ID NO: 104 - protein, 167 aa, synthetic construct
LPSVAGVPVPVLLTPPLPLPPPSAPLPEWVREALAELEGSPRETGRLFDENGNRIGPLITAGEEDPELRAEVERLLRADPFFRAHFPPDARFRTALGVEAKAAAWAVRHNIKNAVLVINADVVGPDAGDPHDTPRIAVPLILPEGWTLTVYRRDETSETLVGERPRP

SEQ ID NO: 105 - protein, 153 aa, synthetic construct
PPVPLPPLPPPAAAGAPAPGLPGFVAAALAELAGETRETGLIFDADGRRIGPRITAGPGPLAARAQALLRASPHVPALPPDAVYAAARGVEAQAAVWMRENGIKEAHLVTNAPSVSGPPLGSETAVPAILPKGAKLTVWTPGAGEPLVLEGVG

SEQ ID NO: 106 - protein, 153 aa, synthetic construct
PLPLPLPLPLPAAAGAPSPGLPGWVAAALAELAGSRRRTGRLFDENGEQIGPLLTAGPGPLAAAAEELLRASPHVPALPPDARYAAALGVEAQAAVWMRDNGIRRAHLVLNAPFVSGPPLGAATAVPAILPRGAVLTVWYPGAGEPLVLRGVG

SEQ ID NO: 107 - protein, 153 aa, synthetic construct
PVPVVAPAPLPAAAGPPAPGLPGWVARALAELAGEERETGLIFDENGERIGPRLTAGPGPLAERAQELLRASPHVPALPPGERYAAALGVEAQAAVWMRENGIKRAHLVINRDRVSGPPLGEETAVPAILPRGATLTVWYPGAGEPVVLEGVG

SEQ ID NO: 108 - protein, 153 aa, synthetic construct
PPLPLPPLPPPPPLPPPDPTIPPFVYEALAELAGSEKETGLMFDENGNRIGPRITAGPGPLAARAQELLRASPHVPALPPDAVYAAARGVEAQFAVWARENGIKKAHLVTNADSISGPPTGEETAVPAILPKGATLTVWHPGAGEPTVLEGVG

SEQ ID NO: 109 - protein, 153 aa, synthetic construct
GLLVVPPLPPPLPPPPPSPGIPPWVLEALEELADSEKETGLLFDENGNRIGPRITAGPGPLAERAEALLRASPHIPALPPDAVYEAAYHVEAQYAVWCRENGIRRAHLVINADSVAGPPLGDRAAVPAILPRGAVLTVWTPGAGAPVVLRGVG

SEQ ID NO: 110 - protein, 153 aa, synthetic construct
PPVPLPPLPPPPPAAAPDPALPPFVREALAELAGARRRTGLLFDENGRRIGPRVTAGPGPLAERAEELLRASPHVPALPPDARYAAARGVEAQFAVLCRERGIRRAHLVTNAPFVSGPPTGEATAVPAILPRGAVLRVWYPGAGRPLVLEGVG

SEQ ID NO: 111 - protein, 212 aa, synthetic construct
AAEELAALLAALADPRPPPAPAPPAPPRPPLPPRPLPLTPDGKVDLAALRALLLALGVAGLEGLDLTPVPNFDPNKFNPACSEGLAPRKPGEPVKGRLFLPNGVRVNTTPLTSSDGKSAFDLSKIKPEYRDLDPARGDLEGNVAAYATRNNIKELVLYMNAAPSTNNGKGSYYTLNKFLPKGYTLYVWYQDENGKWKLKKYEGTGEAYIPGA

SEQ ID NO: 112 - protein, 212 aa, synthetic construct
AAERLAAALAALLTPAPPPAPAPPAPPPPPVPPLPPPLRPDGKVDLAALRALLRALGVEGLEGLDTTPIPNFDPNKYNAAAAAGLPAQKPGEPTQGNLFLPNGVRVNTTPLTSSNGVSAFDLSEIKPEFRDKDPAQGDLAGNVAAYMIRNNIKEAVLYMNRAPSKNNGKGSYYTLKDFLPKGYTLHVWYRDANGNWQLKTYKGTGAAFIPGA

SEQ ID NO: 113 - protein, 212 aa, synthetic construct
AAADLAALLAALLTPAPPPAPAPPPPPPPPLPPRPLPRTPDGRVDLEALRALLRALGVEGLEGLDTTPVPNFDPTKYNPACREGLAPRKPGEPTQGRLFLPNGVRVNTEPLTASDGVSAFDLADIKPEYRGLDPARGDLIGNVAAYMRRFGIKEAVLYMNRAPSTNNGKGSLYTMKDFLPKGYTLYVWYQDANGNWKLKKYVGTGAAFVPGA

SEQ ID NO: 114 - protein, 212 aa, synthetic construct
AALAAAAAALAALAPAAPPVPVPAPPAPPPPAADVLPRKENGEVDWEALNALLKARGMEGLDGLDPTPVPGFDPARPNPACFEGLAPRVPGEPTRGRLFRPDGRRVNGTPLTASSGKPLWDLSLIKPEFRDLAPARGDLVGNVAAYMRYTDIKEAVLYLNRPPSDLNGKGSLHTAPKFLPKGYTLYVWYRDAEGNVRLRRFEGTGEAFVPGA

SEQ ID NO: 115 - protein, 212 aa, synthetic construct
AALAAAAAAAAAAAAAAPPVPVPPPPPPPPPEEDVLPLKPDGTVDWEAFNKLMESEGFKGLKGLDPTPVPNFDPNKPNPACFEGLEPRKPGEPVKGFLFRPDGTRLNDEPLESSSGKSLFDLSKIKPEYRKLKPAQGDLEGNVAAYMIKRNIKEAVLYMNAPPSDLNGKGSLHTLKDFLPKGYTLYVWYIDENGNVKLKVYKGTGKAFIPGA

SEQ ID NO: 116 - protein, 212 aa, synthetic construct
AAAALAAALLAAAAPAAPPVPVPPPPPPPPPKGSVLPRKPDGTIDWEAFNALLKSLGFEGLDGVDPTPVPNFDPEKYNPACAVGLEPQKPGEPTKGRLFRPDGRRLNSTPLTESSGKPLFDLSKIKPEYRDLAPARGDLEGNVAAYMILRNIKEAVLYMNRAPSTLNGKGSLYTLKDFLPKGYTLYVWYQDENGNWKLKKFEGTGEAFIPGA

SEQ ID NO: 117 - protein, 226 aa, synthetic construct
LEEKLKEAEEKLKEAKEELEKLKKEYEGSKDESPKEKIEELEEKIKELELELKRLEEEKKKLLEKLKKLLEKPGAAEEPTPAVPPPPPPPPYDPEKPDPALAAKIKPAVGDDATEAILYDAQGNLVLGLTKDSTTGPAATAPWKAPYASYTKLRASVLAHAAAAAYKLGLKTVTVYTNKAPDTSADGAANNLADVLPEGATLYVHVVKADGSTELLVYRGTGAAYK

SEQ ID NO: 118 - protein, 226 aa, synthetic construct
LEEELERAREELERAERELEELRARHAGSTSPEPAAELAALEARIAELRARLRRLEEEREELERKLREELEKEEEEEEPEPPVPPPPPPPPFDPAVVDPALAAKIKPKVGNDKTEAILVDENGNEVLGLTTDSSTGPAATAPWKAPYANYTELRNSVYAHAAAEMYRRGLKTVTMYTNQPLDTSATGAANNLANVLPKGATLYIHVVHADGSATLTTVTGTGAAYA

SEQ ID NO: 119 - protein, 226 aa, synthetic construct
LEEKLAAAEAERAAAEAELAALRAAAAGSTDPAPAAALAALEARLAALDAELKRLEAEKKKKEEEEKKEKEKPEPKAKPKPPKPPPPPPPPYDPEVPDPALAAQIPPKVGDAPTTAILYDANGNQVLGLTTDGSTGPAATAPYKAPYASYTNLRNSVLAHAAATARKLGLKTVTVYTNQAPDTSPTGAANNLADVLPEGATLYIHVVHADGSTTLTKVEGTGAAYK

SEQ ID NO: 120 - protein, 172 aa, synthetic construct
AGPAAAAAAAAAAAAAAAAAGAGAGAAGAEPPEVPEAEIAAALAQLPPPVRPGTGERVHGLWRGPDGEWRPITAGENELSKLVNPQLAALGAPGPTPNARGVEMQLAAYMAAHGIRHATVVINAKPGRGPNRADVLVPVLLPAGSTLTVYGVDADGRPVRVVFRGGATPPWQ

SEQ ID NO: 121 - protein, 172 aa, synthetic construct
AGPALAAAAAAALAALAAALGLGPGPGGGEPPVVPEEEIEAMFAKLPPPVVPGTGEKTHGLYRDESGEWKPITEGEDEWSKLVNPILASLGAPGPTPLATDVEMKLAAYMREHGIRHATVVINAEPLQGPDKAEVLVPIILDPGATLTVYGVDADGRRVRVTYRGGATPPWQ

SEQ ID NO: 122 - protein, 172 aa, synthetic construct
GPLEALRELAEALAAALAASGVGAGAGGAEPPEVPEEEIEAALAQLPPPVVPGTGERVHGLYRGEDGEWRPITEGENELSKLVNPLLAALGAPGPTPNATLVEMQLAAYMAANGIRHATVVINAAPPRGPDRAEVLVPIILPEGATLTVYGVDADGRRTRVTYRGGAKPPWQ

SEQ ID NO: 123 - protein, 172 aa, synthetic construct
APPALAAARAAAAAAARAAAGAGGAPALDAPPAVPEAEVAAMAAQLPPRVRPGTGERVHGLWRGADGAWRPITEGPSEWSALVNPLLAALGAPGPTPNATDVEMQLAAHMAATGERHATLVLNATVSTGPGEADELVPILLPPGSTLTVHGYDAAGAPTRVTFRGGATPPWA

SEQ ID NO: 124 - protein, 172 aa, synthetic construct
GTPALAAAAAAAAAAARAAAGAGAAPAAGEPPEVPEAEVEEMRAKLPPPVTPGTGEKVHGLYKGEDGEWKPIVEGENEWSKLVNPLLAALGAPGDTPWATDVEMQLAAYMAAHGIRHATVVINATVATGPGRPDELVPILLPEGATLTVYGVDAAGNPTRTVFRGGAEPPWR

SEQ ID NO: 125 - protein, 172 aa, synthetic construct
APPAAAAAAAAAAAAAAAAAGAGGAPEAGAPPELPEEEVEALRAQLPPPVTPGTGEKVHGLYRGADGEWKPITEGENEWSKLVNPILAAKGAPGPTPNATDVAMQLAARMAAEGIRHATVVINATLATGPGKPDELVPIILAPGSTLTVYGVDANGNPTRTVFRGGATPPWA

SEQ ID NO: 126 - protein, 172 aa, synthetic construct
GGAAAAAAARAAARAALAAAAAAGAPGLSAAPPLPEAEVAAALATLPPPVVPGTGQRVHGLVRDEDGRWRPIVSGENELSALVNPILASLGAPGPTPLATDVELQTAAYMAAEGIRHATVVVNALMRRGPGQPDELVPIILPEGATLTVHGVDAAGRRSRRVFRGGKTPAWA

SEQ ID NO: 127 - protein, 172 aa, synthetic construct
AGPALAAAAEAARRAALAAAAAGGSPGSSLAPEVPEEEVEAALAQLPPPVVPGTGQRVHGLVRLEDGEWRPITSGEDELSALVNPLLAALGAPGPTPLATLVELKLAAYMAARGIKHATVVVNALPLRGPGRPDELVPILLPEGATLTVYGVDAAGRRSRRVFRGGAVPPWQ

SEQ ID NO: 128 - protein, 172 aa, synthetic construct
AGAAAAAAAAAARRAALAAAKAAASPAAAEAPPLPEEEIEAALAKLPPPVVPGTGQRVHGLWRGADGEWRPITSGENALSALVNPLLAALGAPGPTPLSTDVELQLAAYMAATGERHATVVMNALMRRGPGRPDELVPVLLPPGSTLTVYGVDHEGRRSRRTFRGGATPAWA

SEQ ID NO: 129 - protein, 226 aa, synthetic construct
LEEELKKVEKELEEAKKELEELKKKSEGSTDPEWKKKLEELERKIEELELEKEELKKKIAEEEARLAALLNPGAPLPPLPPPVPPPPPPPPFDPTKANPALAAKITPAVGDDPEEAIVYDKNGNLVLPLTRDSDTGLAATAPWKEPYKNYEKLRRSVFAHAAAAMLKRGLKEMTVYTNKPLDTSEDGAAKNLANVLPKGATLYIHLVHADGSTTLTKVEGTGSAYA

SEQ ID NO: 130 - protein, 226 aa, synthetic construct
LEKKLKEAEKKLEEAKKKLKELKEKSKGSTDKEPEKELEELEREIEKLELEIKELKEEKAALEAKLAALLNKGGPAVVLPAPVPPPPPPPPFDPTKTDPALAAKITPKVGDDPTEAILYDANGNLVLPKTYDSDTGEAATAPWKAPYANYTNLRKSVLAHAAAAMRKRGLKTMTMYINAPPDTSPDGSAKNLANVLPKDGTLYIHVVHADGSTTLYVFKGTGSAWA

SEQ ID NO: 131 - protein, 226 aa, synthetic construct
LEEKKEEVEKRLKELEKELKKLKEESEGSTDPEPEEKIRELEREIELLKLELRELEEKIKEEEARLAALLNPGEPAPVLPPPVPPPPPPPPHDPAVPDPALAAKIKPKVGDDEREAILVDADGNLVLPLTTDSADGPAATAPWLPPWADYTELRRDVAAHAAAAAHERGLREVTVYTNQPLSTSPTGAAHNLAAVLPAGGTMYIHLVHADGSTTLTVVRGTGAALP

SEQ ID NO: 132 - protein, 226 aa, synthetic construct
LDAALAAARAALAAAEARLAALAAATAGSTDPAPAAELAALKAELAALRARLAALEAARAAAAAAAAAALAGAAPAPPVPPPPAPPPPPPPFDPTVPNPALAAKITPKVGDDKEEAILVDANGNEVMGLTYDGENGPAATAPWLAPYADYTELRKSVAAHAAAVMRDKGLKTATMYTNKPLSTSATGAAKNLAAVLPKGGTLYIHVVHADGSATLYTVKGTGSAYA

SEQ ID NO: 133 - protein, 226 aa, synthetic construct
LEEELKKAEKELEKAKKELEELKKKTKGSTDPEPKKKIAELEEKIKELEKKLKELEEKKKKKEEELKKLLEKEEPVPYVPPVPVPPPPPAPRDPTKTDPELAAQITPAVGNDKRAAILYDENGNEVLGLTYDGSTGLAATAPWLAPYANYTELRNDVLAQAAAAMYKKGLKTVTVYTNKEPSTSATGAAKNLANVLPKGATLYVHVVHADGTTTLLVYKGTGAAYA

SEQ ID NO: 134 - protein, 226 aa, synthetic construct
LEEKLKEAEKKLKEAEEELKKLKEKNKGSKDPKPKEEIKKLEAEIEKLKAELKELKEQKEAEKAALAALLGAAAPAPPVPAAPAPPPPPAPFDPTKPDPALAAKITPAVGDAPEEAILVDADGKEVLGLTTASATGPAATAPWLAPWADYTALRASVYAHAAAEMYRRGLKTATLYTNKAPSTAPDGAAKNLADVLPAGATLYIHVVHADGSTTLYTVKGTGAAYA

SEQ ID NO: 135 - protein, 226 aa, synthetic construct
LDAELAAARAALAAARARLAALRAAHAGSADPAPLAEIAALEAELARLEARIAALRAERRRRAAARRRRAAPAAPAPRPPPPPPPPPPPPPYDPTEPDPALAALIPPRVGDAPRVAIVVDENGNEVLGLTADGADGPAATAPYKPPCADYTELRRDVFAHAAALMRQRGLKTATVYTNLPPDTSETGAAKNLADVLPKGATLYVHVVHADGSTTLLVYKGTGKCWS

SEQ ID NO: 136 - protein, 226 aa, synthetic construct
LAEALAAAEARLAALKAQLAAAEAAHAGSADPAPLAELAALRAAIARAEREIAELRAAAAAAKAAREALEAAEEPAPPPPPPPPPPPPPPPFDPTVPDPALLARIPPRVGDDPRVAVVYDANGNEVLGLTADGATGPAATAPYKAPWANYTELRADVYAHAAALMHKRGLKTVTVYTNLPPDTSATGAANNLADVLPKDATMYIHVVHADGSATLVVVKGTGSAWS

SEQ ID NO: 137 - protein, 226 aa, synthetic construct
LEARRAEAEAALAAARAELEALRAEHAGSTDPAPAAELAALKARLARLERELAALAEEEAALARQRAAAEAPGAPEPAPPPPPPPPPPPPPFDPTKPDPELLARIPPKVGNAPRQAIVYDENGNEVLGLTEDGEDGEAATAPYKAPWANYTNLRRDVLAHAAALARKRGLKTITVYTNLPPDTSDDGAAKNLSAVLPKGATMYVHVVHADGSATLYVFKGTGSAWS

SEQ ID NO: 138 - protein, 168 aa, synthetic construct
PRPVPPIVLLEPGPAPAPPPPAEGMLPPLPEAEVERLAAQLPPPVVPGTGQRVHGLYVGADGAVREITSGEDELAELVREILAAKGWPEAPENAGDVELKLAALMVKEGIKDVTLVINAAVSTGTPDSADALVPVILPEGSFLRVYGRTAEGRRTVRTFRGGATPPWA

SEQ ID NO: 139 - protein, 168 aa, synthetic construct
PRPLPPIVLLEPGPEPEPPPPEEGRLPPLPEERIAALAAQLPPRVVPGTGEKTHGLWVGPDGEVKSITSGDDELAEAVRRLLAALGWPAAPENADDVELKLAAYMVENGIRDVTLVINSEVSTGTPDSADVLVPVILPEGAYLRVYGFTREGQPTVRTFVGGATPPWA

SEQ ID NO: 140 - protein, 168 aa, synthetic construct
PPPLPAPVLLAPGPEPGPPPPEAGRRPPLPEAEVAALAARLPPLVRPGTGERVHGLYVGEDGEVREIVSGDNALAAAVRALLAALGWPEPPRNAGDVALQLAAYMVENGIKDVELVINAAFGRGTPDSPDVLVPVLLPEGHYLTVHGFDAEGRPVVVTLVGGRTPPWA

SEQ ID NO: 141 - protein, 168 aa, synthetic construct
SLPGALGAAPLPGPAPLPPPPGSDAPLVLPEEEVEALMAKLPPIVKPGTGEKTHGLYVGEDGEVKEITSGENELSKLVKEILKSKGIPETPKNAGDVELQLAAYMVKNGIKHATLVINAAPSTGYPDSADTLVPIILPKGYTLTVHGVTAKGERVVVTYEGGKTPPWS

SEQ ID NO: 142 - protein, 168 aa, synthetic construct
AEEEEEEEEEEPEPEPEPPPPGSPGPIPLPQERIEELAAQLPPLVVPGSGQRTHGLYVDESGKVREIVGGENELAALVRELLAAKGIPETPPNADGVELQLAAYMVANGIKHVTLVINAAPSTGYPYAADALVPVILPAGSVLTVYGRDAQGNPVVVTYVGGATPAWA

SEQ ID NO: 143 - protein, 168 aa, synthetic construct
AALGAAGAAPPPGPAPEPPPPGAPAPRELPEARIEALAARLPPLVRPGTGERVHGLWVGEDGEVREITSGENELAELVLRLLASLGIPEKPPNADGVELQLAAYMVANGIRHATLVINAAPSTGYPYSADTLVPIILPEGWTLTVYGRTADGRPTRVTFRGGATPPWA

SEQ ID NO: 144 - protein, 168 aa, synthetic construct
AAALLELLAAGAAPGPAPLPPGLGLPPPLPEAEIAALAAQLPPPVRPGSGERVHGLYVAEDGQVRSLTGGEDALSALVREILAALGAPEEPPNAGLVELKLAALMVRNGIRGVTLVINAPVDRGEGYSAEELVPVILPKGATLTVHGRDAAGRPVRRTFVGGKKPAWQ

SEQ ID NO: 145 - protein, 168 aa, synthetic construct
AAAALAALLAGAEPAPGPPPPAAGLPPELPAERVAALAAQLPPPVVPGSGELVHGLYVDESGEVREITGGENELSERVVALLAALGAPERPPNAGHVELQLAAYMVENGIRGATLVINAPPGRGEGYSPEVLVPVILPEGAVLRVHGRDADGRPVVREYRGGAAPPWA

SEQ ID NO: 146 - protein, 168 aa, synthetic construct
AAAALLALLAGALPGPGPLPPDLGLPPELPEERIAALAAQLPPPVRPGSGERVHGLYVDEDGAVREIVGGENELSELVKRILAAKGYPEEPENADHVELQLAALMVENGIRGVHLVINAPVGRGEGDSPEVLVPIILPEGATLTVYGRDAAGRPVRRVFRGGAAPPWA

SEQ ID NO: 147 - protein, 167 aa, synthetic construct
LELAEGPPLLLPKEGGAPVPLPLPNVPAWVEAALAELAGSPRETGLLFDENGNRIGPRITAGDEDPELRARVVELLLADPYFRANYPPEAAEEASKLVECKAAAWMVANNIKRAHLVINSDVVGPRRGERRTSPLEAVPHILPEGWTLTVYRRDRTSVTLEGRRPRP

SEQ ID NO: 148 - protein, 167 aa, synthetic construct
PLPLLGPPLPLPLAGPVPLPEPLLDIPEYIKEALEELKGNKRETGLIFDENGNRIGPTITAGDEDAELRAGVRELLLADPYFRAHYPPEAAEEVSKRVEAKAAYWMLKNDIKKAILVINSKVVGPDRGAAAASAEEAVPLILPEGYELTVYRADKTSVTLKGKRPKP

SEQ ID NO: 149 - protein, 167 aa, synthetic construct
PPLLAGPPLPLLPAGSVPVPLDLSAVPEWVLEALAELAGSPEETGLIFDKNGNRIGPRITAGDKDPELRAGVVETLAKNPYAKENYKPEDFEEASKRVECKAAVWMLKNNIKEAILVINSDVVGPDRGARGASPLTFVPLILPEGYTLTVYTADRRSVTLKGKRPKP

SEQ ID NO: 150 - protein, 167 aa, synthetic construct
ALLLPPPPVPLLPLGDVPVPLPDADVPEFVKEALAELKGSKKETGLIFDKNGNRIGPRITAGDEDPELRAGVVALLRADPYFRAHAPADHVWRVALGVEAKAAVWMVKNDIKEAILVINSSSVGANTDDPLDQATLAVPHILPEGYTLTVYTSDEKSVTLVGERPRP

SEQ ID NO: 151 - protein, 167 aa, synthetic construct
AALLPPPPLLPVPLGDVPLPLPLPPAPEFVLEALKELEGSKKETGIIFDLNGNRIGPTITAGDSDPELNAGVVELLKANPYFKATRPADYVWNVAKLVEAKAAVWMVKNNIKEAILVINADSVGANTSDPLDQPSLAVPHILPEGYKLTVYTANKESTVLEGTRPKP

SEQ ID NO: 152 - protein, 167 aa, synthetic construct
PLALPPPPLLVLPLGDEPLPLPAAAVPEWVREALAELAGSERETGLIFDENGNRIGPTITAGDEDPELRAGVVELLRANPYFQAHAPADYVWRSALGVEAKAAVWMVKNNIKKAVLVINAPSVGARTDDPLDQDTLVVPHILPEGYELTVWRSDETSTTLKGTRPKP

SEQ ID NO: 153 - protein, 167 aa, synthetic construct
LVVDLGPEELVVPVGDVPVPVEPGEPPEFIKKALEELKGKEKETGLIFDENGNRIGPEITAGDEDPELRKGVIELLKADPYFKANFPPEAFEIVSKLVEAKAAVYMVKNNIKKAILVINTKVVGSPSNPEKASPLIAVPHILPEGWELTVWRSDETSVTLKGKRPKP

SEQ ID NO: 154 - protein, 167 aa, synthetic construct
PLLLLGPLPLLLPLGDVPVPVPPPEPPPFIKEALEELKGKEKETGLIFDKNGNRIGPEITAGDEDPELRRGVIELLAADPYARANFPPEAFERVAKLVEAKAAVWCVKNNIKEAILVINSDVVGPEEDPEPASPELAVPLILPEGWTLTVWTRNEKSVVLKGKRPRP

SEQ ID NO: 155 - protein, 167 aa, synthetic construct
SVVLLGPLEEVVLLGDEPVEVEPEEPPEYILKALEELKGSEKETGKIFDKNGNRIGPTITAGDSDPELRAAVIELLAADPYFKENYPPEAWERLSKDVACKAAAWMVKNNIKEAILVINSDVVGPPDDPEGASPEDAVPLILPEGYTLTVWRSDKTSVTLKGKRPKP

SEQ ID NO: 156 - protein, 153 aa, synthetic construct
PVVVLPGVPPPPPLAAPSPDIPPWVVEALAELAGSERETGLLFDENGNRIGPRITAGPGPLAERAQELLRASPHVPALPPGARYAAALGVEAQYAVWMRENGIRRAHLVTNAPSVSGPPLGSRTAVPAILPRGATLTVWHPGAGRPVVLEGVG

SEQ ID NO: 157 - protein, 153 aa, synthetic construct
PVVVNLGVPPPAPPAAPSPDIPPWVVEALARLAGKKRETGVLFDENGNRIGPEITAGPGPLAARAQELLRASPHVPALPPDAVYAAAFGVEAQYAVWCRENGIRRAHLVTNRSSVSGPPLGETTAVPAILPRGATLTVWYPGAGRPVVLRGVG

SEQ ID NO: 158 - protein, 153 aa, synthetic construct
PVVPLPGVPPPLPLAAPSPGIPPWVVEALAELAGSRRETGVLFDENGERIAERVTAGPGPLAERAEALLRASPHVPALPPGERYAAALGVEAQFAVLMRDRGVRRAHLVTNRPYVSGPPLGERTAVPAILPRGAVLTVWTPGALEPVVLEGVG

SEQ ID NO: 159 - protein, 196 aa, synthetic construct
EFRAIFDPSAFPKVTYLLYEIKWGSSTKFWRKWCPNTPTQHAEINCLENDFKAIHHRASVRCSIIWFLSWSPCGLCCRLIIHFLRAHPKVSLQIYIGRLFRHRDERNRQGLRDLVSSGVNVYIMSLPAYDYCWRTFVDHQVREDDIYWPCYFTPWMIFYMLEFQCILQVGSTSLFTDLAEELHWLTCFPNRFTRSS

SEQ ID NO: 160 - protein, 192 aa, synthetic construct
MEPWPSLPPKAYNFNFRNLREAPNRNSTYLCFEVEEEQLDSPESSYWGVFRNQPSHLHQVYPEEPLHAEQRFLSWFYDALLSAHAYYRVTWYMSWSPCSECAEEVVSFLGEHENVSLSISASRLYKCDDQDQQEGLRLLDQAGVEVAMMSPDDFEYCWDNFVDNRRRSFRYWKGIRRNYHALDDELKEILWG

SEQ ID NO: 161 - protein, 191 aa, synthetic construct
LTSNSLKSLFCFRWYIPKEVLKEQFDPSKFPRETYLLCKIQWGDNGTSWKHWVKNYNDDDRHAEVCFLEDVFLMKSNNVNCNITLYLSWSPCADCSCSILDFLKQHSNVTISIYVARLYCVNSEENRKGLRRLHHSPQVNLSVMEMRGKCFNCWRTFTRGGDAYDFWPRNNKTEIIVNLVTLRIILEVSRL

SEQ ID NO: 162 - protein, 188 aa, synthetic construct
MEDNPAPSGRSLMDVDTFNSNFGIIWATETYLCYEVEVREEDTWVPVEGLQGFLRNQVYCHAELCFLDRVPSWLLDEGKQYRLMWYISWSPCPDCAPKLVDFLRKNSHVQLRIFAARIYTKLDGYEDGLRRLQDAGAKLAIMTPKELQHCWDTFVDNQGQQFEPELELLEKIRARCQRLENILRIQGN

SEQ ID NO: 163 - protein, 183 aa, synthetic construct
MDRYTFIENFNNQGCPFKTYLCYEVERLDGVIPLDEYKRFVCNKGPDQPHGPHHAEFYFLEQIRSRNLDQNQRYRLTCFISWSPCYSCAHNLTTFLKENRHISLRIFASRLYTSNRFGDYQKGLCELQAAGAQITIMTSQDFEHCWETFVDHRGNPFQPWEGLEAKSQELCEKLQAILRTQQN

SEQ ID NO: 164 - protein, 199 aa, synthetic construct
SLTADSDPLPCSNSLYRPPRSCLMQRKKFLYHYKNVRWAKGRHETYMCYVVKRRESAQTSSLDFGFLRNTARAHAEVVFLDYIRDWPLDPGLTYRLTWFSSWSPCYDCAARVVHFLQARPNVRLRLFIARLYYCSEGMRQPEGLRQLHQAGVGLGIMTYKDYFYCWNNFVANKEKSFKAWEGLHENAVRLSRLLRRILE

SEQ ID NO: 165 - protein, 206 aa, synthetic construct
TSSIKGSRKGDGGLQIIQPKEFKDVYDPSVLPKVTYLLYEIKWGNSTKIWRNWCRNTSTQHAEINFLENAFRERSFNPLTHCSITWFLSWSPCWKCSQSVVEFLKTYPKVNLEMYVARLFWHEDRSNRLGLRDLVMNGVTIRVMDLSAYNYCWRTFVSHQNVDDYWPWHLTPWIMFFSFELQLILQVSRSLGGKDSIQKLSRDMYR

SEQ ID NO: 166 - protein, 363 aa, synthetic construct
MLLSVHKVTLFFSPQIMYPEASHRDGYDPSVLRRVQYMFYEIKRSNSKRSWQSCCHSTRTEHAEIHFIEDVFQEQRSDPSVHCSITWYMSWSPCGDCCKEIRDFLKEQPNVNLVIYVARLYWHKEETNRQGLRSLMNIGVSIRVMDLPERQWCWNNFYSGGAEGGNHVFRLLLIQPKEFKDVYDPSVLPKVTYLLYEIKWGNSTKIWRNWCRNTSTQHAEINFLENAFRERSFNPLTHCSITWFLSWSPCWKCSQSVVEFLKTYPKVNLEMYVARLFWHEDRSNRLGLRDLVMNGVTIRVMDLSAYNYCWRTFVSHQNVDDYWPWHLTPWIMFFSFELQLILQVSRSLGGKDSIQKLSRDMYR

SEQ ID NO: 167 - protein, 268 aa, synthetic construct
MATERAETLVADPTMRRRIKPWEFTAFFDPSQLRKETSLLWEVWWGSSSRLWRSCSQNSVHHVERSFLDQFQAQRHLGPSTTCTITWFLSWSPCWECARAMQVFLDRYPTVKLTIFVARLYWHMDPQNRQGLRNLVQGGVDVRVMGPPEHYYCWRNFVDCSPGEEEEWPRYHPVPRQLFDLELRCICLDLPACLTISPGKHQQLLRFSLTLHSCHYQKLPPALLLPLGLVRPLPPQTSFSAAQGGWGPVTVNGEVQDLNTYGWFWVFR

SEQ ID NO: 168 - protein, 170 aa, synthetic construct
LEDVYDPSALPRVTYLLYEIKWGNSIKIWRNWCRNTSTQHAEINFLENAFKERSLNPLTHCSITWFLSWSPCWKCSQSVVEFLKTYPKVNLEMYVAQLFWHEDECNRQGLRDLVMNGVTIRVMDLSAYNYCWRTFVNHQNGEDYWPWHLTPWIMFFSFELQLILQVSRSL

SEQ ID NO: 169 - protein, 207 aa, synthetic construct
MLEIKEDVVHIFRAFWECGVHRGNWCGDTHILYEFGTSPATTDFRNIVTNDGDGGQHAEIKLIYILDNHIAQMVKCHQRKEIYLNMYMNRSPCYKCAFQLIRFKNYLYNKGYDIRITIKAAQPYMCKRITCSSCVEGTSHSDGLRELNRNGIFVNAWVWADWQFLAAYLNGLVRVCYDEKFPESGHGKRESRSMADEAAQEDFNALR

SEQ ID NO: 170 - protein, 174 aa, synthetic construct
KKRNIVWPAGIYGETWLKCTYLIASIKAIDGGSIFEKTFNNEHGQHVEMVMLHHSEFLKVVKENSNTEITVTLNHSPCSDCANNLKTFYEDYAENIKKIHHPVFIHNHREIEKNKTGLWDLNKAGFTLRAMTGQLWSEAHIDPVSYLNDAERNKIKQRDEETAKYLKDRLSQPN

SEQ ID NO: 171 - protein, 99 aa, synthetic construct
MVNINVTIIHRYKRIAYIGPTICVCMNFPSYFRWVTMYLSYSPCANCADRILQFSCDRPQCDITIAFSCLFRHTEKLHQDGLRRLNAESRITIKVFTSQ

SEQ ID NO: 172 - protein, 92 aa, synthetic construct
AEAHVPNRVVKIANGEEIFNKRFKNIPYDGHVEEIMLNDGGFLKAVAYNSDIEVILTLNYSPCNICAMKLKKFYESYKRKITKFIIQFSFLY

SEQ ID NO: 173 - protein, 200 aa, synthetic construct
AKFVGLVQEAVFGRQEPRAVLEEEAQHVREEEAMMLEAITLDDYEFPTARDVAAYREGRLNCGNEPDCIVFIGIAKWFDRVVAQRVFRHRQEGGSVGRLVAIDMVMFLLTEGYVLEHCSLEIYASRFPSFSELCSSDLLTQAEQFGSVFDKFVVRYASLRDGDSATDLVAALDRLGVSVGPMGDEEWSYLSRLERDWTIP

SEQ ID NO: 174 - protein, 115 aa, synthetic construct
MPVFPNATSFSQSDMNSNTTALMVYESVNGGWVVKGKVTSGKGNHAEEELYEDLKGSNLLKITQGAAILIEITKSPCHYHEGNKCCSHVLAKMKEKGYVSSIEVKYLGIYQYDQS

SEQ ID NO: 175 - protein, 219 aa, synthetic construct
FRKSYFKKPPTTAQSERFFTEKSSVLAILKFNAREILFTTNTAPADKLEFPHTGLFNQNGQKIGAHSEDWITAGFSNLMQGNEYAKVASTLPADQIDRHVASLKINFSPCTGCVRTLVNFNDYLKKTFGDKTIFRVKFLRPYDLPITLKNTESQQSKTFLSTLDELKNKGIVVRLHPDKDKTYTSDEFSRAAVKTHAPKHIDDLTKRWRDLKVARKKAA

SEQ ID NO: 176 - protein, 106 aa, synthetic construct
MTRAIIVNMEHNYASYGHQKPTDDNFETMPHATIQALIKARREDVVGGTIFLARFPCYECAKAIVFAGLTKVIYKEENTGDAMHSEAKMFLEFSGVEVVQNPDLEF

SEQ ID NO: 177 - protein, 245 aa, synthetic construct
MNSLRATGSIDEVLLHGQILSTYQALLALSRFDTVYSVFLPFSLFLDVPTLLNRATINSQPAVRRNLLPQLESNEKQKKLDIFIRSGLHGAEWPTIALCVVRLPMTGKVCVMQNEPGEQHVEERVIEKLEEDHIQVWEEEEETPIKLVIYMNFSTCHDCVEKFLSFTEKNRLHVEIVAAALYNCARKSCSPCNFVVRQKDDYQYQRNTEALKQLAEAGIKLRGFEWNDWSNLVALLNEASDIPGN

SEQ ID NO: 178 - protein, 114 aa, synthetic construct
RNLQYLEKYGKMILYTGKPNTSTSRTGAALMPCNFQLISAKLPILCASCLDLILIYHDVNPLTLDIRSTWLHCLGCWALQNRLKMIKVSGIQLHPMLDHMPLLNIWNQVQVNYA

SEQ ID NO: 179 - protein, 160 aa, synthetic construct
MPIVQNDPTFIRDKEKYFMGVAEEISKGSSHPIVPGGCIIIRDRTIIGDGRSILASCKVEIDCVTYAIATAAAHGTATVGAVVYSTRYPFSASIFQLHLMGIKKIVVKAHEWEPYYKDEFRRAARLARELSIAIEPFFDDDDERFSTNKQAPRFDNREEQ

SEQ ID NO: 180 - protein, 169 aa, synthetic construct
ARFYMSAAVNAATLSHAKKRKVGCIIEQSGNILSFGFNGTPTGEDNNCEDENGVTLSSVIHAEENALRKLERFELCDLHNCNIYCTCEPCYNCAILITNFFRRSAKPKLYFLEQSKRYPGVGSDILNRAGISSTQLFFDTNGNLSDKRPESEASLFTNWGWPPGDAKEE

SEQ ID NO: 181 - DNA, 684 bp, synthetic construct
  source 1..684: mol_type = other DNA
gccgcccccgtgctgccccctcccctgccccccaacctgcccctgaagatcgaccccgaaaccttcaagaagtacttcgaccccaaggtgcagagcagcgaaaccctgctcctgtacgagatcgacttcggcaccgagaacggcgtgatcagaggcagcctgttcagcggcgacaagcacgtgatcctgcagttcatcgagagactggagagcgagttcggcctgacccccgacaccaaggccgagatcaccctgtacctgagccacagcccctgcggcgcctgctgcgacgccctgatcgccttcctgaagagataccccaacatcaaggtgaacatctacgtgagcaagctgtacagacacgagaaccccgagaacagagccggcatccggagactgatcgccgccggcgtgaccgtgaagatcatggacgacgaggactacaagtacatctggaagaacttcgtgaacgtgaagcctagcgagctgaacaactaccccaagtacgaccccttctacatcaactgctacagatacgagctggcctgcatcaagctgggcctgccccccgccctgaaggtgaccctgagccccgaccccaacaacgtgaccttcagcctggagctgaccgaggagcactacgagaagctgcccaaggagctgatcgaggccaccggcgtgaag

SEQ ID NO: 182 - DNA, 684 bp, synthetic construct
  source 1..684: mol_type = other DNA
gccgcccccgtgctgccccctccccccgaccccgacctgcctagaaagatcgagcccgagctgttcgacagatacttcgaccctagagtgcagctggaggaaaccctgctgtgctacgagatcgacttcggcaagcccgacagcgtggagagagacaccctgttcagcggcgacgagcacgtgatcgtgaagttcatcaagagatacgagagcgagtacaacctgacccccgacaccaaggccaacatcaccctgtacctgagccacagcccctgcggcgagtgctgcgccgccatcatcgccttcctgaagaagtaccccaacatcaagctgaacatcgtggtggctagactgagaaaccccgccgaccccgccaacagagccggcatcagagccctgatcgccgccggcgtggaggtgagagtgatgaccgacgaggactacagatacctgtggcagaacttcgtgagaagaaagcccgaggacctggacaagtaccctcagtacgaccctagatacatcaactactacagactggagctggagtgcatcagactcgggctgccccctgccctggtggtggagctgagccccgaccccaacgacgtgaccttcaccctggagctcagacccgagcactacgagagactgccccccgagctgctggccgccaccggcctggag

SEQ ID NO: 183 - DNA, 684 bp, synthetic construct
  source 1..684: mol_type = other DNA
ggcagcctgctcctgccccctccccccgaccccaacctgcccaagaagatcgcccccgaaaccttcaagtactacttcgaccccaaggtgcagagcaaggaaaccctgctcctgtacgagatcgacttcggcaaggaggacagcctgatcagagacagcctgttcagcggcgacgagcacgtgatcctgcagttcatcaagaagctggagagcgagttcggcctgacccccgatacaaaggccaacatcaccctgtacctgagccacagcccctgcggcgcctgctgcgccgccctcatcgccttcaaggccaagtaccccaacatcaagatcaacatctacgtggctcagctgagaaaccacaccgaccccgccaacagagccggcatcagagccctgatcgccgccggcgtgaccgtgaaggtgatggacgacgagatctacaagtacatctgggagaacttcgtggacgtgaagcccgaggacagagacaagtaccctaagtacgaccccaagtacatcaactactacagatacgagctggagtgcatcaagctgggcctggagcccgccctggaggtgaccctgagccctgaccccaataacgtgaccttcaccctgaacctgacccctgagcactacgagaaggtgcccaaggagctgctggagagcctgggcgtgaag

SEQ ID NO: 184 - DNA, 684 bp, synthetic construct
  source 1..684: mol_type = other DNA
ggcgtggtcgtgccccctccccccgtggaccccgacgccgacaagaagatcgcccccgaggacttcgagaagtacttcgaccctagactgctgaccgaggaaaccctgctcctgtacgagatcgacttcggcggccccgacagcatctacagagacaccctgttcagcggcgacaagcacgtggagctgatcttcatcgagagactggagagcgagcccggcctgagccccgacaccaaggccgacatcaccctgtacctgagccacagcccctgcggcgagtgctgcgacgccatcgtggagttcctgaagagataccccaacatcaagctgaacatctacgtggctagactgaacaaccccgccgaccccaagaacagagccggcatcagaaagctgatcgaggccggcgtgaagatcgaggtgatggacgacgagatctacaagtactgctgggacaacttcgtgaacagaccccctagccaagccaacgactaccccaagttcgaccccttctacatcaacctgtacagatacgagctggagtgcatcaagctgggcctgccccccgccgtggtgatcgagaagagccccaaccccgaggacgtgaagttcagcgtgaagctgacccccgagcactacgagaagctgcccaaggagctgatcgacagcctgggcctgaag

SEQ ID NO: 185 - DNA, 684 bp, synthetic construct
  source 1..684: mol_type = other DNA
ggcctgatcatcccccccaagcctagaaaccccgacgaggacaagaagatcgagaaggaaaccttcgagaagtacttcgaccctagaaacttcaccaaggagagactgctcctgtacgagatcgactacggcggccccaacagcatcgagagagacagcctgttcagcggcaaggagcacgtggaaaccatcttcatcaaggagtacgaaaccacccccggcctgagccccgacaccaaggcccacatcaccctgtacctgagctggagcccctgcggcgcctgctgcgccgagatcatcgccttcctgaagaagtaccccaacatcaagctgaccatctacgtgagcaagctgtacaaccaccaagaccccgccaacagagccggcatcagagccctgatcgccgccggcgtggagatcaaaattatggacgacgaggactacaagtacgtgtggaagaacttcgtgagagtgaagcccgaggataagaacaagtaccccgtgttcgaccccttctacatcaacctgtacaagctggagctggagtgcatcaagctgggcctgccccccgccctggtggtggagatcaagcccgaccccaaggacgtgaagttcagcatcaagctgcagcccgagcactacgagaagctgcccaaggagctgctggagagcctgggcctgaag

SEQ ID NO: 186 - DNA, 684 bp, synthetic construct
  source 1..684: mol_type = other DNA
ggcatcgtggtgctgcccaagcccgtggaccccaacgagagcaagaagatcgacaaggaaaccttcgagaagtacttcgaccctagaaacttcaccaaaaagaccctgctgctgtacagcatcgacttcggcggccccgacagcatcgtgctggacaccctgttcagcggcgacaagcacgtgatcgacatcttcatcgagaagctggagagcacccccggcctgagccccgacaccaaggccaccatcaccctgtacctgagccacagcccctgcggcgagtgctgcgacagaatcgtggccttcctgaagagataccccaacatcaagctgaacatcgtggtgagcagactgtacaaccacgccgaccccaagaacagagccggcatccggagactgatcgccgccggcgtgaccatcagaatcatggaccaagaggactacgaggagatctggaagaagttcgtgaaggtgaagcccgaggacaaggacaagtaccccgtgttcgacgagaagtacatcaactactacaagctggagctggagtgcatcaagctgggcctgccccccgccctggtggtggagatcaagcccaaccccgaggacgtgaagttcagcatcaagctgacccccgagcactacgagaagctgcccaaggagctgctggagagcctgggcctgaag

SEQ ID NO: 187 - DNA, 684 bp, synthetic construct
  source 1..684: mol_type = other DNA
gccctgctcctggtgccccctcccgagcccaaggagaaggagaacctgatcaagcccgaggagttcaagaagtacttcgaccctagactgctcctgaaggaaaccctgctcctgtacgagatcgagtacggcggccccggcaccgtggagagaagcgtgctgttcagcggcgacgagcacgtgatcctgcagttcatcaagagattcgaggcctcccccggcctgagccccgacaccaaggcccacatcaccctgtacctgagctggagcccctgcggcgagtgctgcaaggccctggtggccttcaagaaaaagtaccccaacatcaagatcaccatctacgtgagcagactgagaaaccacaccaaccccgagaacagagagggcatcagactgctgatcgaggccggcatcgagatcaaggtgatgacccacgaggacctgaagtacgtgtgggagaacttcgtggacaagcccgagagcgagaagaacagctaccccatctacgacgagaagtacattacactgtacgaatacgagctggagtgcatcaagctgggcctggagcccgccctgatcgtggaggtgagccccgaccccaacaacgtgaccttcagcctgaacctgaagcccgagcactacgagaagctgcccaaggagctgctggagagcctgggcgtgaag

SEQ ID NO: 188 - DNA, 684 bp, synthetic construct
  source 1..684: mol_type = other DNA
gccctgctcctggtgccccctccccccgaccccgccctggccaacctggtgcctcctgaggagttcaagaagtacttcaaccccctgaacctgctgaagagaaccctgctcctgtacgagatcaagtacggcggccccggcaccgaggagagaggcgtgctgttcagcggcgacgagcacgtgatcgacatcttcatcaagagattcaccgctagacccgacctgaccaaggacaccaaggccaagatcaccctgtacctgagccacagcccctgcggcgcctgctgcgacagaatcgtggccttcctgaaggagtaccccaacatcgagctggacatcgtggtgagcaagctgagaaaccacgccgaccccgagaacagagccggcatccggagactgatcgccgccggcgtgaccgtgagaatcgccacccaagaggactacgagtacatgtggaagaacttcgtggacgtgaagcccgaggacaagaacaagtaccccgtgtaccccgaggagtacctgacctactacagattcgagctggagtgcatcaagctgggcctgccccccgccctggtggtgaagaaaaagcccgaccccaacgacgtggagttcaccctggagctgacccccgagcactacgagaagctgccccccgagctgctggcctccaccggcgtgaag

SEQ ID NO: 189 - DNA, 684 bp, synthetic construct
  source 1..684: mol_type = other DNA
gccctgctcctgctgccccctcccctgccccctagcctgagcaacctggtgccccccgagctgttcaagagatacttcgaccctagaatcctgagcaaggaaaccctgctcctgtacgagatcgactacggcggccccaacaccatcgagagaaagaccctgttcagcggcgacaagcacgtgatcgacatcttcatcgaggagctggagaacagacccggcctgtccaaggatacaaagatgaacatcacactgtacctgagctggagcccctgcggcaagtgctgcgacagaatcgtggagttcaagaaaaagtaccccaacatcaagatcaacatcgtggtgagcagactgagaaaccacaccgaccccgagaacagagccggcatcagaaagctggtggacgccggcgtgaaggtgagaatcgccacccaagacgacctgaagtacatgtggaagaacttcgtgaacgtgccccctagcgagctgaacagctaccccgtgtacgaccccttctacatcaccctgtacgagttcgagctggagtgcatcaagctgggcctgccccccgccctggtgatcacacagagccccgaccccaacaacgtgaccttcagcatcaacctggagcccatccactacgagaagctgcccaaggagatgctggaggccctgggcgtggag

SEQ ID NO: 190 - DNA, 684 bp, synthetic construct
  source 1..684: mol_type = other DNA
ggcggcctgctgccccctccccccgaggaccccaacctgcccaagaagatcccccccgaggaatttgagagatacttcgaccctcggagactgctgagagaaaccctgctgtgctacgagatcgactacggcggcgacgagccccccgagctggacaccctgttcagcggcgaggagcacgtgatcctgcagttcatcaagagattcgaggccgaccctagattcagcaaggacaccaaggccaccatcaccctgtacctgagccacagcccctgcggcgagtgctgcgccgccctgatcgccttcaagaaaaagtaccccaacgtgaagatcaacatctacgtggctagactgcgggaccacgccgaccccgccaacagagccggcatcagagccctggtggccgccggcgtgaccatcagaatcatggacgacgagatcctgaagtacgtgtggaagaagttcgtgaacagaaagcccgaggacctggacaagtaccccgtgtacgaccccaagtacctgacctactacagagtggagctggagtgcatcctgctgggcctgcccccctgcctggaggtggtgagaacccccgaccccgacgacctggtgctgagactgagactgcagcccgagcactacgagaagctgcccaaggagctgctggacagcctgggcatcaag

SEQ ID NO: 191 - DNA, 684 bp, synthetic construct
  source 1..684: mol_type = other DNA
ggcggcctgctcctgccccctcccctggaccccaacctgagcagaaagatcaagcccgaggagttcgaggagtacttcgaccctagaagagagagcaagaagagactgctgtgcgtggagattgatagaggcggcgacgagccccccgagagattcaccatgttcgagggcgacgagcacgtggagattcagttcatcaaggagttcaccgccgaccctagattcagccccgacgacaaggtgagaatcagactgtacctgagccacagcccctgcggcgagtgctgcgccgccatcatcgccttcaaggccgcctaccccaacgtggaaatcgagatcgtggtggctagactgtacaaccccgaggaccccgccaacagagccggcatcagagccctggtggccgccggcgtgaccatcagaatcgccgaccaagaggactacaagtacgtgtggaagaacttcgtgctgaagaagcccgaggacaaggacaagtaccccgtgttcgaccctagatacctgagactgtacgaggtggagctggagtgcatcctgctgggcctgcccccctgcgtgagaagaatcctgacccccgaccccgacaacctggtgttcgagctgctcctgacccccgagcactacgctagactgcctagagagctgctggaggccctgggcgtggcc

SEQ ID NO: 192 - DNA, 684 bp, synthetic construct
  source 1..684: mol_type = other DNA
gccggccccctgccccctccccctcccgaccccgacgagcccctgaagatcccctacgaggagttcgagtactacttcaaccccaccatcctgaccaaaaagaccctgctgctggtggagatcgagagaggcggccccatcccccccatcagattcagcctgttcagcggcgatcagcacgtgatcgtgcagttcatcgagagattcgaggccgaccctagcgtgagccccgacgacaaggccaccatcaccctgtacctgagccacagcccctgtggggagtgctgtgacgccctggtggccttcctggctagacaccccaacgtgaccatcaacatcgtggtggctagactgagaaaccaccgggaccccgccaacagagccggcctgcggagactgcgggacgccggcgtgaccatcagagtgatggaccaagaggactacgaggagatctacaagaacttcgtgaacagaaagcccgaggacagagacaagtaccccatctacgacaagaagtacatcacctactacgaggtggagctggagtgcatcttcctgggcctgcccccctgcgtggaggagatcatcacccccgaccccaacaacctgatcttcaagatcaagctgacccccgagcactacgagaagctgcccaaggagctgctggacagcctgggcatcaag

SEQ ID NO: 193 - DNA, 684 bp, synthetic construct
  source 1..684: mol_type = other DNA
agcggcggcctgctgccccctcccgtgccccccaacaacaagaacctgattccccccgagctgttcgaagagtacttcgaccccaccaacctgagcaagagaaccctgctcctgtacgagatcgagttcgagggcaccaacaagatcgagagaggcgtgctgttcagcggcgacgagcacgtgatcctgcagttcatcaaggagttcgagagcaagtacaagatcgaccccaatcagaagatcaagatcaccctgtacctgagccacagcccctgcggcgcctgctgcgacgccctggtggccttcctgaagaagcaccccaacatcgagatcgagatctacgtggctagactgagaaaccatcagctgcccgagaacagagccggcatccggagactggtggacgccggcgtgaaggtgaagatcatggaccaagagatctacaagtactgctgggacaacttcgtggacaagagccccgagaacaaggacaactaccccgtgtacgaccctagatacctggcctactacagactcgaactggagtgtatcctgctgggcctgcccccctgcctggaggtgaccgtggaccccgagcccggcaaggtgaccttcaccctgaacctgacccccgagcactacgagaagatcccccccgagctcctggccgccaccggcatcaag

SEQ ID NO: 194 - DNA, 684 bp, synthetic construct
  source 1..684: mol_type = other DNA
ggcgggggcctgctgccccccatccccgaccccaacaccaagaacctgatcaagcccgaggacttcgagaagtacttcaaccccaagaatctgagcaaaaagaccctgctgctgtacgagatcaagttcgagggcaccgacaacatcgagaagaaggtgctgttcagcggcaagaagcacgtgatcctgatcttcatcgaggagttcgagaagaaccacaagatcgacaagaatcagaagatcaacatcaccctgtacctgagccacagcccctgcggcgagtgctgcgacgccatcgtgaagttcctgaagaagtaccccaacatcaagctgaacatctacgtggccaagctgtacaaccacagcgaccccaagaacagagccggcatcagaaagctgatcgaggccggcgtgaacatcaagatcatggatcagaacgactacaagtacatctgggagaacttcgtggacaagcccgagagcgagaagaacgactaccccgtgtacgaccccaagtacctgaactactacaagctggagctggagtgcatcaagctgggcctgcccccctgcctgaccgtgaccgtgacccccgagcccggcaaggtgaccttcaccctgaacctgcagcccgagcactacgagaagctgccccaagagctgatcgacagcaccggcctgaag

SEQ ID NO: 195 - DNA, 684 bp, synthetic construct
  source 1..684: mol_type = other DNA
ggcggcctgctcctgccccctcctagaaaccccaacgacaagaacctgatcaagcccgaggagttcaaggaggccttcgaccccaagaacctgagcaagagaaccctgctcctgtacgagatcaaggtggagggcaccgacaaggtggagagaggcgtgctgtacagcggcgacaagcacgtgtacctgcagttcatcgaggagttcgagagcaagcacaagctggaccccaatcagaagatcaagatcaccctgtacctgagctggagcccctgcggcgagtgctgcgacgccatcgtggccttcctgaagaagcaccccaacatcagcctggagatcgtggtggccaagctgagaaaccacgaggaccccaaaaatagagccggcatccggagactgatcgccgccggcgtgaccatcagaatcatggaccaagaggactacaagtacatctgggacaacttcgtggacaagagccccgagaacaagaacgactaccccgtgtacgacgagaagtacctgaactactacaagctggagctggagtgcatcaagctgggcctgcccccctgcctgaaggtgaccgtggaccccacccccggcaaggtgaccttcgagctgatcctggaggaagagcactacaagaagatccccaaggagctgctggagagcaccggcatcaag

SEQ ID NO: 196 - DNA, 714 bp, synthetic construct
  source 1..714: mol_type = other DNA
atgggcgagcactggcagtacgccggcagcggcgagtacatcccccaagatcagttcgaggagaacttcgaccctagcgtgctgctgagcgaaacccatctcctgagcgagctcacctggggcggcagaccctacaagcactggtacgagaacaccgagcactgccacgccgagatccacttcctggagaacttcagcagcaagaacggcagctgcatcatcacctggtatctgagctggagcccctgcgccgagtgcagcgctagaatcgccgacttcatgaaggagaacaccaacgtgaagctgaacatccacgtggctagactgtacctgcacgacgacaagcacacaagacaaggcctgagatacctgatgaagatgaagagagtgaccattcagatcatgaccatccccgactacaagtactgctggaacaccttcctggaggacgacggcgaggatgagagcgacgactacggcggctacgccggcgtgcacgaggacgaggacgaatccgatgacgatgactacctgcccacccacttcgccccctggatcatgctgtacagcctggagctgagctgcatcctgcaagacttcgccccctgcctgaagatcatccaaggcaaccacatgagccccacctttcagctgcacgtgcaagaccaagagcagaagagactgctggagcccgccaacccctggggcgccgac

SEQ ID NO: 197 - DNA, 588 bp, synthetic construct
  source 1..588: mol_type = other DNA
atggccgccgtgagcaacagaggcagagcccctagacagaacaatcagagatggaagatgcagcccgacgacttcaagagcaactacctgcctagccaacaccccaaggtggtgtacctgatgtacgccatcaagtggagaagaggcaccatctggagaggctggtgcagcaacaaccccacacagcacgccgaggtgaacttcctggagaactgcttcaagcccctgagcagcgcctcctgcagcatcacctgggtgctgagcaccaccccctgcggcaagtgcagcaagagaatcctggagttcctgagaatccaccccaacgtgaccctgaagatcttcgccgccaagatcttcaagcacctggacatcagaaacagacaaggcctgagaaacctgaccgagaagggcgtgatcatcagaatcatgagccccgccgactacagcagcagctggaagagattcgtggcctatcagcacggcgaggaggactactggccctggaacttcatcctgcacatcctgctgaactgcatcgagctgcacagaatcatcctgggcctgccccccgtgctgaacctggagtac

SEQ ID NO: 198 - DNA, 765 bp, synthetic construct
  source 1..765: mol_type = other DNA
atgttcctgctgagcggcatcgaaaccgagagagagaagcacacccccgtgaagcaagacaccgcctgggaggccgtggtgaaggccatggccaccggctggaaccgggaccacgagagcagaggcatcaccaagggcggcaagatcagccaagaaaccttcatcgacacctacgaccctagcgtgctgagaagagagcagtacatgctgtacgagatcaagtggagcaacaacaaaagacctagacagagctgctgccacagcacacagaccgagcacgccgagatctacttcctggaggacgtgtttcagaagcagagaagcgaccctagcgcccactgcttcatcacctggttcgtgagctggagcccctgcggcgactgctgcaagcagatcagagacttcctgaaggagcagcccaacgtgaagctgttcatctacgtggctagaatctactggcacaagagagagatcaacagacaaggcctgagaaacctgatgaacatcggcgtgagcatcagagtgatgtacctgcccgtgtacagatactgctggagaaccttcgtggacaaggaggacaagtacgaggactactggcctagacacttcgccccctggatcatgctgtacagcctggagctgcagagcatcctgcagaacatccctagctgcctgaagatcagcagcttcaagaatcagagccccgtgttcagcctgtgcgtggacgagaagcagaagagagccctggccctggccaacccctac

SEQ ID NO: 199 - DNA, 546 bp, synthetic construct
  source 1..546: mol_type = other DNA
aaggccgccatcctgctgagcaacctgttcttcagatggcagatggagcccgaggcctttcagagaaacttcgaccctagagagttccccgagtgcaccctgctcctgtacgagatccactgggacaacaacacaagcagaaactggtgcaccaacaagcccggcctgcacgccgaggagaacttcctgcagatcttcaacgagaagatcgacatcaagcaagacaccccctgcagcatcacctggttcctgagctggagcccctgctacccctgcagccaagccatcatcaagttcctggaggcccaccccaacgtgagcctggagatcaaggccgctagactgtacatgcatcagatcgactgcaacaaggagggcctgagaaacctgggcagaaacagagtgagcatcatgaacctgcccgactacagacactgctggaccaccttcgtggtgcctagaggcgccaacgaggactactggccccaagacttcctgcccgccatcaccaactacagcagagagctggacagcatcctgcaagac

SEQ ID NO: 200 - DNA, 582 bp, synthetic construct
  source 1..582: mol_type = other DNA
cactaccccgagattcagcccaaggagttcaaggacgtgtacgaccctagcgtgctgcccaaggtgacctacctgctgtacgagatcaagtggggcaacagcaccaagatctggagaaactggtgcagaaacacaagcacacagcacgccgagatcaacttcctggagaacgccttcagagagcggagcttcaaccccctgacccactgcagcatcacctggttcctgagctggagcccctgctggaagtgcagccaaagcgtggtggagttcctcaagacctaccccaaggtgaacctggagatgtacgtggctagactgttctggcacgaggacagaagcaacagactgggcctgcgggacctggtgatgaacggcgtgaccatcagagtgatggacctgagcgcctacaactactgctggagaaccttcgtgagccatcagaacgtggacgactactggccctggcacctgaccccctggatcatgttcttcagcttcgagctgcagctgatcctgcaagtgagcagaagcctgggcggcaaggacagcattcagaagctgagcagagacatgtacaga

SEQ ID NO: 201 - DNA, 717 bp, synthetic construct
  source 1..717: mol_type = other DNA
tgcaccggccacaagggccccaaggctagatggcagattcagcccaaggagttcagagccatcttcgaccctagcgccttccccaaggtgacctacctgctgtacgagatcaagtggggcagcagcaccaagttctggagaaagtggtgcctgaacacccccacacagcacgccgagatcaactgcctggagaacgacttcaaggccatccaccacggccctagcgtgcactgcagcatcatctggttcctgagctggagcccctgcggcctgtgctgcagactgatcatccacttcctgagagcccaccccaaggtcaccctgcagatctacatcggcagactgttcagacaccgggacgagagaaacagacaaggcctgcgggacctggtgagcagcggcgtgaacgtgtacatcatgagcctgcccgcctacgactactgctggagaaccttcgtggaccacagagtgagagaggacgacatctactggccctgctacttcaccccctggatgatcttctacatgctggagtttcagtgcatcctgcaaggcctgcccccctgcctgcagatctggctgaagaactacaccggcagccccgtgtttcagctggtgctgcaagagagccacagaaagatcctgtggcagagcctgcacaccgccaacagccctcagcagagactgatcttcagacctagacctagcaccgagcac

SEQ ID NO: 202 - DNA, 774 bp, synthetic construct
  source 1..774: mol_type = other DNA
cacaccgccgaccaaggcagaagaatcaagagctgggagttcacatccttcttcgaccctagccaactgagaaaggagacaagcctgctgagcgaggtgtggtggggcagcagcagcagactgtggagaagctgcagccaaaacagcacccaccacgtggagcagagcttcctggaccacttccaagctcagagacacctgggccctagcaccacctgcaccatcacctggttcctgagctggagcccctgctgggagtgcgctagagccatcagaaccttcctggacaagtaccccaccgtgaagctgaccatctacgtggctagactgtactggcacatggaccctcagaacagacaaggcctgagaaacctggtgaagggcggcgtggacgtgagaatcatgggccctagcgagcactactactgctggagaaacttcgtggactacagcagaggcgaggaagaggagtggcctagataccacagagtgcccatccacctgttcgacctggagctgcactgcatctgcctggacctgcccgcctgcctgaccatcagccccgtgaagcatcagcagctgctgagcttcagcctgaccctgcacacctgtcactatcagaaactgccccccgctctgctcctgctcctcggcctggtcagacccccccctccccaaacaagcctctccgtcgcccacagagggagaggccccggcaccgtcaacggcggcgagtgggaccccaacaactggggctggttctgggtgctgaga

SEQ ID NO: 203 - DNA, 648 bp, synthetic construct
  source 1..648: mol_type = other DNA
agaagaatcgagccctgggagttcgaggacttcttcgaccctagacagttcagacccgaaacctgcctgctgtacgaggtgagatggggcagcagcagaaacgcctggagaagcaccgctagaaacaccacaagacacgccgaggtgaacttcctggagagattcgccgccgagagacacttcgacaagcccgtgagctgcagcatcacctggttcctgagctggagcccctgctgggagtgcagccaagccattggggccttcctgagccaacacccccaagtgaccctggccatccacgtgacaagactgttccaccacgaggacgagcagaacagacaaggcctgcgggacctgctggctagaggcgtgaccctgcaagtgatgggcgacagcgagtacgcccactgctggagaaccttcgtgaacagcccccccggcgccgagggccactaccctagataccctagcgacttcacaagactgtacgccctggagctgcactgcatcatcctgggcctgcccccctgcctggagatcctgagaagatatcagaatcagttcaccctgttcagactggtgcctcagaactgccactatcagatgatcccccacctgaacttcttcgtggtgagacactacttcttc

SEQ ID NO: 204 - DNA, 786 bp, synthetic construct
  source 1..786: mol_type = other DNA
atgcaccacagcgctagactgccccccaactgcatcgtgagcagatatgctaacgccccctggaccgtgctgcccctgcccctgccccctaccgaagctcccgccaccggcgacgacaccctcagaagaagaatcgagccctgggagttcgaggccttcttcaacccccaagagctgagaagagaggcctgcctgctgtatcagatcacctggagcagccacaaggtgtggagagaaaccgccaagaacaccgtggacagccacgtggaggtgaacttcattcagaacctgaccgccggcagatactgcagacctagcacaagatgcagcatcctgtggttcctgagctggagcccctgcagcagctgcagcaaggccatcagactgttcctgagccaacaccccggcgtgagcctggtgatctacgtggctagactgtttcagcacatggaccctcagaacagacaaggcctgagagagctgatccacagcggcgtgaccatccaagtgatgcggccccaagagtacgactactgctggaagaacttcgtgaactacccccccggccaagaggagcactggcctagataccccgtgcagtgcatgaccctgtacaacctggagctgtactgcatcatccacaacctgcccccctgcgtgagaatcagcaagcagagacagagccaactggccttcttcagcctgggcctggagaacgtgcactatcagagaatcccccctcccctgctcctgctgaccggcctggtgttcgtgttcccctggaag

SEQ ID NO: 205 - DNA, 705 bp, synthetic construct
  source 1..705: mol_type = other DNA
atgaacagcaagaccggccctagcgtgggcgacgccaccctcagaagaagaatcaagccctgggagttcgtggccttcttcaacccccaagagctgagaaaggaaacctgcctgctgtacgagatcaagtggggcaatcagaatatctggagacacagcaatcaaaatacaagccaacacgccgagatcaacttcatggagaagttcaccgccgagagacacttcaacagcagcgtgagatgcagcatcacctggttcctgagctggagcccctgctgggagtgcagcaaggccatcagaaagttcctggaccactaccccaacgtgaccctggccatcttcatcagcagactgtactggcacatggatcagcagcacagacaaggcctgaaggagctggtgcacagcggcgtgaccattcagatcatgagctacagcgagtaccactactgctggagaaacttcgtggactacccccaaggcgaggaggactactggcccaagtacccctacctgtggatcatgctgtacgtgctggagctgcactgcatcatcctgggcctgcccccctgcctgaagatcagcggcagccacagcaaccaactggccctgttcagcctggacctgcaagactgccactatcagaagatcccctacaacgtgctggtggccaccggcctggtgcagcccttcgtgacctggaga

SEQ ID NO: 206 - DNA, 708 bp, synthetic construct
  source 1..708: mol_type = other DNA
atggcctccgagaagggccctagcaacaaggactacaccctcagaagaagaatcgagccctgggagttcgaggtgttcttcgacccccaagagctgagaaaggaggcctgcctgctgtacgagatcaagtggggcgcctcctccaagacctggagaagcagcggcaagaacaccaccaaccacgtggaggtgaacttcctggagaagctgacaagcgagggcagactgggccctagcacctgctgcagcatcacctggttcctgagctggagcccctgctgggagtgcagcatggccatcagagagttcctgagccaacaccccggcgtgaccctgatcatcttcgtggctagactgtttcagcacatggacagaagaaacagacaaggcctgaaggacctggtgacaagcggcgtgaccgtgagagtgatgagcgtgagcgagtactgctactgctgggagaacttcgtgaactacccccccggcaaggccgctcagtggcctagatacccccctagatggatgctgatgtacgccctggagctgtactgcatcatcctgggcctgcccccctgcctgaagatcagccggagacatcagaagcagctgaccttcttcagcctgacccctcagtactgccactacaagatgatccccccctacatcctgctggccaccggcctgctgcagcctagcgtgccctggaga

SEQ ID NO: 207 - DNA, 801 bp, synthetic construct
  source 1..801: mol_type = other DNA
atgggcacccccaaggacaccaagagcagacacctggccggcagatgccacgagctgcacaacagcagaggcagaagccccgagagcgacaccatggcctccgagaagggccctagcaacaaggactacaccctcagaagaagaatcgagccctgggagttcgaggtgttcttcgacccccaagagctgagaaaggaggcctgcctgctgtacgagatcaagtggggcgcctcctccaagacctggagaagcagcggcaagaacaccaccaaccacgtggaggtgaacttcctggagaagctgacaagcgagggcagactgggccctagcacctgctgcagcatcacctggttcctgagctggagcccctgctgggagtgcagcaccgccatcagagagttcctgagccaacaccccggcgtgaccctggtgatcttcgtggctagactgtttcagcacatggacagaagaaacagacaaggcctgaaggacctggtgacaagcggcgtgaccgtgcaagtgatgagcgtgagcgagtactgctactgctgggagaacttcgtgaactacccccccggcaaggccgctcagtggcctagatacccccctagatggatgctgatgtacgccctggagctgtactgcatcatcctgggcctgcccccctgcctgaagatcagccggagacatcagaagcagctgaccttcttcagcctgacccctcagtactgccactacaagatgatccccccctacatcctgctggccaccggcctgctgcagcctagcgtgccctggaga

SEQ ID NO: 208 - DNA, 591 bp, synthetic construct
  source 1..591: mol_type = other DNA
aagcccgagaagatgaaggaggaggacttctactattacttctacaacgccgaggacagaaccggcagaaccaccaactacctgtgctacgtgctgaagaaaaagctgagcgccaccacaaccgaaaccgagagcggctggctgaaggacgagaacggcgtgcacgtgtacgacaagttcctggagtacttcgagaagctgaacctggaccccgacctggagtacgacatcaccctgtacgcctccttcagcccctgctacgagtgcgccaagagaatcgccgagtggctggccaagcaccccaacgtgaagctgaccatcaaggcctccaacctgtacaactgcggccccgacagccccaaccccgagggcctgagactgctgaaggaggccggcgtgaccatcagcatcatggactacgaggactacaagtacgtgtgggaaaccttcgtggacaacaagggcaaggagtacgtgcccttcgagggcttcgaggagaacagcaagaagctgcaagccaaactggagagcatcctgaccccccccgaggaggccgctgccctggccgagctgctggccgccctgggcctg

SEQ ID NO: 209 - DNA, 591 bp, synthetic construct
  source 1..591: mol_type = other DNA
aagcccgagaagatggacgaggccaccttcagatactacttctacaacgccgccgacaacaccggcagaaccaagagatacctgtgctacgagctgagaaagcagctgagcgccaccgaaaccgaggtggagagcggctggctggaggacgaggacggcgtgtgcgtgtacgacaagttcctggagtacctggaggagctggacctggaccccgacctggagtacgacgtgaccctgtacgcctcctggagcccctgctacgagtgcgccgctagaatcgccgccttcctggccgcccaccccaacgtgagactgaccatcaaggccgctagactgtataactgcgaccccgacagccccaaccccgccgggctgagactgctggccgctgccggggtgaccatcagcatcatgaccgaggccgactacgagtacgtgtggaacaccttcgtggacaacaacggcaaggcttacgtggctcccgccggcctggccgaaaacagcgctagactccaagctcagctcgacgccattctcacaccccccgaggccgctgccgctgccgctgccgccctggccgccgctggcctg

SEQ ID NO: 210 - DNA, 591 bp, synthetic construct
  source 1..591: mol_type = other DNA
aagcccgagaagatgcccgaggccctgttctactattacttcaacaacgccgaggacgtgaccggcagaaccacaagatacctgtgctacgtgctggagacacagctgagcgccaccacaaccgaggtgcagaagggctggctgcgggacgaggacggcgtgcacgtgtacgacaagttcctggcctacctggagaagctgaacctggaccccgataagacctacaacgtgaccctgtacgcctccttcagcccctgctacgagtgcgccaagagaatcgccgcctggctgaaggcccaccccaacgtgagcctgaccatcaagaccgctagactgtacgactgcgacgagaacagccccaaccccgagggcctgagactgctgaaggaggccggcgtgaagatcagcatcatgaccgaggccgactacaagtactgctgggacaccttcgtggacaacaacggcaagccctacgtgccctaccccggcctggaggagaacagcaagaagctgcaagagaagctggagaagatcctgaccccccccgaggaagaggccgctgccgctgccctggccgccgctctggggctc

SEQ ID NO: 211 - DNA, 591 bp, synthetic construct
  source 1..591: mol_type = other DNA
aagagcgaaaagatgcccgaggaagagttctactattacttctacaacgccaagaacaacgagggcagaaccgtgagatacctgtgctacgagctgtacgaggagaagggccccgagaaggtgctgctggacaagggctggctgaagagcgagaacggcgagcacgtgatcaacctgctcctgaagaagctggagaagctgaagctggaccccaagaaaaagtaccacctgaagctgtacatgagcttcagcccctgctacgagtgctgcaagaagctggccgagtggaagaaaaagcaccccaacgtgagcatcaccatcaagatggccaacatctacgtgaaccccgaggaccctcgggaccccgagggcctgagactgctgaaggaggccggcgtggagatcagcatcatgaccctggccgactacgagtacgtgtggaacaccttcgtggacaacaacggcaagccctacgtgcccttccccggcctggaggagaacagcaaggccctccaagccgaactggacgctatcctgaccccccccgaggctgctgccgctgccgctgccgctgccgccgctgccgggctc

SEQ ID NO: 212 - DNA, 591 bp, synthetic construct
  source 1..591: mol_type = other DNA
aagagccccaagatgcccgaggaagagttctactattacttctacaacgccgccgacaccaccggcagaaccgtgagatacctgtgctacgagctgtacgtgaaggagggcgagaaagaggtgctgctggacaagggctggctgaagagcgagaacggcgtgcacgtggagctgctcctgctgaagtacctgagcaagctggacctggaccccgagaaggagtaccacctggtgctgtacatgagcttcagcccctgctacgagtgctgcaaggccctggccgagtggctgaagaagcaccccaacctgaaggtgaccatcaagatggctagaatctacgtggaccccgaggaccctagacagcccgagggcctgagactgctggccgaggccggcgtgaccatcagcatcatgaccgaggccgactacgagtacgtgtggaacaccttcgtggagaacaacgggaagccctatgtcccctaccccggccaagccgaaaacagcgctagactggctgctgaactggccgccattctgacccctcctgccgctgccgctgctgccgctgccgctgccgctgccgccggcctc

SEQ ID NO: 213 - DNA, 591 bp, synthetic construct
  source 1..591: mol_type = other DNA
aagagcaagaagatcgacgaaaagaccttctactattacttcaagcccgagagaaacaccaccggcagaaccaagagactgctgtgctacgagctgtacaaggaggagggccccgagaaggtgctgctggacagcggctggctggagagcgagaacggcaagcacgtgatcgacctgctcctggagaagctgagcaagctgaacctggaccccaacaagaagtaccacctgatcctgtacctgagcttcagcccctgctacgagtgcgccaagagactggccgagtggaagaaggagcaccccaacatcagcatcaccatcaagatggctagaatctacagaaaccccgaggaccctcgggaccccgagggcctgagactgctgaaggaggccggcgtgaagatcagcatcatgaccaaggaggacttcgagtacgtgtggaacaccttcgtggacaacaacggcaaggagtacaagcccttcgagggcttcgaggagaacagcaaggagctgcagaaggaactggacgccatcctgaccccccccgagctggccgctgccgctgccgccctggccgccctgctgggcctg

SEQ ID NO: 214 - DNA, 591 bp, synthetic construct
  source 1..591: mol_type = other DNA
aagagccccaagatgaagaaggacgacttcgagttctacttcaagaacgtgaaggacgccaccggcagaaccaaggagtacctgtgctacgaggtggtgaagcagctgagcgaaaccgaggaagaggtggagaagggctacctggtgagcgagaacggcgtgcacgtgtacgacctgttcctggagaaactggagaagctgaacctggaccccaacaagaagtacaacgtgaccctgtacgcctccttcagcccctgctacgagtgcgccaagagactggccgccttcctggccaagcaccccaacgtgagcctgaagatcaagaccgctagactgtacaacgtggagcccggcagccccaacgccgagggcctgagactgctgaaggccgccggcgtggagatcagcatcatgaccgaggccgacttcgagtacgtgtggaacacctacgtggacaacaacggcgaggcctacgtgccctgggagggccaagaggagaacagcaaggagctgcaagccaagctggaggagatcctgaccccccccgaggagagagccgctgccgccgctctggccgctgccctgggcctg

SEQ ID NO: 215 - DNA, 591 bp, synthetic construct
  source 1..591: mol_type = other DNA
aagagccccaagctgcccgaggacgagttcaagttctacttcagaaacgaggaggaccccaccggcagaaccaagagatacctgctgtacgagctgaccacacagctgagcgaaaccaccgaagaggtggagaagggctacctggtgagcgaggacggcgtgcacgtgatcaacaagttcctggagtacctgagcaagaagaacctggaccccaacaagaagtacaacgtgaccctgtacctgagccacagcccctgctacgagtgcgccaaggccctggccgagttcctgaagaagaaccccaacgtgagcctgaagatcgtgtgcagcagactgtacaacgtggagcccggcacccccaacgccgagggcctgagactgctgaaggaggccggcgtggagatcagcatcgccaccaaggccgactacgagtacgcctggaacaccttcgtggacaacaacggcaagccctacgtgccctgggagggcgaggaggaaaacagcaagaagctgcagaagaagctggaggagatcctgacccccgaggaagagaagaaaaaggaggaagaggagagaaagaaaaagggcctg

SEQ ID NO: 216 - DNA, 591 bp, synthetic construct
  source 1..591: mol_type = other DNA
aagagcgaaaagctggacgaaaagaccttctacttcgagttcaagcccgccgagaacagagagggcagaaacacaagatacctgtgctacgaggtcaagaccaagctgagcgaaaccgaggaagaggtggagaagggctacctgaagagcgagaacggcgtgcacgtgtacgacctgttcctggagaagctggagaagaagaacctggaccccgacaaggagtacgacgtgaccctgtacagcagcttcagcccctgctacgagtgctgcaagagactggccgagttcctggccaagcaccccaacgtgaagctgaccatcgtgacaagcaacctgtacaacgtggagcccggcagccccaaccccgagggcctgagactgctggccgctgctggcgtgaccatcagaatcgccacaagagccgacgccaagcacgtgtgggaaaccttcgtggacaacaacggcaagcccttcgtgccctgggaaggcttcgaggagaactccgcccggctcgccgctagactggccgccatcctgacccctcctgaagccgctgctgctgctgctgccgccgctgccgctgctggcaga

SEQ ID NO: 217 - DNA, 591 bp, synthetic construct
  source 1..591: mol_type = other DNA
cctagccccaagatgcccgaggccgtgttctacagagagttcgcccccgtggccgaccccaccggcagaaccacaacctacctgtgctacgtgctgaccctgaaggaccccgagaaggacgtggtgctggacaagggctggctgagagacaaggacggcgtgcacgtggagctcctgctcctggagaagctgagcaagctgcccctggaccccaacaagaagtaccacctggagctgtacctgagccacagcccctgctacgagtgcgccaaggccatcgccgagtggctgaagaagaaccccaacatcagcctgaccatccacatggctagaatctacgtggaccccaacgaccctagatgggccgagggcctgagactgctggccgaggccggcgtgaagatcagcatcatgacaagagccgacttcgagtacgtgtggaacaccttcgtggacaacaacggcagaccctacgtgccctttcccggcctggaggagaacagcgctagactggctgctgaactcgccgccatcctcacacctcccgaggccgctgccgctgccgccgctgctgctgctgctgccggcctg

SEQ ID NO: 218 - DNA, 591 bp, synthetic construct
  source 1..591: mol_type = other DNA
aagagccccaagctgcccgaggccaccttctactattacttcaaccccgtggagaaccccaccggcagaaccgagagatacctgctgtacgagctgaccctgctgaaccccgacaagaacgtggtgctggacaagggctggctgaaggacgagaacggcaagcacgtgtacgacatcctgctggagaagctgagcaagctgaacctggaccctagcaagaagtacaagctgaccctgtacacatccttcagcccctgctacgagtgcgccaagagactggccgagtggctgaagaagcaccccaacgtgagcgtggagatcaagatgagcagaatctacatcgaccccaacgaccctagatggaaggagggcctgagactgctgaaggaggccggcgtgaagatcagcatcatgaccgaggaggactacaagtacgcctgggaaaccttcgtggacaacaagggcaagaagtacgtgcccttcaagggcttcgaggagaacagcaagaagctgcagaaggagctggacgagatcctgaagcccgaggaggaaaagaaaaaggaggaagaggagaagaaaaagctgggcctg

SEQ ID NO: 219 - DNA, 591 bp, synthetic construct
  source 1..591: mol_type = other DNA
gagagccccctgatgcccaaggaaaccttctactattacttcgaccccgtggccaaccccgagggcagaaccgaaacctacctgtgctacgagctgaccgagctgaaccccgagaaggacgtggtgctggagagcggctggctgaaggacgagaacggcaagcacgtgtacctgctcctgctggagaagctgagcaagctgcccctggaccccgacaagagataccacctgagactgtacgcttcccatagcccctgctatgagtgcgctgaggctatcgccgcctggctggccgcccaccccaacgtgagcctggagatcgccatggctagaatctacgtggaccccaacgaccctagatgggccgagggcctgagactgctggccgaggccggcgtggagatcagagtgatgaccgccgccgacgccgagtacgtgtggaacacattcgtggataataacggcgaaccctacgtcgccccccccggcttcgaggagaacgccgctagactgcaagccgagctggacgagatcctgaccccccccgaggagaaggctgccgctgctgccgctgccgctgccgctggcctg

SEQ ID NO: 220 - DNA, 591 bp, synthetic construct
  source 1..591: mol_type = other DNA
agaagcagcaagatgcccgaggagctgttctacaagtacttctaccccaagaaggacagaaccggcctgaccacaacctacctgtgctacgagctgagaacccaagtggacgccaccacctacgaggtggagaagggctggatcgaggacgaggacggcgtgcacgcctacctgctgttcctggagaagttcagcgccctgaacctggaccccgacaagaagtacgacgtgaccctgtacgcctccttcagcccctgctacgagtgcgccaaggccctggccgagttcctgaaggcccaccccaacgtgagcatcaccatcaaggtggccaacctgtacaactgcgagcccggcagccccaacaaggagggcctgagactgctgaaggaggccggcgtgaccatcagcatcatggacgaggccgactacaagtacgtgtggaacaccttcgtggacaacaagggcaaggagtacgtgccccccgagggcctgaaggagaacagcgaggagctgcagaagaagctggaggagatcctgaccccccccgaggtggaggaggctagacggagagccctgagagaggccggcctg

SEQ ID NO: 221 - DNA, 591 bp, synthetic construct
  source 1..591: mol_type = other DNA
aagagccccctgatcgacgaaaagaccttctactattacttcaagcccgaggagaacgccgagggcagaaccacaacctacctgtgctacgagctcaagaccaaggtggacgagaacacctacgagatcgagagcggctggctggaggacgagaacggcaagtgcgtgtacctgctgttcctggagaagttcgagaagctgaacctggaccccgacaaggagtacaacatcaccctgtacagcagcttcagcccctgctacgagtgcgccgaggccatcgccgcctggctgaagaagcaccccaacgtgaagctgaccatcaagacaagcagactgtacaactgcgagcccggcagccccaacaaggagggcctgagactgctgaaggaggccggcgtggagatcagcatcatgacctacgaggacgccaagtacgtgtggaacacctacgtgaagaacaacggcaaggagtacgtgcccttcgagggcttcgaggagagaagcaagaagctgcaagccaagctggaggagatcctgaccccccccgaggagaaggccgctgccgccgctctggccgctgccctgggcctg

SEQ ID NO: 222 - DNA, 591 bp, synthetic construct
  source 1..591: mol_type = other DNA
aagagccccaagatcgacgaggaaaccttctacagatacttcaacaacgaggaggacagaaagggcctgaacaagacctacctgtgctacgagatcaagaccaaggtggacgagaacacctacgaggtggagaagggctggctggaggacgaggacggcaagcacgtggtggacctgttcctggagtacctgagcaagctgaacctggaccccgacaaggagtacgacgtgaccctgtacatgagcttcagcccctgctacgagtgcgccaagaagctggccgagttcctgaagaagaaccccaacgtgaagatcaccatcaaggtggccaacctgtacaactgcgagcccggcagccccaacaaggagggcatcagactgctgaaagaggccggcgtgaaggtgagcatcatgaccgaggaggactacaagtacctgtggaacaccttcgtggacaacaacggcaaggagtacgtgcccttcgagggcttcgaggagagaagcaagaagctgcaagccgagctggagaagatcctgaccccccccgaggagaagaaaaagctggaggagctgctgaaggagctgggcctg

SEQ ID NO: 223 - DNA, 636 bp, synthetic construct
  source 1..636: mol_type = other DNA
gaggctctcgccgccctggccgccctgctggctgccctcacacagcccgggcctgctgcccctgtccctcccgcccccccccctgctcccggcgcttgtcctcccgtgcctctgaagcccgatggctccgtggacctggccgccctgaacgccgagctggccgcccggggcgtgaccggcctggacggggtggaccctacccctgtccccgggtttgaccccgccgctttccaccccgcctgctccgccggcctggccccccggcaacccgggcagcccaccaccggcaagctgttcctgcccgacggccggagactgaacagcacccccctgaccggcaccggcggcaagagcgcctgggacctgtcctgcatcaagcctgagtaccgggatctggcccccgcccggggcgatctcgccggcaacgtggccgcctacgctagaagaaccgacatcaaggagctggtgctgtacatgaacgccgcccctagcaccctgaacggcaagggcagcctgtacagactgcacgacttcctgctgcccggctacaccctgtacgtgtggtggcaagacgcccaaggcaactggcagctgaccgtgtacaagggcaccggcgaggcctacgtgcccggcgtg

SEQ ID NO: 224 - DNA, 636 bp, synthetic construct
  source 1..636: mol_type = other DNA
gccgctgccgctgctgccgctgccgccgctgctgctgccgctgcccccgggcccggcgcccctgtcccccccgcccctgcccccgctcccggggccgctgctccccctcctagacggcccgacggcagcgtcgatagagctgctctggccgctagattcgccgctagagggctgaccggcctcgatggggtggaccccacccccgtgcccaacttcgaccccaacaagtacaaccccgccctgagcgagggcctggagcctagaaagcccggcgagcccgtgagaggcaagctgtacctgcccgacggcaagagactgaacaagacccccctgaccggcaccggcggcacaagcgcctgggacctggccgagatcagacccgagtaccgggacctggcccccgctagaggcgacctggagggcaacgtggccgccctgatgcgggacctggacatcaaggaggccgtgctggtgatgaacgccgcccctagcgacctgaacggcagaggcagcctgcacagactgaaggacttcctcagacccggctacaccctgcacgtgtggtacagagatgagaacggcgagtggaagctgaaggtgttcgagggcaccggcgaggccttcgtgcccggcgtg

SEQ ID NO: 225 - DNA, 636 bp, synthetic construct
  source 1..636: mol_type = other DNA
gccctcctggcccctctggccgctgccctcgccgccctgacagcccctggccccgctgcccctgtcccccctccccctcctcctgtccctggcgcctgtacccccgtccccctcagacccgacgggaccgtggatctggccgccctgaaggctgagctggccgctagaggcctgaccggcctcgatgggcgggaccccacccccgtgcctggcttcgaccccaacaagttccaccccgcctgcgtggagggcctggagcctcagaagcccggcgagcccacaagaggcttcctgttcctgcccgacggccggagactgaacagcacccccctgaccggcaccggcggcaagagctgctgggacctgagctgcattaagcccgagttcagagatctggctcccgctcaaggcgacctggagggctgtgtggccgcctgggctagaagaaccggcctgaaggagctggtgctgtacatgaacagagcccctagcaagctgaacggcaagggcagcctgtacaccctgaaggacttcctgctgcccggctacaccctgcacgtgtggtatcaagacgaggagggcaactggaagctgaaggtgttcaagggcaccggcgccgcctacgtgcccggcgtg

SEQ ID NO: 226 - DNA, 636 bp, synthetic construct
  source 1..636: mol_type = other DNA
gagaaggagagactggctgaaattctggctaagctcctgaaccctagcccccctcccgctcccgctcctcctaaacctccccctccccctcctccccctagacccctccccctgaagcccgatggcaccgtggacctggaggccctgagagccctgctgaaggccctgggcgtgaagggcctggagggcctggacctgacccccatccccaacttcgaccccaacaagtacaaccccgccgccaaggagggcctgccccctagaaagcccggcgagcccaccctgggcaacctgttcacccccgacggcgtgaaggtgaacaccaagcccctggccgcctccgacggcaagagcgcctgggacctgagcctgatcaagcccgagtaccgggacctggcccccgctagaggcgacctggagggcaacgtggccgccctgatgtacaagttcaacatcaaggaggccgtgctgtacatgaacgccgcccctagcaagctgaacggcaagggcagcctgtacaccctgaacaacttcctgcccaagggctacaccctgtacgtgtggtatcaagacgagaacggcaactggaagctcaagacctacaccggcaccggcgaggccttcatccccggcgcc

SEQ ID NO: 227 - DNA, 636 bp, synthetic construct
  source 1..636: mol_type = other DNA
gagagagagagactggctagacggctgcggagactcagacggccccggcccccccccgcccctcggccccctagacctagacggcggcccctgcccccccggcctctgcctagacggcctgatgggaaggtggacctcagagccctgagagccctgctgagagccctcggcgtggaaggcctggagggcctcgactggacccccgtgcccggcttcgaccctaacaagtacaaccccgcttgcagcgccggcctccccgctcagaagcccggcgagcccgtgcaaggcagactgttcagacccgacggcgtgagagtgaacgccgagcccctgacaagcagcgacggggtgagcgccttcgacctgagcctgatcaagcccgagttccgggacctggcccccgctagaggcgacctggccggcaacgtggccgcctacgcctgggagaacaacatcaaggagctggtgctgtacatgaacagagcccctagcaagctgaacggcaagggcagcctgtacaccctgaagaacttcctgcccaagggctacaccctgtacgtgtggtatcaagacgccaacggcaactggaagctgaagaagtacgagggcaccggcgaggccttcatccccggcgcc

SEQ ID NO: 228 - DNA, 636 bp, synthetic construct
  source 1..636: mol_type = other DNA
aagaaggaggagctggccaagaagctggagaagctgctgaaccccaaaaagccccccaagcccaagcccaagaagcccaagagaaagcccatcaagcctagaccccctcccctggacgagaagggcaagatcgacttcgaggccctgagaaagctgctgatcgagctgggcgtgaagggcctggagggcctggacaagacccccatccccaacttcgaccccaccaagtacaaccccaaggcctccgagggcctcgctcctcagaagcccggcgagcctacccaaggcagactgttcagccccaacggcgtgagactgaacaagaagcccctgacaagcagcgacggcgtgagcgcctgggacctgagcaagatcaagcccgagtacagagacaaggaccccgccaagggcgacctggtgggcaacgtggccgcctacgcctacagaaacaacatcaaggagctggtgctgtacatgaacagagcccctagcaccaacaacggcaagggcagcctgtacaccgccaaggacttcctgaagaagggctacaccctgtacgtgtggtacagagatgccaacggcaactggaagctgaaggtgtacaagggcaccggcaaggccttcatccccggcgcc

SEQ ID NO: 229 - DNA, 516 bp, synthetic construct
  source 1..516: mol_type = other DNA
gctcctgccgaagccgccgctgctgctgccgctgccgctgctgccctcgctgccgctctcggcctcggcggcccccccgccggcgccgccccccccgctctccccgaggaagaggtggacagagctctggctcagctgcctgcccccgtggtccccgggaccggccaaaaggtgcatggcctgtggaagggccccgacggcgagtggaagcccatcaccgagggcgagaacgagtacagcaagctggtgaatcccatcctggccgctaaaggggctcccggccccacccccctcgccgacctggtggagatgcagctggccgcctacatggccgccaacggcatccggcacgccaccgtggtgatgaacgccctgcccgaaaccggccccggcaaggccgacgaactcgtgcccatcatcctgccccctggcagcaccctgaccgtgcacggcatcgacgccgacggcaagagaagcagagtgaccttccacggcggcgccaagcccccctggcag

SEQ ID NO: 230 - DNA, 516 bp, synthetic construct
  source 1..516: mol_type = other DNA
gctcctgccgctgccgctgccgctgctgctgctgccgccgctgccctggctgccgccctcggcgtgggcggcccccccgccggccctgagccccctcccctccctgaggaagagatcgctgccgccctggccaccctcccccctcccgtggtccccgggaccgggcagagagtgcacggcctgtggagaggccccgacggcagatggcggcccatcaccgagggcgaggacgagagatccgccctggtgaaccccctgctggccgctagaggggcccctgggcctacacctctcgctaccgacgtggccctgaagctggccgcctacatggccgcccacggcatcagagccgctaccgtggtcgtcaacgccgagatgctgcggggccccggccgggccgatgaactggtccctgtgatcctgcctcccggggccgtgctgaccgtgcacggcgtggacgccgccggcagaagaagcagaagagtgttcagaggcggcgccacccccccctgggcc

SEQ ID NO: 231 - DNA, 516 bp, synthetic construct
  source 1..516: mol_type = other DNA
gtgcctgccgccgctctggctgctgccgctgccgctgccgctgctctcgccgctgctctcggcctcggcggccccgccgctggcgctgccccccccgctctgcccgaggctgagattgatgccctcctggctcagctgcccgcccccgtggtccccggcaccgggcagagagtgcacggcctgtaccgggacgcctccggcgagtggcggcccatcaccgagggcgaggacgaactgagcaagctggtcaaccccctgctggctgccctgggggctcccggccccacacctctggccaccctggtggagatgaagctggccgcctacatggccgccaacggcatcaagcacgccaccgtggtgatcaacgccctgatggagagaggccccggcaaggccgacgagctcgtgcccatcattctgcccgagggcgccacactgaccgtgcacggcatcgacgccgacggcagaagaagcagagaggtgttcagaggcggcgccaagcccccctgggcc

SEQ ID NO: 232 - DNA, 516 bp, synthetic construct
  source 1..516: mol_type = other DNA
gccgctgccgctgccgccgctgccgctgccgctgccctggctgccctggccgccgctctcggggtcggcgctcctcccgctggcgccgagcctcccgtggtgcctcaagccgagatcgacgccatgctcgctcaactgcctgcccccgtcgtgcccgggaccgggcagaaggtgcacggcctggtgaagggcgccgacgggcagtggcggcccatcgtgagcggcgagaacgagctgtccaagctggtgaaccccctcctggcttccctgggcgcccccgggcccacccccaacagcaccgccgtggagctgcagaccgccgcctacatggccgccaacggcatcaagcacgctaccgtcgtcgtgaacgccctgcccgccaagggccccgggaagcctgacgagctggtgcccatcatcctgcccgagggcagcaccctgaccgtgtacggcatcgacgccaacggcaacagaagcaaggtgaccttcaagggcggcaagaagcccgcctggcag

SEQ ID NO: 233 - DNA, 516 bp, synthetic construct
  source 1..516: mol_type = other DNA
gccgccgctgccgctgccgctgctgctgccgctgccgccgctgccgctgccgctgccctcggcgtcggggccccccccgccggggctgagcctcccgagctgcccgaggctgaggtggaggccctcctcgcccaactgccccctcctgtcagacccgggaccgggcagcgggtgcacgggctctggagaggcgccgacggcgagtggcggcccatcacaagcggcgaggacgagctgagcagactggtcaaccctctcctggctgctagaggcgcccccggccccacacctagagctacagacgtggagctgaagctggccgcctacatggccgccgagggcatcagacacgccaccgtggtgctgaacgccctgcccgctagaggccccggcaagcctgacgagctggtgcccatcctcctccccgaaggcgccacactcacagtgcacggcgtggacgccgccggcagaagaagcagagtgacctacagaggcggcgccacccccccctgggcc

SEQ ID NO: 234 - DNA, 516 bp, synthetic construct
  source 1..516: mol_type = other DNA
gccgctgccgctgccgctgctgccgctgccgctgccgccgctgctgccgctgccgctctcggcgtcggcgctccccccgccgggctggaaccccctagagtgcctgccgctgaagtggaacgggccctggcccaactcccccctcccgtcgtgcccggcaccggccaactggtgcacggcctgtggagaggccccgacggcagatggcggcccatcacaagcggcgagaacgagctgtccaagctggtcaaccccattctggccgctaagggggctcccggggatacccccctgagcaccctggtggagctgcagctggccgcctacatggccgccgagggcatcagacacgccacagtcgtggtgaacgccctgccccccaccggccctggcaaacctgatgagctcgtccccatcatcctgcccgagggcgcctccctgaccgtgtacggcgtggacgccgccggcaacagaagcgaggtgaccttcagaggcggcgccacccccgcctgggcc

SEQ ID NO: 235 - DNA, 636 bp, synthetic construct
  source 1..636: mol_type = other DNA
agcctcctcgctcccctcctcgctgctctcgctcccctggctgccgctccccctgcccccgcccctcccgtcccccccgctccccctccccctccccctagcgtcctgcccctgaaccccgatgggaccctggacctggaggccctgaaggccctgttcaaggccaagggcctgaccggcctggacggcttcagcgaaacccccgtgcccaacttcgaccccgacaagttcaaccccgcctgcttcgagggcctggcccctcagaagcccggcgagcccgtgagaggcagactgttcctgcccgacggcaagagactgaacagcgagcccctgacaagcaccaacggcacaagcatcttcgacctgagcctgatcaagcccgaatacagagatctggcccctgcccggggcgacctggccggcgccgtggccgcctacgccatctacctggacatcaaggagctggtgctggccatgaacagagcccctagcaagctgaacggcaagggcagctactacaagctgaaggacttcctgaaggagggctacaccctgtacgtgtggtacagagatgccaacggcaactggaagctgaagaagttcgagggcaccggcaaggccttcatccccggcgcc

SEQ ID NO: 236 - DNA, 636 bp, synthetic construct
  source 1..636: mol_type = other DNA
gctgccctggccgccctcgccgctgccgctgccgctctggccgctgcccctcctgcccccgctccccctagaccccccaagcctcccgcccctcctcctagcgtgctgcccctcagacccgatgggaccctggatctggaggccctgagagccgagtacagagccaagggcctgaccggcctggacggcgtgagcaccacccccgtgcccggcttcgaccccaccaagtacaaccccgcctgcttcgagggcctggagcctcagaagcccggcgagcccgtgaagggcagactgttcagacccgacggcacaagagtgaacagcacccccctgaccgacaccggcggcacaagcctgtgggacctgagcagaatcagacctgagtatcgggacctcgcccccgctcggggcgacctggccggcaacgtggccgccctgatgcgggacctgggcatcagagaggccgtgctggctatgaacgccgccccctccgacctgaacggcagaggcagcctgcacaccctgcacgacttcctgctgcccggctacaccctgtacgtgtggtatcaagacgagaacggcaactggaagctcaagacctacagaggcaccggcgccgccttcagacccggcgag

SEQ ID NO: 237 - DNA, 636 bp, synthetic construct
  source 1..636: mol_type = other DNA
gccgctgctgctgccgctgccgaagccgctgctaaggccgctgccgccccccccgcccccgcccctcccgtgcctcccgccccccctccccctcccccctccgtgctgcccctgaagcccgatgggacactcgatctggccgccctcaaggctctgttcgctgccaaagggctcaccggcctggacgggttcagcgaaacccccgtgcccgacttcgaccccgacaagtacaaccccgcctgcttcgagggcctggagcctagaaagcccggcgagcccacaagaggcaacctgttcctgcccgacggcaagagagtgaacagcgagcccctgacaagcaccggcggcacaagcctgtgggacctgagcaagatcaagcccgagtaccgggacctggcccccgctagaggcgacctgctgggctgcgtggccgccctgatgacctacctggactggaaggaggccgtgctgtgcatgaacagagcccctagcaagctgaacggcaagggcagcctgtacaccgccaacaagttcctgaagaagggctacaccctgtacgtgtggtacagagatgccaacggcaactggaagctgaagaagttcgagggcaccggcgaggcctacatccccggcgcc

SEQ ID NO: 238 - DNA, 678 bp, synthetic construct
  source 1..678: mol_type = other DNA
ctcgacgctgccctcgccgccgctagagccgctctggctgctgccaaagctcagctcgctgcccacgccgctgccgctgccgggagcaccgaccccgcccctgctgctgagctggctgctctcagagctcagctggccgccctggaggcccaactcgccgctctcgaagcccaaagaaaggccctggccgctgccgccgaggccctggctgccgcccctgccggccccctgaaaccccctcccgctcctcctcctcctcctccccctcccccttttgatcccacaaaggccgatcccgccaaggccgctctgatcccccccaaggtgggcgacgcccctagagtggccatcgtgttcgacgagaatggcaacgaagtgatgggcctgaccgccgatggcgccaccgggcccgccgctaccgccccttggaaggctccctacgcttcctacaccgccctgcgggccgatgtgctggcccatgctgccgccctggcctatcagaagggcctcaagaccgtgaccgtgtacacaaacgctgctcccgacacaagcgccaccggggctgccgctaacctggccgacgtgctgcccaagggcgccaccctgtacatccacctggtgcacgccgacggcagcaccaccctgaccaccgtgaagggcaccggcgccgcttgggcc

SEQ ID NO: 239 - DNA, 678 bp, synthetic construct
  source 1..678: mol_type = other DNA
ctggaggccgccctggccgctgccgaggctgagctggctcggctggaggccgagctcgccgctcaccgggccgctgccaagggctccaccgaccctgcccccgctgccgaactggccgccctggaggctagaatcgccgaggctagacggagactggccgctctggccgaggagaagaaaaaggccgaggaagagaagctgaagaaggagctgaagcccgagaaaaaggaaaagaagccccctaagcctgaacctccccctccccctcccgctcccgatcccgccgtcaccgatcctgcctgggctgctctgattccccctgccgtgggcgacgaccccgaaaccgccatcgtgcgggacgagaatggcaatgaggtgctcgggctgaccaccgccggcgccaccggccccgccgccacagctccctggctggccccctacagcaccttcgctagactgagagcctccgtgttcgcccacgccgctgccctgatgtatcagaagggcctcaagaccgccaccgtgtacacaaacaagccccccgataccgcccccgacggcgccgctaagaacctggccgacgtgctgcccaagggcgctaccctgtacgtgcacctggtgcacgccgacggcagcaccaccctctacgtgtacaagggcaccggcaaggcctgggcc

SEQ ID NO: 240 - DNA, 678 bp, synthetic construct
  source 1..678: mol_type = other DNA
ctggaggaaagactggaggccgccctggctgccctcgctgctgctgaggctgaactggccgccctgagagccgcctccgctgggtccgccgaccctgcccccgccgctagactcgccgccctcgaagccgagatcgctagactgagagaggagatcgctagactggaagctgagctggaagaaagaagaagagaggaagagagactcagaagcgagcctgaggaacctgaggaacctccccctctccccgccccccctccccctccccctccctttgatcccgagaagcccaatcccgagctcgctgccaaaatccctcccgccgtgggcgacgacgctagaaccgccgtggtgtacgacgagaacggcaacgaggtgctgggcctgaccacagacggcgctaccggccctgctgccacagccccttggaaggccccctacgcctcctacaccgagctgagagccgacgtgctggcccacgccgctgccctggcctatcagagaggcctcaagaccatcaccgtgtacatcaacaaagcccctagcacaagcgccaccggcgccgccaagaacctggccgccgtgctgcccaagggcgccaccatgtacatccacctggtgcacgccgacggcaccacaaccctgtacgtggtgaagggcaccggcagcgcctgggcc

SEQ ID NO: 241 - DNA, 504 bp, synthetic construct
  source 1..504: mol_type = other DNA
cccgccgctgcccctgctcctcccgctcctgctcctggcggcgctcccctccccccccctcctccttcccctgcccctagacccctgcccgacgccgaggtggaagccctcgccgctcagctccctgcccccgtcgtgcccggctccgggcagctggtccatgggctgtacgtggccgaggacggcgaggtgcacagcatcacaagcggcgagagcgccctgagcgagagagtgatcgccctgctggccgccaagggctggcctagacggcctagaaacgccggcgacgtggtgctgcagctggccgcctacatggtggcccacggcatcaagcacgtgacactggtcattaacgctgctcctagcaccggcacacccgactccgccgacaccctggtgcccgtcctcctccccgagggggccaccctggaggtccacggcagaaccgccgacggcgcccccgtgagaagagtgttcagaggcggcgccgcccccccctgggcc

SEQ ID NO: 242 - DNA, 504 bp, synthetic construct
  source 1..504: mol_type = other DNA
cccgctgccgcccctgccgcccctgcccccgcccctggcggcgctcccctgcccctgcctccccctagccccgcccccaagcctctgcccgaagaggagatcgaaagactcgctgctcagctgcctcctcccgtggtgcccgggaccgggcagctcgtgcacggcctgtacgtgggcgaggacggcgaggtgaaggagatcacaagcggcgagagcgagctggccgagctggtgagagagatcctcgccgccaaggggtggccccatgctcctagcaacgccgggggggtggagctgcagctggccgcctacatggtggccaacggcatcaagcacgtgaccctcgtgatcaacgccgcccctagcaccggcacacccgacagcgccgacaccctggtgcccatcatcctgcccaagggcgccaccctgaccgtgtacggcagaaccgccgacggcaagcccgtgagagtgacctacgagggcggcgccacccccccctgggcc

SEQ ID NO: 243 - DNA, 504 bp, synthetic construct
  source 1..504: mol_type = other DNA
agcgcccccgcccctgcccctcctgctcctgcccctgggggcgctcctgctcctctgccccctcctacccctgctcctaaggtcctccccgaagaggagattgccgccctggccgccacactgcctgcccccgtggtccccggcagcggccaacgggtccacggcagatgggtgggcgaggacggcgaagtgagatccctcacaagcggcgaggacgccctggccgagagagtcagagccctgctggccgctcaaggcatccccgaggcccccgaaaatgctggcggcgtggagctgaagctggccgccgagatggtggctagaggcatcaagcacgccaccctggtgatcaacgccgcccctagcaccggcacaccttacagcgccgacaccctggtccccgtgatcctgccccccggcgccaccctgaccgtctatggcagaacagccgccggcgagcccgtggtcgtgacctacgagggcggcgccacccccgcctgggcc

SEQ ID NO: 244 - DNA, 504 bp, synthetic construct
  source 1..504: mol_type = other DNA
cctcccgccccccctgtgcctgaagaggtcccccctggccccgggcccctccccgtccccgaggccggccccgaacctagacctctgcctgaggccgagatcgaggccctcatggctcagctgcctcctcccgtcgtgcccgggaccggccaaagaacccacggcctgtacgtgggcgaggatggcgaggtgcggagcattacctccgggtgggatgccctggccgacgccgtcagagccctcctggctgccctgggctggcctagagcccctagaaacgcccacggcgtggagctgaagctggccgcctacatggtggctcatggcatccggcacgccaccctggtgctcaatgccgctcctagcagaggcagacccgatagcgctgacgccctggtccccgtgctgctgcccgaaggggctgtgctggaggtgtacggcgtgaccgccgagggcagaagagtgagagtggtgtacagaggcggcgctagacccgcctgggcc

SEQ ID NO: 245 - DNA, 504 bp, synthetic construct
  source 1..504: mol_type = other DNA
ccccctccccctccccggcccgaagtgctgcccgagggcgccgctcccctccctgtgcccgagggggcccccctgcctagacctctgcccgctgctgaggtcgaaagactcgcctcccaactgccccctcccgtcgtgcccgggagcgggcagaaagtccacggcctgtacgtggacgcctccggggccgtgagaagcatcacaagcggggacgatgccgatgccgctgctgtcagagccctgctggctgccctggggtggcccgaggagcctacaagagccgacggcgtggagctgaagctggccgctagaatggtggctgagggggtccggcacgtcaccctcgtgatgaacgctgcccctagcaccggcgggcctgatagcgccgacgccctggtgcccgtgctcctgcctgagggggccgagctgctggtgtacggcagaaccgccgacggcagacccgtgagagtgctgttcagaggcggcgctagacccgcctgggcc

SEQ ID NO: 246 - DNA, 504 bp, synthetic construct
  source 1..504: mol_type = other DNA
ccccccgagcccgtgaaacctagagaactccccgccggcgctggcgccgtccctgtgcccgctgccggccccgcccctagacccctgcccgaggagagagtcgaggaactcttcgcccggctgccccctcctgtggtgcccgggagcgggcagagagtgcacggcctgtacgtggacgagtccggcgaggtgagagaaatcgtgagcggcgacgatgccctggccgccctggtgagagaaagactcgccgccctgggctggcccgaggagcctagaagagccgacgacgtggagctgaagctggccgctagaatggtggccgagggcatcagacacgtgaccctggtggtgaatgccgcccctagcagaggcgagcccgactccgccgacacactggtccctatcctgctgcccgagggcgccgtgctgagagtgtacggcagaaccgccgccggcgagagagtggtcgtggagtacagaggcggcgccaagcccccctggagc

SEQ ID NO: 247 - DNA, 501 bp, synthetic construct
  source 1..501: mol_type = other DNA
gccgccatcgtgaccgcccctgtcgtgcctctggtgtgggtcggcgacacacccgtcgtcgtgcctgatgggaccccccctgcctatattctggaggccctggccgagctcgccggcgagcctagagaaaccggcctgctgttcgacaagaacggcaacagaattggccctagaattaccgctggggacgaggaccccgagctgagagccggcgtggtggagctgctgagagccgacccctggtttagagcccaccacccccctgacaccgtgctgcccgtggctagaagagtggaggccaaggccgccgtgtgggccgtgagaaacgacatcaaggaggccatcctggtcatcaacagcgactccgtggggcctcccgctcctgaccccggcctgggccctgagacagccgtgccccacatcctgcccgagggctacaccctgaccgtgtggacaagcgacaagacaagcgtgaccctggtgggcaaaagacctagaccc

SEQ ID NO: 248 - DNA, 501 bp, synthetic construct
  source 1..501: mol_type = other DNA
gccgctgtcgtgaccgctcccgtccccgtcgtggtctgggtgggcgacgtccccgtcctcgtgcccaccgctaagcctcctgcctacgtgctggctgccctggccgagctggccggcaagaagagagaaaccggcctgatcttcgacagaaacggcaaccggatcggccccgagatcaccgctggggaagaggaccccgagctcagagctggcgtggtcgagctgctgcgggctgacccctggttccgggcccaccatccccccgataccgtgctgagagccgccctggacgtggcctgcaaggccgccgtgtgggccgtgagacacaacatcagagaggccatcctggtcaccaatagagatacagtcgggccccccgctcccgaccccgggctcgacgctagaacagccgtgccccacatcctgcccgagggctggaccctgaccgtgtggagaaaggacaagacaagcgaaaccctggtgggcagacggcctagaccc

SEQ ID NO: 249 - DNA, 501 bp, synthetic construct
  source 1..501: mol_type = other DNA
cctctggtcgtgctggcccccgtcccccctctggtgtgggtcggcgacgtgcccgtcgtcgtgcccaccggggagcctcctgccttcgtcagagaggccctggccgagctggagggcgaggagagagaaaccggcctgctgttcgacaagaacggcaacagaatcggccctagaatcgtggccggcagagaggaccccgagctgagagccgatgtggtggctctgctcagagctgatccctggttcagagctcatcatccccccgataccgtgctgcccgtggccgaggacgtggaggccaaggccgctgcctggatggtgaagaacaacatcaaggaggccatcctggtcatcaacagagatacagtcgggcctcctgctcccgctcctggcctgggccccgaagtcgccgtgccccacatcctgcccgagggctacaccctgaccgtgtacagacgggacggcacaagcgtgaccctggagggcaaaagacccaagccc

SEQ ID NO: 250 - DNA, 501 bp, synthetic construct
  source 1..501: mol_type = other DNA
gcccccctccccctgggcgtccctcctgtcctggtgcctgccccccccctccccctccctccccccgacgctcccacacctgcctgggtcaaagaggccctcgccgagctggccggcagcgagagagaaaccggcctgatcttcgacaagaacggcaaccggatcggccctagaatcaccgccggcgacgaggaccccgagctgagagccggcgtgcaagagctgctggccgccgacccctacttcagagcccacttcccccccgacagaagattcagagtggctagactggtggaggccaaggccgccgtgtgggccgtgaagaacaacatcaaggaagccgtgctggtcaccaatgctcctgtcgtcgggcccgcctccggcgacccccacgatacccctctcgtggccgtgcccctgatcctgcccgagggctggaccctgaccgtgtacagaagcgacgagacaagcgtggtgctgaagggcacaagacccctgccc

SEQ ID NO: 251 - DNA, 501 bp, synthetic construct
  source 1..501: mol_type = other DNA
gcccccgctcccgccgggacacctgtcgtcgccgtgcctgctccccccctccccctccctgcccccgccgaacccacccccgagtgggtgaagaaggccctggaggagctgaagggcagcaagaaggaaaccggcctgatcttcgacgagaacggcaacagaatcggccctagaatcaccgccggcgacgaggaccccgagctgagagccctggtgcaagagctgctgagagccgacccctacttcagagagcacttcccccccgacagacagttcagagtggctagaggcgtgaccgccaaggccgctgcctggtgcgtgaagaacaacatcaagaacgccgtgctggtgatcaacagcgacgtggtgggccccgaaaccggcgacccctacgacaccgacagagtggccgtgcccctcatcctgcccgagggctggaccctgaccgtgtacagaagcgacgagacaagcgtggtgctgaagggcaaaagacccctgccc

SEQ ID NO: 252 - DNA, 501 bp, synthetic construct
  source 1..501: mol_type = other DNA
ctgccctccgtggccggcgtccctgtgcctgtcctcctgaccccccccctccctctgcctccccctagcgctcccctgcccgaatgggtcagagaggccctggccgaactggagggcagccctagagaaaccggcagactgttcgacgagaacggcaacagaatcggccccctgatcaccgccggcgaggaggaccccgagctcagagccgaggtggagagactgctgagagccgaccccttcttcagagcccacttcccccccgacgctagattcagaaccgccctgggcgtggaggccaaggccgctgcctgggccgtgagacacaacatcaagaatgctgtgctcgtcatcaacgctgacgtggtggggcccgacgctggcgacccccacgacacccctagaatcgccgtgcccctgatcctgcccgagggctggaccctgaccgtgtacagacgggacgagacaagcgaaaccctggtgggcgaaagacctagaccc

SEQ ID NO: 253 - DNA, 459 bp, synthetic construct
  source 1..459: mol_type = other DNA
ccccccgtccctctgccccctctgccccctcccgccgctgccggcgcccctgctcctgggctgcctggctttgtggccgccgctctcgccgagctggccggcgagacaagagaaaccggcctgattttcgacgctgatggcagacggatcggccctagaattaccgccggccccggccctctcgccgctagagctcaagccctgctgagagcttccccccacgtgcctgccctgccccccgacgctgtgtacgccgccgctcggggcgtggaggcccaagccgccgtgtggatgagagagaacggcatcaaggaggcccatctggtgaccaacgctccctccgtcagcggcccccccctcggcagcgaaaccgccgtccccgccatcctgcccaagggcgccaagctgacagtgtggacccccggcgccggcgagcccctggtgctggagggcgtgggc

SEQ ID NO: 254 - DNA, 459 bp, synthetic construct
  source 1..459: mol_type = other DNA
cctctgcccctccccctgcccctgcctctgcccgctgctgccggggccccctcccccggcctccccgggtgggtggccgctgccctggccgagctggccggcagcagacggagaaccggcagactgttcgacgagaacggggagcaaatcgggcctctcctgaccgctggccctggccccctggctgctgctgccgaggagctgctcagagcctcccctcacgtccctgctctgccccctgacgctagatacgccgctgccctgggcgtggaggcccaagccgccgtgtggatgagagacaacggcatcagaagagctcacctggtcctgaatgctccctttgtgtccgggccccctctgggcgccgccaccgccgtccctgccatcctgcctagaggcgccgtcctcaccgtgtggtatcccggcgccggcgagcccctggtgctgagaggcgtgggc

SEQ ID NO: 255 - DNA, 459 bp, synthetic construct
  source 1..459: mol_type = other DNA
cctgtccctgtggtggctcccgcccccctgcccgctgccgctgggccccctgcccccggcctccctggctgggtcgcccgggccctggccgaactggccggcgaggagagagaaaccggcctgatcttcgacgagaacggcgagagaatcggccctcggctcaccgctggccctggccctctcgccgagagagctcaagagctcctgagagcctcccctcatgtgcctgccctccctcccggcgagcggtatgccgccgctctcggcgtggaggcccaagccgccgtgtggatgagagagaacggcatcaagagagcccacctggtgatcaatagagatcgggtgagcggcccccccctgggcgaggaaaccgctgtgcccgctatcctcccccggggcgccaccctgacagtgtggtatcccggggccggcgagcccgtggtgctggagggcgtgggc

SEQ ID NO: 256 - DNA, 459 bp, synthetic construct
  source 1..459: mol_type = other DNA
ccccccctccctctgccccccctgccccctcccccccctctgcctccccctgaccccaccatccctcccttcgtgtacgaggccctggccgagctggccggcagcgagaaggaaaccggcctgatgttcgacgagaacggcaaccggatcggccctagaatcaccgccgggcctggccccctcgctgctagagctcaagagctgctgagagcctcccctcatgtgcccgccctccctcccgacgccgtgtatgctgctgcccggggggtggaggctcagttcgccgtgtgggctagagagaacggcatcaagaaggcccacctggtgaccaacgccgatagcatcagcggcccccctaccggggaggaaaccgccgtgcctgccatcctgcccaaaggcgccaccctgaccgtgtggcaccccggcgccggcgagcccaccgtgctggagggcgtgggc

SEQ ID NO: 257 - DNA, 459 bp, synthetic construct
  source 1..459: mol_type = other DNA
ggcctgctcgtcgtgccccccctgcctcctcccctgcctccccctccccctagccctggcattcctccttgggtgctggaggccctggaggagctggccgacagcgagaaggaaaccggcctgctgttcgacgagaacggcaacagaatcgggcctagaattacagctgggcctggccccctggccgagcgggccgaagctctgctgagagcctcccctcacatccccgctctgccccctgatgccgtgtacgaggccgcctaccacgtggaggctcagtacgccgtgtggtgcagagagaacggcatcagaagagcccatctggtgattaacgccgacagcgtggctggccctcctctgggcgatagagctgccgtgcccgccatcctgccccggggcgccgtgctcacagtgtggacccccggcgccggcgcccccgtggtgctgagaggcgtgggc

SEQ ID NO: 258 - DNA, 459 bp, synthetic construct
  source 1..459: mol_type = other DNA
ccccccgtccccctgccccccctgccccctcctcctcctgccgctgctcccgaccctgctctgcctcccttcgtgcgggaggccctggctgagctggccggcgctagacggagaaccggcctgctgttcgacgagaacggcagaagaatcggcccccgggtcaccgctggccccggccctctggccgaaagagctgaggagctgctccgggcttccccccacgtgcctgccctgccccccgatgctcggtatgccgctgctagaggcgtggaggctcagttcgccgtgctgtgcagagagagaggcatcagacgggcccacctggtcaccaacgccccctttgtctccgggccccccaccggggaggccaccgctgtccccgccatcctgcctagaggcgccgtgctgagagtgtggtatcccggcgccggcagacccctggtgctggagggcgtgggc

SEQ ID NO: 259 - DNA, 636 bp, synthetic construct
  source 1..636: mol_type = other DNA
gccgctgaggagctggccgccctgctcgccgccctggccgaccctagaccccctcccgcccctgctccccccgcccctcctcggccccccctgccccctcggcccctgcccctgacacccgatggcaaggtggacctggctgctctcagagctctcctgctggctctgggcgtggccggcctggaggggctggacctgacccccgtgcccaacttcgaccccaacaagttcaaccccgcctgcagcgagggcctggcccctagaaagcccggcgagcccgtgaagggcagactgttcctgcccaacggcgtgagagtgaacaccacccccctgacaagcagcgacggcaagagcgccttcgacctgagcaagatcaagcccgagtaccgggacctggaccccgctagaggcgacctggagggcaacgtggccgcctacgccacaagaaacaacatcaaggagctggtgctgtacatgaacgccgcccctagcaccaacaacggcaagggcagctactacaccctgaacaagtttctgcccaagggctacaccctgtacgtgtggtatcaagacgagaacggcaagtggaagctgaagaagtacgagggcaccggcgaggcctacatccccggcgcc

SEQ ID NO: 260 - DNA, 636 bp, synthetic construct
  source 1..636: mol_type = other DNA
gctgctgaaagactcgctgccgctctcgctgccctgctgacccctgccccccctcccgctcctgctcctcccgccccccctccccctcccgtgcctcccctgccccctcctctcagacccgatgggaaggtggacctggccgccctgagagccctcctcagagccctgggcgtggagggcctggaggggctggacaccacccccatccccaacttcgatcccaacaagtacaatgccgctgctgccgctggcctgcctgcccaaaagcctggcgagcccacccaaggcaacctgttcctgcccaacggcgtgagagtgaacaccacccccctgacaagcagcaacggggtgagcgccttcgacctgagcgagatcaagcccgagttcagagacaaggaccccgcccaaggcgacctggccggcaacgtggccgcctacatgatcagaaacaacatcaaggaggccgtgctgtacatgaacagagcccctagcaagaacaacggcaagggcagctactacaccctgaaggacttcctgcccaagggctacaccctgcacgtgtggtacagagatgccaacggcaactggcagctcaagacctacaagggcaccggcgccgccttcatccccggcgcc

SEQ ID NO: 261 - DNA, 636 bp, synthetic construct
  source 1..636: mol_type = other DNA
gccgctgccgacctggctgccctcctggccgccctgctcacacccgccccccctcccgcccctgctccccctccccctccccctccccctctgccccctagacctctccctagaacccccgacggcagagtggacctggaggccctgagagccctgctgagagccctcggggtggaaggcctggagggcctggacaccacccccgtgcccaacttcgaccccaccaagtacaaccccgcctgcagagagggcctggcccctagaaagcccggcgagcccacccaaggcagactgttcctgcccaacggcgtgagagtgaacaccgagcccctgaccgcctccgacggcgtgagcgccttcgacctggccgacatcaagcccgagtacagaggcctggaccccgctagaggcgacctgatcggcaacgtggccgcctacatgagaagattcggcatcaaggaggccgtgctgtacatgaacagagcccctagcaccaacaacggcaagggcagcctgtacaccatgaaggacttcctgcccaagggctacaccctgtacgtgtggtatcaagacgccaacggcaactggaagctgaagaagtacgtgggcaccggcgccgccttcgtgcccggcgcc

SEQ ID NO: 262 - DNA, 636 bp, synthetic construct
  source 1..636: mol_type = other DNA
gctgctctggccgctgccgctgccgctctggccgccctggcccccgccgccccccccgtgcccgtccctgccccccccgctccccctccccctgccgccgacgtgctgcctagaaaggagaacggcgaggtggactgggaggccctgaacgccctgctgaaggctagagggatggagggcctggacgggctcgatcccacccccgtgcccggcttcgatcccgcccggcccaaccccgcttgcttcgagggcctggctcctagagtgcccggcgagcccacaagaggcagactgttcagacccgacggcagaagagtgaacggcacccccctgaccgcctcctccggcaagcccctgtgggacctgagcctgatcaagcccgagttccgggacctggcccctgctagaggcgacctggtgggcaacgtggccgcctacatgagatacaccgacatcaaggaggccgtgctgtacctgaacagaccccctagcgacctgaacggcaagggcagcctgcacaccgcccccaagttcctgcccaagggctacaccctgtacgtgtggtacagagatgccgagggcaacgtgagactgagaagattcgagggcaccggcgaggccttcgtgcccggcgcc

SEQ ID NO: 263 - DNA, 636 bp, synthetic construct
  source 1..636: mol_type = other DNA
gctgctctcgctgctgctgccgctgccgctgccgccgctgccgctgccgccccccctgtgcctgtcccccctccccctcccccccctccccccgaggaggacgtgctgcccctgaagcccgacgggacagtggactgggaggccttcaacaagctgatggagagcgagggcttcaagggcctgaagggcctggaccccacccccgtgcccaacttcgaccccaacaagcccaaccccgcctgcttcgagggcctggagcctagaaagcccggcgagcccgtgaagggcttcctgttcagacctgacggcacaagactgaacgacgagcccctggagagcagcagcggcaagagcctgttcgacctgagcaagatcaagcccgagtacagaaagctgaagcccgcccaaggcgacctggagggcaacgtggccgcctacatgatcaagagaaacatcaaggaggccgtgctgtacatgaacgccccccctagcgacctgaacggcaagggcagcctgcacaccctgaaggacttcctgcccaagggctacaccctgtacgtgtggtacatcgacgagaacggcaacgtgaagctgaaggtgtacaagggcaccggcaaggccttcatccccggcgcc

SEQ ID NO: 264 - DNA, 636 bp, synthetic construct
  source 1..636: mol_type = other DNA
gccgctgctgccctcgctgctgctctgctggccgctgccgctcccgccgccccccccgtccctgtccccccccctcctcccccccctccccccaagggcagcgtgctgcctagaaagcccgacggcaccatcgactgggaggccttcaacgccctgctgaagagcctgggcttcgagggcctggacggcgtggaccccacccccgtgcccaacttcgaccccgagaagtacaaccctgcttgcgccgtcggcctggagcctcagaagcccggcgagcccaccaagggcagactgttcagacccgacggccggagactgaacagcacccccctgaccgagagcagcggcaagcccctgttcgacctgagcaagatcaagcccgagtaccgggacctggcccccgctagaggcgacctggagggcaacgtggccgcctacatgatcctgagaaacatcaaggaggccgtgctgtacatgaacagagcccctagcaccctgaacggcaagggcagcctgtacaccctgaaggacttcctgcccaagggctacaccctgtacgtgtggtatcaagacgagaacggcaactggaagctgaagaagttcgagggcaccggcgaggccttcatccccggcgcc

SEQ ID NO: 265 - DNA, 678 bp, synthetic construct
  source 1..678: mol_type = other DNA
ctggaggagaagctgaaggaagccgaagagaagctcaaagaagccaaggaagagctggaaaagctgaagaaggagtacgagggcagcaaggacgagagccccaaggagaagatcgaggagctggaggagaagatcaaagagctggagctggagctgaagagactggaggaagagaagaaaaagctgctggagaaactgaagaagctgctcgaaaaacccggcgctgctgaagagcccacccccgccgtgccccctccccctcctccccctccttacgaccccgagaagcccgaccccgctctggctgctaagatcaagcccgccgtgggcgacgacgccaccgaggccatcctgtacgacgcccaaggcaatctggtcctcggcctcaccaaggatagcaccaccggccctgctgccaccgctccctggaaggccccctacgcctcctacaccaagctgagagcctccgtgctggcccacgccgctgccgccgcttataaactcggcctcaagaccgtgaccgtgtacaccaacaaggcccccgacacaagcgccgatggcgctgccaacaacctggccgacgtgctgcccgagggcgccaccctgtacgtgcacgtggtgaaggccgacggcagcaccgagctgctggtgtacagaggcaccggcgccgcctataag

SEQ ID NO: 266 - DNA, 678 bp, synthetic construct
  source 1..678: mol_type = other DNA
ctggaggaagagctggagagagctagagaggaactggagcgggccgagcgggagctggaggagctcagagctagacacgctggcagcacctcccctgagcccgctgctgaactggccgccctggaggctagaatcgccgagctgagagcccggctccggagactggaggaagagagagaggagctggagcggaaactcagagaagagctggagaaggaggaggaagaagaggagcctgaaccccctgtccctcctcctccccctccccccccttttgaccctgccgtcgtggaccccgccctggccgccaagatcaagcccaaggtgggcaacgacaagaccgaggccatcctggtggacgagaacggcaatgaggtcctgggcctcaccaccgatagcagcaccggccccgctgccaccgctccctggaaggccccctacgccaactacaccgagctgagaaacagcgtgtacgcccacgccgctgccgagatgtacagaagaggcctcaagaccgtgaccatgtataccaatcagcccctggacacaagcgccaccggcgctgctaacaacctggccaacgtgctgcccaagggcgccaccctgtacatccacgtggtgcacgccgacggcagcgccaccctgaccaccgtgaccggcaccggcgccgcctacgcc

SEQ ID NO: 267 - DNA, 678 bp, synthetic construct
  source 1..678: mol_type = other DNA
ctggaggaaaaactggccgccgctgaggctgaaagagccgctgccgaggctgagctggccgccctcagagctgccgctgccggcagcaccgaccctgctcctgctgccgctctggctgccctcgaagctagactggccgctctggacgccgagctgaagagactggaggccgagaagaaaaagaaggaggaagaggagaagaaggagaaggagaagcccgagcctaaagccaaacccaaacctcccaaaccccctccccctccccctcccccctacgatcccgaggtgcctgaccctgctctggccgctcagatcccccctaaggtgggggacgctcccaccaccgccatcctgtacgacgccaatgggaaccaagtgctcgggctgacaaccgatggcagcaccggccccgccgccacagccccttacaaggccccctacgcctcctacaccaacctgagaaacagcgtgctggcccacgctgccgccaccgctagaaagctgggcctcaagaccgtgaccgtgtacaccaaccaagcccccgacacaagccccaccggcgccgccaacaacctggccgatgtcctgcccgagggcgccaccctgtacatccacgtggtgcacgccgatggcagcaccaccctgaccaaggtggagggcaccggcgccgcttacaag

SEQ ID NO: 268 - DNA, 516 bp, synthetic construct
  source 1..516: mol_type = other DNA
gccgggcctgctgctgccgctgctgctgctgctgccgccgctgccgccgctgccgctgctggggccggggctggcgccgccggcgccgagcctcctgaagtgcccgaggctgagattgccgctgccctggctcagctgcctccccccgtcagacccggcaccggcgaaagagtgcacggcctgtggagagggcctgacggcgagtggcggcccatcaccgccggcgagaacgagctgagcaagctcgtgaatcctcaactggctgctctcggggcccctggccccacccccaacgctagaggcgtggagatgcagctggccgcctacatggccgcccacggcatcagacacgccaccgtggtgatcaacgccaaacctggccgggggcctaatcgggccgacgtgctcgtccccgtcctgctgcctgccggcagcaccctgaccgtctatggcgtggacgccgacggcagacccgtgagagtggtgttcagaggcggcgccacccccccctggcag

SEQ ID NO: 269 - DNA, 516 bp, synthetic construct
  source 1..516: mol_type = other DNA
gccggccccgctctggctgccgctgccgctgccgccctggctgccctggctgccgccctcggcctgggccccgggcccggggggggcgagccccccgtggtgcctgaggaagagatcgaggccatgttcgccaagctgccccctcccgtggtgcctgggaccggcgaaaagacccacggcctgtaccgggacgagagcggcgagtggaagcccatcaccgagggcgaggacgagtggagcaaactggtgaaccccattctggcctccctcggcgcccctggccccacacccctcgccaccgacgtggagatgaagctggccgcctacatgagagagcacggcatcagacacgccaccgtggtgatcaacgccgagcccctgcaaggccccgacaaggccgaggtgctggtgcccatcatcctggaccccggcgccaccctgaccgtgtacggcgtggacgccgacggcagaagagtgagagtgacctacagaggcggcgccacccccccctggcag

SEQ ID NO: 270 - DNA, 516 bp, synthetic construct
  source 1..516: mol_type = other DNA
ggccctctggaggccctgagagagctggccgaggccctggccgctgccctggccgcctccggcgtgggcgctggcgctggcggcgctgaacctcccgaggtgcccgaagaggagatcgaggccgccctggcccaactccccccccctgtggtgcccgggaccggcgagagagtgcacggcctgtacagaggcgaggacggcgagtggcggcccatcaccgagggcgagaacgagctgagcaagctcgtgaaccccctcctggccgccctgggggcccccggccctacccctaacgccaccctggtggagatgcagctggccgcctacatggccgccaacggcatcagacacgccaccgtggtgatcaacgccgccccccctagaggccccgacagagccgaggtgctggtgcccatcatcctgcccgagggcgccaccctgaccgtgtacggcgtggacgccgacggcagaagaacaagagtgacctacagaggcggcgccaagcccccctggcag

SEQ ID NO: 271 - DNA, 516 bp, synthetic construct
  source 1..516: mol_type = other DNA
gcccctcccgccctggccgctgctagagccgccgctgccgctgccgctagagccgctgccggcgctgggggcgctcccgctctggacgcccctcccgctgtgcctgaagccgaagtggctgccatggccgctcagctgccccctagagtgcggcccggcaccggcgagagagtgcacggcctctggagaggcgccgacggcgcctggcggcccattaccgaagggcctagcgaatggagcgctctggtcaaccctctgctcgctgccctcggcgcccccggccccacccccaatgccaccgacgtggagatgcaactcgccgcccatatggccgccaccggcgagcggcacgccaccctggtgctcaacgccacagtcagcaccggccccggcgaagctgacgaactggtgcctatcctcctgccccccggcagcacactcaccgtgcatggctacgacgctgctggcgcccccacaagagtgaccttcagaggcggcgccacccccccctgggcc

SEQ ID NO: 272 - DNA, 516 bp, synthetic construct
  source 1..516: mol_type = other DNA
ggcacacccgctctggccgctgccgccgctgctgctgccgctgccgctagagctgccgccggcgctggcgccgcccctgctgccggcgagccccccgaggtgcccgaggccgaggtggaggagatgagagccaagctgccccctcccgtgacccccggcaccggcgagaaggtgcacggcctgtacaagggcgaggacggcgagtggaagcccatcgtggagggcgagaacgagtggagcaaactggtgaaccccctcctggccgccctgggcgcccccggcgacaccccctgggccaccgacgtggagatgcagctggccgcctacatggccgcccacggcatcagacacgccaccgtcgtgatcaacgccacagtggccaccggccccggcagacctgacgagctggtgcccatcctgctgcccgaaggcgccacactgaccgtgtacggcgtggacgccgccggcaaccccacaagaaccgtgttcagaggcggcgccgagcccccctggaga

SEQ ID NO: 273 - DNA, 516 bp, synthetic construct
  source 1..516: mol_type = other DNA
gctcctcccgctgccgccgctgccgccgctgccgctgccgccgctgccgctgccgccgctggggccggcggcgctcctgaagccggcgccccccccgaactccccgaagaagaggtggaagccctgagagctcagctgccccctcccgtcacccctgggaccggggagaaggtgcacggcctgtacagaggcgccgacggcgagtggaagcccatcaccgagggcgagaacgagtggagcaaactggtgaaccctatcctggccgctaagggggcccctggccctacccccaatgccaccgacgtggccatgcagctggccgctagaatggccgccgagggcatcagacacgccaccgtcgtcatcaatgctaccctggccaccggccccggcaagcccgatgagctcgtccctatcatcctggcccccggcagcaccctgaccgtgtacggcgtggacgccaacggcaaccccacaagaaccgtgttcagaggcggcgccacccccccctgggcc

SEQ ID NO: 274 - DNA, 516 bp, synthetic construct
  source 1..516: mol_type = other DNA
ggcggggctgctgccgctgccgccgctcgggccgctgctcgggctgccctcgccgccgctgctgctgccggcgcccccgggctgagcgccgccccccccctccccgaggctgaggtggctgccgctctcgctacactgcctccccccgtggtccccggcaccgggcagagagtgcacggcctggtgcgggacgaggacggcagatggcggcccatcgtgagcggcgagaacgagctctccgccctcgtgaaccccattctggcctccctgggggcccccgggcccacccctctcgccaccgacgtggagctgcaaaccgccgcctacatggccgccgagggcatcagacacgccaccgtggtcgtgaacgccctgatgagaagaggccccgggcagcccgacgagctggtgcccatcatcctccctgagggcgccaccctgaccgtccacggcgtggacgccgccggcagaagaagcagaagagtgttcagaggcggcaagacccccgcctgggcc

SEQ ID NO: 275 - DNA, 516 bp, synthetic construct
  source 1..516: mol_type = other DNA
gccgggcctgctctcgctgccgctgccgaggccgctagacgggccgccctggctgccgctgccgccggcgggagccccgggagcagcctcgcccccgaggtgcccgaagaggaagtcgaagctgccctggctcagctgccccctcccgtggtccctgggaccgggcagagagtgcacggcctggtgagactggaggacggcgagtggcggcccatcacaagcggcgaggacgaactgtccgccctcgtgaaccctctgctggccgctctcggggcccccgggcccacccccctggccaccctggtggagctgaagctggccgcctacatggccgctagaggcatcaaacacgccaccgtggtcgtgaacgccctgcccctgagaggccccggcagacccgacgaactcgtgcccatcctgctgcccgaaggcgccaccctgaccgtgtacggcgtggacgccgccggcagaagaagcagaagagtgttcagaggcggcgccgtgcccccctggcag

SEQ ID NO: 276 - DNA, 516 bp, synthetic construct
  source 1..516: mol_type = other DNA
gccggcgccgctgccgctgccgctgctgccgccgctagacgggccgctctggccgctgctaaagccgctgcctcccccgccgctgccgaagccccccctctccccgaggaagaaatcgaggccgctctggccaaactgccccctcccgtcgtccccgggaccgggcagagagtgcacgggctgtggagaggcgccgacggcgagtggcggcccatcacaagcggcgagaacgccctcagcgctctcgtcaaccctctgctcgctgccctcggggcccctggccccacccccctcagcaccgacgtggagctgcagctggccgcctacatggccgccaccggcgagagacacgccaccgtggtgatgaacgccctgatgagaagaggccctggcagacccgacgagctggtccctgtcctgctgccccctggctccaccctgaccgtgtacggcgtggaccacgagggcagaagaagcagaagaaccttcagaggcggcgccacccccgcctgggcc

SEQ ID NO: 277 - DNA, 678 bp, synthetic construct
  source 1..678: mol_type = other DNA
ctggaagaggagctgaagaaagtggagaaggagctggaagaagccaaaaaggaactggaagagctgaaaaagaagagcgagggcagcaccgaccccgagtggaagaaaaagctggaggagctggagcggaagatcgaggaactggagctggagaaggaggagctcaagaaaaagatcgccgaggaagaggctagactggctgccctgctcaatcccggcgcccctctgccccctctgcctccccctgtgcctcccccccctcctccccctccctttgaccccaccaaggctaaccctgctctcgccgccaagatcacccccgccgtgggcgacgaccccgaggaggccatcgtgtacgacaagaacggcaacctggtgctgcccctgacaagagacagcgacaccggcctggccgccaccgccccctggaaggagccctacaagaactacgagaagctgagaagaagcgtgtttgcccacgccgctgccgccatgctgaagagaggcctgaaggagatgaccgtgtacaccaacaagcccctggacacaagcgaggacggcgccgccaagaacctggccaacgtgctgcccaagggcgccaccctgtacatccacctggtgcacgccgacggcagcaccaccctgaccaaggtggagggcaccggcagcgcctacgcc

SEQ ID NO: 278 - DNA, 678 bp, synthetic construct
  source 1..678: mol_type = other DNA
ctggaaaagaagctgaaggaagctgagaagaagctggaagaagccaagaagaaactgaaggagctgaaagaaaagagcaagggcagcaccgacaaggagcccgagaaggagctggaggagctggagagagagatcgagaagctggagctggagatcaaggagctgaaggaggaaaaggccgccctggaggccaagctggctgctctgctgaacaaggggggccctgccgtcgtgctgcccgcccccgtccctccccctcccccccctccccctttcgatcccaccaagaccgaccccgccctggccgctaagattacccccaaggtgggcgacgaccccaccgaggccatcctgtacgacgccaacggcaacctggtcctgcccaagacctacgacagcgacaccggggaggctgctacagccccctggaaggccccctacgccaactacaccaacctgagaaagagcgtgctggcccacgccgctgccgccatgagaaagagaggcctcaagaccatgaccatgtacatcaacgccccccccgacacaagccccgacggcagcgccaagaacctggccaacgtgctgcccaaggacggcaccctgtacatccacgtggtgcacgccgacggcagcaccaccctgtacgtgttcaagggcaccggcagcgcctgggcc

SEQ ID NO: 279 - DNA, 678 bp, synthetic construct
  source 1..678: mol_type = other DNA
ctggaggagaagaaggaagaggtggagaagagactgaaggagctggagaaggagctgaagaagctgaaggaggagagcgagggcagcaccgaccccgagcccgaggaaaagatcagagagctcgaaagagagatcgagctgctgaagctggagctgcgggagctggaggagaagatcaaggaggaagaggcccggctggctgccctgctgaatcccggcgagcccgctcctgtcctcccccctcctgtcccccctccccctccccctcccccccacgatcctgccgtgcccgaccccgctctcgccgccaagatcaagcccaaggtgggcgacgacgagagagaggccatcctggtggacgccgacggcaatctcgtgctgcccctgaccacagactccgccgacggccccgccgccacagccccttggctgcccccctgggccgactataccgagctcagacgggacgtggccgcccacgccgctgccgctgcccacgagagaggcctgagagaggtgaccgtgtacacaaaccaacctctgtccacaagccccaccggcgctgcccacaatctggctgccgtcctgcccgccggcggcaccatgtacatccacctggtgcacgccgacggcagcacaaccctgaccgtggtgagaggcaccggggccgccctgcct

SEQ ID NO: 280 - DNA, 678 bp, synthetic construct
  source 1..678: mol_type = other DNA
ctcgacgccgccctggccgctgctagagccgctctggctgccgccgaggctagactggccgccctggccgctgccacagccggcagcaccgaccccgcccctgctgccgaactggccgctctcaaagccgagctggccgccctccgggctagactggccgctctggaggctgctcgggctgccgctgccgctgccgctgctgctgctctggctggggctgcccccgctccccctgtccccccccctcccgccccccctccccctccccccccttttgatcccaccgtgcccaaccccgctctggccgctaagatcacccccaaggtgggcgacgacaaggaggaggccatcctggtggacgccaacggcaacgaggtgatgggcctgacctacgacggcgagaacgggcctgccgccacagccccctggctggccccctacgccgactacaccgagctgagaaagagcgtggccgcccacgccgctgccgtgatgagagacaagggcctcaagaccgccaccatgtacaccaataagcccctgagcacaagcgccaccggcgccgccaagaacctggctgccgtcctgcccaagggcggcaccctgtacatccacgtggtgcacgccgacggcagcgccaccctgtacaccgtgaagggcaccggcagcgcctacgcc

SEQ ID NO: 281 - DNA, 678 bp, synthetic construct
  source 1..678: mol_type = other DNA
ctggaagaggaactgaaaaaggccgagaaggagctggaaaaagccaaaaaggagctggaggagctcaagaagaaaaccaagggcagcaccgaccccgagcccaagaaaaagatcgccgagctggaggagaagatcaaagagctggagaagaaactgaaggaactggaggagaagaaaaagaaaaaggaggaagagctgaagaaactcctggagaaggaggagcccgtgccctacgtgcctcccgtgcctgtgcctcctcctcctcccgcccctcgggaccccaccaaaaccgatcctgaactggccgctcagatcacccccgccgtgggcaacgacaagagagccgccatcctgtacgacgagaacggcaacgaggtgctgggcctgacatacgatgggagcaccggcctggccgctaccgccccttggctggccccctacgccaactacaccgagctgagaaacgacgtgctggcccaagccgctgccgccatgtacaagaagggcctcaagaccgtgaccgtgtacacaaacaaggaacctagcacctccgccaccggcgctgccaagaatctggccaacgtgctgcccaagggcgccaccctgtacgtgcacgtggtgcacgccgacggcaccacaaccctgctggtgtacaagggcaccggcgccgcctacgcc

SEQ ID NO: 282 - DNA, 678 bp, synthetic construct
  source 1..678: mol_type = other DNA
ctggaggagaaactgaaggaggccgagaaaaagctcaaggaagctgaggaagagctgaaaaagctgaaggagaaaaacaagggcagcaaggaccccaagcccaaggaggagatcaagaagctggaggccgagatcgagaagctcaaggccgaactgaaggagctgaaagagcaaaaagaggccgagaaggctgccctggctgccctgctcggggccgccgctcccgctccccccgtgcctgccgcccccgccccccctccccctcctgcccccttcgaccccaccaagcctgaccctgctctggccgccaagatcacccccgccgtgggggacgctcctgaagaggccatcctggtggacgccgatggcaaagaggtgctgggcctcaccaccgcttccgctaccggccccgccgccaccgccccttggctcgctccctgggccgattacaccgccctgagagcctccgtgtacgcccacgccgctgccgagatgtacagaagaggcctcaagaccgccaccctgtacaccaacaaagctcctagcaccgctcccgatggcgccgccaagaacctcgccgacgtgctgcctgccggcgccaccctgtacattcacgtggtgcacgctgacggctccacaaccctctacaccgtgaagggcaccggcgccgcctacgcc

SEQ ID NO: 283 - DNA, 678 bp, synthetic construct
  source 1..678: mol_type = other DNA
ctcgacgccgagctggccgccgctagagctgccctggctgccgctagagctagactcgccgccctgagagccgcccacgccggctccgctgaccctgcccctctggccgaaattgccgccctggaggccgagctggctagactggaggctagaatcgccgccctgagagccgagagaagaagaagagctgccgctagaagaagaagagccgctcctgctgcccctgccccccggcctcctcctccccctccccctcccccccctccccctccttacgatcctacagagcccgaccccgccctggccgctctgatcccccctagagtgggggacgcccctagagtcgctatcgtggtggacgagaacggcaacgaggtgctggggctgaccgctgatggcgccgatggccccgctgctaccgccccttacaagcccccctgcgccgactacaccgagctcagacgggacgtgttcgcccacgccgctgccctgatgagacagagaggcctcaagaccgccaccgtgtacacaaacctcccccccgacacaagcgaaaccggggctgccaaaaacctggccgacgtgctgcccaagggcgccaccctgtacgtgcacgtggtgcacgccgacggcagcaccaccctgctggtgtacaagggcaccggcaagtgctggagc

SEQ ID NO: 284 - DNA, 678 bp, synthetic construct
  source 1..678: mol_type = other DNA
ctcgccgaggccctggctgctgctgaagcccggctcgctgctctgaaggctcaactggccgccgctgaagctgcccatgccggcagcgccgatcctgctcctctcgccgagctggccgccctgagagccgccatcgctcgggccgaacgggagatcgccgagctcagagccgctgccgccgctgccaaagccgctagagaggccctggaggctgctgaggagcctgcccctccccctccccctccccctccccctccccctcctccccccccttttgaccccaccgtgcccgatcccgccctgctggctagaatcccccctagagtgggcgacgaccctagagtggccgtggtgtacgacgccaacggcaacgaagtgctgggcctgaccgctgacggcgctaccggccccgccgccacagctccctacaaggccccctgggccaactacaccgagctcagagccgacgtgtacgcccacgccgctgccctgatgcacaagagaggcctcaagaccgtgaccgtctataccaacctgcctcccgatacaagcgccaccggcgctgccaacaacctggccgacgtgctgcccaaggacgccaccatgtacatccacgtggtgcacgccgacggcagcgccaccctggtggtcgtgaagggcaccggcagcgcctggagc

SEQ ID NO: 285 - DNA, 678 bp, synthetic construct
  source 1..678: mol_type = other DNA
ctggaggctagaagagccgaggccgaagccgccctggctgctgctagagctgagctggaagccctccgggccgagcacgccgggtccacagatcccgctcctgctgccgagctggccgccctcaaggctcggctcgctcggctggaacgggagctcgccgccctggccgaggaggaagccgccctggctagacaaagagccgctgccgaagctcctggcgctcctgaacctgccccccctccccctccccctccccctccccctccccctccctttgaccccaccaagcccgaccccgagctgctggctagaatcccccccaaggtgggcaacgcccctagacaagccatcgtgtacgacgagaatggcaacgaggtcctggggctgaccgaggacggcgaggacggcgaggccgccaccgccccctacaaggccccctgggccaactacaccaacctcagacgggacgtgctggcccacgccgctgccctggctagaaagagaggcctcaagaccatcaccgtgtataccaacctcccccccgacacaagcgacgacggcgccgctaagaacctgagcgccgtgctgcccaagggcgccaccatgtacgtgcacgtggtgcacgccgacggcagcgccaccctgtacgtgttcaagggcaccggcagcgcctggagc

SEQ ID NO: 286 - DNA, 504 bp, synthetic construct
  source 1..504: mol_type = other DNA
cctagacctgtccctcctatcgtcctgctggaacccgggcctgctcccgctcctccccctcccgctgaagggatgctcccccctctgcccgaggctgaagtggaaagactggccgctcagctgccccctcctgtcgtgcccggcaccgggcagagagtgcacgggctgtacgtgggcgccgacggcgccgtgagagagattacaagcggcgaggacgagctggccgagctggtgcgggagatcctggccgctaaagggtggcccgaggcccccgagaatgccggcgacgtggagctgaagctggccgccctgatggtgaaggagggcatcaaggacgtgaccctggtgatcaacgccgccgtgtccaccggcacccctgatagcgctgacgccctggtgcccgtgatcctgcccgagggcagcttcctgagagtgtacggcagaaccgccgagggcagaagaaccgtgagaaccttcagaggcggcgccacccccccctgggcc

SEQ ID NO: 287 - DNA, 504 bp, synthetic construct
  source 1..504: mol_type = other DNA
cctagacccctgcctcctattgtcctcctggagcctgggcccgaacctgaacctccccctcccgaggaagggcggctgcctcctctccccgaggagagaatcgctgccctggctgctcagctgccccctagagtcgtgcccgggaccggcgagaaaacccacggcctgtgggtgggccccgacggcgaggtgaagagcattacaagcggggacgacgaactcgccgaggccgtgcggagactgctggccgccctggggtggcctgctgcccctgagaacgccgacgacgtggagctgaagctggccgcctacatggtggagaacggcatccgggacgtgaccctggtgatcaacagcgaggtgagcaccggcacccctgacagcgccgacgtgctcgtgcccgtgatcctgcccgagggcgcctacctgagagtgtacggcttcacaagagaggggcagcccaccgtgagaaccttcgtgggcggcgccacccccccctgggcc

SEQ ID NO: 288 - DNA, 504 bp, synthetic construct
  source 1..504: mol_type = other DNA
ccccctcccctgcctgctcccgtcctcctcgctcccggccccgagcccgggcctcctccccccgaggccggccggcggccccccctgcccgaagccgaagtggctgccctcgccgctagactgccccccctcgtcagacccggcaccggcgagagagtgcacggcctgtacgtgggcgaggacggcgaggtgagagagatcgtgtccggcgataatgctctggccgccgctgtgcgggccctgctggccgctctggggtggcccgagcccccccggaacgctggcgacgtggccctgcagctggccgcctacatggtggagaacggcatcaaggacgtggagctggtgattaacgccgccttcggcagaggcacccctgattcccctgatgtgctggtccccgtgctgctgcccgagggccactacctgaccgtgcacggctttgatgccgagggcagacccgtggtcgtcacactggtgggcggcagaacccccccctgggcc

SEQ ID NO: 289 - DNA, 504 bp, synthetic construct
  source 1..504: mol_type = other DNA
agcctgcccggggctctgggggccgcccccctccccggccccgctcccctccctcctccccccgggagcgacgcccccctggtcctgcccgaggaagaggtggaggccctgatggccaagctgccccccatcgtgaagcccggcaccggcgaaaagacccacggcctgtacgtgggcgaggacggcgaggtgaaggagatcacaagcggcgagaacgagctgagcaagctggtcaaggagatcctgaagagcaagggcatccccgaaacccccaagaacgccggcgacgtggagctgcagctggccgcctacatggtgaagaacggcatcaagcacgccaccctcgtgattaacgccgcccctagcaccggctaccccgacagcgccgacaccctggtgcccatcatcctgcccaagggctacaccctgaccgtgcacggcgtgaccgccaagggcgagagagtggtcgtgacctacgagggcggcaagacccccccctggagc

SEQ ID NO: 290 - DNA, 504 bp, synthetic construct
  source 1..504: mol_type = other DNA
gccgaggaagaggaagaagaggaagaggaagagcctgaacctgaacccgaacctcccccccctgggagccccggccccatccctctgccccaagagcggatcgaagagctggccgctcagctcccccccctggtcgtccccggcagcgggcagagaacccacggcctgtacgtggacgagagcggcaaggtgagagagatcgtgggcggcgagaacgagctggccgctctggtcagagagctgctggctgccaagggcattcccgaaaccccccccaatgccgacggcgtggagctgcagctggccgcctacatggtggccaacggcatcaagcacgtcacactggtgatcaacgccgctccctccaccggctacccctacgctgccgacgccctggtgcccgtgattctgcccgccggcagcgtgctgaccgtgtacggccgggacgcccaaggcaaccccgtggtcgtgacctacgtgggcggcgccacccccgcctgggcc

SEQ ID NO: 291 - DNA, 504 bp, synthetic construct
  source 1..504: mol_type = other DNA
gccgccctgggcgctgccggcgctgctcctccccctgggcccgctcccgagcctcctcctcctggcgctcctgcccctagagaactgcccgaggctagaatcgaagccctggccgctagactgccccccctcgtcagacccgggaccggcgagcgggtgcatggcctgtgggtgggcgaggacggcgaggtgagagagatcacaagcggcgagaacgagctggccgagctggtgctgagactgctggcctccctgggcattcctgagaagcctcccaacgccgacggcgtggagctgcagctggccgcctacatggtggccaacggcatcagacacgccaccctggtgatcaacgccgcccctagcaccggctacccctacagcgccgacaccctggtgcccatcatcctgcccgagggctggaccctgaccgtgtacggcagaaccgccgacggcagacccacaagagtgaccttcagaggcggcgccacccccccctgggcc

SEQ ID NO: 292 - DNA, 504 bp, synthetic construct
  source 1..504: mol_type = other DNA
gccgctgccctgctggaactcctggccgccggcgccgcccccgggcccgctcctctcccccccgggctgggcctgcccccccctctgcccgaggccgagatcgctgccctggccgcccaactccctcctcccgtcagacccggcagcggcgagcgggtgcatggcctgtacgtggccgaggacggccaagtgagaagcctgaccggcggcgaggacgccctgagcgccctggtgagagagattctggccgccctgggcgctcctgaggaacctcccaatgccggcctggtggagctgaagctggctgccctgatggtgagaaacggcatcagaggcgtgaccctggtgatcaacgcccccgtggacagaggcgagggctacagcgccgaggagctggtgcccgtgatcctgcccaagggcgccaccctgacagtccacggccgggacgccgccggcagacccgtgagaagaaccttcgtgggcggcaagaagcccgcctggcag

SEQ ID NO: 293 - DNA, 504 bp, synthetic construct
  source 1..504: mol_type = other DNA
gccgccgctgccctggccgctctgctggccggggctgagcccgctcccgggcctcccccccctgctgccggcctccctcccgaactgcctgccgagagagtggctgccctcgccgctcagctccctcctcctgtcgtgcctggcagcggcgagctggtgcacggcctgtacgtggacgagagcggcgaggtgagagagattaccggcggggaaaacgaactgagcgaacgggtggtcgctctgctggccgctctgggcgctcccgaaagaccccccaacgccggccacgtggagctgcagctggccgcctacatggtggagaacggcattagaggcgccaccctggtgatcaacgcccctcccggcagaggcgagggctacagccccgaggtgctggtgcccgtgatcctgcccgaaggggccgtgctgagagtgcacggccgggacgccgacggcagacccgtggtgagagaatacagaggcggcgccgcccccccctgggcc

SEQ ID NO: 294 - DNA, 504 bp, synthetic construct
  source 1..504: mol_type = other DNA
gccgctgccgctctcctcgctctcctcgccggcgccctccctgggcccgggcctctcccccccgatctgggcctgccccccgaactccccgaggagagaatcgctgccctggccgctcagctgccccctcccgtccggcccggcagcggcgagagagtgcacggcctgtacgtggacgaggacggcgccgtgagagagatcgtgggcggcgagaacgagctgagcgagctggtgaagagaatcctggccgccaagggctaccccgaggagcccgagaacgccgaccacgtggagctgcagctggccgccctgatggtggagaacggcatcagaggcgtgcacctggtgatcaacgcccccgtgggcagaggcgaaggcgacagccccgaggtgctggtgcccatcatcctgcccgagggcgccaccctgaccgtgtacggccgggacgccgccggcagacccgtgagaagagtgttcagaggcggcgccgcccccccctgggcc

SEQ ID NO: 295 - DNA, 501 bp, synthetic construct
  source 1..501: mol_type = other DNA
ctggaactggctgaaggccctcccctgctcctgcccaaggaagggggcgctcctgtccctctgcccctgcccaacgtgcccgcctgggtcgaggccgctctcgccgaactcgccggcagccctagagaaaccggcctgctgttcgacgagaacggcaacagaatcggccctagaatcaccgccggcgacgaggaccccgagctgagagctagagtggtggagctgctcctggccgacccctacttcagagccaactacccccccgaggccgccgaggaggcctccaagctggtggagtgcaaggccgctgcctggatggtggccaacaacatcaagagagcccacctggtgatcaacagcgacgtggtgggccctagaagaggcgagagaagaacaagccccctggaggccgtgccccacatcctgcccgagggctggaccctgaccgtgtacagaagagacagaacaagcgtgaccctggagggcagacggcctagaccc

SEQ ID NO: 296 - DNA, 501 bp, synthetic construct
  source 1..501: mol_type = other DNA
cccctgcccctgctcgggcctcctctgcctctccccctggctggccccgtccccctgcccgagcccctgctggacatccccgagtacatcaaggaggccctggaggagctgaagggcaacaagagagaaaccggcctgatcttcgacgagaacggcaacagaatcggccccaccatcaccgccggcgacgaggacgccgagctgagagccggcgtgagagagctgctcctggccgacccctacttcagagcccactacccccccgaggccgccgaagaggtgagcaagagagtggaggccaaggccgcctactggatgctgaagaacgacatcaagaaggccatcctggtgattaactccaaggtggtcggccccgacagaggcgctgccgctgcctccgccgaagaggccgtgcccctgatcctgcccgagggctacgagctgaccgtgtacagagccgacaagacaagcgtgaccctgaagggcaaaagacccaagccc

SEQ ID NO: 297 - DNA, 501 bp, synthetic construct
  source 1..501: mol_type = other DNA
cctcccctgctggctggccctcctctccctctgctgcccgctggcagcgtccccgtccccctcgacctgagcgccgtccccgagtgggtcctcgaagccctggccgaactcgccggcagccccgaggaaaccggcctgatcttcgacaagaacggcaacagaatcggccctagaatcaccgccggcgacaaggaccccgagctgagagccggcgtggtggaaaccctggccaagaacccctacgccaaggagaactacaagcccgaggacttcgaggaggcctccaagagagtggagtgcaaggccgccgtgtggatgctgaagaacaacatcaaggaggccatcctggtgatcaacagcgacgtggtcggccccgatagaggcgctagaggcgcctcccctctcaccttcgtgcccctgatcctgcccgagggctacaccctgaccgtgtacaccgccgacagaagaagcgtgaccctgaagggcaaaagacccaagccc

SEQ ID NO: 298 - DNA, 501 bp, synthetic construct
  source 1..501: mol_type = other DNA
gctctcctcctccccccccctcctgtgcctctcctgcccctcggcgatgtccccgtccctctgcccgacgctgacgtgcccgagttcgtgaaggaggccctggccgagctgaagggcagcaagaaggaaaccggcctgatcttcgacaagaacggcaacagaatcggccctagaatcaccgccggcgacgaggaccccgagctgagagccggcgtggtggccctgctgagagccgacccctactttagagcccacgctcccgccgaccacgtgtggcgggtggccctgggggtggaggccaaggccgccgtgtggatggtgaagaacgacatcaaggaggccatcctggtgatcaacagcagcagcgtgggcgccaacaccgacgaccccctggaccaagctaccctggctgtgccccacatcctgcccgagggctacaccctgaccgtgtacacaagcgacgagaagagcgtgaccctggtgggcgaaagacctagaccc

SEQ ID NO: 299 - DNA, 501 bp, synthetic construct
  source 1..501: mol_type = other DNA
gccgccctgctgccccctccccctctcctgcccgtgcccctcggggacgtgcccctccctctgcccctgcctcctgcccccgagttcgtgctggaggccctgaaggagctggagggcagcaagaaggaaaccggcatcatcttcgacctgaacggcaacagaatcggccccaccatcaccgccggcgacagcgaccccgagctgaacgccggcgtggtggagctgctgaaggccaacccctacttcaaggccacaagacccgccgactacgtgtggaacgtggccaagctggtggaggccaaggccgccgtgtggatggtgaagaacaacatcaaggaggccatcctggtgatcaatgccgacagcgtgggcgctaatacaagcgaccccctggatcagccctccctcgccgtgccccacatcctgcccgagggctacaagctgaccgtgtacaccgccaacaaggagagcaccgtgctggagggcacaagacccaagccc

SEQ ID NO: 300 - DNA, 501 bp, synthetic construct
  source 1..501: mol_type = other DNA
cccctggccctgcccccccctcccctgctcgtcctccctctcggcgacgaacccctgcctctgcccgccgctgccgtgcctgaatgggtcagagaggccctcgctgagctggccggcagcgagagagaaaccggcctgatcttcgacgagaacggcaacagaatcggccccaccatcaccgccggcgacgaggaccccgagctgagagccggcgtggtggagctgctgagagccaacccctacttccaagcccacgcccccgccgactacgtctggagaagcgccctgggcgtggaggccaaggccgccgtgtggatggtgaagaacaacatcaagaaggccgtgctggtgatcaacgcccctagcgtgggcgctagaaccgacgaccccctggaccaagacaccctggtggtgccccacatcctgcccgagggctacgagctgaccgtgtggagaagcgatgagacaagcaccaccctgaagggcacaagacccaagccc

SEQ ID NO: 301 - DNA, 501 bp, synthetic construct
  source 1..501: mol_type = other DNA
ctggtggtggacctgggccctgaagagctcgtggtgcccgtcggcgacgtccctgtccctgtggagcccggcgagccccccgagttcatcaagaaggctctggaggagctgaaggggaaagagaaggaaaccggcctgatcttcgacgagaacggcaacagaatcggccccgagatcaccgccggcgacgaggaccccgagctgagaaagggcgtgatcgagctgctgaaggccgacccctacttcaaggccaacttcccccccgaggccttcgagatcgtgagcaagctggtggaggccaaggccgccgtgtacatggtgaagaacaacattaagaaggccattctggtgatcaacaccaaggtggtgggcagccctagcaatcccgagaaggcctcccccctgatcgccgtgccccacatcctgcccgagggctgggagctgaccgtgtggagaagcgacgagacaagcgtgaccctgaagggcaaaagacccaagccc

SEQ ID NO: 302 - DNA, 501 bp, synthetic construct
  source 1..501: mol_type = other DNA
cccctcctgctcctgggccctctccctctcctcctgcctctgggcgacgtccccgtgcctgtgcctccccctgaaccccctcccttcatcaaggaggctctggaggagctgaagggcaaggaaaaggaaaccggcctgatcttcgacaagaacggcaacagaatcggccccgagatcaccgccggcgacgaggaccccgagctgagaagaggcgtgatcgagctgctggccgccgacccctacgctagagccaacttcccccccgaggccttcgagagagtggccaagctggtggaggccaaggccgccgtgtggtgcgtgaagaacaacatcaaggaagccatcctggtgattaacagcgacgtggtgggccccgaagaggaccccgagcccgcctcccccgagctggccgtccctctgatcctgcccgagggctggaccctgaccgtgtggacaagaaacgagaagagcgtggtgctcaagggcaaaagacctagaccc

SEQ ID NO: 303 - DNA, 501 bp, synthetic construct
  source 1..501: mol_type = other DNA
agcgtggtgctgctgggccctctggaggaagtggtgctgctcggcgacgaacccgtcgaagtggagcccgaggagccccccgagtacatcctgaaggccctggaggagctgaagggcagcgagaaggaaaccggcaagatcttcgacaagaacggcaacagaatcggccccaccatcaccgccggcgacagcgaccccgagctgagagccgccgtgatcgagctgctggccgccgacccctacttcaaggagaactacccccccgaggcctgggagagactgagcaaggacgtggcctgcaaggccgctgcctggatggtgaagaacaacatcaaggaggccatcctggtgatcaattccgacgtggtgggcccccccgacgaccccgagggcgcctcccccgaggatgccgtgcccctgatcctgcccgagggctacaccctgaccgtgtggagaagcgacaagacaagcgtgaccctgaagggcaaaagacccaagccc

SEQ ID NO: 304 - DNA, 459 bp, synthetic construct
  source 1..459: mol_type = other DNA
cctgtggtcgtgctgcctggcgtgcctcctccccctcccctcgccgcccctagccccgacatccctccctgggtggtcgaagctctcgctgaactggccggcagcgagagagaaaccggcctgctgttcgacgagaacggcaatcggatcgggcctagaatcacagctggccctggccccctcgccgagcgggctcaagagctgctgagagcttccccccatgtccctgccctcccccctggcgctagatacgccgctgccctgggcgtggaggctcagtacgccgtgtggatgagagagaacggcatcagaagagcccacctggtgacaaatgcccctagcgtgtccgggccccccctgggcagcagaacagccgtgcctgccatcctgcctagaggggccaccctgaccgtctggcatcctggcgccggcagacccgtggtgctggagggcgtgggc

SEQ ID NO: 305 - DNA, 459 bp, synthetic construct
  source 1..459: mol_type = other DNA
cccgtcgtcgtcaacctgggggtgccccctcccgcccctcccgctgctcctagccccgacatccccccttgggtggtggaggccctggctagactggccggcaagaagagagaaaccggcgtgctgttcgacgagaacggcaacagaatcggccccgagatcacagctggccctggccccctggctgctagagctcaagagctcctgagagcctccccccacgtccctgctctgccccctgacgctgtgtacgctgccgccttcggcgtggaggctcagtacgccgtgtggtgcagagagaacggcatcagaagagcccacctggtgaccaaccggagctccgtcagcggcccccccctgggcgaaacaaccgccgtgcccgccatcctccctagaggcgccacactgaccgtgtggtatcccggcgccggcagacccgtggtgctgagaggcgtgggc

SEQ ID NO: 306 - DNA, 459 bp, synthetic construct
  source 1..459: mol_type = other DNA
cccgtcgtccctctgcccggcgtcccccctcctctgcccctggctgctcctagccccggcatccctccctgggtggtcgaagctctggccgagctggccggcagcagaagagaaaccggcgtgctgttcgacgagaacggggaacggattgccgaaagagtgacagctggccccgggcccctcgctgagagagccgaagctctgctgagagcttccccccatgtccctgccctgcctcccggcgagagatacgccgctgctctgggcgtggaggctcagttcgccgtgctgatgagagacagaggcgtgagaagagcccacctggtgaccaaccggccttacgtgtccgggcctcctctgggcgagcggaccgccgtgcccgctatcctccctagaggcgccgtgctgaccgtgtggacccccggcgccctggagcccgtggtgctggagggcgtgggc

SEQ ID NO: 307 - DNA, 588 bp, synthetic construct
  source 1..588: mol_type = other DNA
gagttcagagccatcttcgaccctagcgccttccccaaggtgacctacctgctgtacgagatcaagtggggcagcagcaccaagttctggagaaagtggtgccccaacacccccacacagcacgccgagatcaactgcctggagaacgacttcaaggccatccaccacagagcctccgtgagatgcagcatcatctggttcctgagctggagcccctgcggcctgtgctgcagactgatcatccacttcctgagagcccaccccaaggtgagcctgcagatctacatcggcagactgttcagacaccgggacgagagaaacagacaaggcctgcgggacctggtgagcagcggcgtgaacgtgtacatcatgagcctgcccgcctacgactactgctggagaaccttcgtggaccaccaagtgagagaggacgacatctactggccctgctacttcaccccctggatgatcttctacatgctggagtttcagtgcatcctgcaagtgggcagcacaagcctgttcaccgacctggccgaggagctgcactggctgacctgcttccccaacagattcacaagaagcagc

SEQ ID NO: 308 - DNA, 576 bp, synthetic construct
  source 1..576: mol_type = other DNA
atggagccctggcctagcctgccccccaaggcctacaacttcaacttcagaaacctgagagaggcccccaacagaaacagcacctacctgtgcttcgaggtggaggaagagcagctggacagccccgagagcagctactggggcgtgttcagaaatcagcctagccacctgcaccaagtgtaccccgaggagcctctgcacgccgagcagagattcctgagctggttctacgacgccctgctgagcgcccacgcctactacagagtgacctggtacatgagctggagcccctgcagcgagtgcgccgaagaggtggtgagcttcctgggcgagcacgagaacgtgagcctgagcatcagcgcctcccggctgtacaagtgcgacgaccaagatcagcaagagggcctgagactgctggaccaagccggcgtggaggtggccatgatgagccccgacgacttcgagtactgctgggacaacttcgtggacaacagacggcggagcttcagatactggaagggcatcagaagaaactaccacgccctggacgacgagctgaaggagatcctgtggggc

SEQ ID NO: 309 - DNA, 573 bp, synthetic construct
  source 1..573: mol_type = other DNA
ctgacaagcaacagcctgaagagcctgttctgcttcagatggtacatccccaaagaggtgctgaaggagcagttcgaccctagcaagttccctagagaaacctacctgctgtgcaagattcagtggggcgacaacggcacaagctggaagcactgggtgaagaactacaacgacgatgacagacacgccgaggtgtgcttcctggaggacgtgttcctgatgaagagcaacaacgtgaactgcaacatcaccctgtacctgagctggagcccctgcgccgactgcagctgcagcatcctggacttcctgaagcagcacagcaacgtgaccatcagcatctacgtggctagactgtactgcgtgaacagcgaggagaacagaaagggcctgcggagactgcaccacagcccccaagtgaacctgagcgtgatggagatgagaggcaagtgcttcaactgctggagaaccttcacaagaggcggcgacgcctacgacttctggcctagaaacaacaagaccgagatcatcgtgaacctggtgaccctgagaatcatcctggaggtgagcagactg

SEQ ID NO: 310 - DNA, 564 bp, synthetic construct
  source 1..564: mol_type = other DNA
atggaggacaaccccgcccctagcggcagaagcctgatggacgtggacaccttcaacagcaacttcggcatcatctgggccaccgaaacctacctgtgctacgaggtggaggtgagagaggaggacacctgggtgcccgtggagggcctgcaaggctttctgagaaaccaagtgtactgccacgccgagctgtgcttcctggacagagtgcctagctggctgctggacgagggcaagcagtacagactgatgtggtacatcagctggagcccctgccccgactgcgcccccaagctggtggacttcctgagaaagaacagccacgtgcagctgagaatcttcgccgctagaatctacaccaagctggacggctacgaggacggcctgcggagactgcaagacgccggcgccaagctggccatcatgacccccaaggagctgcagcactgctgggacaccttcgtggacaaccaagggcagcagttcgagcccgagctggagctgctggagaagatcagagctagatgtcagagactggagaacatcctgagaatccaaggcaac

SEQ ID NO: 311 - DNA, 549 bp, synthetic construct
  source 1..549: mol_type = other DNA
atggacagatacaccttcatcgagaacttcaacaaccaaggctgccccttcaagacctacctgtgctacgaggtggagagactggacggcgtgatccccctggacgagtacaagagattcgtgtgcaacaagggccccgatcagccccacggcccccaccacgccgagttctacttcctggagcagatcagaagcagaaacctggatcagaatcagagatacagactgacctgcttcatcagctggagcccctgctacagctgcgcccacaacctgaccaccttcctgaaggagaacagacacatcagcctgagaatcttcgcctcccggctgtacacaagcaacagattcggcgactatcagaagggcctgtgcgagctgcaagccgccggcgctcagatcaccatcatgacaagccaagacttcgagcactgctgggaaaccttcgtggaccacagaggcaacccctttcagccctgggagggcctggaggccaagagccaagagctgtgcgagaagctgcaagccatcctgagaacacagcagaac

SEQ ID NO: 312 - DNA, 597 bp, synthetic construct
  source 1..597: mol_type = other DNA
agcctgaccgccgacagcgaccccctgccctgcagcaacagcctgtacagaccccctagaagctgcctgatgcagagaaagaagttcctgtaccactacaagaacgtgagatgggccaagggcagacacgaaacctacatgtgctacgtggtgaagagaagagagagcgctcagacaagcagcctggacttcggcttcctgagaaacaccgctagagcccacgccgaggtggtgttcctggactacatcagagactggcccctggaccccggcctgacctacagactgacctggttcagcagctggagcccctgctacgactgcgccgctagagtggtgcacttcctgcaagctagacccaacgtgagactgagactgttcatcgctagactgtactactgcagcgagggcatgagacagcccgagggcctgagacagctgcaccaagccggcgtgggcctgggcatcatgacctacaaggactacttctactgctggaacaacttcgtggccaacaaggagaagagcttcaaggcctgggagggcctgcacgagaacgccgtgagactcagcagactgctgagaagaatcctggag

SEQ ID NO: 313 - DNA, 618 bp, synthetic construct
  source 1..618: mol_type = other DNA
acaagcagcatcaagggcagcagaaagggcgacggcggcctgcagatcattcagcccaaggagttcaaggacgtgtacgaccctagcgtgctgcccaaggtgacctacctgctgtacgagatcaagtggggcaacagcaccaagatctggagaaactggtgcagaaacacaagcacacagcacgccgagatcaacttcctggagaacgccttcagagagcggagcttcaaccccctgacccactgcagcatcacctggttcctgagctggagcccctgctggaagtgcagccaaagcgtggtggagttcctcaagacctaccccaaggtgaacctggagatgtacgtggctagactgttctggcacgaggacagaagcaacagactgggcctgcgggacctggtgatgaacggcgtgaccatcagagtgatggacctgagcgcctacaactactgctggagaaccttcgtgagccatcagaacgtggacgactactggccctggcacctgaccccctggatcatgttcttcagcttcgagctgcagctgatcctgcaagtgagcagaagcctgggcggcaaggacagcattcagaagctgagcagagacatgtacaga

SEQ ID NO: 314 - DNA, 1089 bp, synthetic construct
  source 1..1089: mol_type = other DNA
atgctgctgagcgtgcacaaggtgaccctgttcttcagccctcagatcatgtaccccgaggcctcccaccgggacggctacgaccctagcgtgctgcggagagtgcagtacatgttctacgaaatcaagagaagcaacagcaagagaagctggcagagctgctgccacagcacaagaaccgagcacgctgagatccacttcatcgaggacgtgttccaagagcagagaagcgatcctagcgtccactgctccatcacctggtacatgagctggagcccttgcggcgactgctgcaaggagatcagagacttcctgaaggagcagcccaacgtgaacctggtgatctatgtggcccggctgtactggcacaaggaggaaaccaacagacaaggcctgagaagcctgatgaacatcggcgtgagcatccgggtcatggacctccccgagagacagtggtgctggaacaacttctacagcggcggcgccgagggcggcaaccacgtgttcagactgctcctgattcagcccaaggagttcaaggacgtgtacgacccctccgtcctgcccaaggtgacctacctgctgtacgagatcaagtggggcaacagcaccaagatctggagaaactggtgcagaaacacaagcacccaacacgccgagattaattttctggagaacgccttcagagagcggagcttcaaccccctgacccactgcagcatcacctggttcctcagctggagcccctgctggaaatgcagccaaagcgtggtggagttcctcaagacctaccccaaggtgaacctggagatgtacgtggctagactgttttggcacgaggacagaagcaacagactgggcctgcgggacctggtgatgaacggcgtgaccatcagagtgatggacctcagcgcctacaactactgctggagaaccttcgtgagccatcagaacgtggacgactactggccctggcacctgaccccctggatcatgttcttcagcttcgagctgcagctgatcctgcaagtgagcagaagcctgggcggcaaggacagcattcagaagctgagcagagacatgtacaga

SEQ ID NO: 315 - DNA, 804 bp, synthetic construct
  source 1..804: mol_type = other DNA
atggccaccgagagagccgaaaccctggtggccgaccccaccatgcggagaagaatcaagccctgggagttcaccgccttcttcgaccctagccaactgagaaaggagacaagcctgctgtgggaggtgtggtggggcagcagcagcagactgtggagaagctgcagccaaaacagcgtgcaccacgtggagcggagcttcctggatcagttccaagctcagagacacctgggccctagcaccacctgcaccatcacctggttcctgagctggagcccctgctgggagtgcgctagagccatgcaagtgttcctggacagataccccaccgtgaagctgaccatcttcgtggctagactgtactggcacatggaccctcagaacagacaaggcctgagaaacctggtgcaaggcggcgtggacgtgagagtgatgggcccccccgagcactactactgctggagaaacttcgtggactgcagccccggcgaggaagaggagtggcctagataccaccccgtgcctagacagctgttcgacctggagctgagatgcatctgcctggacctgcccgcctgcctgaccatcagccccggcaagcatcagcagctgctgagattcagcctgaccctgcacagctgccactaccaaaagctccctcctgccctgctcctgcccctgggcctggtcagacccctgccccctcagacatccttcagcgctgcccaaggcggctggggccccgtgaccgtgaacggcgaggtgcaagacctgaacacctacggctggttctgggtgttcaga

SEQ ID NO: 316 - DNA, 510 bp, synthetic construct
  source 1..510: mol_type = other DNA
ctggaggacgtgtacgaccctagcgccctgcctagagtgacctacctgctgtacgagatcaagtggggcaacagcatcaagatctggagaaactggtgcagaaacacaagcacacagcacgccgagatcaacttcctggagaacgccttcaaggagagaagcctgaaccccctgacccactgcagcatcacctggttcctgagctggagcccctgctggaagtgcagccaaagcgtggtggagttcctcaagacctaccccaaggtgaacctggagatgtacgtggctcagctgttctggcacgaggacgagtgcaacagacaaggcctgcgggacctggtgatgaacggcgtgaccatcagagtgatggacctgagcgcctacaactactgctggagaaccttcgtgaaccatcagaacggcgaggactactggccctggcacctgaccccctggatcatgttcttcagcttcgagctgcagctgatcctgcaagtgagcagaagcctg

SEQ ID NO: 317 - DNA, 621 bp, synthetic construct
  source 1..621: mol_type = other DNA
atgctggagatcaaggaggacgtggtgcacatcttcagagccttctgggagtgcggcgtgcacagaggcaactggtgcggcgacacccacatcctgtacgagttcggcacaagccccgccaccaccgacttcagaaacatcgtgaccaacgacggcgacggcgggcagcacgccgagatcaagctgatctacatcctggacaaccacatcgctcagatggtgaagtgccatcagagaaaggagatctacctgaacatgtacatgaacagaagcccctgctacaagtgcgcctttcagctgatcagattcaagaactacctgtacaacaagggctacgacatcagaatcaccatcaaggccgctcagccctacatgtgcaagagaatcacctgcagcagctgcgtggagggcacaagccacagcgacggcctgagagagctgaacagaaacggcatcttcgtgaacgcctgggtgtgggccgactggcagttcctggccgcctacctgaacggcctggtgagagtgtgctacgacgagaagttccccgagagcggccacggcaagagagagagcagaagcatggccgacgaggccgcccaagaggacttcaacgccctgaga

SEQ ID NO: 318 - DNA, 522 bp, synthetic construct
  source 1..522: mol_type = other DNA
aagaagagaaacatcgtgtggcccgccggcatctacggcgaaacctggctgaagtgcacctacctgatcgcctccatcaaggccatcgacggcggcagcatcttcgaaaagaccttcaacaacgagcacgggcagcacgtggagatggtgatgctgcaccacagcgagttcctgaaggtggtgaaggagaacagcaacaccgagatcaccgtgaccctgaaccacagcccctgcagcgactgcgccaacaacctcaagaccttctacgaggactacgccgagaacatcaagaagatccaccaccccgtgttcatccacaaccacagagagatcgagaagaacaagaccggcctgtgggacctgaacaaggccggcttcaccctgagagccatgaccgggcagctgtggagcgaggcccacatcgaccccgtgagctacctgaacgacgccgagagaaacaagatcaagcagcgggacgaggaaaccgccaagtacctgaaggacagactgagccaacccaac

SEQ ID NO: 319 - DNA, 297 bp, synthetic construct
  source 1..297: mol_type = other DNA
atggtgaacatcaacgtgaccatcatccacagatacaagagaatcgcctacatcggccccaccatctgcgtgtgcatgaacttccctagctacttcagatgggtgaccatgtacctgagctacagcccctgcgccaactgcgccgacagaatcctgcagttcagctgcgacagacctcagtgcgacatcaccatcgccttcagctgcctgttcagacacaccgagaagctgcaccaagacggcctgcggagactgaacgccgagagcagaatcaccatcaaggtgttcacaagccaa

SEQ ID NO: 320 - DNA, 276 bp, synthetic construct
  source 1..276: mol_type = other DNA
gccgaggcccacgtgcccaacagagtggtgaagatcgccaacggcgaggagatcttcaacaagagattcaagaacatcccctacgacggccacgtggaggagatcatgctgaacgacggcggcttcctgaaggccgtggcctacaacagcgacatcgaggtgatcctgaccctgaactacagcccctgcaacatctgcgccatgaagctgaagaagttctacgagagctacaagagaaagatcaccaagttcatcattcagttcagcttcctgtac

SEQ ID NO: 321 - DNA, 600 bp, synthetic construct
  source 1..600: mol_type = other DNA
gccaagttcgtgggcctggtgcaagaggccgtgttcggcagacaagagcctagagccgtgctggaggaagaggctcagcacgtgagagaggaagaggctatgatgctggaggccatcaccctggacgactacgagttccccaccgctcgggacgtggccgcctacagagagggcagactgaactgcggcaacgagcccgactgcatcgtgttcatcggcatcgccaagtggttcgacagagtggtggctcagagagtgttcagacacagacaagagggcggcagcgtgggcagactggtggccatcgacatggtgatgttcctgctgaccgagggctacgtgctggagcactgcagcctggagatctacgcctcccggttccctagcttcagcgagctgtgcagcagcgacctgctgacccaagccgagcagttcggcagcgtgttcgacaagttcgtggtgagatacgcttccctcagagatggggacagcgccaccgacctggtcgccgctctggatagactgggcgtcagcgtgggccccatgggcgacgaggagtggagctacctgagcagactggagagagactggaccatcccc

SEQ ID NO: 322 - DNA, 345 bp, synthetic construct
  source 1..345: mol_type = other DNA
atgcccgtgttccccaacgccacatccttcagccaaagcgacatgaacagcaacaccaccgccctgatggtgtacgagagcgtgaacggcggctgggtggtgaagggcaaggtgacaagcggcaagggcaaccacgccgaggaagagctgtacgaggacctgaagggcagcaacctgctgaagatcacccaaggcgccgccatcctgatcgagatcaccaagagcccctgccactaccacgagggcaacaagtgctgcagccacgtgctggccaagatgaaggagaagggctacgtgagcagcatcgaggtgaagtacctgggcatctatcagtacgatcagagc

SEQ ID NO: 323 - DNA, 657 bp, synthetic construct
  source 1..657: mol_type = other DNA
ttcagaaagagctacttcaagaagccccccaccaccgctcagagcgagagattcttcaccgagaagagcagcgtgctggccatcctgaagttcaacgctagagagatcctgttcaccaccaacaccgcccccgccgacaagctggagttcccccacaccggcctgttcaatcagaacgggcagaagatcggcgcccacagcgaggactggatcaccgccggcttcagcaacctgatgcaaggcaacgagtacgccaaggtggcctccaccctgcccgccgatcagatcgacagacacgtggcctccctgaagatcaacttcagcccctgcaccggctgcgtgagaaccctggtgaacttcaacgactacctgaaaaagaccttcggcgacaagaccatcttcagagtgaagttcctcagaccctacgacctgcccatcaccctgaagaacaccgagagccaacagagcaagaccttcctgagcaccctggacgagctgaagaacaagggcatcgtggtgagactgcaccccgacaaggacaagacctacacaagcgacgagttcagcagagccgccgtcaagacccacgcccccaagcacatcgacgacctgaccaagagatggcgggacctgaaggtggctagaaagaaggccgcc

SEQ ID NO: 324 - DNA, 318 bp, synthetic construct
  source 1..318: mol_type = other DNA
atgacaagagccatcatcgtgaacatggagcacaactacgcctcctacggccatcagaagcccaccgacgacaacttcgaaaccatgccccacgccaccatccaagccctgatcaaggctagaagagaggacgtggtgggcggcaccatcttcctggctagattcccctgctacgagtgcgccaaggccatcgtgttcgccggcctgaccaaggtgatctacaaggaggagaacaccggcgacgccatgcacagcgaggccaagatgttcctggagttcagcggcgtggaggtggtgcagaaccccgacctggagttc

SEQ ID NO: 325 - DNA, 735 bp, synthetic construct
  source 1..735: mol_type = other DNA
atgaacagcctgagagccaccggcagcatcgacgaggtgctgctgcacgggcagatcctgagcacctaccaagccctgctggccctgagcagattcgacaccgtgtacagcgtgttcctgcccttcagcctgttcctggacgtgcccaccctgctgaacagagccaccatcaacagccaacccgccgtgagaagaaacctgctgcctcagctggagagcaacgagaagcagaagaagctggacatcttcatcagaagcggcctgcacggcgccgagtggcccaccatcgccctgtgcgtggtgagactgcccatgaccggcaaggtgtgcgtgatgcagaacgagcccggcgagcagcacgtggaggagagagtgatcgagaagctggaggaggaccacatccaagtgtgggaggaagaggaagaaacccccatcaagctggtgatctacatgaacttcagcacctgccacgactgcgtggagaagttcctgagcttcaccgagaagaacagactgcacgtggagatcgtggccgctgccctgtacaactgcgctagaaagagctgcagcccctgcaacttcgtggtgagacagaaggacgactatcagtatcagagaaacaccgaggccctgaagcagctggccgaggccggcatcaagctgagaggcttcgagtggaacgactggagcaacctggtggccctgctgaacgaggcctccgacatccccggcaac

SEQ ID NO: 326 - DNA, 342 bp, synthetic construct
  source 1..342: mol_type = other DNA
agaaacctgcagtacctggagaagtacggcaagatgatcctgtacaccggcaagcccaacacaagcacaagcagaaccggcgccgccctgatgccctgcaactttcagctgatcagcgccaagctgcccatcctgtgcgcctcctgcctggacctgatcctgatctaccacgacgtgaaccccctgaccctggacatcagaagcacctggctgcactgcctgggctgctgggccctgcagaacagactgaagatgatcaaggtgagcggcattcagctgcaccccatgctggaccacatgcccctgctgaacatctggaaccaagtgcaagtgaactacgcc

SEQ ID NO: 327 - DNA, 480 bp, synthetic construct
  source 1..480: mol_type = other DNA
atgcccatcgtgcagaacgaccccacctttatccgggacaaggagaagtacttcatgggcgtggccgaggagatcagcaagggcagcagccaccccatcgtgcccggcggctgcatcatcattagagacagaaccatcatcggcgacggcagaagcatcctggcctcctgcaaggtggagatcgactgcgtgacctacgccattgctaccgctgccgcccacgggaccgccaccgtgggcgccgtggtgtacagcacaagataccccttcagcgcctccatctttcagctgcacctgatgggcatcaagaagatcgtggtgaaggcccacgagtgggagccctactacaaggacgagttcagaagagccgctagactggctagagagctgagcatcgccatcgagcccttcttcgacgatgacgacgagagattcagcaccaacaagcaagcccctagattcgacaacagagaggagcag

SEQ ID NO: 328 - DNA, 507 bp, synthetic construct
  source 1..507: mol_type = other DNA
gctagattctacatgagcgccgccgtgaacgccgccaccctgagccacgccaagaagagaaaggtgggctgcatcatcgagcagagcggcaacatcctgagcttcggcttcaacggcacccccaccggcgaggacaacaactgcgaggacgagaacggcgtgaccctgagcagcgtgatccacgccgaggagaacgccctgagaaagctggagagattcgagctgtgcgacctgcacaactgcaacatctactgcacctgcgagccctgctacaactgcgccatcctgatcaccaacttcttcagaagaagcgccaagcccaagctgtacttcctggagcagagcaagagataccccggcgtgggcagcgacatcctgaacagagccggcatcagcagcacacagctgttcttcgacaccaacggcaacctgagcgacaaaagacccgagagcgaggcctccctgttcaccaactggggctggccccccggcgacgccaaggaggag

SEQ ID NO: 329 - protein, 207 aa, synthetic construct
MLEIKEDVVHIFRAFWECGVHRGNWCGDTHILYEFGTSPATTDFRNIVTNDGDGGQHAEIKLIYILDNHIAQMVKCHQRKEIYLNMYMNRSPCYKCAFQLIRFKNYLYNKGYDIRITIKAAHPYMCKRITCSSCVEGTSHSDGLRELNRNGIFVNAWVWADWQFLAAYLNGLVRVCYDEKFPESGHGKRESRSMADEAAQEDFNALR

SEQ ID NO: 330 - DNA, 10729 bp, synthetic construct
  source 1..10729: mol_type = other DNA
gctagcgccaccatgaaacggacagccgacggaagcgagttcgagtcaccaaagaagaagcggaaagtcatgctggagatcaaggaggacgtggtgcacatcttcagagccttctgggagtgcggcgtgcacagaggcaactggtgcggcgacacccacatcctgtacgagttcggcacaagccccgccaccaccgacttcagaaacatcgtgaccaacgacggcgacggcgggcagcacgccgagatcaagctgatctacatcctggacaaccacatcgctcagatggtgaagtgccatcagagaaaggagatctacctgaacatgtacatgaacagaagcccctgctacaagtgcgcctttcagctgatcagattcaagaactacctgtacaacaagggctacgacatcagaatcaccatcaaggccgctcagccctacatgtgcaagagaatcacctgcagcagctgcgtggagggcacaagccacagcgacggcctgagagagctgaacagaaacggcatcttcgtgaacgcctgggtgtgggccgactggcagttcctggccgcctacctgaacggcctggtgagagtgtgctacgacgagaagttccccgagagcggccacggcaagagagagagcagaagcatggccgacgaggccgcccaagaggacttcaacgccctgagatctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 331 - DNA, 10729 bp, synthetic construct
  source 1..10729: mol_type = other DNA
gctagcgccaccatgaaacggacagccgacggaagcgagttcgagtcaccaaagaagaagcggaaagtcatgctggagatcaaggaggacgtggtgcacatcttcagagccttctgggagtgcggcgtgcacagaggcaactggtgcggcgacacccacatcctgtacgagttcggcacaagccccgccaccaccgacttcagaaacatcgtgaccaacgacggcgacggcgggcagcacgccgagatcaagctgatctacatcctggacaaccacatcgctcagatggtgaagtgccatcagagaaaggagatctacctgaacatgtacatgaacagaagcccctgctacaagtgcgcctttcagctgatcagattcaagaactacctgtacaacaagggctacgacatcagaatcaccatcaaggccgctcatccctacatgtgcaagagaatcacctgcagcagctgcgtggagggcacaagccacagcgacggcctgagagagctgaacagaaacggcatcttcgtgaacgcctgggtgtgggccgactggcagttcctggccgcctacctgaacggcctggtgagagtgtgctacgacgagaagttccccgagagcggccacggcaagagagagagcagaagcatggccgacgaggccgcccaagaggacttcaacgccctgagatctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctggaattcgagcccaagaagaagaggaaagtctagctcgagaataaaggaaatttattttcattgcaatagtgtgttggaattttttgtgtctctcagttaacaaaaaagcaccgactcggtgccactttttcaagttgataacggactagccttattttaacttgctatttctagctctaaaacgaggcggggtggagggggtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcgaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

(19) United States
(12) Patent Application Publication
(10) Pub. No.: US 2026/0347823 A1
(43) Pub. Date: Nov. 5, 2026

(54) DEAMINASE, FUSION PROTEIN, NUCLEIC ACID, PHARMACEUTICAL COMPOSITION AND USE THEREOF

(71) Applicant: GUANGZHOU REFORGENE MEDICINE CO., LTD., Guangzhou, Guangdong (CN)

(72) Inventors: Junbin LIANG, Guangzhou (CN); Liancheng HUANG, Guangzhou (CN); Xingxiang LIANG, Guangzhou (CN); Hui XU, Guangzhou (CN); Zhengxia YANG, Guangzhou (CN); Zheyuan OU, Guangzhou (CN); Jinxiu CAI, Guangzhou (CN); Desheng HUANGFU, Guangzhou (CN)

(73) Assignee: GUANGZHOU REFORGENE MEDICINE CO., LTD., Guangzhou, Guangdong (CN)

(21) Appl. No.: 19/698,836

(22) Filed: Jun. 4, 2026

Related U.S. Application Data
(63) Continuation of application No. PCT/CN2024/137235, filed on Dec. 5, 2024.

(30) Foreign Application Priority Data
Dec. 5, 2023 (CN) 202311657105.X
Apr. 3, 2024 (CN) 202410403512.6
Jul. 4, 2024 (CN) 202410893197.X

(51) Int. Cl. C12N 9/78; A61K 38/50; C12N 15/11

(52) U.S. Cl. CPC C12N 9/78; A61K 38/50; C12N 15/11; C12Y 305/04001

(57) ABSTRACT

A deaminase, a fusion protein and the use thereof. The deaminase contains an amino acid sequence having at least 50% sequence identity to any one of SEQ ID NOs: 1-6, 39-164 and 169, and the deaminase can deaminize at least one nucleotide in a target polynucleotide. The deaminase can be fused with a nucleic acid-binding polypeptide to obtain various base editors for the sequence-specific base conversion of the target polynucleotide. The performance of the editors can be further improved by means of fusing one or two UGIs, fusing a Gam protein and other strategies.

### CROSS-REFERENCE TO RELATED APPLICATIONS

This application is a continuation of International Patent Application No. PCT/CN2024/137235, filed on Dec. 5, 2024, which claims priority to Chinese Patent Application No. 202410893197.X, filed on Jul. 4, 2024, Chinese Patent Application No. 202410403512.6, filed on Apr. 3, 2024, and Chinese Patent Application No. 202311657105.X, filed on Dec. 5, 2023, the entire contents of each of which are hereby incorporated by reference.

### SEQUENCE LISTING

The instant application contains a Sequence Listing which is submitted electronically in XML format and is hereby incorporated by reference in its entirety. The XML copy, created on Jun. 12, 2026, is named “2026-06-12-Sequence List-20954-0010US00,” and is 488,335 bytes in size.

### TECHNICAL FIELD

The present disclosure generally relates to the field of gene editing, and in particular to deaminase, fusion protein, nucleic acid, pharmaceutical composition, and use thereof.

### BACKGROUND

A CRISPR-Cas system is an adaptive immune defense formed by bacteria and archaea during long-term evolution and can be used to combat invading viruses and exogenous DNA. In 2016, David Liu's laboratory developed cytosine base editors (CBEs). Currently, discovering more suitable cytosine deaminases for constructing base editors and expanding a gene editing toolbox have become important research directions in gene editing.

### SUMMARY

The present disclosure provides a deaminase, a fusion protein, a nucleic acid, a pharmaceutical composition, and use thereof. The deaminase provided in the present disclosure has high deamination editing efficiency and a wide editing window, and the constructed fusion protein also has high editing efficiency and a wide editing window. Some deaminase fusion proteins provided in the present disclosure have editing window positions different from those of existing fusion proteins.

One aspect of the present disclosure provides a deaminase, comprising an amino acid sequence having at least 50% sequence identity to a sequence set forth in any one of SEQ ID NO: 1-6, 39-164, and 169.

In some embodiments of the present disclosure, the deaminase is capable of deaminating at least one nucleotide in a polynucleotide.

In some embodiments of the present disclosure, the deaminase is capable of deaminating at least one nucleotide in a target polynucleotide.

In some embodiments of the present disclosure, the deaminase deaminates the at least one nucleotide in the polynucleotide.

In some embodiments of the present disclosure, the deaminase deaminates the at least one nucleotide in the target polynucleotide.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in any one of SEQ ID NO: 1-6, 39-164, and 169.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in any one of SEQ ID NO: 1, 3-6, 43, 46, 48, 51, 63, 114, 126, 160, 163, 164, and 169.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in any one of SEQ ID NO: 3, 5, 6, 126, 160, 163, 164, and 169.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 3.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 5.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 6.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 126.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 160.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 163.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 164.

In some embodiments of the present disclosure, the deaminase comprises an amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% sequence identity to the sequence set forth in SEQ ID NO: 169.

In some embodiments of the present disclosure, the deaminase comprises a mutation at a position corresponding to amino acid residue 122, 114, 176, 2, and/or 68 of a reference sequence set forth in SEQ ID NO: 169.

In some embodiments of the present disclosure, the deaminase comprises a mutation at a position corresponding to any one, two, three, four, or five amino acid residues selected from position 122, position 114, position 176, position 2, and position 68 of a reference sequence set forth in SEQ ID NO: 169.

In some embodiments of the present disclosure, the deaminase comprises a mutation at a position corresponding to any one, two, three, four, or five amino acid residues selected from position 122, position 191, position 114, position 2, and position 89 of the reference sequence set forth in SEQ ID NO: 169.

Non-limitingly, the mutation may be a substitution, insertion, and/or deletion of an amino acid residue; further, the mutation may be a substitution of an amino acid residue. Non-limitingly, the corresponding position may be identified by performing sequence alignment of the amino acid sequence of the deaminase with the reference sequence set forth in SEQ ID NO: 169. In some embodiments of the present disclosure, the deaminase comprises a mutation at a position corresponding to amino acid residue 122 of the reference sequence set forth in SEQ ID NO: 169. In some embodiments of the present disclosure, the deaminase comprises a mutation at a position corresponding to amino acid residue 191 of the reference sequence set forth in SEQ ID NO: 169. In some embodiments of the present disclosure, the deaminase comprises a mutation at a position corresponding to amino acid residue 114 of the reference sequence set forth in SEQ ID NO: 169. In some embodiments of the present disclosure, the deaminase comprises a mutation at a position corresponding to amino acid residue 2 of the reference sequence set forth in SEQ ID NO: 169. In some embodiments of the present disclosure, the deaminase comprises a mutation at a position corresponding to amino acid residue 176 of the reference sequence set forth in SEQ ID NO: 169. In some embodiments of the present disclosure, the deaminase comprises a mutation at a position corresponding to amino acid residue 68 of the reference sequence set forth in SEQ ID NO: 169. In some embodiments of the present disclosure, the deaminase comprises a mutation at a position corresponding to amino acid residue 89 of the reference sequence set forth in SEQ ID NO: 169.

In some embodiments of the present disclosure, the deaminase has a mutation to H at the position corresponding to the amino acid residue 122 of the reference sequence set forth in SEQ ID NO: 169. In some embodiments of the present disclosure, the deaminase has a mutation to R at the position corresponding to the amino acid residue 191 of the reference sequence set forth in SEQ ID NO: 169. In some embodiments of the present disclosure, the deaminase has a mutation to T at the position corresponding to the amino acid residue 114 of the reference sequence set forth in SEQ ID NO: 169. In some embodiments of the present disclosure, the deaminase has a mutation to M at the position corresponding to the amino acid residue 2 of the reference sequence set forth in SEQ ID NO: 169. In some embodiments of the present disclosure, the deaminase has a mutation to D at the position corresponding to the amino acid residue 89 of the reference sequence set forth in SEQ ID NO: 169. In some embodiments of the present disclosure, the deaminase has a mutation to F at the position corresponding to the amino acid residue 176 of the reference sequence set forth in SEQ ID NO: 169. In some embodiments of the present disclosure, the deaminase has a mutation to I at the position corresponding to the amino acid residue 68 of the reference sequence set forth in SEQ ID NO: 169. In some embodiments of the present disclosure, the deaminase has a mutation to I at the position corresponding to the amino acid residue 68 and a mutation to T at the position corresponding to the amino acid residue 114 of the reference sequence set forth in SEQ ID NO: 169.

In some embodiments of the present disclosure, the deaminase comprises an amino acid sequence having 100% sequence identity to the sequence set forth in any one of SEQ ID NO: 1-6, 39-164, and 169.

In some embodiments of the present disclosure, the deaminase comprises an amino acid sequence having 100% sequence identity to the sequence set forth in any one of SEQ ID NO: 1, 3-6, 43, 46, 48, 51, 63, 114, 126, 160, 163, 164, and 169.

In some embodiments of the present disclosure, the deaminase comprises an amino acid sequence set forth in any one of SEQ ID NO: 1-6, 39-164, and 169.

In some embodiments of the present disclosure, the deaminase comprises an amino acid sequence set forth in any one of SEQ ID NO: 1, 3-6, 43, 46, 48, 51, 63, 114, 126, 160, 163, 164, and 169.

In some embodiments of the present disclosure, the amino acid sequence of the deaminase is set forth in any one of SEQ ID NO: 1-6, 39-164, and 169.

In some embodiments of the present disclosure, the amino acid sequence of the deaminase is set forth in any one of SEQ ID NO: 1, 3-6, 43, 46, 48, 51, 63, 114, 126, 160, 163, 164, and 169.

In some embodiments of the present disclosure, the amino acid sequence of the deaminase is not any one of SEQ ID NO: 1-5 and 54-60.

In some embodiments of the present disclosure, the deaminase is a Q122H, S191R, 1114T, L2M, or N89D mutant of deaminase-145 (the amino acid sequence set forth in SEQ ID NO: 169).

In some embodiments of the present disclosure, the deaminase may deaminate a base in a nucleic acid sequence together with a nucleic acid-binding polypeptide. For example, the deaminase may deaminate a base in a DNA sequence together with a TALEN or a ZFN.

In some embodiments of the present disclosure, the deaminase may form a complex with the nucleic acid-binding polypeptide to deaminate the base in the nucleic acid sequence.

In some embodiments of the present disclosure, the deaminase is capable of deaminating a base in a target polynucleotide together with a nucleic acid-binding polypeptide and a guide RNA (gRNA).

In some embodiments of the present disclosure, the deaminase is capable of forming a complex together with the nucleic acid-binding polypeptide and the guide RNA (gRNA), the guide RNA guides the complex to bind to the target polynucleotide and deaminate the base in the target polynucleotide sequence.

The deaminase and the nucleic acid-binding polypeptide in the complex may be two separate molecules, or may be a single molecule formed by linking the deaminase and the nucleic acid-binding polypeptide (for example, forming a fusion protein).

A non-limiting example is: the deaminase may be used to replace a deaminase domain in a tBE base editing system as described in patent application WO2022206986A1 or WO2023155901A1, thereby obtaining a new base editing system.

In some embodiments, the deaminase comprises a homologous or heterologous domain. In some embodiments, the deaminase comprises a nucleic acid-binding polypeptide. In some embodiments, the deaminase comprises a cellular localization signal; further, the deaminase comprises a nuclear localization signal, a nuclear export signal, a mitochondrial localization signal, or a chloroplast localization signal. In some embodiments, the deaminase comprises a uracil glycosylase inhibitor (UGI) domain.

One aspect of the present disclosure provides a fusion protein, wherein the fusion protein comprises the deaminase as described in the present disclosure.

In some embodiments of the present disclosure, the fusion protein comprises: a) a nucleic acid-binding polypeptide that binds to a target polynucleotide; and b) the deaminase as described in the present disclosure.

The fusion protein may be used for targeted editing of a nucleic acid (e.g., DNA nucleic acid, RNA nucleic acid) in vitro, ex vivo, or in vivo, for example, for generating mutant cells. These mutant cells can be in a plant or an animal. The fusion protein may also be used to introduce targeted mutations, for example, for correction of genetic defects in vitro in mammalian cells (e.g., in cells obtained from a subject, the cells subsequently being reintroduced into the same or another subject); and to introduce targeted mutations, for example, in disease-associated genes in a mammalian subject to correct genetic defects or introduce mutations. The fusion protein may also be used to introduce targeted mutations in plant cells, for example, for introducing beneficial or agronomically valuable traits or alleles. The targeted editing is, for example, deaminating at least one nucleotide in a nucleic acid.

Optionally, the nucleic acid-binding polypeptide is a DNA binding polypeptide.

Optionally, the nucleic acid-binding polypeptide is an RNA binding polypeptide.

In some embodiments of the present disclosure, the nucleic acid-binding polypeptide is a TALEN nuclease, a zinc finger nuclease (ZFN), a CRISPR-Cas nuclease, or a meganuclease.

In some embodiments of the present disclosure, the nucleic acid-binding polypeptide is an RNA-guided nuclease. Optionally, the DNA binding polypeptide is a CRISPR-associated nuclease, i.e., a Cas enzyme (also referred to as a Cas protein, CRISPR-Cas nuclease).

In some embodiments of the present disclosure, the nucleic acid-binding polypeptide is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9, Cas10, Cas10d, Cas12a/Cpf1, Cas12b/C2c1, Cas12c/C2c3, Cas12d/CasY, Cas12e/CasX, Cas12f/CasZ, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Cas13a, Cas13b, Cas13c, Cas13d, Cas13e, Cas13f, TnpB, IscB, IsrB, and Fancor; or a fragment of any one of them. A non-limiting example of the fragment is, for example, a nucleic acid binding domain fragment.

In some embodiments of the present disclosure, the nucleic acid-binding polypeptide is selected from Cas9, Cas12, Cas13, TnpB, IscB, IsrB, and a Fancor nuclease; or a fragment of any one of them, the fragment including but not limited to the nucleic acid binding domain fragment.

In some embodiments of the present disclosure, the Cas9 is selected from SpCas9, SaCas9, Nme2Cas9, Nme3Cas9, CjCas9, NmCas9, FnCas9, PpnCas9, FrCas9, SauCas9, SauriCas9, ScaCas9, St1Cas9, BlatCas9, CdiCas9, GeoCas9, or a fragment of any one of them, and a mutant of any one of them or a fragment of the mutant.

In some embodiments of the present disclosure, the nucleic acid-binding polypeptide is selected from AsCpf1, enAsCas12a (addgene plasmid #196724), dFnCas12a (addgene plasmid #136379), ErCas12a, LbCas12a D832A, LbCas12a H759A, LbCas12a E795L, FnCas12a3, FnCas12a D917A, AsCas12a R1226A, AsCas12a D908A, AsCas12a E174R/S542R, AsCas12a (S542R/K548V/N552R), PrCas12a, PxCas12a, PcCas12a, PdCas12a, Mb2Cas12a, Mb3Cas12a, MICas12a, CMaCas12a, CMtCas12a, HkCas12a, Lb5Cas12a, ErCas12a, TsCas12a, FnCpf1, LbCas12a, dLbCpf1, ttHsCas12a, AaCas12b, AaCas12b D570A, AaCas12b Q119F/E475R/E758R, BhCas12b, BvCas12b, BrCas12b, AkCas12b, AmCas12b, BsCas12b, OspCas12c, Cas12c2 (addgene plasmid #183072), Cas12c_4 (addgene plasmid #183071), Cas12c1 (addgene plasmid #120872), CasY1 (from Katanobacteria), CasY.2 (from Vogelbacteria), CasY.3 (from Vogelbacteria), CasY.4 (from Parcubacteria), CasY.5 (from Komeilibacteria), CasY6 (from Kerfeldbacteria), PlmCasX, DpbCasX, UnlCas12f, CnCas12f1, enRhCas12f1, AsCas12f1, SpaCas12f1, Cas12g1 (addgene plasmid #120879), Cas12 h of WO2021113522A1 (SEQ ID NO: 1 of that patent application), Cas12i1 (addgene plasmid #171670), Cas12i2 (addgene plasmid #188275), Cas12i1 (addgene plasmid #120882), Cas12i2 (addgene plasmid #120883), Cas12i proteins designated Cas12f.4/Cas12f.5/Cas12f.6 in CN111757889B, dSiCas12i(D1049A), SiCas12i, Si2Cas12i, WiCas12i, Wi2Cas12i, Wi3Cas12i, SaCas12i, Sa2Cas12i, Sa3Cas12i, WaCas12i, Wa2Cas12i, xCas12i, hfCas12Max, Cas12i-Max (addgene plasmid #188276), Cas12i1 D647A (addgene plasmid #171671), Cas12i-HiFi (addgene plasmid #188269), Cas12i1 D647A, Cas12j3 (addgene plasmid #188497), Cas12j2 (addgene plasmid #188498), AsCas12j-2 (addgene plasmid #191655), Cas12j-8 (addgene plasmid #194966), ShCas12k, N7Cas12k, AcCas12k, Cas12k-TniQ (addgene plasmid #181787), Cas12k-TnsC (addgene plasmid #181789), Cas12l, MmCas12m, MmCas12m AZF (H549A, C552A), dCas12m-AZF (D485A, H549A, C552A), AcCas12n, dAcCas12n(D240), TnpB Actinomadura_cellulosilytica_strain_DSM_45823, TnpB Actinomadura_namibiensis_strain_DSM_44197, TnpB Actinomadura_umbrina_strain_DSM_43927_$, TnpB Actinoplanes_lobatus_strain_DSM_43150 (TnpB-1 and TnpB-2), TnpB Alicyclobacillus_macrosporagiidus_strain_DSM_17980, TnpB Haloactinospora_alba_Strain_DSM_45015, TnpB Lipingzhangella_halophila_strain_DSM_102030. TnpB Meiothermus_Silvanus_DSM_9946, TnpB QNFX01000004, ISDra2 TnpB(PDB: 8H1J), KralscB-1, AwalscB, OgeulscB, GtFz1 (from Guillardia theta), SpuFz1 (from Spizellomyces punctatus), NlovFz2 (from Percolozoa Naegleria lovaniensis), MmeFz2 (from Mercenaria mercenaria), a fragment of any one of them, and a mutant of any one of them or a fragment of the mutant.

In some embodiments of the present disclosure, the DNA binding polypeptide is capable of cleaving one strand of a double-stranded nucleic acid molecule (e.g., a double-stranded DNA molecule). Optionally, the DNA binding polypeptide is an RNA-guided nuclease having nickase activity.

In some embodiments of the present disclosure, the Cas enzyme cleaves a target strand of a double-stranded nucleic acid molecule, meaning that the Cas enzyme cleaves a strand that base-pairs with (is complementary to) a gRNA (e.g., an sgRNA) bound to the Cas enzyme.

In some embodiments of the present disclosure, the nucleic acid-binding polypeptide is an RNA-guided nuclease with inactivated nuclease activity.

In some embodiments of the present disclosure, the nucleic acid-binding polypeptide is a variant of an RNA-guided nuclease with completely inactivated nuclease activity compared to a wild-type form of the RNA-guided nuclease, such as a dCas enzyme, including but not limited to Cas9, Cas12, Cas13, TnpB, IscB, IsrB, and Fancor each of which has completely inactivated nuclease activity (which may be referred to as dead Cas9, dead Cas12, dead Cas13, dead TnpB, dead IscB, dead IsrB, and dead Fancor); or a fragment of any one of them or a mutant of any one of them, such as a SpRY Cas9 mutant.

In some embodiments of the present disclosure, the nucleic acid-binding polypeptide is a variant of an RNA-guided nuclease with partially inactivated nuclease activity compared to a wild-type form of the RNA-guided nuclease, such as an nCas enzyme, for example, a polypeptide fragment that retains the nuclease activity of cleaving a single strand of double-stranded DNA, including but not limited to nickase Cas9 and nickase Cas12.

In some embodiments of the present disclosure, the fusion protein further comprises one, two, three, four, or more Uracil Olycosylase Inhibitors (UGIs). In some embodiments of the present disclosure, the fusion protein comprises one UGI. In some embodiments of the present disclosure, the fusion protein comprises two UGIs. In some embodiments of the present disclosure, the fusion protein comprises three UGIs. The UGI may inhibit human UDG activity.

In some embodiments of the present disclosure, the UGI comprises an amino acid sequence having at least 60%, at least 65%, at least 70%, at least 75%, at least 80%, at least 85%, at least 90%, at least 95%, at least 96%, at least 97%, at least 98%, at least 99%, or at least 99.5% sequence identity to an amino acid sequence set forth in SEQ ID NO: 38. In some embodiments, the UGI comprises a fragment or a homolog of the sequence set forth in SEQ ID NO: 38.

In a preferred embodiment of the present disclosure, the fusion protein comprises one or more nuclear export signals (NESs), for example, one, two, three, four, or more NESs.

In a preferred embodiment of the present disclosure, the fusion protein comprises one or more nuclear localization signals (NLSs), for example, one, two, three, four, or more NLSs.

Nuclear localization signals are known in the art and generally comprise a stretch of basic amino acids (see, e.g., Lange et al., J. Biol. Chem. (2007) 282:5101-5105). In specific embodiments, the fusion protein comprises two, three, four, five, six, or more nuclear localization signals. The one or more nuclear localization signals may be heterologous NLSs. Non-limiting examples of nuclear localization signals of RNA-guided nucleases useful for the present disclosure are the nuclear localization signals of SV40 large T-antigen, nucleoplasmin, and c-Myc (see, e.g., Ray et al., (2015) Bioconjug Chem 26(6):1004-7). The fusion protein may comprise one or more NLS sequences at its N-terminus, C-terminus, or both N-terminus and C-terminus. For example, the fusion protein may comprise two NLS sequences in the N-terminal region and four NLS sequences in the C-terminal region.

Optionally, the NLS comprises an amino acid sequence set forth in SPKKKRKVEAS (SEQ ID NO: 14), GPKKKRKVAAA (SEQ ID NO: 15), PKKKRKV (SEQ ID NO: 16), KRPAATKKAGQAKKKK (SEQ ID NO: 17), PAAKRVKLD (SEQ ID NO: 18), RQRRNELKRSP (SEQ ID NO: 19), NQSSNFGPMKGGNFGGRSSGPYGGGGQYFAKPRNQGGY (SEQ ID NO: 20), RMRIZFKNKGKDTAELRRRRVEVSVELRKAKKDEQILKRRNV (SEQ ID NO: 21), VSRKRPRP (SEQ ID NO: 22), PPKKARED (SEQ ID NO: 23), POPKKKPL (SEQ ID NO: 24), SALIKKKKKMAP (SEQ ID NO: 25), DRLRR (SEQ ID NO: 26), PKQKKRK (SEQ ID NO: 27). RKLKKKIKKL (SEQ ID NO: 28), REKKKFLKRR (SEQ ID NO: 29), KRKGDEVDGVDEVAKKKSKK (SEQ ID NO: 30), RKCLQAGMNLEARKTKK (SEQ ID NO: 31), and PAAKKKKLD (SEQ ID NO: 32).

In some embodiments of the present disclosure, the nucleic acid-binding polypeptide, the deaminase, the one or more UGIs, and the one or more NLSs are linked directly or via linkers.

In some embodiments of the present disclosure, when the nucleic acid-binding polypeptide, the deaminase, the one or more UGIs, and the one or more NLSs are linked via the linkers, these linkers may be the same or different.

In some embodiments of the present disclosure, the linkers comprise a sequence set forth in any one of SEQ ID NO: 33-37 and EFE, or a fragment of any one of them or any one variant of any one of them.

In some embodiments of the present disclosure, the linkers comprise amino acids set forth in SGGSSGGSSGSETPGTSESATPESSGGSSGGS (SEQ ID NO: 33) and/or SGGSGGSGGS (SEQ ID NO: 34). In some embodiments of the present disclosure, the deaminase and the nucleic acid-binding polypeptide are linked via SEQ ID NO: 33; and/or the nucleic acid-binding polypeptide and the UGI are linked via SEQ ID NO: 34.

In a preferred embodiment of the present disclosure, the fusion protein comprises the following structures sequentially from N-terminus to C-terminus: a deaminase and a nucleic acid-binding polypeptide. Preferably, the fusion protein comprises the following structures sequentially from N-terminus to C-terminus: a deaminase, a linker 1, and a nucleic acid-binding polypeptide.

More preferably, the fusion protein comprises the following structures sequentially from N-terminus to C-terminus: a deaminase, a linker 1, a nucleic acid-binding polypeptide, a linker 2, and a UGI.

Further preferably, the fusion protein comprises the following structures sequentially from N-terminus to C-terminus: a deaminase, a linker 1, a nucleic acid-binding polypeptide, a linker 2, a UGI, and an NLS.

Still further preferably, the fusion protein comprises the following structures sequentially from N-terminus to C-terminus: an NLS, a deaminase, a linker 1, a nucleic acid-binding polypeptide, a linker 2, a UGI, and an NLS.

In a specific embodiment of the present disclosure, the fusion protein comprises the following structures sequentially from N-terminus to C-terminus: an SV 40 NLS, a deaminase, a linker 1, a nucleic acid-binding polypeptide, a linker 2, a UGI, and an SV 40 NLS.

The numbers in the linker 1 and the linker 2 are only used to distinguish nouns of the same type, and do not indicate any actual meaning.

In some embodiments of the present disclosure, the linker 1 and/or the linker 2 are/is optionally selected from sequences set forth in SEQ ID NO: 33-37 and EFE.

In some embodiments of the present disclosure, the linker 1 and/or the linker 2 are/is SEQ ID NO: 33.

In some embodiments of the present disclosure, the linker 1 and/or the linker 2 are/is SEQ ID NO: 34.

In the present disclosure, the SV 40 refers to Simian vacuolating virus 40.

In some embodiments of the present disclosure, a structure of the fusion protein is deaminase-XTEN linker-dead Cas.

In some embodiments of the present disclosure, the structure of the fusion protein is deaminase-XTEN linker-dead Cas-UGI.

In some embodiments of the present disclosure, the structure of the fusion protein is deaminase-XTEN linker-nickase Cas-UGI.

In some embodiments of the present disclosure, the fusion protein is obtained by replacing a deaminase in YE1-BE3, YE2-BE3, EE-BE3, YEE-BE3, or HF-CBE3 in the prior art with the deaminase of the present disclosure.

In some embodiments of the present disclosure, the structure of the fusion protein is deaminase-XTEN linker-dead Cas-UGI-UGI.

In some embodiments of the present disclosure, the structure of the fusion protein is deaminase-XTEN linker-nickase Cas-UGI-UGI.

In some embodiments of the present disclosure, the structure of the fusion protein is Gam-deaminase-XTEN linker-nickase Cas9-UGI-UGI. The Gam is a Gam protein from bacteriophage Mu or an analog or homolog thereof.

In some embodiments of the present disclosure, the fusion protein is capable of forming a complex with a guide RNA (gRNA), and the gRNA guides the complex to bind to a target polynucleotide and deaminates a base in a target polynucleotide.

Another aspect of the present disclosure provides an isolated nucleic acid encoding the deaminase as described in the present disclosure or the fusion protein as described in the present disclosure.

In some embodiments of the present disclosure, the nucleic acid is codon-optimized, for example, prokaryotic codon-optimized for a prokaryotic expression system, or eukaryotic codon-optimized for a eukaryotic expression system.

In some embodiments of the present disclosure, the isolated nucleic acid comprises a sequence set forth in any one of SEQ ID NO: 165-296 and 313-328. In some embodiments of the present disclosure, the sequence of the isolated nucleic acid is set forth in any one of SEQ ID NO: 165-296 and 313-328.

Another aspect of the present disclosure provides an expression vector, wherein the expression vector comprises the nucleic acid described in the present disclosure.

A person skilled in the art may select an appropriate expression vector, for example, the expression vector may include, but is not limited to, a viral expression vector, a bacterial expression vector, a fungal expression vector, an animal expression vector (e.g., insect, drosophila, nematode, fish, zebrafish, mammal, mouse, rat, rabbit, pig, monkey, human, etc.), a plant expression vector, and the like.

In some embodiments of the present disclosure, the expression vector comprises a regulatory sequence. The regulatory sequence is used to regulate the expression of the deaminase or the fusion protein.

In some embodiments of the present disclosure, the regulatory sequence is any one or more selected from: a promoter, an enhancer, an internal ribosome entry site, and a transcription termination signal; the promoter is, for example, a constitutive promoter, an inducible promoter, a broad-spectrum promoter, or a tissue-specific promoter, and/or the transcription termination signal is, for example, a polyadenylation signal or a poly-U sequence.

In some embodiments, the expression vector comprises a pol Ill promoter (e.g., U6 and H1 promoters), a pol II promoter (e.g., retroviral Rous sarcoma virus (RSV), a LTR promoter (optionally with an RSV enhancer), a cytomegalovirus (CMV) promoter (optionally with a CMV enhancer), an SV40 promoter, a dihydrofolate reductase promoter, a β-actin promoter, a phosphoglycerate kinase (PGK) promoter, or an EF1α promoter.

In some embodiments, the promoter is a constitutive promoter, which is continuously active and not regulated by external signals or molecules. Suitable constitutive promoters include, but are not limited to, CMV, RSV, SV40, EF1α, CAG, and β-actin promoters. In some embodiments, the promoter is an inducible promoter regulated by an external signal or molecule (e.g., a transcription factor).

In some embodiments, the promoter is a tissue-specific promoter, which may be used to drive tissue-specific expression of the deaminase or the fusion protein. Suitable muscle-specific promoters include, but are not limited to, CK8, MHCK7, a myoglobin promoter (Mb), a desmin promoter, a muscle creatine kinase promoter (MCK) and variants thereof, and a SPc5-12 synthetic promoter. Suitable immune cell-specific promoters include, but are not limited to, a 829 promoter (B cells), a CD14 promoter (monocytes), a CD43 promoter (leukocytes and platelets), CD68 (macrophages), and an SV40/CD43 promoter (leukocytes and platelets). Suitable blood cell-specific promoters include, but are not limited to, a CD43 promoter (leukocytes and platelets), a CD45 promoter (hematopoietic cells), INF-β (hematopoietic cells), a WASP promoter (hematopoietic cells), an SV40/CD43 promoter (leukocytes and platelets), and an SV40/CD45 promoter (hematopoietic cells). Suitable pancreas-specific promoters include, but are not limited to, an elastase-1 promoter. Suitable endothelial cell-specific promoters include, but are not limited to, a Fit-1 promoter and an IOAM-2 promoter. Suitable neuronal tissue/cell-specific promoters include, but are not limited to, a GFAP promoter (astrocytes), a SYN1 promoter (neurons), and NSE/RU5′ (mature neurons). Suitable kidney-specific promoters include, but are not limited to, an Nphsl promoter (podocytes). Suitable bone-specific promoters include, but are not limited to, an OG-2 promoter (osteoblasts, odontoblasts). Suitable lung-specific promoters include, but are not limited to, an SP-B promoter (lung). Suitable liver-specific promoters include, but are not limited to, an SV40/Alb promoter. Suitable heart-specific promoters include, but are not limited to, α-MHC.

Another aspect of the present disclosure provides a pharmaceutical composition, wherein the pharmaceutical composition comprises the deaminase described in the present disclosure, the fusion protein described in the present disclosure, the nucleic acid described in the present disclosure, or the expression vector described in the present disclosure, and a pharmaceutically acceptable delivery carrier.

In some embodiments of the present disclosure, the delivery carrier is selected from: a viral vector, virus-like particles (VLPs), nanoparticles such as lipid nanoparticles (LNPs), liposomes, extracellular vesicles (EVs) such as exosomes and microvesicles, and a gene gun.

In some embodiments of the present disclosure, the viral vector is an adeno-associated virus, an adenovirus, a lentivirus, or a herpes virus.

In some embodiments of the present disclosure, the adeno-associated virus is a recombinant adeno-associated virus vector of serotype AAV1, AAV2, AAV3, AAV4, AAV5, AAV6, AAV7, AAV8, AAV9, AAV10, AAV11, AAV12, AAV13, AAV PHP.B, AAV PHP.B2, AAV PHP.B3, AAV PHP.A, AAV PHP.eB, AAV PHP.eS, AAV2.7m8, AAV8.7m8, AAV ShH10, AAVrh10, orAAVrh74.

In some embodiments of the present disclosure, the deaminase, the fusion protein, the nucleic acid, or the expression vector described in the present disclosure is packaged in an AAV vector, for example, packaged into a capsid of the AAV1, AAV2, AAV3, AAV4, AAV5, AAV6, AAV7, AAV8, AAV9, AAV10, AAV11, AAV12, AAV13, AAV PHP.B, AAV PHP.B2, AAV PHP.B3, AAV PHP.A, AAV PHP.eB, AAV PHP.eS, AAV2.7m8, AAV8.7m8, AAV ShH10, AAVrh10, orAAVrh74.

In some embodiments of the present disclosure, the lentiviral vector is pseudotyped with an envelope protein. Preferably, the nucleic acid is linked to an aptamer sequence.

In some embodiments of the present disclosure, in the VLPs, the nucleic acid is linked to a gene encoding a gag protein.

Another aspect of the present disclosure provides an expression system, wherein the expression system comprises the expression vector described in the present disclosure, the pharmaceutical composition described in the present disclosure, or the nucleic acid described in the present disclosure exogenously integrated in a genome. The expression system may be a host cell, which may express the fusion protein described in the present disclosure, and the fusion protein may cooperate with a gRNA, so that the fusion protein may be localized to a target polynucleotide to achieve base editing of the target polynucleotide.

In some embodiments of the present disclosure, the host cell may be a eukaryotic cell and/or a prokaryotic cell. The eukaryotic cell includes a fungus, a plant cell, an animal cell, for example, a mammalian cell, such as a mouse cell and a human cell, etc., for example, a mouse brain neuroblastoma cell, a human embryonic kidney cell, a human cervical cancer cell, a human colon cancer cell, or a human osteosarcoma cell, etc., more specifically, an N2a cell, a HEK293FT cell, a Hela cell, an HCT116 cell, or a U2OS cell, etc.

Another aspect of the present disclosure provides a system for modifying a target polynucleotide, the system comprising: a) one or more guide RNAs (gRNAs) or one or more nucleic acids encoding the one or more gRNAs, the one or more gRNAs being capable of hybridizing with the target polynucleotide.

The system further comprises any one or more of the following (a)-(f: b) the deaminase described in the present disclosure, or a nucleic acid encoding the deaminase; and a nucleic acid-binding polypeptide, or a nucleic acid encoding the nucleic acid-binding polypeptide; c) the fusion protein described in the present disclosure, or a nucleic acid encoding the fusion protein; d) the expression vector described in the present disclosure; e) the pharmaceutical composition described in the present disclosure; f) the expression system described in the present disclosure.

The target polynucleotide may also be referred to as a target nucleic acid.

In some embodiments of the present disclosure, the deaminase in b) and the nucleic acid-binding polypeptide form a complex to deaminate a base in a nucleic acid sequence.

In some embodiments of the present disclosure, b) together with a) forms a complex to deaminate a base in the target polynucleotide.

In some embodiments of the present disclosure, b), c), d), e), and/or f) are guided by a) to the target polynucleotide to deaminate the base in the target polynucleotide.

Another aspect of the present disclosure provides a method for modifying a target polynucleotide, the method comprising contacting the target polynucleotide with the deaminase described in the present disclosure, the fusion protein described in the present disclosure, the nucleic acid described in the present disclosure, the expression vector described in the present disclosure, the pharmaceutical composition described in the present disclosure, the expression system described in the present disclosure, or the system for modifying a target polynucleotide described in the present disclosure.

In some embodiments of the present disclosure, the expression vector or expression system comprises a nucleotide sequence encoding the deaminase or the fusion protein.

In some embodiments of the present disclosure, the modification is that at least one nucleotide in the target polynucleotide is deaminated.

Optionally, the method is for non-therapeutic or non-diagnostic purposes.

Based on common knowledge in the art, the above preferred conditions may be arbitrarily combined to obtain various preferred examples of the present disclosure.

The reagents and raw materials used in the present disclosure are all commercially available.

The present disclosure has the following advantageous effects: (1) the fusion proteins of some deaminases provided by the present disclosure have relatively high editing efficiency; (2) the fusion proteins obtained from some deaminases provided by the present disclosure have a relatively wide editing window; (3) the fusion proteins obtained from some deaminases of the present disclosure have a narrow editing window.

### BRIEF DESCRIPTION OF THE DRAWINGS

FIG. 1 is a plasmid map of CBE-27-pCDNA3.1.

FIG. 2 is a schematic diagram illustrating a Sanger analysis result for CBE-27 single base editing in Example 2.

FIG. 3 is a schematic diagram illustrating a Sanger analysis result for CBE-29 single base editing in Example 2.

FIG. 4 is a schematic diagram illustrating a Sanger analysis result for NC group single base editing in Example 2.

FIG. 5 is a schematic diagram illustrating a Sanger analysis result for CBE-136 single base editing in Example 3.

FIG. 6 is a schematic diagram illustrating a Sanger analysis result for CBE-140 single base editing in Example 3.

FIG. 7 is a schematic diagram illustrating a Sanger analysis result for CBE-139 single base editing in Example 4.

FIG. 8 is a schematic diagram illustrating a Sanger analysis result for CBE-145 single base editing in Example 4.

FIG. 9 is a schematic diagram illustrating a Sanger analysis result for CBE-141 single base editing in Example 4.

FIG. 10 is a schematic diagram illustrating a Sanger analysis result for CBE-143 single base editing in Example 4.

FIG. 11 is a schematic diagram illustrating validation results for single base editors containing deaminase-145 mutants in Example 5.

FIG. 12 is a schematic diagram illustrating validation results for single base editors containing deaminase-145 mutants in Example 6.

### DETAILED DESCRIPTION

### Terms

### Sequence Identity

As used herein, the term ‘identity’ (or ‘percent identity’) refers to sequence matching between two polypeptides or between two nucleic acids. When a position in two sequences being compared is occupied by the same base or amino acid monomer subunit (e.g., when a position in each of two DNA molecules is occupied by adenine, or a position in each of two polypeptides is occupied by lysine), the sequences or molecules are identical at that position. The ‘percent identity’ between two sequences is a function of the number of matching positions shared by the two sequences divided by the number of positions being compared, multiplied by 100%. For example, if six out of ten positions of two sequences match, then the two sequences have 60% sequence identity. Typically, comparison is performed when aligning two sequences to produce maximum sequence identity. Such an alignment may be performed using publicly available and commercially available alignment algorithms and programs, such as, but not limited to, CLUSTERal Ω, MAFFT, Probcons, T-Coffee, Probalign, BLAST, which may be reasonably selected by a person of ordinary skill in the art. The person of ordinary skill in the art can determine suitable parameters for aligning sequences, (e.g., including any algorithms needed to achieve optimal alignment or optimal comparison for the full length of the sequences being compared, and any algorithms needed to achieve optimal alignment or optimal comparison for a local portion of the sequences being compared).

### Deaminase

The term ‘deaminase’ refers to an enzyme that catalyzes a deamination reaction (i.e., removal of an amino group from a base of a nucleotide). In some embodiments, the deaminase is a cytidine deaminase. The cytidine deaminase catalyzes cytidine deamination to produce uridine, or catalyzes deoxycytidine deamination to produce deoxyuridine; that is, catalyzes cytosine group deamination to produce uracil group. The deaminase may act on DNA and/or RNA.

The terms deaminase and deaminase polypeptide may be used interchangeably.

In some embodiments, the deaminase may further comprise a homologous or heterologous domain. Non-limiting examples include: the deaminase further comprising a nucleic acid-binding polypeptide (e.g., the deaminase comprising a Cas9 nickase domain, the deaminase comprising a Cas12 domain, the deaminase comprising a TnpB domain, or the deaminase comprising a Talen domain), the deaminase further comprising a cellular localization signal (e.g., an NLS, a nuclear export signal, a mitochondrial localization signal, or a chloroplast localization signal), or the deaminase further comprising a UGI domain.

### Deamination

In some embodiments, the deamination refers to catalyzing the deamination of the base in the DNA and/or RNA molecule. In some embodiments, the deamination refers to catalyzing the deamination of the base in a DNA molecule. In some embodiments, the deamination refers to catalyzing the deamination of the base in an RNA molecule. In some embodiments, the deamination refers to catalyzing the cytidine deamination to produce uridine, or catalyzing the deoxycytidine deamination to produce deoxyuridine. In some embodiments, the deamination refers to catalyzing the cytosine group deamination to produce the uracil group.

In some embodiments, an expected edited base is upstream of a PAM site. In some embodiments, the expected edited base is the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, or 40th nucleotide upstream of the PAM site. In some embodiments, the expected edited base is downstream of the PAM site. In some embodiments, the expected edited base is the 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, 24th, 25th, 26th, 27th, 28th, 29th, 30th, 31st, 32nd, 33rd, 34th, 35th, 36th, 37th, 38th, 39th, or 40th nucleotide downstream of the PAM site. The edited base is a base that undergoes deamination. The position of the edited base is an editing window. In some embodiments, a target sequence comprises the editing window. In some embodiments, the editing window comprises 1 to 10 nucleotides. In some embodiments, the length of the editing window is 1 to 9, 1 to 8, 1 to 7, 1 to 6, 1 to 5, 1 to 4, 1 to 3, 1 to 2, or 1 nucleotide(s). In some embodiments, the length of the editing window is 1, 2, 3, 4, 5, 6, 7, 8, 9, 10, 11, 12, 13, 14, 15, 16, 17, 18, 19, or 20 nucleotide(s).

### Fusion Protein

In the present disclosure, when referring to ‘a fusion protein comprising XXXX deaminase’ or using a similar description, it is intended to mean that the amino acid sequence of the fusion protein comprises the amino acid sequence constituting the deaminase.

In the present disclosure, when referring to ‘a fusion protein comprising XXXX nucleic acid-binding polypeptide’ or using a similar description, it is intended to mean that the amino acid sequence of the fusion protein comprises the amino acid sequence constituting the nucleic acid-binding polypeptide.

### Proteins, Peptides, And Polypeptides

The terms ‘protein’, ‘peptide’, and ‘polypeptide’ may be used interchangeably in the present disclosure and refer to polymers of amino acid residues linked together by peptide (amide) bonds. The term refers to a protein, peptide, or polypeptide of any size, structure, or function. Generally, a protein, peptide, or polypeptide has a length of at least three amino acids. A protein, peptide, or polypeptide may refer to a single protein or a collection of proteins. One or more amino acids in a protein, peptide, or polypeptide may be modified, for example, by adding a chemical entity (e.g., a carbohydrate group, a hydroxyl group, a phosphate group, a farnesyl group, an isofarnesyl group, a fatty acid group, a linker for conjugation, functionalization, or other modifications, or the like). A protein, peptide, or polypeptide may also be a single molecule or a multimolecular complex. A protein, peptide, or polypeptide may merely be a fragment of a naturally occurring protein or peptide. A protein, peptide, or polypeptide may be naturally occurring, recombinant, or synthetic, or any combination thereof.

### Nucleic Acid-Binding Polypeptide

Optionally, the nucleic acid-binding polypeptide is a DNA binding polypeptide.

Optionally, the nucleic acid-binding polypeptide is an RNA binding polypeptide.

Optionally, the nucleic acid-binding polypeptide is a TALEN nuclease, a ZFN, a Cas enzyme, or a meganuclease.

In some embodiments of the present disclosure, the nucleic acid-binding polypeptide is an RNA-guided nuclease (RGN).

In some embodiments of the present disclosure, the DNA binding polypeptide may be the RNA-guided nuclease.

In some embodiments of the present disclosure, the RNA binding polypeptide may be the RNA-guided nuclease.

### RNA-Guided Nuclease

The term RNA-guided nuclease refers to a polypeptide that binds to a specific target polynucleotide in a sequence-specific manner, and the polypeptide is guided to the target polynucleotide by a gRNA molecule that complexes with the polypeptide and hybridizes with the target polynucleotide. Although the RNA-guided nuclease can cleave the target sequence upon binding, the term RNA-guided nuclease also includes a nuclease-inactivated RNA-guided nuclease that is capable of binding but not cleaving the target sequence. The cleavage of the target sequence by the RNA-guided nuclease may result in single-stranded or double-stranded breaks. The RNA-guided nuclease that is capable of cleaving only one strand of a double-stranded nucleic acid molecule is referred to as a nickase in the present disclosure.

Optionally, the DNA binding polypeptide may be a CRISPR-associated nuclease, i.e., a Cas enzyme (also referred to as a Cas protein).

In some embodiments of the present disclosure, the nucleic acid-binding polypeptide is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9, Cas10, Cas10d, Cas12a/Cpf1, Cas12b/C2c1, Cas12c/C2c3, Cas12d/CasY, Cas12e/CasX, Cas12f/CasZ, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Cas13a, Cas13b, Cas13c, Cas13d, Cas13e, Cas13f, TnpB, IscB, IsrB, and a Fancor nuclease; or a fragment of any one of them. A non-limiting example of the fragment may be a nucleic acid binding domain fragment.

In some embodiments of the present disclosure, the nucleic acid-binding polypeptide is selected from Cas9, Cas12, Cas13, TnpB, IscB, IsrB, a Fancor nuclease, or a fragment of any one of them, including but not limited to the nucleic acid binding domain fragment.

In some embodiments of the present disclosure, the Cas9 is selected from SpCas9, SaCas9, Nme2Cas9, Nme3Cas9, CjCas9, NmCas9, FnCas9, PpnCas9, FrCas9, SauCas9, SauriCas9, ScaCas9, St1Cas9, BlatCas9, CdiCas9, and GeoCas9, a fragment of any one of them, and a mutant of any one of them or a fragment of the mutant.

In some embodiments of the present disclosure, the nucleic acid-binding polypeptide is selected from AsCpf1, enAsCas12a (addgene plasmid #196724), dFnCas12a (addgene plasmid #136379), ErCas12a, LbCas12a D832A, LbCas12a H759A, LbCas12a E795L, FnCas12a3, FnCas12a D917A, AsCas12a R1226A, AsCas12a D908A, AsCas12a E174R/S542R, AsCas12a (S542R/K548V/N552R), PrCas12a, PxCas12a, PcCas12a, PdCas12a, Mb2Cas12a, Mb3Cas12a, MICas12a, CMaCas12a, CMtCas12a, HkCas12a, Lb5Cas12a, ErCas12a, TsCas12a, FnCpf1, LbCas12a, ttHsCas12a, AaCas12b, AaCas12b D570A, AaCas12b Q119F/E475R/E758R, BhCas12b, BvCas12b, BrCas12b, AkCas12b, AmCas12b, BsCas12b, OspCas12c, Cas12c2 (addgene plasmid #183072), Cas12c_4 (addgene plasmid #183071), Cas12c1 (addgene plasmid #120872), CasY.1 (from Katanobacteria), CasY.2 (from Vogelbacteria), CasY3 (from Vogelbacteria), CasY.4 (from Parcubacteria), CasY5 (from Komeilibacteria), CasY6 (from Kerfeldbacteria), PlmCasX, DpbCasX, Un1Cas12f, CnCas12f1, enRhCas12f1, AsCas12f1, SpaCas12f1, Cas12g1 (addgene plasmid #120879), Cas12 h of WO2021113522A1 (SEQ ID NO: 1 in the patent application), Cas12i1 (addgene plasmid #171670), Cas12i2 (addgene plasmid #188275), Cas12i1 (addgene plasmid #120882), Cas12i2 (addgene plasmid #120883), Cas12i protein designated Cas12f.4/Cas12f.5/Cas12f.6 in CN111757889B, dSiCas12i(D1049A), SiCas12i, Si2Cas12i, WiCas12i, Wi2Cas12i, Wi3Cas12i, SaCas12i, Sa2Cas12i, Sa3Cas12i, WaCas12i, Wa2Cas12i, xCas12i, hfCas12Max, Cas12i-Max (addgene plasmid #188276), Cas12i1 D647A (addgene plasmid #171671), Cas12i-HiFi (addgene plasmid #188269), Cas12i1 D647A, Cas12j3 (addgene plasmid #188497), Cas12j2 (addgene plasmid #188498), AsCas12j-2 (addgene plasmid #191655), Cas12j-8 (addgene plasmid #194966), ShCasI2k, N7Cas12k, AcCas12k, Cas12k-TniQ (addgene plasmid #181787), Cas12k-TnsC (addgene plasmid #181789), Cas12l, MmCas12m, MmCas12m AZF (H549A, C552A), dCas12m-AZF (D485A, H549A, C552A), AcCas12n, dAcCas12n(D240), TnpB Actinomadura_cellulosilytica_strain_DSM_45823, TnpB Actinomadura_namibiensis_strain_DSM_44197, TnpB Actinomadura_umbrina_strain_DSM_43927, TnpB Actinoplanes_lobatus_strain_DSM_43150 (TnpB-1 and TnpB-2), TnpB Alicyclobacillus_macrosporagiidus_strain_DSM_17980, TnpB Haloactinospora_alba_Strain_DSM_45015, TnpB Lipingzhangella_halophila_strain_DSM_102030, TnpB Meiothermus_Silvanus_DSM_9946, TnpB QNFX01000004, ISDra2 TnpB(PDB: 8H1J), KralscB-1, AwalscB, OgeulscB, GtFz1 (from Guillardia theta), SpuFz1 (from Spizellomyces punctatus), NlovFz2 (from Percolozoa Naegleria lovaniensis) and MmeFz2 (from Mercenaria mercenaria), a fragment of any one of them, and a mutant of any one of them, or a fragment of the mutant.

In some embodiments of the present disclosure, the DNA binding polypeptide can cleave one strand of a double-stranded nucleic acid molecule (e.g., a double-stranded DNA molecule). Optionally, the DNA binding polypeptide is an RNA-guided nuclease having nickase activity.

In some embodiments of the present disclosure, the Cas enzyme cleaves a target strand of a double-stranded nucleic acid molecule, meaning that the Cas enzyme cleaves a strand that is base-paired with (is complementary to) a gRNA (e.g., an sgRNA) bound to the Cas enzyme.

In some embodiments of the present disclosure, the nucleic acid-binding polypeptide may be an RNA-guided nuclease having inactivated nuclease activity.

In some embodiments of the present disclosure, the nucleic acid-binding polypeptide is a variant of an RNA-guided nuclease with completely inactivated nuclease activity compared to a wild-type form of the RNA-guided nuclease, for example, a dCas enzyme, including but not limited to Cas9, Cas12, Cas13, TnpB, IscB, IsrB, and Fancor, each of which has completely inactivated nuclease activity (which may be referred to as dead Cas9, dead Cas12, dead Cas13, dead TnpB, dead IscB, dead IsrB, and dead Fancor).

In some embodiments of the present disclosure, the nucleic acid-binding polypeptide may be a variant of an RNA-guided nuclease with partially inactivated nuclease activity compared to a wild-type form of the RNA-guided nuclease, such as an nCas enzyme, for example, a polypeptide fragment that retains the nuclease activity of cleaving a single strand of double-stranded DNA, including but not limited to nickase Cas9 and nickase Cas12.

A target polynucleotide binds with the nucleic acid-binding polypeptide provided in the present disclosure, and hybridizes with the gRNA associated with the nucleic acid-binding polypeptide. Then, if the nucleic acid-binding polypeptide has nuclease activity, the target sequence may subsequently be cleaved by the nucleic acid-binding polypeptide. For example, the nucleic acid-binding polypeptide may optionally cleave any one single strand of double-stranded DNA or cleave both single strands, or may bind to the target polynucleotide without cleaving any single strand; simultaneously with or after the nucleic acid-binding polypeptide binds or cleaves the target polynucleotide, the deaminase deaminates at least one base of the target polynucleotide.

In some embodiments, the binding depends on the recognition of a PAM sequence by the RNA-guided nuclease. In some embodiments, the binding does not depend on the recognition of a PAM sequence by the RNA-guided nuclease. The PAM sequences recognized by many nucleases described in the present disclosure are known.

The term ‘cleave’ or ‘cleavage’ refers to the hydrolysis of at least one phosphodiester bond within the backbone of the target polynucleotide, which may result in single-stranded or double-stranded breaks within the target sequence. The presently disclosed nucleic acid-binding polypeptide may function as an endonuclease or an exonuclease (removing consecutive nucleotides from the 5′ and/or 3′ ends of a polynucleotide), thereby cleaving nucleotides within the polynucleotide. In other embodiments, the disclosed nucleic acid-binding polypeptide may cleave nucleotides of the target sequence at any position within the polynucleotide, thereby acting as both an endonuclease and an exonuclease. The cleavage of the target polynucleotide by the presently disclosed nucleic acid-binding polypeptide may result in staggered breaks or blunt ends.

### Nuclear Localization Signal, Nuclear Localization Sequence, NLS

The terms nuclear localization signal, nuclear localization sequence, and NLS may be used interchangeably. The presently disclosed fusion protein may comprise at least one nuclear localization signal (NLS) to enhance the delivery of the nucleic acid-binding polypeptide to the cell nucleus. The nuclear localization signal is known in the art and typically comprises a stretch of basic amino acids (see, e.g., Lange et al., J. Biol. Chem. (2007) 282:5101-5105). In specific embodiments, the fusion protein comprises two, three, four, five, six, or more nuclear localization signals. One or more nuclear localization signals may be heterologous nuclear localization signals. Non-limiting examples of nuclear localization signal applicable to the presently disclosed RGNs are the NLSs of SV40 large T antigen, nucleoplasmin, and c-Myc (see, e.g., Ray et al., (2015) Bioconjug Chem 26(6):1004-7).

### Linker

As used in the present disclosure, the term ‘linker’ refers to a chemical group or molecule connecting two molecules or portions (e.g., a binding domain and a cleavage domain of a nuclease). Typically, a linker is located between or on both sides of two groups, molecules, or other portions, and is linked to each group, molecule, or portion via a covalent bond, thereby connecting the two. In some embodiments, the linker is an amino acid or a plurality of amino acids (e.g., a peptide or a protein). In some embodiments, the linker may be an organic molecule, a group, a polymer, or a chemical moiety. In some embodiments, the linker has a length of 5 to 100 amino acids (e.g., having a length of 5, 6, 7, 8, 9, 10, 11, 12, 13, 14, 15, 16, 17, 18, 19, 20, 21, 22, 23, 24, 25, 26, 27, 28, 29, 30, 30 to 35, 35 to 40, 40 to 45, 45 to 50, 50 to 60, 60 to 70, 70 to 80, 80 to 90, 90 to 100, 100 to 150, or 150 to 200 amino acids). Longer or shorter linkers may also be considered.

In some embodiments, the linker comprises a sequence set forth in any one of SEQ ID NOs: 33 to 37 and EFE, or a fragment or any one variant of any one of them.

In some embodiments, the linker comprises a (GGGGS)n (SEQ ID NO: 35), (G)n, (EAAAK)n (SEQ ID NO: 36), (XP)n, or (SGG)n motif, or any combination thereof, wherein n is independently an integer from 1 to 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if there is more than one linker or more than one linker motif, then n is independently any combination thereof. Other suitable linker motifs and linker configurations are apparent to those skilled in the art. In some embodiments, suitable linker motifs and configurations include those described in Chen et al., Fusion protein linkers: property, design and functionality (Adv Drug Deliv Rev. 2013; 65(10):1357-69), the entire contents of which are incorporated herein by reference. Based on the present disclosure, other suitable linker sequences are apparent to those skilled in the art.

### UGI

The fusion protein of the present disclosure may comprise suitable UGI sequences, and additional suitable UGI sequences are known to those skilled in the art and include, for example, those disclosed in Wang et al., 1989 J. Biol. Chem. 264:1163-1171; Lundquist et al., 1997 J. Biol. Chem. 272:21408-21419; Ravishankar et al., 1998 Nucleic Acids Res. 26:4880-4887; and Putnam et al., 1999 J. Mol. Biol. 287:331-346 (1999), the entire contents of each of which are incorporated herein by reference.

Guide RNA, gRNA, Guide Polynucleotide

The terms guide RNA, gRNA, and guide polynucleotide may be used interchangeably. In some embodiments of the present disclosure, the gRNA comprises a guide sequence and a scaffold sequence. The scaffold sequence interacts with an RNA-guided nuclease. The scaffold sequence is a region of the gRNA that typically remains constant when designing the gRNA molecule. For example, the scaffold sequence may refer to the portion of a gRNA molecule other than the guide sequence. For example, when the nucleic acid-binding polypeptide is Cas9, Cas12, or Cas13, the gRNA may be a crRNA or a complex of crRNA and tracrRNA (e.g., a single molecule gRNA chimerically formed by crRNA and tracrRNA).

In some embodiments of the present disclosure, the guide sequence hybridizes with the target polynucleotide. The guide sequence refers to a continuous nucleotide sequence in the gRNA, which has partial or complete complementarity with the target polynucleotide (specifically, which has partial or complete complementarity with a target sequence in the target polynucleotide [target nucleic acid]), and may hybridize with the target sequence in the target polynucleotide via base pairing facilitated by an RNA-guided nuclease. Complete complementarity of the guide sequence with the target sequence as described in the present disclosure is not required, as long as there is sufficient complementarity to cause hybridization and facilitate the formation of the gene editing complex. As used in the present disclosure, the term ‘target sequence’ refers to a small segment of sequence in the target polynucleotide, which may be complementary to the guide sequence of the gRNA molecule (fully or partially complementary). The gene editing complex is sequence-specifically localized to the target sequence via the guide sequence and performs a corresponding function at or near this position. The length of the target sequence is often dozens of nucleotides (nt) (e.g., it may be 10 to 60 nt, 15 to 50 nt, 15 to 40 nt, 15 to 35 nt, 20 to 35 nt, 20 to 30 nt, about 10 nt, about 20 nt, about 30 nt, about 40 nt, about 50 nt, or about 60 nt).

In some embodiments of the present disclosure, the guide sequence has at least 50% sequence identity with the target polynucleotide. In some embodiments of the present disclosure, the guide sequence has at least 60%, at least 70%, at least 80%, at least 85%, at least 90%, at least 95%, or 100% sequence identity with the target polynucleotide.

The length of the guide sequence is often dozens of nucleotides (nt) (e.g., it may be 10 to 60 nt, 15 to 50 nt, 15 to 40 nt, 15 to 35 nt, 20 to 35 nt, 20 to 30 nt, about 10 nt, about 20 nt, about 30 nt, about 40 nt, about 50 nt, or about 60 nt).

In some embodiments of the present disclosure, the guide sequence is located at the 3′ end or the 5′ end of the scaffold sequence. In some embodiments of the present disclosure, the guide sequence is located at the 3′ end of the scaffold sequence. In some embodiments of the present disclosure, the guide sequence is located at the 5′ end of the scaffold sequence.

In some embodiments of the present disclosure, the gRNA comprises an aptamer sequence.

In some embodiments of the present disclosure, the aptamer sequence is inserted into the loop of the stem-loop structure of the secondary structure of the gRNA.

In some embodiments of the present disclosure, the guide RNA comprises modified nucleotides. The modifications include but are not limited to 2′-O-methyl modification, 2′-O-methyl-3′-thiophosphorothioate modification, or 2′-O-methyl-3′-thioPACE modification. In some embodiments of the present disclosure, the gRNA comprises modified nucleotide, wherein the modified nucleotide is selected from deoxyribonucleotide and locked nucleic acid (LNA). In some embodiments, the gRNA comprises at least one chemically modified nucleotide.

In some embodiments, the gRNA is a hybrid RNA-DNA guide, meaning that a portion of the RNA nucleotides in the gRNA is replaced by DNA nucleotides. In some embodiments, the gRNA is a hybrid RNA-LNA guide, meaning that a portion of the RNA nucleotides in the gRNA is replaced by LNA nucleotides.

In some embodiments of the present disclosure, the target RNA is located in the nucleus and/or the cytoplasm of a eukaryotic cell.

### Target Polynucleotide

According to the context, the term ‘target polynucleotide’ may be used interchangeably with ‘target nucleic acid’ and ‘target gene’.

The deaminase or the fusion protein of the present disclosure may deaminate a base in the target polynucleotide, catalyzing the deamination of cytidine to uridine, or catalyzing the deamination of deoxycytidine to deoxyuridine. It may be used to achieve base conversion on DNA molecules or base conversion on RNA molecules. For example, it may be used to edit exon regions, intron regions, and/or regulatory regions (e.g., promoter regions, enhancer regions) of genes; and it may be used to edit precursor messenger RNA (pre-mRNA) molecules, mature messenger RNA (mRNA) molecules, or non-coding RNA molecules.

Some disease-causing mutations are disclosed in a patent application with Publication No. CN108513575A, which is incorporated herein by reference. These mutations may be corrected by the deaminase or the fusion protein of the present disclosure, thereby treating the corresponding diseases.

The deaminase or the fusion protein of the present disclosure may edit genes (normal gene copies or mutant gene copies) in Schedule 1, or their corresponding pre-mRNA molecules or mature mRNA molecules, thereby treating the corresponding diseases or disorders.

When referring to an RNA sequence, T in the sequence may be used interchangeably with ‘u’. When referring to a guide sequence, T in the sequence may be used interchangeably with ‘u’.

The present disclosure is further illustrated by the following examples, but the present disclosure is not limited to the scope of the described examples. The experimental methods in the following examples where specific conditions are not specified are carried out in accordance with conventional methods and conditions, or as instructed by the product manuals.

### EXAMPLES

The inventors obtained deaminases-23 to deaminases-34 through bioinformatics analysis and screening.

In addition, deaminases-8 to deaminases-22 and deaminases-36 to deaminases-134 were obtained through structure-based de novo design.

### Experimental Example 1: Synthesis of Expression Plasmids for Different Cytosine Deaminases

Expression cassettes for cytosine base editors composed of nSpRYCas9, UGI, and different cytosine deaminase sequences were designed and synthesized (using the nucleic acid sequences encoding the deaminase as shown in the sequence listing), and expression cassettes for sgRNAs targeting the VEGFA gene were simultaneously synthesized. Then, the two types of cassettes were inserted into a pCDNA3.1 expression vector, thereby obtaining expression vector plasmids for cytosine base editors comprising different deaminases. The number ‘n’ of a deaminase-n is identical to the number ‘n’ in an editor CBE-n and is identical to the number ‘n’ in a vector plasmid CBE-n-pCDNA3.1. For example, deaminase-27 may be constructed to obtain editor CBE-27, and its expression vector plasmid is CBE-27-pCDNA3.1. Other examples described below also adopt the same representation manner.

### Amino Acid Sequences

This example screened and tested a plurality of cytosine deaminase proteins, and the amino acid sequences of a portion of the deaminases are as follows.

Deaminase-27 (SEQ ID NO: 3):
HYPEIQPKEFKDVYDPSVLPKVTYLLYEIKWGNSTKIWRNWCRNTSTQHA
EINFLENAFRERSFNPLTHCSITWFLSWSPCWKCSQSVVEFLKTYPKVNL
EMYVARLFWHEDRSNRLGLRDLVMNGVTIRVMDLSAYNYCWRTFVSHQNV
DDYWPWHLTPWIMFFSFELQLILQVSRSLGGKDSIQKLSRDMYR
Deaminase-29 (SEQ ID NO: 5):
HTADQGRRIKSWEFTSFFDPSQLRKETSLLSEVWWGSSSRLWRSCSQNST
HHVEQSFLDHFQAQRHLGPSTTCTITWFLSWSPCWECARAIRTFLDKYPT
VKLTIYVARLYWHMDPQNRQGLRNLVKGGVDVRIMGPSEHYYCWRNFVDY
SRGEEEEWPRYHRVPIHLFDLELHCICLDLPACLTISPVKHQQLLSFSLT
LHTCHYQKLPPALLLLLGLVRPPPPQTSLSVAHRGRGPGTVNGGEWDPNN
WGWFWVLR
Deaminase-48 (SEQ ID NO: 6)
RSSKMPEELFYKYFYPKKDRTGLTTTYLCYELRTQVDATTYEVEKGWIED
EDGVHAYLLFLEKFSALNLDPDKKYDVTLYASFSPCYECAKALAEFLKAH
PNVSITIKVANLYNCEPGSPNKEGLRLLKEAGVTISIMDEADYKYVWNTF
VDNKGKEYVPPEGLKENSEELQKKLEEILTPPEVEEARRRALREAGL
Deaminase-136 (SEQ ID NO: 57):
MEPWPSLPPKAYNFNFRNLREAPNRNSTYLCFEVEEEQLDSPESSYWGVF
RNQPSHLHQVYPEEPLHAEQRFLSWFYDALLSAHAYYRVTWYMSWSPCSE
CAEEVVSFLGEHENVSLSISASRLYKCDDQDQQEGLRLLDQAGVEVAMMS
PDDFEYCWDNFVDNRRRSFRYWKGIRRNYHALDDELKEILWG
Deaminase-140 (SEQ ID NO: 58):
SLTADSDPLPCSNSLYRPPRSCLMQRKKFLYHYKNVRWAKGRHETYMCYV
VKRRESAQTSSLDFGFLRNTARAHAEVVFLDYIRDWPLDPGLTYRLTWFS
SWSPCYDCAARVVHFLQARPNVRLRLFIARLYYCSEGMRQPEGLRQLHQA
GVGLGIMTYKDYFYCWNNFVANKEKSFKAWEGLHENAVRLSRLLRRILE

### Nucleic Acid Sequences

CBE-27-pCDNA3.1 Plasmid (SEQ ID NO: 10):
GCTAGCGCCACCatgaaacggacagccgacggaagcgagttcgagtcaccaaagaagaagcggaaagtccactaccc
cgagattcagcccaaggagttcaaggacgtgtacgaccctagcgtgctgcccaaggtgacctacctgctgtacgagatcaagtggg
gcaacagcaccaagatctggagaaactggtgcagaaacacaagcacacagcacgccgagatcaacttcctggagaacgccttc
agagagcggagcttcaaccccctgacccactcagcatcacctggttcctgagctggagcccctgctggaagtgcagccaaagcgt
ggtggagttcctcaagacctaccccaagatgaacctggagatatacgtggctagactgttctggcacgaggacagaagcaacagac
taggcctacaggacctagtgatgaacggcgtgaccatcagagtgatggacctgagcgcctacaactactgctggagaaccttcatga
gccatcagaacgtggacgactactcgccctggcacctgaccccctggatcatattcttcagcttcgagctgcagctgatcctgcaagtg
agcagaagcctgggcggcaaggacagcattcagaagctgagcagagacatgtacagatctggaggatctagcggaggatcctct
ggcagcgagacaccaggaacaagcgagtcagcaacaccagagagcagtggcggcagcagcggcggcagcgacaagaagta
cagcatcggcctggccatcggcaccaactctgtgggctgggccgtgatcaccgacgagtacaaggtgcccagcaagaaattcaag
gtgctgggcaacaccgaccggcacagcatcaagaagaacctgatcggagccctgctgttcgacagcggcgaaacagccgagag
aacccggctgaagagaaccgccagaagaagatacaccagacggaagaaccggatctgctatctgcaagagatcttcagcaacg
agatggccaaggtggacgacagcttcttccacagactggaagagtccttcctggtggaagaggataagaagcacgagcggcaccc
catcttcggcaacatcgtggacgaggtggcctaccacgagaagtaccccaccatctaccacctgagaaagaaactggtggacagc
accgacaaggccgacctgcggctgatctatctggccctggcccacatgatcaagttccggggccacttcctgatcgagggcgacctg
aaccccgacaacagcgacgtggacaagctgttcatccagctggtgcagacctacaaccagctgttcgaggaaaaccccatcaacg
ccagcggcgtggacgccaaggccatcctgtctgccagactgagcaagagcagacggctggaaaatctgatcgcccagctgcccg
gcgagaagaagaatggcctgttcggaaacctgattgccctgagcctgggcctgacccccaacttcaagagcaacttcgacctggcc
gaggatgccaaactgcagctgagcaaggacacctacgacgacgacctggacaacctgctggcccagatcggcgaccagtacgc
cgacctgtttctggccgccaagaacctgtccgacgccatcctgctgagcgacatcctgagagtgaacaccgagatcaccaaggccc
ccctgagcgcctctatgatcaagagatacgacgagcaccaccaggacctgaccctgctgaaagctctcgtgcggcagcagctgcct
gagaagtacaaagagattttcttcgaccagagcaagaacggctacgccggctacattgacggggagccagccaggaagagttct
acaagttcatcaagcccatcctggaaaagatggacggcaccgaggaactgctcgtgaagctgaacagagaggacctgctgcgga
agcagcggaccttcgacaacggcagcatcccccaccagatccacctgggagagctgcacgccattctgcggcggcaggaagattt
ttacccattcctgaaggacaaccgggaaaagatcgagaagatcctgaccttccgcatcccctactacgtgggccctctggccagggg
aaacagcagattcgcctggatgaccagaaagagcgaggaaaccatcaccccctggaacttcgaggaagtggtggacaagggcg
cttccgcccagagcttcatcgagcggatgaccaacttcgataagaacctgcccaacgagaaggtgctgcccaagcacagcctgctg
tacgagtacttcaccgtgtataacgagctgaccaaagtgaaatacgtgaccgagggaatgagaaagcccgccttcctgagcggcga
gcagaaaaaggccatcgtggacctgctgttcaagaccaaccggaaagtgaccgtgaagcagctgaaagaggactacttcaagaa
aatcgagtgcttcgactccgtggaaatctccggcgtggaagatcggttcaacgcctccctgggcacataccacgatctgctgaaaatt
atcaaggacaaggacttcctggacaatgaggaaaacgaggacattctggaagatatcgtgctgaccctgacactgtttgaggacag
agagatgatcgaggaacggctgaaaacctatgcccacctgttcgacgacaaagtgatgaagcagctgaagcggcggagatacac
cggctggggcaggctgagccggaagctgatcaacggcatccgggacaagcagtccggcaagacaatcctggatttcctgaagtcc
gacggcttcgccaacagaaacttcatgcagctgatccacgacgacagcctgacctttaaagaggacatccagaaagcccaggtgtc
cggccagggcgatagcctgcacgagcacattgccaatctggccggcagccccgccattaagaagggcatcctgcagacagtgaa
ggtggtggacgagctcgtgaaagtgatgggccggcacaagcccgagaacatcgtgatcgaaatggccagagagaaccagacca
cccagaagggacagaagaacagccgcgagagaatgaagcggatcgaagagggcatcaaagagctgggcagccagatcctga
aagaacaccccgtggaaaacacccagctgcagaacgagaagctgtacctgtactacctgcagaatgggcgggatatgtacgtgga
ccaggaactggacatcaaccggctgtccgactacgatgtggaccatatcgtgcctcagagctttctgaaggacgactccatcgacaa
caaggtgctgaccagaagcgacaagaaccggggcaagagcgacaacgtgccctccgaagaggtcgtgaagaagatgaagaa
ctactggcggcagctgctgaacgccaagctgattacccagagaaagttcgacaatctgaccaaggccgagagaggcggcctgag
cgaactggataaggccggcttcatcaagagacagctggtggaaacccggcagatcacaaagcacgtggcacagatcctggactc
ccggatgaacactaagtacgacgagaatgacaagctgatccgggaagtgaaagtgatcaccctgaagtccaagctggtgtccgatt
tccggaaggatttccagttttacaaagtgcgcgagatcaacaactaccaccacgcccacgacgcctacctgaacgccgtcgtggga
accgccctgatcaaaaagtaccctaagctggaaagcgagttcgtgtacggcgactacaaggtgtacgacgtgcggaagatgatcgc
caagagcgagcaggaaatcggcaaggctaccgccaagtacttcttctacagcaacatcatgaactttttcaagaccgagattaccct
ggccaacggcgagatccggaagcggcctctgatcgagacaaacggcgaaaccggggagatcgtgtgggataagggccgggatt
ttgccaccgtgcggaaagtgctgagcatgccccaagtgaatatcgtgaaaaagaccgaggtgcagacaggcggcttcagcaaag
agtctatcagacccaagaggaacagcgataagctgatcgccagaaagaaggactgggaccctaagaagtacggcggcttcctgt
ggcccaccgtggcctattctgtgctggtggtggccaaagtggaaaagggcaagtccaagaaactgaagagtgtgaaagagctgctg
gggatcaccatcatggaaagaagcagcttcgagaagaatcccatcgactttctggaagccaagggctacaaagaagtgaaaaag
gacctgatcatcaagctgcctaagtactccctgttcgagctggaaaacggccggaagagaatgctggcctctgccaagcagctgca
gaagggaaacgaactggccctgccctccaaatatgtgaacttcctgtacctggccagccactatgagaagctgaagggctcccccg
aggataatgagcagaaacagctgtttgtggaacagcacaagcactacctggacgagatcatcgagcagatcagcgagttctccaa
gagagtgatcctggccgacgctaatctggacaaagtgctgtccgcctacaacaagcaccgggataagcccatcagagagcaggc
cgagaatatcatccacctgtttaccctgaccagactgggagcccctagagccttcaagtactttgacaccaccatcgaccccaagcag
tacagaagcaccaaagaggtgctggacgccaccctgatccaccagagcatcaccggcctgtacgagacacggatcgacctgtctc
agctgggaggtgacagcggcgggagcgggggagcggggggagcactaatctgagcgacatcattgagaaggagactgggaa
acagctggtcattcaggagtccatcctgatgctgcctgaggaggtggaggaagtgatcggcaacaagccagagtctgacatcctggt
gcacaccgcctacgacgagtccacagatgagaatgtgatgctgctgacctctgacgcccccgagtataagccttgggccctggtcat
ccaggattctaacggcgagaataagatcaagatgctggaattcgagcccaagaagaagaggaaagtctagCTCGAGAATA
AAGGAAATTTATTTTCATTGCAATAGTGTGTTGGAATTTTTTGTGTCTCTCAGTTAACAAAAAA
GCACCGACTCGGTGCCACTTTTTCAAGTTGATAACGGACTAGCCTTATTTTAACTTGCTATTT
CTAGCTCTAAAACGAGGCGGGGTGGAGGGGGTCGGTGTTTCGTCCTTTCCACAAGATATAT
AAAGCCAAGAAATCGAAATACTTTCAAGTTACGGTAAGCATATGATAGTCCATTTTAAAACATA
ATTTTAAAACTGCAAACTACCCAAGAAATTATTACTTTCTACGTCACGTATTTTGTACTAATATC
TTTGTGTTTACAGTCAAATTAATTCCAATTATCTCTCTAACAGCCTTGTATCGTATATGCAAATA
TGAAGGAATCATGGGAAATAGGCCCTCTTCGAATAGCAGGCATGCTGGGGATGCGGTGGG
CTCTATGGCTTCTGAGGCGGAAAGAACCAGCTGGGGCTCTAGGGGGTATCCCCACGCGCC
CTGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTGCTTTACG
GCACCTCGACCCCAAAAAACTTGATTAGGGTGATGGTTCACGTAGTGGGCCATCGCCCTGA
TAGACGGTTTTTCGCCCTTTGACGTTGGAGTCCACGTTCTTTAATAGTGGACTCTTGTTCCA
AACTGGAACAACACTCAACCCTATCTCGGTCTATTCTTTTGATTTATAAGGGATTTTGCCGAT
TTCGGCCTATTGGTTAAAAAATGAGCTGATTTAACAAAAATTTAACGCGAATTAATTCTGTGGA
ATGTGTGTCAGTTAGGGTGTGGAAAGTCCCCAGGCTCCCCAGCAGGCAGAAGTATGCAAA
GCATGCATCTCAATTAGTCAGCAACCAGGTGTGGAAAGTCCCCAGGCTCCCCAGCAGGCA
GAAGTATGCAAAGCATGCATCTCAATTAGTCAGCAACCATAGTCCCGCCCCTAACTCCGCCC
ATCCCGCCCCTAACTCCGCCCAGTTCCGCCCATTCTCCGCCCCATGGCTGACTAATTTTTTT
TATTTATGCAGAGGCCGAGGCCGCCTCTGCCTCTGAGCTATTCCAGAAGTAGTGAGGAGGC
TTTTTTGGAGGCCTAGGCTTTTGCAAAAAGCTCCCGGGAGCTTGTATATCCATTTTCGGATC
TGATCAAGAGACAGGATGAGGATCGTTTCGCATGATTGAACAAGATGGATTGCACGCAGGT
TCTCCGGCCGCTTGGGTGGAGAGGCTATTCGGCTATGACTGGGCACAACAGACAATCGGC
TGCTCTGATGCCGCCGTGTTCCGGCTGTCAGCGCAGGGGCGCCCGGTTCTTTTTGTCAAG
ACCGACCTGTCCGGTGCCCTGAATGAACTGCAGGACGAGGCAGCGCGGCTATCGTGGCT
GGCCACGACGGGCGTTCCTTGCGCAGCTGTGCTCGACGTTGTCACTGAAGCGGGAAGGG
ACTGGCTGCTATTGGGCGAAGTGCCGGGGCAGGATCTCCTGTCATCTCACCTTGCTCCTG
CCGAGAAAGTATCCATCATGGCTGATGCAATGCGGCGGCTGCATACGCTTGATCCGGCTAC
CTGCCCATTCGACCACCAAGCGAAACATCGCATCGAGCGAGCACGTACTCGGATGGAAGC
CGGTCTTGTCGATCAGGATGATCTGGACGAAGAGCATCAGGGGCTCGCGCCAGCCGAACT
GTTCGCCAGGCTCAAGGCGCGCATGCCCGACGGCGAGGATCTCGTCGTGACCCATGGCG
ATGCCTGCTTGCCGAATATCATGGTGGAAAATGGCCGCTTTTCTGGATTCATCGACTGTGGC
CGGCTGGGTGTGGCGGACCGCTATCAGGACATAGCGTTGGCTACCCGTGATATTGCTGAAG
AGCTTGGCGGCGAATGGGCTGACCGCTTCCTCGTGCTTTACGGTATCGCCGCTCCCGATTC
GCAGCGCATCGCCTTCTATCGCCTTCTTGACGAGTTCTTCTGAGCGGGACTCTGGGGTTCG
AAATGACCGACCAAGCGACGCCCAACCTGCCATCACGAGATTTCGATTCCACCGCCGCCTT
CTATGAAAGGTTGGGCTTCGGAATCGTTTTCCGGGACGCCGGCTGGATGATCCTCCAGCG
CGGGGATCTCATGCTGGAGTTCTTCGCCCACCCCAACTTGTTTATTGCAGCTTATAATGGTT
ACAAATAAAGCAATAGCATCACAAATTTCACAAATAAAGCATTTTTTTCACTGCATTCTAGTTG
TGGTTTGTCCAAACTCATCAATGTATOTTATCATGTCTGTATACCGTCGACCTCTAGCTAGAG
CTTGGCGTAATCATGGTCATAGCTGTTTCCTGTGTGAAATTGTTATCCGCTCACAATTCCACA
CAACATACGAGCCGGAAGCATAAAGTGTAAAGCCTGGGGTGCCTAATGAGTGAGCTAACTC
ACATTAATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCGCTCTTCCGCTT
CCTCGCTCACTGACTCGCTGCGCTCGGTCGTTCGGCTGCGGCGAGCGGTATCAGCTCACT
CAAAGGCGGTAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTTTTCCATAG
GCTCCGCCCCCCTGACGAGCATCACAAAAATCGACGCTCAAGTCAGAGGTGGCGAAACCC
GACAGGACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACA
CTAGAAGAACAGTATTTGGTATCTGCGCTCTGCTGAAGCCAGTTACCTTCGGAAAAAGAGTT
GGTAGCTCTTGATCCGGCAAACAAACCACCGCTGGTAGCGGTTTTTTTGTTTGCAAGCAGC
AGATTACGCGCAGAAAAAAAGGATCTCAAGAAGATCCTTTGATCTTTTCTACGGGGTCTGAC
GCTCAGTGGAACGAAAACTCACGTTAAGGGATTTTGGTCATGAGATTATCAAAAAGGATCTT
CACCTAGATCCTTTTAAATTAAAAATGAAGTTTTAAATCAATCTAAAGTATATATGAGTAAACTT
GGTCTGACAGTTACCAATGOTTAATCAGTGAGGCACCTATCTCAGCGATCTGTCTATTTCGTT
CATCCATAGTTGCCTGACTCCCCGTCGTGTAGATAACTACGATACGGGAGGGCTTACCATCT
GGCCCCAGTGCTGCAATGATACCGCGAGACCCACGCTCACCGGCTCCAGATTTATCAGCAA
TAAACCAGCCAGCCGGAAGGGCCGAGCGCAGAAGTGGTCCTGCAACTTTATCCGCCTCCA
TCCAGTCTATTAATTGTTGCCGGGAAGCTAGAGTAAGTAGTTCGCCAGTTAATAGTTTGCGC
AACGTTGTTGCCATTGCTACAGGCATCGTGGTGTCACGCTCGTCGTTTGGTATGGCTTCATT
CAGCTCCGGTTCCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGAC
TGGTGAGTACTCAACCAAGTCATTCTGAGAATAGTGTATGCGGCGACCGAGTTGCTCTTGC
CCGGCGTCAATACGGGATAATACCGCGCCACATAGCAGAACTTTAAAAGTGCTCATCATTGG
AAAACGTTCTTCGGGGCGAAAACTCTCAAGGATCTTACCGCTGTTGAGATCCAGTTCGATG
TAACCCACTCGTGCACCCAACTGATOTTCAGCATCTTTTACTTTCACCAGCGTTTCTGGGTG
AGCAAAAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTCATGAGCG
GATACATATTTGAATGTATTTAGAAAAATAAACAAATAGGGGTTCCGCGCACATTTCCCCGAA
AAGTGCCACCTGACGTCGACGGATCGGGAGATCTCCCGATCCCCTATGGTGCACTCTCAGT
ACAATCTGCTCTGATGCCGCATAGTTAAGCCAGTATCTGCTCCCTGCTTGTGTGTTGGAGGT
CGCTGAGTAGTGCGCGAGCAAAATTTAAGCTACAACAAGGCAAGGCTTGACCGACAATTGC
ATGAAGAATCTGCTTAGGGTTAGGCGTTTTGCGCTGCTTCGCGATGTACGGGCCAGATATAC
GCGTTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAA
CGACCCCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCATTAT
GCCCAGTACATGACCTTATGGGACTTTCCTACTTGGCAGTACATCTACGTATTAGTCATCGCT
ATTACCATGGTGATGCGGTTTTGGCAGTACATCAATGGGCGTGGATAGCGGTTTGACTCACG
GGGATTTCCAAGTCTCCACCCCATTGACGTCAATGGGAGTTTGTTTTGGCACCAAAATCAAC
GGGACTTTCCAAAATGTCGTAACAACTCCGCCCCATTGACGCAAATGGGCGGTAGGCGTGT
ACGGTGGGAGGTCTATATAAGCAGAGCTCTCTGGCTAACTAGAGAACCCACTGCTTACTGG
CTTATCGAAATTAATACGACTCACTATAGGGAGACCCAAGCTG
CBE-29-pCDNA3.1 Plasmid (SEQ ID NO: 12):
GCTAGCGCCACCatgaaacggacagccgacggaagcgagttcgagtcaccaaagaagaagcggaaagtccacaccgc
cgaccaaggcagaagaatcaagagctgggagttcacatccttcttcgaccctagccaactgagaaaggagacaagcctgctgagc
gaggtgtggtggggcagcagcagcagactgtggagaagctgcagccaaaacagcacccaccacgtggagcagagcttcctgga
ccacttccaagctcagagacacctgggccctagcaccacctgcaccatcacctggttcctgagctggagcccctgctgggagtgcgc
tagagccatcagaaccttcctggacaagtaccccaccgtgaagctgaccatctacgtggctagactgtactggcacatggaccctca
gaacagacaaggcctgagaaacctggtgaagggcggcgtggacgtgagaatcatgggccctagcgagcactactactgctggag
aaacttcgtggactacagcagaggcgaggaagaggagtggcctagataccacagagtgcccatccacctgttcgacctggagctg
cactgcatctacctggacctgcccacctgcctaaccatcagccccgtgaagcatcagcagctgctgagcttcagcctgaccctgcaca
cctgtcactatcagaaactgccccccgctctgctcctgctcctcggcctggtcagacccccccctccccaaacaagcctctccgtcgcc
cacagaggagaggccccggcaccgtcaacggcggcgagtgggaccccaacaactggggctggttctgggtgctgagatctgga
ggatctagcggaggatcctctggcagcgagacaccaggaacaagcgagtcagcaacaccagagagcagtggcggcagcagcg
gcggcagcgacaagaagtacagcatcggcctggccatcggcaccaactctgtgggctgggccgtgatcaccgacgagtacaagg
tgcccagcaagaaattcaaggtgctgggcaacaccgaccggcacagcatcaagaagaacctgatcggagccctgctgttcgaca
gcggcgaaacagccgagagaacccggctgaagagaaccgccagaagaagatacaccagacggaagaaccggatctgctatct
gcaagagatcttcagcaacgagatggccaaggtggacgacagcttcttccacagactggaagagtccttcctggtggaagaggata
agaagcacgagcggcaccccatcttcggcaacatcgtggacgaggtggcctaccacgagaagtaccccaccatctaccacctga
gaaagaaactggtggacagcaccgacaaggccgacctgcggctgatctatctggccctggcccacatgatcaagttccggggcca
cttcctgatcgagggcgacctgaaccccgacaacagcgacgtggacaagctgttcatccagctggtgcagacctacaaccagctgtt
cgaggaaaaccccatcaacgccagcggcgtggacgccaaggccatcctgtctgccagactgagcaagagcagacggctggaaa
atctgatcgcccagctgcccggcgagaagaagaatggcctgttcggaaacctgattgccctgagcctgggcctgacccccaacttca
agagcaacttcgacctggccgaggatgccaaactgcagctgagcaaggacacctacgacgacgacctggacaacctgctggccc
agatcggcgaccagtacgccgacctgtttctggccgccaagaacctgtccgacgccatcctgctgagcgacatcctgagagtgaac
accgagatcaccaaggcccccctgagcgcctctatgatcaagagatacgacgagcaccaccaggacctgaccctgctgaaagctc
tcgtgcggcagcagctgcctgagaagtacaaagagattttcttcgaccagagcaagaacggctacgccggctacattgacggcgga
gccagccaggaagagttctacaagttcatcaagcccatcctggaaaagatggacggcaccgaggaactgctcgtgaagctgaaca
gagaggacctgctgcggaagcagcggaccttcgacaacggcagcatcccccaccagatccacctgggagagctgcacgccattc
tgcggcggcaggaagatttttacccattcctgaaggacaaccgggaaaagatcgagaagatcctgaccttccgcatcccctactacg
tgggccctctggccaggggaaacagcagattcgcctggatgaccagaaagagcgaggaaaccatcaccccctggaacttcgagg
aagtggtggacaagggcgcttccgcccagagcttcatcgagcggatgaccaacttcgataagaacctgcccaacgagaaggtgct
gcccaagcacagcctgctgtacgagtacttcaccgtgtataacgagctgaccaaagtgaaatacgtgaccgagggaatgagaaag
cccgccttcctgagcggcgagcagaaaaaggccatcgtggacctgctgttcaagaccaaccggaaagtgaccgtgaagcagctg
aaagaggactacttcaagaaaatcgagtgcttcgactccgtggaaatctccggcgtggaagatcggttcaacgcctccctgggcaca
taccacgatctgctgaaaattatcaaggacaaggacttcctggacaatgaggaaaacgaggacattctggaagatatcgtgctgacc
ctgacactgtttgaggacagagagatgatcgaggaacggctgaaaacctatgcccacctgttcgacgacaaagtgatgaagcagct
gaagcggcggagatacaccggctggggcaggctgagccggaagctgatcaacggcatccgggacaagcagtccggcaagaca
atcctggatttcctgaagtccgacggcttcgccaacagaaacttcatgcagctgatccacgacgacagcctgacctttaaagaggaca
tccagaaagcccaggtgtccggccagggcgatagcctgcacgagcacattgccaatctggccggcagccccgccattaagaagg
gcatcctgcagacagtgaaggtggtggacgagctcgtgaaagtgatgggccggcacaagcccgagaacatcgtgatcgaaatgg
ccagagagaaccagaccacccagaagggacagaagaacagccgcgagagaatgaagcggatcgaagagggcatcaaaga
gctgggcagccagatcctgaaagaacaccccgtggaaaacacccagctgcagaacgagaagctgtacctgtactacctgcagaa
tgggcgggatatgtacgtggaccaggaactggacatcaaccggctgtccgactacgatgtggaccatatcgtgcctcagagctttctg
aaggacgactccatcgacaacaaggtgctgaccagaagcgacaagaaccggggcaagagcgacaacgtgccctccgaagag
gtcgtgaagaagatgaagaactactggcggcagctgctgaacgccaagctgattacccagagaaagttcgacaatctgaccaagg
ccgagagaggcggcctgagcgaactggataaggccggcttcatcaagagacagctggtggaaacccggcagatcacaaagcac
gtggcacagatcctggactcccggatgaacactaagtacgacgagaatgacaagctgatccgggaagtgaaagtgatcaccctga
agtccaagctggtgtccgatttccggaaggatttccagttttacaaagtgcgcgagatcaacaactaccaccacgcccacgacgccta
cctgaacgccgtcgtgggaaccgccctgatcaaaaagtaccctaagctggaaagcgagttcgtgtacggcgactacaaggtgtacg
acgtgcggaagatgatcgccaagagcgagcaggaaatcggcaaggctaccgccaagtacttcttctacagcaacatcatgaactttt
tcaagaccgagattaccctggccaacggcgagatccggaagcggcctctgatcgagacaaacggcgaaaccggggagatcgtgt
gggataagggccgggattttgccaccgtgcggaaagtgctgagcatgccccaagtgaatatcgtgaaaaagaccgaggtgcagac
aggcggcttcagcaaagagtctatcagacccaagaggaacagcgataagctgatcgccagaaagaaggactgggaccctaaga
agtacggcggcttcctgtggcccaccgtggcctattctgtgctggtggtggccaaagtggaaaagggcaagtccaagaaactgaag
agtgtgaaagagctgctggggatcaccatcatggaaagaagcagcticgagaagaatcccatcgactttctggaagccaagggcta
caaagaagtgaaaaaggacctgatcatcaagctgcctaagtactccctgttcgagctggaaaacggccggaagagaatgctggcc
tctgccaagcagctgcagaagggaaacgaactggccctgccctccaaatatgtgaacttcctgtacctggccagccactatgagaag
ctgaagggctcccccgaggataatgagcagaaacagctgtttgtggaacagcacaagcactacctggacgagatcatcgagcaga
tcagcgagttctccaagagagtgatcctggccgacgctaatctggacaaagtgctgtccgcctacaacaagcaccgggataagccc
atcagagagcaggccgagaatatcatccacctgtttaccctgaccagactgggagcccctagagccttcaagtactttgacaccacc
atcgaccccaagcagtacagaagcaccaaagaggtgctggacgccaccctgatccaccagagcatcaccggcctgtacgagac
acggatcgacctgtctcagctgggaggtgacagcggcgggagcggcgggagcggggggagcactaatctgagcgacatcattga
gaaggagactgggaaacagctggtcattcaggagtccatcctgatgctgcctgaggaggtggaggaagtgatcggcaacaagcca
gagtctgacatcctggtgcacaccgcctacgacgagtccacagatgagaatgtgatgctgctgacctctgacgcccccgagtataag
ccttgggccctggtcatccaggattctaacggcgagaataagatcaagatgctggaattcgagcccaagaagaagaggaaagtcta
gCTCGAGAATAAAGGAAATTTATTTTCATTGCAATAGTGTGTTGGAATTTTTTGTGTCTCTCAG
TTAACAAAAAAGCACCGACTCGGTGCCACTTTTTCAAGTTGATAACGGACTAGCCTTATTTTA
ACTTGCTATTTCTAGCTCTAAAACGAGGCGGGGTGGAGGGGGTCGGTGTTTCGTCCTTTCC
ACAAGATATATAAAGCCAAGAAATCGAAATACTTTCAAGTTACGGTAAGCATATGATAGTCCAT
TTTAAAACATAATTTTAAAACTGCAAACTACCCAAGAAATTATTACTTTCTACGTCACGTATTTT
GTACTAATATCTTTGTGTTTACAGTCAAATTAATTCCAATTATCTCTCTAACAGCCTTGTATCGT
ATATGCAAATATGAAGGAATCATGGGAAATAGGCCCTCTTCGAATAGCAGGCATGCTGGGGA
TGCGGTGGGCTCTATGGCTTCTGAGGCGGAAAGAACCAGCTGGGGCTCTAGGGGGTATCC
CCACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGC
CACGTTCGCCGGCTTTCCCCGTCAAGCTCTAAATCGGGGGCTCCCTTTAGGGTTCCGATTT
AGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGA
CTCTTGTTCCAAACTGGAACAACACTCAACCCTATCTCGGTCTATTCTTTTGATTTATAAGGG
ATTTTGCCGATTTCGGCCTATTGGTTAAAAAATGAGCTGATTTAACAAAAATTTAACGCGAATT
AATTCTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAGGCTCC
CCAGCAGGCAGAAGTATGCAAAGCATGCATCTCAATTAGTCAGCAACCATAGTCCCGCCCC
TAACTCCGCCCATCCCGCCCCTAACTCCGCCCAGTTCCGCCCATTCTCCGCCCCATGGCTG
ACTAATTTTTTTTATTTATGCAGAGGCCGAGGCCGCCTCTGCCTCTGAGCTATTCCAGAAGTA
GTGAGGAGGCTTTTTTGGAGGCCTAGGCTTTTGCAAAAAGCTCCCGGGAGCTTGTATATCC
ATTTTCGGATCTGATCAAGAGACAGGATGAGGATCGTTTCGCATGATTGAACAAGATGGATT
GCACGCAGGTTCTCCGGCCGCTTGGGTGGAGAGGCTATTCGGCTATGACTGGGCACAACA
GACAATCGGCTGCTCTGATGCCGCCGTGTTCCGGCTGTCAGCGCAGGGGCGCCCGGTTC
TTTTTGTCAAGACCGACCTGTCCGGTGCCCTGAATGAACTGCAGGACGAGGCAGCGCGGC
TATCGTGGCTGGCCACGACGGGCGTTCCTTGCGCAGCTGTGCTCGACGTTGTCACTGAAG
CGGGAAGGGACTGGCTGCTATTGGGCGAAGTGCCGGGGCAGGATCTCCTGTCATCTCACC
TTGCTCCTGCCGAGAAAGTATCCATCATGGCTGATGCAATGCGGCGGCTGCATACGCTTGA
TCCGGCTACCTGCCCATTCGACCACCAAGCGAAACATCGCATCGAGCGAGCACGTACTCG
GATGGAAGCCGGTCTTGTCGATCAGGATGATCTGGACGAAGAGCATCAGGGGCTCGCGCC
AGCCGAACTGTTCGCCAGGCTCAAGGCGCGCATGCCCGACGGCGAGGATCTCGTCGTGA
CCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGAT
ATTGCTGAAGAGCTTGGCGGCGAATGGGCTGACCGCTTCCTCGTGCTTTACGGTATCGCCG
CTCCCGATTCGCAGCGCATCGCCTTCTATCGCCTTCTTGACGAGTTCTTCTGAGCGGGACT
CTGGGGTTCGAAATGACCGACCAAGCGACGCCCAACCTGCCATCACGAGATTTCGATTCCA
CCGCCGCCTTCTATGAAAGGTTGGGCTTCGGAATCGTTTTCCGGGACGCCGGCTGGATGAT
CCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACTGCA
TTCTAGTTGTGGTTTGTCCAAACTCATCAATGTATCTTATCATGTCTGTATACCGTCGACCTCT
AGCTAGAGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATGAGTG
AGCTAACTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAGGTGG
CGAAACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTTTTTTTGTTTG
CAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAA
AGGATCTTCACCTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGCGATCTGTC
TATTTCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCAGATT
TATCAGCAATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTAT
GGCTTCATTCAGCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCT
TTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACGG
AAATGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACATTT
CCCCGAAAAGTGCCACCTGACGTCGACGGATCGGGAGATCTCCCGATCCCCTATGGTGCA
CTCTCAGTACAATCTGCTCTGATGCCGCATAGTTAAGCCAGTATCTGCTCCCTGCTTGTGTG
TTGGAGGTCGCTGAGTAGTGCGCGAGCAAAATTTAAGCTACAACAAGGCAAGGCTTGACCG
ACAATTGCATGAAGAATCTGCTTAGGGTTAGGCGTTTTGCGCTGCTTCGCGATGTACGGGC
CAGATATACGCGTTGACATTGATTATTGACTAGTTATTAATAGTAATCAATTACGGGGTCATTAG
TTCATAGCCCATATATGGAGTTCCGCGTTACATAACTTACGGTAAATGGCCCGCCTGGCTGA
CCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTAC
ATCAAGTGTATCATATGCCAAGTACGCCCCCTATTGACGTCAATGACGGTAAATGGCCCGCC
TGGCATTATGCCCAGTACATGACCTTATGGGACTTTCCTACTTGGCAGTACATCTACGTATTA
GTCATCGCTATTACCATGGTGATGCGGTTTTGGCAGTACATCAATGGGCGTGGATAGCGGTT
TGACTCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAATGGGCG
GTAGGCGTGTACGGTGGGAGGTCTATATAAGCAGAGCTCTCTGGCTAACTAGAGAACCCAC
TGCTTACTGGCTTATCGAAATTAATACGACTCACTATAGGGAGACCCAAGCTG

Note: the above-mentioned CBE-27-pCDNA3.1 and CBE-29-pCDNA3.1 vector sequences are specially labeled. The lowercase italicized portion is the coding sequence of the cytosine base editor fusion protein, the lowercase underlined portion is the deaminase coding sequence, and the bold, underlined uppercase portion encodes the guide sequence of a gRNA targeting VEGFA.

The sequences of the expression plasmids for cytosine base editors comprising other deaminases are consistent with the sequences of the above-mentioned deaminases-27 and deaminases-29, merely replacing the deaminase coding sequence with their respective specific sequences (selected from the corresponding deaminase coding sequences in the sequence listing).

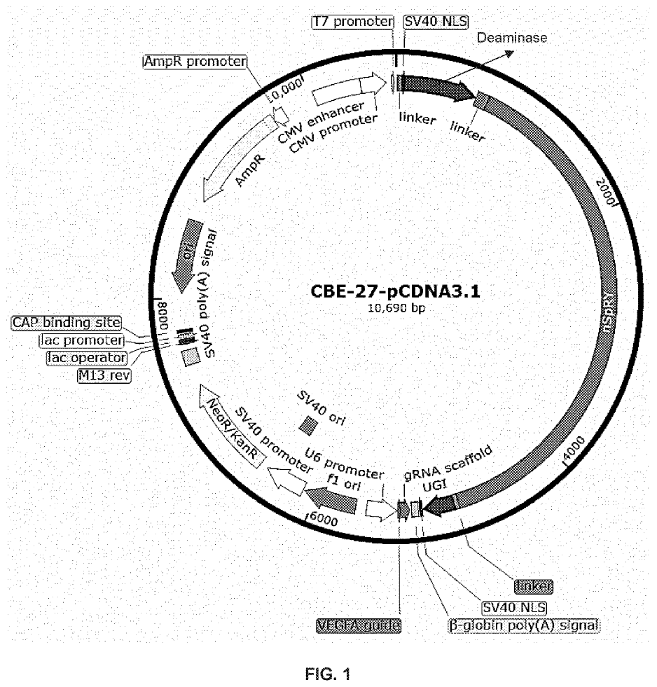

A plasmid map of CBE-27-pCDNA3.1 is exemplarily shown in FIG. 1.

Meanwhile, the deaminase reported in the literature (Huang J et al. Discovery of deaminase functions by structure-based protein clustering. Cell. 2023 Jul. 20; 186(15):3182-3195.e14. doi: 10.1016/j.cell.2023.05.041. Epub 2023 Jun. 27. PubMed Identifier (PMID): 37379837.) was used as a control, wherein the coding sequence of deaminase-03 (numbered as Sdd5 in the literature) reported in the literature is as follows.

CBE-03 (SEQ ID NO: 7):
ATGGGCCCCCTCCTGGACGGGATTGCTGCTCGGCTGGAGGCCGTGAGAGC
CGCCCTGCTGGGGGAGGGCGGGGGCGCCGGGGACGACGAGCCCCCCGCCG
TGCCCTGGGACAGAGTGGAGAGACTGAGAAGAGAGCTGCCCCCTCCCGTG
GTGCCCAACACCGGGCAAAAGACCCACGGCAGATGGATCGGCCCCGACGG
CCAAGCTAGACCCATCGTGAGCGGCCGGGACGACAAGAGCGTGCTGGTGA
ACCCCCTGCTGAGAGGCAAGGGCGCCCCCGGCCCCACAAGAAGAGACAGC
GACGTGGAGATGAAGCTGGCCGCCCACATGGCCGCTAGAGGCATCAGACA
CGCCACCGTGGTGATCAACAACACCCCCTGCAGAGGCCCCCTGAGATGCG
ACACCCTGGTGCCCATCCTGCTGCCCGAGGGCAGCACCCTGACCGTGCAC
GGCATCAACGAGAACGGCACAAGAACAAGAATCAGATACACCGGCGGCGC
TAGACCCTGGTGGAGC

An expression plasmid for a base editor fused with deaminase-03, namely CBE-03-pCDNA3.1, was constructed using the same method as described above for subsequent control tests.

### Experimental Example 2: Detection of Editing Efficiency of Base Editors Comprising Different Cytosine Deaminases

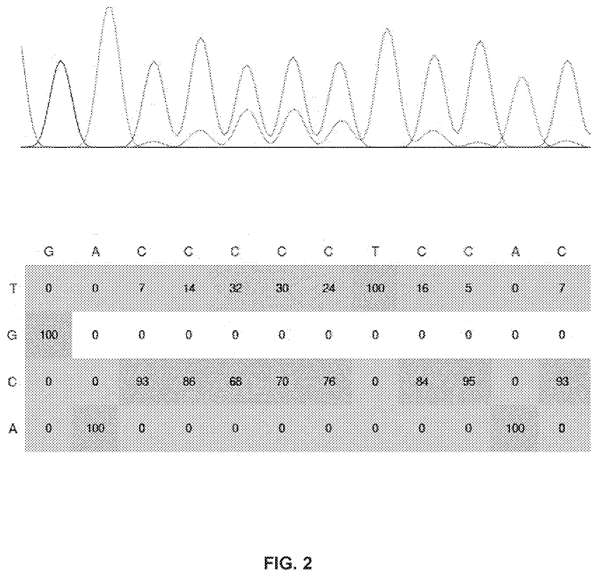

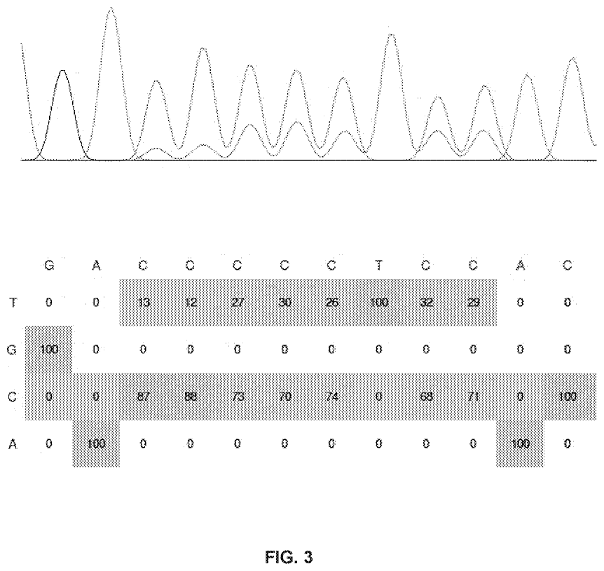

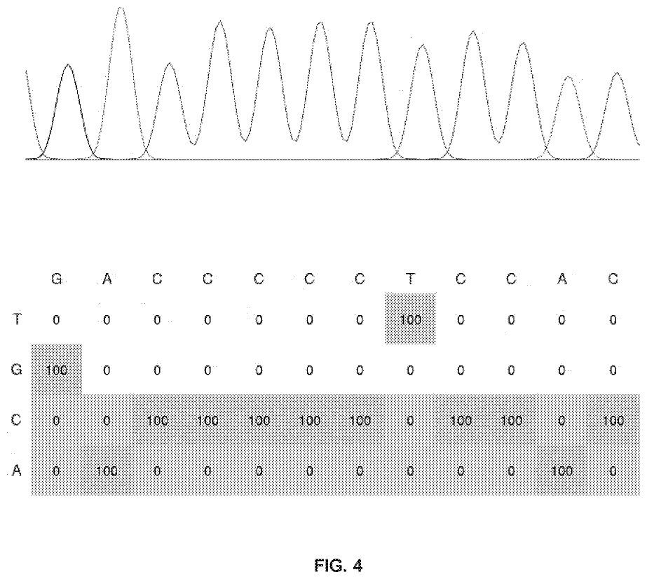

a. Cell Seeding: The 293T cell line was digested when reaching 70%-80% confluency, followed by plating. The seeding density for 24-well plates was 5×10⁵ cells per well.
b. Transfection: Transfection was performed after 12-14 hours of cell seeding. For each well of the 24-well plate, 1.5 μl of Polyethylenimine (PEI) (Yeasen Bio, 40815ES03, Polyethylenimine Linear (PEI) MW25000) was added to 100 μl of Opti-MEM, and 500 ng of each of the cytosine base editor plasmids from Example 1 was added separately. After mixing and incubation at room temperature for 20 minutes, the mixture was added to the 293T cells for cell transfection. After overnight transfection, fresh culture medium (Dulbecco's Modified Eagle Medium (DMEM)+10% FBS) was replaced for continued culture. The negative control group (NC) was not transfected with plasmids.
c. Cell Lysis, Polymerase Chain Reaction (PCR) Amplification Near the Editing Region, Sanger Sequencing: After 72 hours of culture, the cells were washed with PBS, and then 100 μl of cell lysis solution (Viagen, 302-C, DirectPCR® Lysis Reagent (Cell)) was added for lysis, thereby obtaining a lysis solution containing genomic DNA. The region near the genomic DNA target site was amplified, and the PCR product was subjected to Sanger sequencing.
d. Data Analysis: The sequencing chromatograms and the sgRNA guide sequences were submitted to an editing efficiency analysis website (https://moriaritylab.shinyapps.io/editr_v10/) for single-base editing efficiency analysis, thereby obtaining the cytosine editing efficiency of different deaminases at different positions on the target site.
e. Table 1 and FIGS. 2-4 illustrate the editing efficiency of different deaminases for C-to-T base mutation at different positions on the target site.

TABLE 1
Editing Efficiency of Various Base Editors

| | Target Base | | | | | | | | | |
|---|---|---|---|---|---|---|---|---|---|---|
| Editor | G | A | C3 | C4 | C5 | C6 | C7 | T | C9 | C10 |
| CBE-03 | 0% | N/A | 15% | 19% | 24% | 17% | 13% | N/A | 19% | 22% |
| CBE-08 | 0% | N/A | 0% | 0% | 0% | 0% | 0% | N/A | 0% | 0% |
| CBE-09 | 0% | N/A | 0% | 0% | 0% | 0% | 0% | N/A | 0% | 0% |
| CBE-10 | 0% | N/A | 0% | 0% | 0% | 0% | 0% | N/A | 0% | 0% |
| CBE-11 | 0% | N/A | 0% | 0% | 0% | 0% | 0% | N/A | 0% | 0% |
| CBE-12 | 0% | N/A | 0% | 1% | 1% | 1% | 0% | N/A | 0% | 2% |
| CBE-15 | 0% | N/A | 0% | 7% | 0% | 1% | 1% | N/A | 0% | 1% |
| CBE-17 | 0% | N/A | 3% | 2% | 3% | 3% | 2% | N/A | 2% | 3% |
| CBE-20 | 0% | N/A | 1% | 1% | 1% | 1% | 0% | N/A | 0% | 1% |
| CBE-24 | 0% | N/A | 0% | 1% | 2% | 3% | 2% | N/A | 0% | 0% |
| CBE-25 | 0% | N/A | 0% | 0% | 0% | 0% | 0% | N/A | 0% | 0% |
| CBE-27 | 0% | N/A | 7% | 14% | 32% | 30% | 24% | N/A | 16% | 5% |
| CBE-28 | 0% | N/A | 0% | 0% | 0% | 0% | 1% | N/A | 3% | 1% |
| CBE-29 | 0% | N/A | 13% | 12% | 27% | 30% | 26% | N/A | 32% | 29% |
| CBE-38 | 0% | N/A | 0% | 0% | 0% | 0% | 0% | N/A | 0% | 1% |
| CBE-39 | 0% | N/A | 0% | 0% | 0% | 0% | 0% | N/A | 0% | 0% |
| CBE-48 | 0% | N/A | 8% | 8% | 7% | 8% | 7% | N/A | 5% | 10% |
| CBE-90 | 0% | N/A | 0% | 0% | 0% | 0% | 0% | N/A | 0% | 1% |
| CBE-102 | 0% | N/A | 0% | 0% | 0% | 0% | 0% | N/A | 0% | 0% |
| NC | 0% | N/A | 0% | 0% | 0% | 0% | 0% | N/A | 0% | 0% |

| | Target Base | | | | | | | | | |
|---|---|---|---|---|---|---|---|---|---|---|
| Editor | A | C12 | C13 | C14 | C15 | G | C17 | C18 | T | C20 |
| CBE-03 | N/A | 1% | 0% | 0% | 0% | 0% | 4% | 0% | N/A | 0% |
| CBE-08 | N/A | 0% | 0% | 0% | 0% | 0% | 0% | 0% | N/A | 0% |
| CBE-09 | N/A | 0% | 0% | 0% | 0% | 0% | 0% | 0% | N/A | 0% |
| CBE-10 | N/A | 0% | 0% | 0% | 0% | 0% | 0% | 0% | N/A | 0% |
| CBE-11 | N/A | 0% | 0% | 0% | 0% | 0% | 0% | 0% | N/A | 0% |
| CBE-12 | N/A | 0% | 3% | 2% | 2% | 0% | 1% | 3% | N/A | 0% |
| CBE-15 | N/A | 0% | 1% | 0% | 2% | 0% | 0% | 2% | N/A | 0% |
| CBE-17 | N/A | 2% | 2% | 2% | 2% | 0% | 2% | 2% | N/A | 1% |
| CBE-20 | N/A | 0% | 1% | 1% | 1% | 0% | 1% | 1% | N/A | 0% |
| CBE-24 | N/A | 0% | 0% | 0% | 0% | 0% | 0% | 0% | N/A | 0% |
| CBE-25 | N/A | 0% | 0% | 0% | 0% | 0% | 0% | 0% | N/A | 0% |
| CBE-27 | N/A | 7% | 0% | 0% | 0% | 0% | 0% | 0% | N/A | 0% |
| CBE-28 | N/A | 0% | 0% | 0% | 0% | 0% | 0% | 0% | N/A | 0% |
| CBE-29 | N/A | 0% | 0% | 0% | 0% | 0% | 0% | 0% | N/A | 0% |
| CBE-38 | N/A | 0% | 0% | 0% | 0% | 0% | 1% | 1% | N/A | 0% |
| CBE-39 | N/A | 0% | 0% | 0% | 0% | 0% | 0% | 0% | N/A | 0% |
| CBE-48 | N/A | 7% | 8% | 8% | 8% | 0% | 6% | 7% | N/A | 5% |
| CBE-90 | N/A | 0% | 0% | 0% | 0% | 0% | 0% | 0% | N/A | 0% |
| CBE-102 | N/A | 0% | 0% | 8% | 0% | 0% | 0% | 0% | N/A | 0% |
| NC | N/A | 0% | 0% | 0% | 0% | 0% | 0% | 0% | N/A | 0% |

As can be seen from the data in Table 1, de aminases-12, 15, 17, 20, 24, 27 to 29, 38, 48, 90, and 102 are active after being constructed into base editors. Furthermore, the activities of deaminase-27 and deaminase-29 are superior to that of deaminase-03.

The editing window of CBE-48 may be very wide, thereby facilitating its function in scenarios with specific requirements. The editing window of CBE-102 is very narrow, and its specificity is very good, and the position of the editing window is also significantly different from that of other deaminases.

### Experimental Example 3: Editing Efficiency Detection of Base Editors Comprising Different Cytosine Deaminases

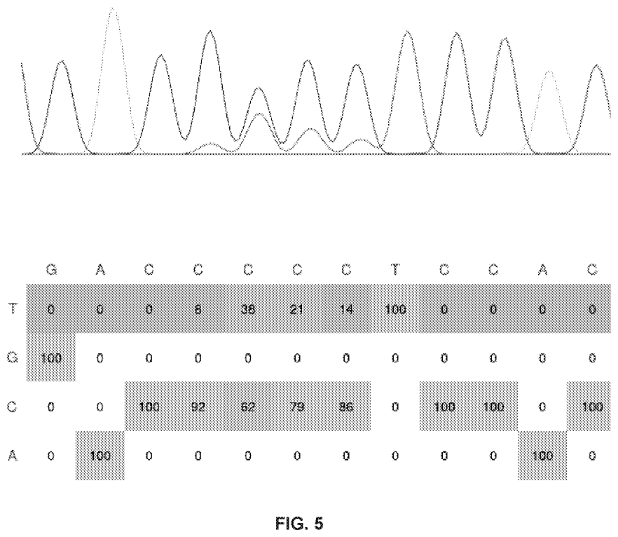

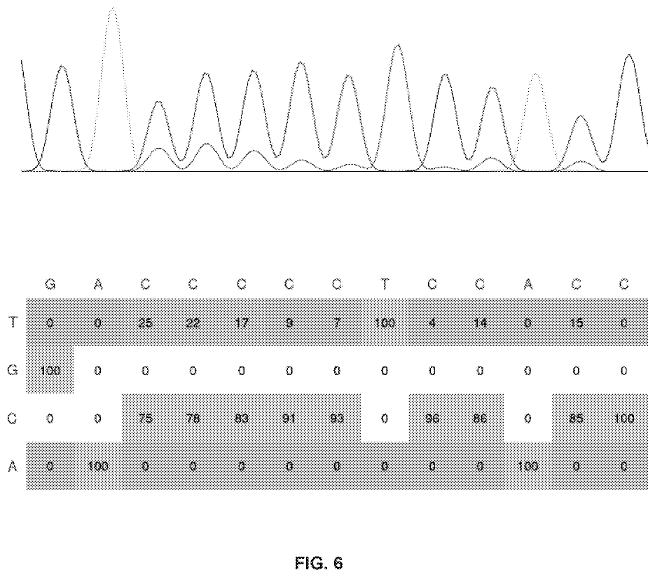

The same manner as in Example 2 was employed to detect the editing efficiency of each base editor in Table 2, and the results shown were obtained. CBE-136 and CBE-140 exhibit high editing efficiency (see FIG. 5 and FIG. 6).

TABLE 2
Editing Efficiency of Base Editors

| Target Base | CBE-136 | CBE-137 | CBE-140 |
|---|---|---|---|
| G | 0% | 0% | 0% |
| A | N/A | N/A | N/A |
| C3 | 0% | 0% | 25% |
| C4 | 8% | 0% | 22% |
| C5 | 38% | 0% | 17% |
| C6 | 21% | 0% | 9% |
| C7 | 14% | 0% | 7% |
| T | N/A | N/A | N/A |
| C9 | 0% | 0% | 4% |
| C10 | 0% | 0% | 14% |
| A | N/A | N/A | N/A |
| C12 | 0% | 0% | 15% |
| C13 | 0% | 0% | 0% |
| C14 | 0% | 0% | 0% |
| C15 | 0% | 0% | 0% |
| G | 0% | 0% | 0% |
| C17 | 0% | 0% | 0% |
| C18 | 0% | 0% | 0% |
| T | N/A | N/A | N/A |
| C20 | 0% | 0% | 0% |

### Experimental Example 4. Detection of Editing Efficiency of Base Editors Comprising Different Cytosine Deaminases

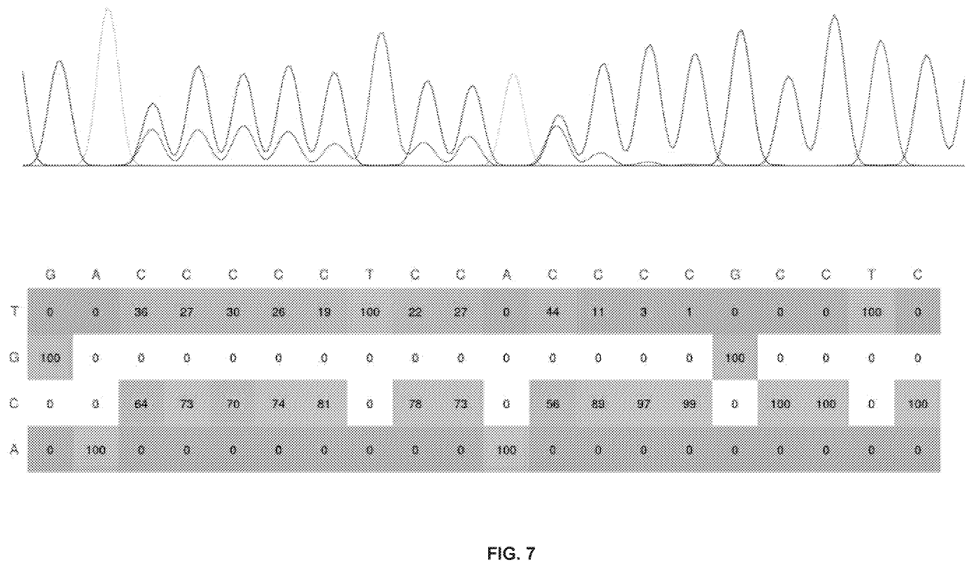

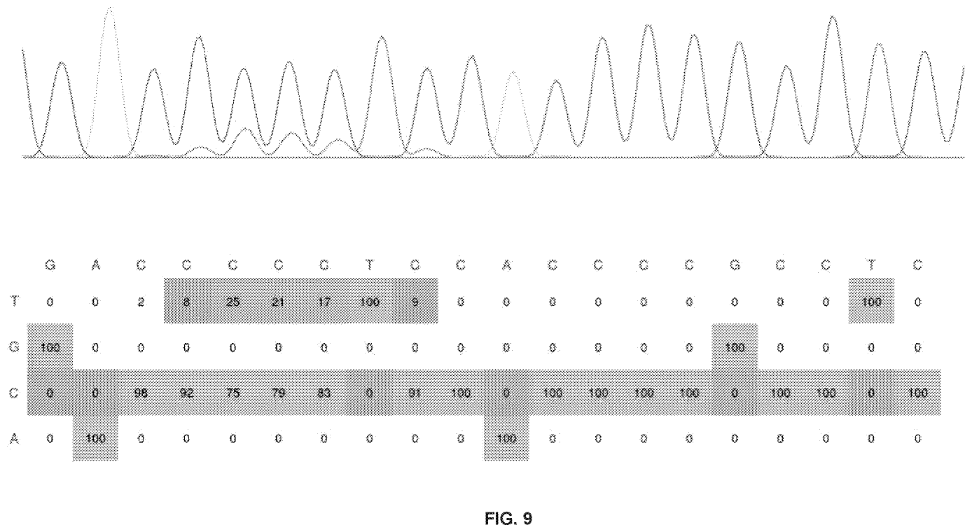

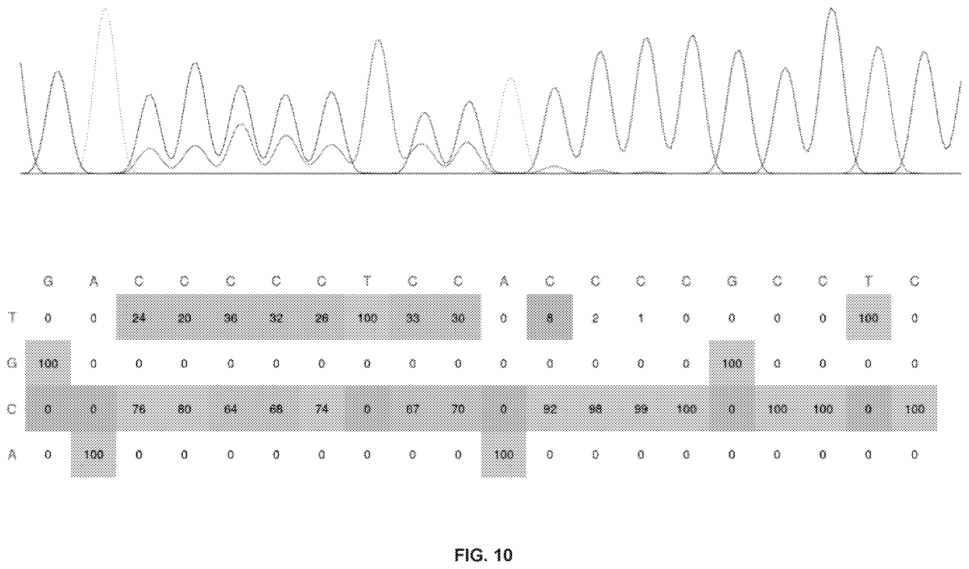

The editing efficiency of the editors comprising deaminase-139, deaminase-145, deaminase-141, and deaminase-143 was detected using the same method as in Example 2. The results are shown in FIGS. 7-10.

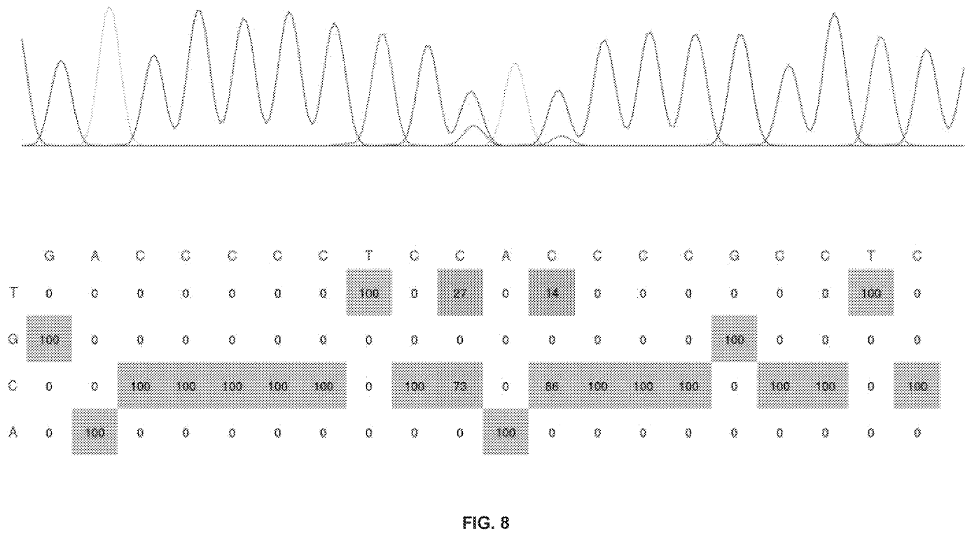

It can be seen from FIG. 8 that CBE-145 has a very narrow editing window and excellent specificity.

### Experimental Example 5. Obtaining and Verifying a Mutant of Deaminase-145

### (1) Screening for Highly Efficient and Highly Specific Mutants of CBE-145

The amino acid sequence of wild-type CBE-145 (207 amino acids) is shown as follows:

(SEQ ID NO: 169)
MLEIKEDVVHIFRAFWECGVHRGNWCGDTHILYEFGTSPATTDFRNIVTN
DGDGGQHAEIKLIYILDNHIAQMVKCHQRKEIYLNMYMNRSPCYKCAFQL
IRFKNYLYNKGYDIRITIKAAQPYMCKRITCSSCVEGTSHSDGLRELNRN
GIFVNAWVWADWQFLAAYLNGLVRVCYDEKFPESGHGKRESRSMADEAAQ
EDFNALR.

Phages expressing deaminase-145 were prepared according to the method described in the literature (Neugebauer M E, Hsu A, Arbab M, et al. Evolution of an adenine base editor into a small, efficient cytosine base editor with low off-target activity[J]. Nature biotechnology, 2023, 41(5): 673-685). Deaminase-145 was evolved by the PANCE method using the screening system and strains of the literature. Finally, the following deaminase-145 mutants were screened out: deaminase-145-P5 (Q122H), deaminase-145-P1 (S191R), deaminase-145-P2 (1114T), deaminase-145-P4 (L2M), and deaminase-145-P6 (N89D).

The amino acid sequence of mutant deaminase-145-P5 (Q122H) is:

(SEQ ID NO: 329)
MLEIKEDVVHIFRAFWECGVHRGNWCGDTHILYEFGTSPATTDFRNIVTN
DGDGGQHAEIKLIYILDNHIAQMVKCHQRKEIYLNMYMNRSPCYKCAFQL
IRFKNYLYNKGYDIRITIKAAHPYMCKRITCSSCVEGTSHSDGLRELNRN
GIFVNAWVWADWQFLAAYLNGLVRVCYDEKFPESGHGKRESRSMADEAAQ
EDFNALR.

### (2) Construction of Vector Plasmids for Verification

Verification vectors were obtained by synthesis from an outsourced service company. These include the CBE-145 expression plasmid vector (SEQ ID NO: 330), which is the same as those described in the preceding Example 1 and Example 4, and expression plasmid vectors comprising deaminase-145 mutants (CBE-145-P5, CBE-145-P1, CBE-145-P2, CBE-145-P4, and CBE-145-P6). The sequence of the vector plasmid CBE-145-P5 for deaminase-145-P5 is SEQ ID NO: 331. The Cas protein portion of the editor fusion protein uses nSPRY The gRNA targets the VEGFA target site, and the spacer is GACCCCCTCCACCCCGCCTC (SEQ ID NO: 332).

(3) Transfection of 293T Cells with the Vectors to be Verified

The different verification vectors and control vectors were transfected into 293T cells.

Transfection was performed in a 24-well plate in accordance with the Lipofectamine 2000 (Thermo) instructions, and detection was performed after 72 hours.

### (4) Detection of Changes in the VEGFA Target Gene

The cells 72 hours after transfection were used with a genomic DNA extraction kit (Tiangen Biotech) to extract the genome of the cells. The extracted products were subjected to PCR amplification, and the primers are shown as follows: VEGFA-CBE-F3: ACTTGAATCGGGCCGACG (SEQ ID NO: 333); VEGFA-CBE-R3: CCTTGGCATGGTGGAGGTAGAG (SEQ ID NO: 334).

Amplification was performed using 2X Pro Taq Premix Ver.2 (Accurate Biotech). The product length was 866 bp, and the products were sent to Genewiz for sequencing.

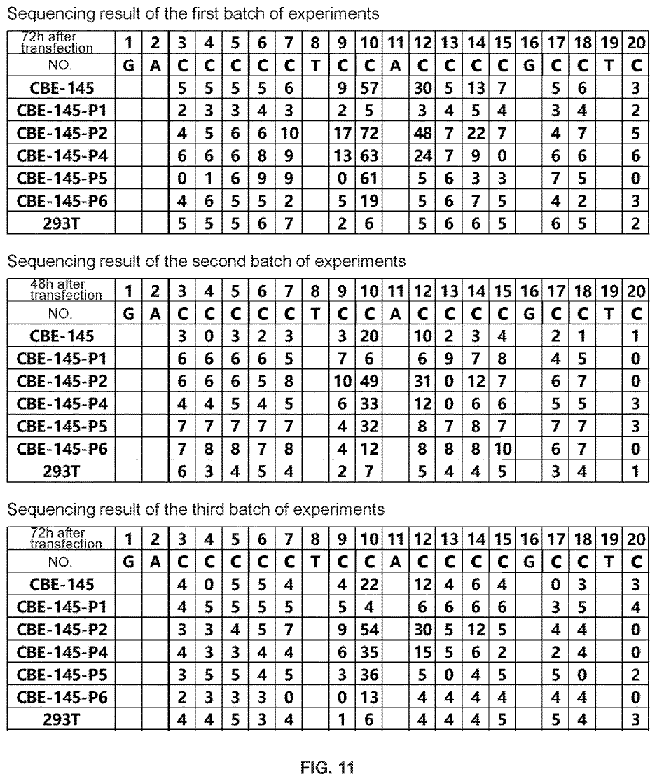

The sequencing results were analyzed. The results of 3 batches are shown in FIG. 11: In the figure, 1-20 represent the nucleotide position numbers of the spacer, and the base C that can be edited by CBE is indicated, and the numbers below the base C represent the percentage of C edited to T (in %). The 293T group serves as an unedited negative control. The present disclosure considers 10% as having an editing effect.

In the experiments of 3 batches, the CBE-145 containing wild-type deaminase had an editing effect on the C at positions 10 and 12 (the result of one batch also showed a weak editing effect at the position 14), converting it to T. CBE-145-P5 (Q122H) had an editing effect only at the position 10, with a narrower editing window. Furthermore, in the results of 3 batches, the editing effect of CBE-145-P5 at the position 10 was superior to that of the CBE-145 containing wild-type deaminase. Therefore, the efficiency and specificity of deaminase-145-P5 were both superior to those of deaminase-145.

In the results of 3 batches, the editing effects of CBE-145-P2 and CBE-145-P4 at the position 10 were both superior to that of the CBE-145 containing wild-type deaminase. Therefore, the efficiencies of deaminase-145-P2 and -P4 were both superior to that of deaminase-145.

### Experimental Example 6. Verification of More Mutants of Deaminase-145

### (1) Screening for Highly Efficient and Highly Specific CBE-145 Mutants

Phages expressing deaminase-145 were prepared in a manner of the literature (Neugebauer M E, Hsu A, Arbab M, et al. Evolution of an adenine base editor into a small, efficient cytosine base editor with low off-target activity[J]. Nature Biotechnology, 2023, 41(5): 673-685), and the deaminase-145 was evolved by the PANCE method using the screening system and strains of the literature, thereby ultimately screening out the deaminase-145 mutants: deaminase-145-P5 (Q122H); deaminase-145-P1 (S191R); deaminase-145-P2 (1114T); deaminase-145-P3 (C176F); deaminase-145-P4 (L2M); deaminase-145-P6 (N89D): deaminase-145-P7 (N681); deaminase-145-P8 (Q78*, i.e., the codon of the 78th amino acid Q was mutated into a stop codon); and deaminase-145-P11 (N681 and 1114T mutations).

### (2) Construction of Vector Plasmids for Verification

Verification vectors were obtained by synthesis from an outsourced service company. These include the CBE-145 expression plasmid vector (SEQ ID NO: 330), which is the same as those described in the preceding Example 1 and Example 4, and expression plasmid vectors comprising the deaminase-145 mutants (CBE-145-P5, CBE-145-P1, CBE-145-P2, CBE-145-P3, CBE-145-P4, CBE-145-P6, CBE-145-P7, CBE-145-P8, and CBE-145-P11). The sequence of the vector plasmid CBE-145-P5 for the deaminase-145-P5 is SEQ ID NO: 331. The Cas protein portion of the editor fusion protein uses nSPRY The Grna targets the VEGFA target site, and the spacer is GACCCCCTCCACCCCGCCTC (SEQ ID NO: 332).

(3) Transfection of 293T Cells with the Vectors to be Verified

The different verification vectors and control vectors were transfected into the 293T cells.

Transfection was performed in a 24-well plate in accordance with the Lipofectamine 2000 (Thermo) instructions, and detection was performed after 72 hours.

### (4) Determination of Changes in the VEGFA Target Gene

The cells 72 hours after transfection were used with a genomic DNA extraction kit to extract the genome of the cells. The extracted products were subjected to PCR amplification, and the primers are shown as follows: VEGFA-CBE-F3: ACTTGAATCGGGCCGACG (SEQ ID NO: 333); VEGFA-CBE-R3: CCTTGGCATGGTGGAGGTAGAG (SEQ ID NO: 334).

Amplification was performed using 2X Pro Taq Premix Ver.2 (Accurate Biology), the product length was 866 bp, and the products were sent to GeneWiz for sequencing.

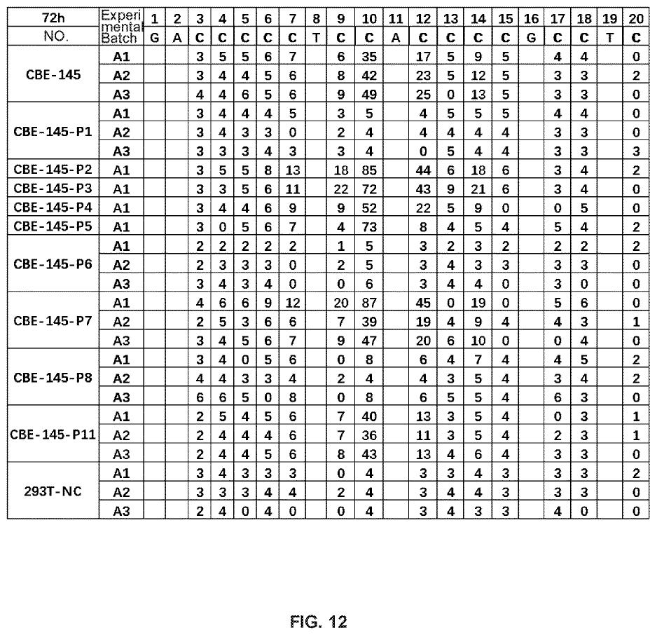

The sequencing results were analyzed. The results of 3 batches are shown in FIG. 12: In the figure, 1 to 20 represent the nucleotide position numbers of the spacer, and the numbers below the base represent the percentage of C edited to T (in %). The 293T group serves as an unedited negative control. The present disclosure considers 10% as having an editing effect.

The results of FIG. 12 show that the editing effects of CBE-145-P2, CBE-145-P3, CBE-145-P4, and CBE-145-P5 at the position 10 are all superior to that of the CBE-145 containing the wild-type deaminase. Therefore, the efficiencies of the deaminase-145-P2/P3/P4/P5 variants are all superior to that of the deaminase-145.

The deaminase-145-P1, deaminase-145-P6, and deaminase-145-PB mutants almost completely lost their activity.

The deaminase-145-P7 and deaminase-145-P11 mutants still have relatively strong activity. The deaminase-145-P11 mutant has editing specificity at the position 10 that is superior to the wild type.

APPENDIX TABLE 1
Target nucleic acids/target genes and corresponding diseases or disorders
(when there are two or more target genes in a specific cell, it indicates
simultaneously targeting the two or more target genes):

| Target site (gene) | Disease name |
|---|---|
| 17β-HSD13 | Non-alcoholic |
| | steatohepatitis (NASH) |
| 17β-HSD13 | Metabolic dysfunction- |
| | associated fatty liver |
| | disease (MAFLD) |
| 17β-HSD5, PSMA | Castration-resistant |
| | prostate cancer |
| 23S rRNA | Klebsiella pneumoniae |
| | infection |
| 4-1BB, BCMA | Refractory plasma cell |
| | myeloma |
| 4-1BB, BCMA | Relapsed Multiple |
| | myeloma |
| 4-1BB, BCMA | Multiple myeloma |
| 4-1BB, CD19 | Myasthenia gravis |
| 4-1BB, CD19 | Small lymphocytic |
| | lymphoma |
| 4-1BB, CD19 | Systemic sclerosis |
| 4-1BB, CD19 | Systemic lupus |
| | erythematosus |
| 4-1BB, CD19 | Idiopathic |
| | inflammatory |
| | myopathy |
| 4-1BB, CD19 | Dermatomyositis |
| 4-1BB, CD19 | Chronic lymphocytic |
| | leukemia |
| 4-1BB, CD19 | Lymphocytic leukemia |
| 4-1BB, CD19 | Lupus nephritis |
| 4-1BB, CD19 | Non-Hodgkin |
| | lymphoma |
| 4-1BB, CD19, | Chronic lymphocytic |
| CD3ζ, EGFR | leukemia (CLL) |
| 4-1BB, CD19, | Acute lymphoblastic |
| CD3ζ, EGFR | leukemia (ALL) |
| 4-1BB, CD19, | Non-Hodgkin |
| CD3ζ, EGFR | lymphoma |
| 4-1BB, CD40 | Biliary tract tumor |
| 4-1BB, CD40L, | Metastatic |
| CTLA4 | hepatocellular |
| | carcinoma |
| 4-1BB, CD40L, | Microsatellite-stable |
| CTLA4 | colorectal cancer |
| | (MSS CRC) |
| 4-1BB, CD40L | Advanced malignant |
| CTLA4 | solid tumor |
| 4-1BB, CD40L, | Solid tumor |
| CTLA4 | |
| 4-1BB, CD40L, | Locally advanced |
| CTLA4 | head and neck |
| | squamous cell |
| | carcinoma (HNSCC) |
| 4-1BB, CD40L, | Locally advanced |
| CTLA4 | hepatocellular |
| | carcinoma (HCC) |
| 4-1BB, CD40L, | Recurrent head and |
| CTLA4 | neck squamous cell |
| | carcinoma (Recurrent |
| | HNSCC) |
| 4-1BB, EBV protein | Nasopharyngeal |
| | carcinoma (NPC) |
| 4-1BB, EBV protein | Niemann-Pick disease |
| | type C |
| 4-1BB, HPV E7, | Advanced cancer |
| IL2RA | |
| 4-1BB, HPV E7, | Head and neck tumor |
| L2RA | |
| 4-1BB, HPV E7, | Human papillomavirus |
| IL2RA | type 16 positive (HPV- |
| | 16 positive) |
| 4-1BB, HPV E7, | Cervical cancer |
| IL2RA | |
| 4-1BB, HPV E7, | Anal tumor |
| L2RA | |
| 4-1BB, HPV E7, | HPV16 infection |
| IL2RA | |
| 4-1BB, IL-12R | Advanced malignant |
| | solid tumor |
| 4-1BB, IL-12R | Head and neck |
| | squamous cell |
| | carcinoma (HNSCC) |
| 4-1BB, IL-12R | Triple-negative breast |
| | cancer (TNBC) |
| 4-1BB, IL-12R | Cutaneous melanoma |
| 4-1BB, IL-12R | Urothelial carcinoma |
| 4-1BB, IL-12R | Melanoma |
| 4-1BB, IL-12R | Non-small cell lung |
| | cancer (NSCLC) |
| 4-1BB, IL-12R | Bladder urothelial |
| | carcinoma |
| 4-1BBL, CD19, | Tumor |
| PSCA | |
| 4-1BBL, IL15R | Solid tumor |
| 5T4 | Solid tumor |
| 5T4 | Locally advanced |
| | malignant solid tumor |
| 5T4 | Acute myeloid |
| | leukemia (AML) |
| 5T4 | Unresectable |
| | malignant solid tumor |
| A1AT | Liver fibrosis |
| A1AT | Liver disease |
| A1AT | Scar |
| A1AT | Alpha-1 antitrypsin |
| | deficiency |
| A1R | Asthma |
| ABCA4 | Mucopolysaccharidosis |
| | type I (MPS I) |
| ABCA4 | Cystic fibrosis |
| ABCA4 | Huntington's disease |
| ABCA4 | Alzheimer's disease |
| ABCA4 | Stargardt disease type 4 |
| ABCA4 | Friedreich's ataxia |
| ABCD1 | Adrenoleukodystrophy |
| | (ALD) |
| ABCD1 | Hypertriglyceridemia |
| AC | Heart failure |
| ACE2 | Primary sclerosing |
| | cholangitis |
| ACE2 | Coronavirus infection |
| AChE | Myasthenia gravis |
| AChE | Inflammatory bowel |
| | disease |
| AChE | Amyotrophic lateral |
| | sclerosis |
| ACTG2 | Megacystis- |
| | microcolon-intestinal |
| | hypoperistalsis |
| | syndrome (MMIHS) |
| ACVR2B | Cachexia |
| ADA | Severe combined |
| | immunodeficiency |
| ADA | Adenosine deaminase |
| | deficiency |
| ADAM8 | Tumor |
| ADAMTS5 | Osteoarthritis |
| ADAMTS5, TNF | Rheumatoid arthritis |
| ADAMTS5, TNF | Osteoarthritis |
| ADAR | Novel coronavirus |
| | infection |
| ADAR | Decompensated liver |
| | cirrhosis |
| ADAR | Immunoglobulin A |
| | nephropathy |
| ADARB1 | Amyotrophic lateral |
| | sclerosis |
| ADP | Tumor |
| ADP, thymidine | Pancreatic cancer |
| kinase | |
| aFGF | Peripheral arterial |
| | occlusive disease |
| aFGF | Eczema |
| aFGF | Chronic limb- |
| | threatening ischemia |
| aFGF | Intermittent |
| | claudication |
| aFGF | Arterial occlusive |
| | disease |
| AFP | Liver cancer |
| AGER | Inflammation |
| AGER | Asthma |
| AGL | Glycogen storage |
| | disease type III (GSD III) |
| AGRE2, CLL-1 | Relapsed acute |
| | myeloid leukemia |
| AGT | Preeclampsia |
| AGT | Heart failure with |
| | reduced ejection |
| | fraction |
| AGT | Chronic heart failure |
| AGT | Hypertension |
| AGT | Alzheimer's disease |
| AGT, ANGPTL3 | Cardiovascular |
| | disease |
| AGT, APOC3 | Hypertension |
| AGT, APOC3 | Hypertriglyceridemia |
| AIPL1 | Cone dystrophy |
| AIPL1 | Retinitis pigmentosa |
| AIPL1 | Leber congenital |
| | amaurosis |
| Akt, PTEN | Tumor |
| Akt-1 | Metastatic renal cell |
| | carcinoma |
| Akt-1 | Secondary malignant |
| | neoplasm of pancreas |
| Akt-1 | Pancreatic cancer |
| Akt-1 | Advanced renal cell |
| | carcinoma |
| Akt-1 | Advanced |
| | hepatocellular |
| | carcinoma |
| Akt-1 | Advanced malignant |
| | solid tumor |
| Akt-1 | Renal cell carcinoma |
| ALAS1 | Acute hepatic |
| | porphyria |
| ALAS1 | Hepatic porphyria |
| ALDH2 | Esophageal cancer |
| ALDH2 | Alcohol use disorder |
| ALDH2 | Glioblastoma |
| ALDH2 | Glioma |
| ALDH2 | Osteoporosis |
| ALK5 | Ocular disease |
| ALPP | Endometrial cancer |
| ALPP | Ovarian cancer |
| ALPPL2 | Solid tumor |
| AMHR2 | Tumor |
| AML1-ETO fusion | Acute myeloid |
| protein | leukemia |
| Ang2 | Retinal disorder |
| Ang2 | Prostate cancer |
| Ang2 | Sepsis |
| Ang2 | Acute lung injury |
| Ang2 | Acute lung injury |
| Ang2, VEGF | Wet age-related |
| | macular degeneration |
| Angiostatin | Corneal transplant |
| | rejection |
| ANGPTL3 | Primary |
| | hypercholesterolemia |
| ANGPTL3 | Hypertriglyceridemia |
| ANGPTL3 | Homozygous familial |
| | hypercholesterolemia |
| ANGPTL3 | Type II |
| | hyperlipoproteinemia |
| ANO5 | Limb-girdle muscular |
| | dystrophy |
| AP4M1 | Autosomal recessive |
| | spastic paraplegia |
| | type 50 |
| APN | B-cell lymphoma |
| APN, DPP-4 | Organ transplant |
| | rejection |
| APOA1 | Hypercholesterolemia |
| APOB | Heterozygous familial |
| | hypercholesterolemia |
| APOB | Neonatal disease |
| APOB | Congenital |
| | malformation |
| APOB | Coronary artery |
| | disease |
| APOB | Coronary heart |
| | disease |
| APOB | High and low density |
| | lipoprotein |
| | cholesterolemia |
| APOB | Hypercholesterolemia |
| APOB | Homozygous familial |
| | hypercholesterolemia |
| APOB | Type II |
| | hyperlipoproteinemia |
| APOB | Type IIa |
| | hyperlipoproteinemia |
| APOC3 | Combined lipase |
| | deficiencies |
| APOC3 | Dyslipidemia |
| APOC3 | Hemophilia B |
| APOC3 | Familial |
| | chylomicronemia |
| | syndrome |
| APOC3 | Hyperlipidemia |
| APOC3 | Hypertriglyceridemia |
| APOC3 | Non-alcoholic |
| | steatohepatitis |
| APOC3 | Atherosclerosis |
| APOC3 | Type I |
| | hyperlipoproteinemia |
| APOC3, PCSK9 | Dyslipidemia |
| APOC3, PCSK9 | Metabolic disease |
| APOC3, TMPRSS6 | Hemochromatosis |
| APOC3, TMPRSS6 | Hypertriglyceridemia |
| ApoE2 | Alzheimer's disease |
| APP | Down syndrome |
| APP | Cerebral amyloid |
| | angiopathy |
| APP | Alzheimer's disease |
| APRIL | Refractory plasma cell |
| | myeloma |
| APRIL | Relapsed Multiple |
| | myeloma |
| AQP1 | Parotid gland disease |
| AQP1 | Xerostomia |
| AR | Androgenic alopecia |
| AR | Castration-resistant |
| | prostate cancer |
| AR | Prostate cancer |
| AR | Prostate cancer |
| AR | Bone cancer |
| AR, mTOR | Benign prostatic |
| | hyperplasia |
| AR, mTOR | Prostate cancer |
| AR, PRLR, | Tumor |
| aromatase | |
| ARHGAP45 | Primary myelofibrosis |
| ARHGAP45 | Hematologic |
| | malignancy |
| ARHGAP45 | Solid tumor |
| ARHGAP45 | Chronic myeloid |
| | leukemia |
| ARHGAP45 | Lymphoma |
| ARHGAP45 | Acute myeloid |
| | leukemia |
| ARHGAP45 | Acute lymphoblastic |
| | leukemia |
| ARHGAP45 | Myelodysplastic |
| | syndrome |
| ARHGAP45 | Myelofibrosis |
| ARHGAP45 | Multiple myeloma |
| ASA | Metachromatic |
| | leukoencephalopathy |
| ASC2 | Inflammation |
| ASGR1, L-lactate | End-stage renal |
| dehydrogenase | disease |
| ASGR1, L-lactate | Primary hyperoxaluria |
| dehydrogenase | type 2 |
| ASGR1, L-lactate | Primary hyperoxaluria |
| dehydrogenase | type 1 |
| ASGR1, L-lactate | Primary hyperoxaluria |
| dehydrogenase | |
| ASN | Metastatic pancreatic |
| | ductal |
| | adenocarcinoma |
| ASN | Advanced pancreatic |
| | ductal |
| | adenocarcinoma |
| ASS | Citrullinemia |
| ASXL1, RUNX1, | Acute myeloid |
| p53 | leukemia |
| AT III | Hemophilia B |
| AT III | Hemophilia A |
| AT III | Venous |
| | thromboembolism |
| AT III | Atherosclerosis |
| AT III | Hemorrhage |
| ATM | Ataxia-telangiectasia |
| ATOH1 | Hearing loss |
| ATP7A | Menkes syndrome |
| ATP7B | Wilson's disease |
| ATXN1 | Spinocerebellar ataxia |
| ATXN2 | Amyotrophic lateral |
| | sclerosis |
| ATXN3 | Machado-Joseph |
| | disease |
| ATXN7 | Ataxia |
| Autotaxin | Idiopathic pulmonary |
| | fibrosis |
| AXL | Sarcoma |
| AXL | Ovarian cancer |
| AXL | Osteosarcoma |
| B2M, HLA-B | Head and neck tumor |
| B2M, HLA-B | Renal tumor |
| B2M, HLA-B | Breast cancer |
| B2M, HLA-B | Lymphoma |
| B2M, HLA-B | Colorectal cancer |
| B2M, HLA-B | Melanoma |
| B4GALT1 | Cardiovascular |
| | disease |
| B7-H4 | Solid tumor |
| BAFF | Autoimmune disease |
| BAFF | Systemic lupus |
| | erythematosus |
| BAFF | Refractory non- |
| | Hodgkin lymphoma |
| BAFF | Refractory plasma cell |
| | myeloma |
| BAFF | Sjogren's syndrome |
| BAFF | Relapsed non-Hodgkin |
| | lymphoma |
| BAFF | Relapsed Multiple |
| | myeloma |
| BAFF | B-cell lymphoma |
| BAFF | B-cell malignancy |
| BAFF, CD19 | Autoimmune disease |
| BAFF-R | Refractory |
| | transformed chronic |
| | lymphocytic leukemia |
| BAFF-R | Refractory small |
| | lymphocytic lymphoma |
| BAFF-R | Refractory mantle cell |
| | lymphoma |
| BAFF-R | Refractory diffuse |
| | large B-cell lymphoma |
| BAFF-R | Refractory chronic |
| | lymphocytic leukemia |
| BAFF-R | Refractory follicular |
| | lymphoma |
| BAFF-R | Diffuse large B-cell |
| | lymphoma |
| BAFF-R | Relapsed transformed |
| | chronic lymphocytic |
| | leukemia |
| BAFF-R | Relapsed mantle cell |
| | lymphoma |
| BAFF-R | Relapsed chronic |
| | lymphocytic leukemia |
| BAFF-R | Relapsed follicular |
| | lymphoma |
| BAFF-R | Relapsed marginal |
| | zone lymphoma |
| BAFF-R | Refractory marginal |
| | zone lymphoma |
| BAFF-R | B-cell malignancy |
| BAG3 | Dilated |
| | cardiomyopathy |
| BCL11A | Sickle cell disease |
| BCL11A | B-thalassemia |
| BCL11A, | Sickle cell disease |
| Hemoglobins | |
| BCL11A, β-globin | Transfusion- |
| | dependent |
| | β-thalassemia |
| BCL11A, β-globin | Sickle cell disease |
| BCL11A, β-globin | β-thalassemia |
| Bcl-2 | Metastatic renal cell |
| | carcinoma |
| Bcl-2 | Metastatic breast |
| | cancer |
| Bcl-2 | Intraocular lymphoma |
| Bcl-2 | Recurrent small cell |
| | lung cancer |
| Bcl-2 | Small lymphocytic |
| | lymphoma |
| Bcl-2 | Small intestine cancer |
| Bcl-2 | Gastrointestinal |
| | stromal tumor |
| Bcl-2 | Advanced malignant |
| | solid tumor |
| Bcl-2 | Peripheral T-cell |
| | lymphoma |
| Bcl-2 | Mantle cell lymphoma |
| Bcl-2 | Breast cancer |
| Bcl-2 | Prostate cancer |
| Bcl-2 | Cutaneous T-cell |
| | lymphoma |
| Bcl-2 | Refractory acute |
| | myeloid leukemia |
| Bcl-2 | Refractory non- |
| | Hodgkin lymphoma |
| Bcl-2 | Male breast tumor |
| Bcl-2 | Immunoblastic large |
| | cell lymphoma |
| Bcl-2 | Diffuse large B-cell |
| | lymphoma |
| Bcl-2 | Merkel cell carcinoma |
| Bcl-2 | Chronic lymphocytic |
| | leukemia |
| Bcl-2 | Follicular lymphoma |
| Bcl-2 | Lymphocytic leukemia |
| Bcl-2 | Lymphoblastic |
| | lymphoma |
| Bcl-2 | Reye's syndrome |
| Bcl-2 | Macroglobulinemia |
| Bcl-2 | Locally advanced |
| | breast cancer |
| Bcl-2 | Glioblastoma |
| Bcl-2 | Plasmacytoma |
| Bcl-2 | Acute myeloid |
| | leukemia |
| Bcl-2 | Acute lymphoblastic |
| | leukemia |
| Bcl-2 | Hodgkin lymphoma |
| Bcl-2 | Refractory |
| | Waldenstrom's |
| | macroglobulinemia |
| Bcl-2 | Melanoma |
| Bcl-2 | Extensive-stage small |
| | cell lung cancer |
| Bcl-2 | Testicular disorders |
| Bcl-2 | Liver cancer |
| Bcl-2 | Recurrent mantle cell |
| | lymphoma |
| Bcl-2 | Recurrent diffuse large |
| | B-cell lymphoma |
| Bcl-2 | Recurrent acute |
| | myeloid leukemia |
| Bcl-2 | Recurrent Hodgkin |
| | lymphoma |
| Bcl-2 | Recurrent non- |
| | Hodgkin lymphoma |
| Bcl-2 | Recurrent adult grade |
| | III lymphogranuloma |
| Bcl-2 | Relapsed marginal |
| | zone lymphoma |
| Bcl-2 | Relapsed B-cell |
| | lymphoma |
| Bcl-2 | Recurrent Grade 3a |
| | Follicular Lymphoma |
| Bcl-2 | Lung cancer |
| Bcl-2 | Non-small cell lung |
| | cancer |
| Bcl-2 | Non-Hodgkin |
| | lymphoma |
| Bcl-2 | Multiple myeloma |
| Bcl-2 | Adult T-cell |
| | leukemia/lymphoma |
| Bcl-2 | Burkitt lymphoma |
| Bcl-2 | Marginal zone B-cell |
| | lymphoma |
| Bcl-2 | Triple-Negative Breast |
| | Cancer |
| Bcl-2 | Type 1 diabetes |
| Bcl-2, c-Myc | Ovarian cancer |
| Bcl-2, c-Myc | Lung cancer |
| Bcl-xl | Tumor |
| Bcl-xl | Colorectal cancer |
| Bcl-xl, Mcl-1 | Head and neck tumor |
| Bcl-xl, Mcl-1 | Bladder cancer |
| BCMA | Autoimmune disease |
| BCMA | Myasthenia gravis |
| BCMA | End-stage renal |
| | disease |
| BCMA | Smoldering Multiple |
| | myeloma |
| BCMA | Systemic lupus |
| | erythematosus |
| BCMA | Peripheral T-cell |
| | lymphoma |
| BCMA | Aquaporin 4 antibody- |
| | positive neuromyelitis |
| | optica spectrum |
| | disorder |
| BCMA | Neuromyelitis optica |
| BCMA | Precursor T-cell acute |
| | lymphoblastic |
| | leukemia/lymphoma |
| BCMA | Immunoblastic |
| | lymphadenopathy |
| BCMA | Chronic inflammatory |
| | demyelinating |
| | polyneuropathy |
| BCMA | Extranodal NK-T cell |
| | lymphoma |
| BCMA | Refractory Multiple |
| | myeloma |
| BCMA | Plasma cell leukemia |
| BCMA | Anaplastic large cell |
| | lymphoma |
| BCMA | Necrotizing myopathy |
| BCMA | Multiple myeloma |
| BCMA | Enteropathy- |
| | associated T-cell |
| | lymphoma |
| BCMA | CD7-positive |
| | hematological |
| | malignancy |
| BCMA, CD138, | Multiple myeloma |
| CD19, CD38 | |
| BCMA, CD16a, IL- | Refractory Multiple |
| 15Rα | myeloma |
| BCMA, CD16a, IL- | Relapsed Multiple |
| 15Rα | myeloma |
| BCMA, CD16a, | Immunoglobulin light |
| NKp46 | chain amyloidosis |
| BCMA, CD16a, | Refractory Multiple |
| NKp46 | myeloma |
| BCMA, CD16a, | Myelodysplastic |
| NKp46 | syndrome |
| BCMA, CD16a, | Relapsed Multiple |
| NKp46 | myeloma |
| BCMA, CD19 | Autoimmune hemolytic |
| | anemia |
| BCMA, CD19 | Vasculitis |
| BCMA, CD19 | Systemic lupus |
| | erythematosus |
| BCMA, CD19 | Precursor B-cell |
| | lymphoblastic |
| | leukemia/lymphoma |
| BCMA, CD19 | Precursor/B-cell |
| | lymphoblastic |
| | leukemia lymphoma |
| BCMA, CD19 | Sjogren's syndrome |
| BCMA, CD19 | Non-Hodgkin |
| | lymphoma |
| BCMA, CD19 | Multiple myeloma |
| BCMA, CD19 | Multiple myeloma |
| BCMA, CD19 | Amyloidosis |
| BCMA, CD19 | Relapsed T-cell acute |
| | lymphoblastic |
| | leukemia |
| BCMA, CD19 | POEMS syndrome |
| BCMA, CD19 | Crouzon syndrome |
| | with acanthosis |
| | nigricans |
| BCMA, CD19 | B-cell lymphoma |
| BCMA, CD19, | Tumor |
| HER2, Trop-2 | |
| BCMA, CD20 | Autoimmune disease |
| BCMA, CD3 | Tumor |
| BCMA, CD38 | Tumor |
| BCMA, CD4 | Tumor |
| BCMA, CD5 | Tumor |
| BCMA, CD7 | Multiple myeloma |
| BCMA, CD70 | Renal cell carcinoma |
| BCMA, CD70 | Refractory plasma cell |
| | myeloma |
| BCMA, CD70 | Multiple myeloma |
| BCMA, CD8 | Tumor |
| BCMA, GPRC5D | Multiple myeloma |
| BCMA, GPRC5D, | Myeloid leukemia |
| NKG2D | |
| BCMA, HPK1 | Myeloproliferative |
| | disorder |
| BCMA, SLAMF7 | Multiple myeloma |
| BCMA, TACI | Refractory plasma cell |
| | myeloma |
| BCMA, TACI | Relapsed Multiple |
| | myeloma |
| BCMA, TGF-β | Refractory B-cell acute |
| | lymphoblastic |
| | leukemia |
| BCMA, TGF-β | Multiple myeloma |
| BCMA, TGF-β | Relapsed T-cell acute |
| | lymphoblastic |
| | leukemia |
| BCMA, VISTA | Multiple myeloma |
| BDNF | Glaucoma |
| BDNF | Alzheimer's disease |
| beclin-1 | Multiple myeloma |
| BEST1 | Vitelliform macular |
| | dystrophy |
| BET, RPE65 | Biallelic RPE65 |
| | Mutation-associated |
| | Retinal Degeneration |
| bFGF | Perioperative ischemia |
| bFGF | Achondroplasia |
| BMP4 | Glioblastoma |
| BMP6 | Sjogren's syndrome |
| BMPR2, VEGF | Fracture |
| BRAF | Tumor |
| BRCA1 | Ovarian cancer |
| BRD4 | Solid tumor |
| BSG | Liver cancer |
| BSG | Recurrent |
| | glioblastoma |
| BSG | Recurrent malignant |
| | glioma |
| BSG, IL-24 | Hepatocellular |
| | carcinoma |
| BTK | Bruton's |
| | agammaglobulinemia |
| BTN3A1 | Acute myeloid |
| | leukemia |
| BTN3A1 | Myelodysplastic |
| | syndrome |
| BTN3A1 | Multiple myeloma |
| C1-INH | Hereditary |
| | angioedema |
| C1-INH | Liver disease |
| C3 | Paroxysmal nocturnal |
| | hemoglobinuria |
| C3 | Neurodegenerative |
| | disease |
| C3 | Immune system |
| | disease |
| C3 | Immunoglobulin A |
| | nephropathy |
| C3 | Liver disease |
| C3 | Complement |
| | dysregulation |
| C3 | C3 glomerulopathy |
| C3, C5 | Geographic atrophy |
| C5 | Myasthenia gravis |
| C5 | Paroxysmal nocturnal |
| | hemoglobinuria |
| C5 | Stargardt disease |
| C5 | Age-related macular |
| | degeneration |
| C5 | Immune system |
| | disease |
| C5 | Immunoglobulin A |
| | nephropathy |
| C5 | Macular degeneration |
| C5 | Geographic atrophy |
| C5, C5AR1 | Sepsis |
| C5, CFB | Genetic disorder |
| C5a | Community-acquired |
| | pneumonia |
| C5a | Lung cancer |
| C9orf72 | Pick's disease |
| | dementia |
| C9orf72 | Amyotrophic lateral |
| | sclerosis |
| C9orf72 | Frontotemporal |
| | dementia |
| CA4 | Ocular hypertension |
| CA8 | Knee arthritis |
| CA8 | Erythromelalgia |
| CADM1 | Hepatocellular |
| | carcinoma |
| CADM1 | Non-small cell lung |
| | cancer |
| CAG repeat | Machado-Joseph |
| expansion | disease |
| CAG repeat | Spinocerebellar ataxia |
| expansion | |
| CAG repeat | Hodgkin lymphoma |
| expansion | |
| CAG repeat | Huntington's disease |
| expansion | |
| CAIX | Advanced renal cell |
| | carcinoma |
| CAIX | Solid tumor |
| CAIX, PDL1 | Tumor |
| CAIX, PSMA | Renal cell carcinoma |
| CAIX, PSMA | Renal clear cell |
| | carcinoma |
| CaN | Liver transplant |
| | rejection |
| CAPN2 | Amyotrophic lateral |
| | sclerosis |
| CAPN3 | Limb-girdle muscular |
| | dystrophy |
| CAPN3 | Limb-girdle muscular |
| | dystrophy type 2A |
| Caspase 2 | Optic nerve injury |
| Caspase 2 | Ischemic optic |
| | neuropathy |
| Caspase 2 | Glaucoma |
| Caspase 2 | Angle-closure |
| | glaucoma |
| CASQ2 | Ventricular tachycardia |
| CAV1 | Amyotrophic lateral |
| | sclerosis |
| Caveolin | Wet age-related |
| | macular degeneration |
| Cbl-b | Endometrial cancer |
| Cbl-b | Pancreatic cancer |
| Cbl-b | Advanced cancer |
| Cbl-b | Renal cell carcinoma |
| Cbl-b | Glioblastoma |
| | multiforme |
| Cbl-b | Cervical cancer |
| Cbl-b | Recurrent melanoma |
| Cbl-b | Platinum-resistant |
| | ovarian cancer |
| Cbl-b | Stage IIB melanoma |
| cccDNA | Hepatitis B |
| CCL19, CD19, IL- | Diffuse large B-cell |
| 7Rα | lymphoma |
| CCL19, IL-7, | Solid tumor |
| MAGEA4 | |
| CCL2 | Diabetic nephropathy |
| CCL21 | Lung cancer |
| CCL4, CTLA4, | Advanced malignant |
| Flt3L, IL-12, PD-1 | solid tumor |
| CCL4, CTLA4, | Head and neck |
| Flt3L, IL-12, PD-1 | squamous cell |
| | carcinoma |
| CCL4, CTLA4, | Triple-negative breast |
| Flt3L, IL-12, PD-1 | cancer |
| CCL4, CTLA4, | Colorectal cancer |
| Flt3L, IL-12, PD-1 | |
| CCL4, CTLA4, | Melanoma |
| Flt3L, IL-12, PD-1 | |
| CCL4, CTLA4, | Liver metastasis |
| Flt3L, IL-12, PD-1 | |
| CCL4, CTLA4, | Liver cancer |
| Flt3L, IL-12, PD-1 | |
| CCL4, CXCL10, IL-12 | Tumor |
| CCL5 | Hepatocellular |
| | carcinoma |
| CCL5, CD19, IL-12, | Solid tumor |
| PD-1, Trop-2 | |
| CCNB1, WT1 | Breast cancer |
| CCND1, ITGB7, | Inflammatory bowel |
| MAdCAM-1, a4B7 | disease |
| CCND1, Raf kinase | Tumor |
| CCR1, EGFR | Glioblastoma |
| CCR2, IL-2, leptin | Solid tumor |
| CCR3, IL-3R, IL-3Rβ | Asthma |
| CCR3, IL-3R, IL-3Rβ | Allergic asthma |
| CCR5 | Breast cancer |
| CCR5 | Lymphoma |
| CCR5 | HIV infection |
| CCR5, CD4 | HIV infection |
| CCR5, TRIM5 | Pancytopenia |
| CCR5, TRIM5 | X-linked severe |
| | combined |
| | immunodeficiency |
| CD103, CD39, CD8 | Tumor |
| CD123 | Acute myeloid |
| | leukemia |
| CD123 | Acute lymphoblastic |
| | leukemia |
| CD123 | Myelodysplastic |
| | syndrome |
| CD123 | Blastic plasmacytoid |
| | dendritic cell neoplasm |
| CD123, CD33 | Tumor |
| CD123, CD33, | Tumor |
| CD38, CD56, CLL-1, MUC1 | |
| CD123, CD33, CLL-1 | Tumor |
| CD123, TIM3 | Acute myeloid |
| | leukemia |
| CD133 | Retinal disease |
| CD133 | Glioblastoma |
| CD133, EGFR | Glioblastoma |
| CD138 | Tumor |
| CD138, NY-ESO-1, | Multiple myeloma |
| SLAMF7, WT1 | |
| CD16a | Pancreatic cancer |
| CD16a | Novel Coronavirus |
| | Infection |
| CD16a | Advanced solid |
| | malignant tumor |
| CD16a | Fallopian tube cancer |
| CD16a | Breast cancer |
| CD16a | Hypoxia |
| CD16a | Ovarian epithelial |
| | carcinoma |
| CD16a | Colorectal cancer |
| CD16a | Acute myeloid |
| | leukemia |
| CD16a | Liver cancer |
| CD16a | Peritoneal cancer |
| CD16a | Lung cancer |
| CD16a | Glioblastoma |
| | multiforme |
| CD16a | B-cell lymphoma |
| CD16a, CD19, IL- | Mantle cell lymphoma |
| 15Rα | |
| CD16a, CD19, IL- | Diffuse large B-cell |
| 15Rα | lymphoma |
| CD16a, CD19, IL- | Chronic lymphocytic |
| 15Rα | leukemia |
| CD16a, CD19, IL- | Follicular lymphoma |
| 15Rα | |
| CD16a, CD19, IL- | Indolent non-Hodgkin |
| 15Rα | lymphoma |
| CD16a, CD19, IL- | Marginal zone B-cell |
| 15Rα | lymphoma |
| CD16a, CD19, IL- | B-cell lymphoma |
| 15Rα | |
| CD16a, CD276, IL-7 | Solid tumor |
| CD16a, HER2 | HER2-positive breast |
| | cancer |
| CD16a, IL-15 | Advanced solid tumor |
| CD16a, IL-15 | Refractory plasma cell |
| | myeloma |
| CD16a, IL-15 | Acute myeloid |
| | leukemia |
| CD16a, IL-15 | Relapsed Multiple |
| | myeloma |
| CD16a, IL-15 | Multiple myeloma |
| CD16a, IL-15, | Pancreatic cancer |
| MICA, MICB | |
| CD16a, IL-15, | Gastroesophageal |
| MICA, MICB | junction cancer |
| CD16a, IL-15, | Advanced solid tumor |
| MICA, MICB | |
| CD16a, IL-15, | Head and neck tumor |
| MICA, MICB | |
| CD16a, IL-15, | Breast cancer |
| MICA, MICB | |
| CD16a, IL-15, | Ovarian cancer |
| MICA, MICB | |
| CD16a, IL-15, | Colorectal cancer |
| MICA, MICB | |
| CD16a, IL-15, | Non-small cell lung |
| MICA, MICB | cancer |
| CD18 | Leukocyte Adhesion |
| | Deficiency Type 1 |
| CD19 | Autoimmune disease |
| CD19 | Myasthenia gravis |
| CD19 | End-stage renal |
| | disease |
| CD19 | Primary progressive |
| | multiple sclerosis |
| CD19 | Graft-versus-host |
| | disease |
| CD19 | Pancreatic cancer |
| CD19 | Inflammation |
| CD19 | Small lymphocytic |
| | lymphoma |
| CD19 | Microscopic |
| | polyangiitis |
| CD19 | Systemic scleroderma |
| CD19 | Systemic lupus |
| | erythematosus |
| CD19 | Gastric cancer |
| CD19 | Idiopathic |
| | inflammatory |
| | myopathy |
| CD19 | Mantle cell lymphoma |
| CD19 | Granulomatosis with |
| | polyangiitis |
| CD19 | Precursor B-cell acute |
| | lymphoblastic |
| | leukemia |
| CD19 | Precursor B-cell |
| | lymphoblastic |
| | leukemia lymphoma |
| CD19 | Dermatomyositis |
| CD19 | Refractory acute |
| | leukemia |
| CD19 | Refractory non- |
| | Hodgkin lymphoma |
| CD19 | Refractory B-cell |
| | lymphoma |
| CD19 | Refractory B-type |
| | acute lymphoblastic |
| | leukemia |
| CD19 | Diffuse scleroderma |
| CD19 | Hairy cell leukemia |
| CD19 | Chronic lymphocytic |
| | leukemia |
| CD19 | Chronic myeloid |
| | leukemia |
| CD19 | Lymphomatoid |
| | granulomatosis |
| CD19 | Lymphoma |
| CD19 | Lupus nephritis |
| CD19 | Antineutrophil |
| | cytoplasmic antibody- |
| | associated vasculitis |
| CD19 | Secondary |
| | progressive multiple |
| | sclerosis |
| CD19 | Acute lymphoblastic |
| | leukemia |
| CD19 | Macular degeneration |
| CD19 | Philadelphia |
| | chromosome-negative |
| | acute lymphoblastic |
| | leukemia |
| CD19 | Non-Hodgkin |
| | lymphoma |
| CD19 | Fanconi anemia |
| CD19 | Multiple sclerosis |
| CD19 | Residual tumor |
| CD19 | AIDS-related |
| | lymphoma |
| CD19 | CD19-positive B-cell |
| | acute lymphoblastic |
| | leukemia |
| CD19 | CD19-positive B-cell |
| | acute lymphoblastic |
| | leukemia |
| CD19 | B-cell lymphoma |
| CD19 | Type 2 diabetes |
| CD19, CD20 | Purpura hepatitis |
| CD19, CD20 | Precursor B-cell acute |
| | lymphoblastic |
| | leukemia |
| CD19, CD20 | Chronic lymphocytic |
| | leukemia |
| CD19, CD20 | Philadelphia |
| | chromosome-negative |
| | acute lymphoblastic |
| | leukemia |
| CD19, CD20, CD22 | Refractory non- |
| | Hodgkin lymphoma |
| CD19, CD20, CD22 | Refractory indolent |
| | non-Hodgkin |
| | lymphoma |
| CD19, CD20, CD22 | Chronic lymphocytic |
| | leukemia |
| CD19, CD20, CD22 | Acute lymphoblastic |
| | leukemia |
| CD19, CD20, CD22 | Relapsed transformed |
| | chronic lymphocytic |
| | leukemia |
| CD19, CD20, CD22 | Relapsed chronic |
| | lymphocytic leukemia |
| CD19, CD20, CD22 | Relapsed acute |
| | lymphoblastic |
| | leukemia |
| CD19, CD20, CD22 | Relapsed non-Hodgkin |
| | lymphoma |
| CD19, CD20, CD22 | Relapsed indolent |
| | non-Hodgkin |
| | lymphoma |
| CD19, CD20, CD22 | Relapsed B-type acute |
| | lymphoblastic |
| | leukemia |
| CD19, CD20, CD22 | B-cell lymphoma |
| CD19, CD22 | Autoimmune disease |
| CD19, CD22 | Purpura hepatitis |
| CD19, CD22 | Hematologic |
| | malignancy |
| CD19, CD22 | Blood disorder |
| CD19, CD22 | Precursor B-cell acute |
| | lymphoblastic |
| | leukemia |
| CD19, CD22 | Precursor B-cell |
| | lymphoblastic |
| | leukemia lymphoma |
| CD19, CD22 | Refractory B-type |
| | acute lymphoblastic |
| | leukemia |
| CD19, CD22 | Relapsed B-type acute |
| | lymphoblastic |
| | leukemia |
| CD19, CD22 | Philadelphia |
| | chromosome-negative |
| | acute lymphoblastic |
| | leukemia |
| CD19, CD22 | Philadelphia |
| | chromosome-positive |
| | acute lymphoblastic |
| | leukemia |
| CD19, CD22 | Non-Hodgkin |
| | lymphoma |
| CD19, CD22 | Residual tumor |
| CD19, CD22 | Ph-like acute |
| | lymphoblastic |
| | leukemia |
| CD19, CD22 | CD22-positive B-cell |
| | precursor acute |
| | lymphoblastic |
| | leukemia |
| CD19, CD22 | CD22-positive acute |
| | lymphoblastic |
| | leukemia |
| CD19, CD22 | CD22-positive B-cell |
| | acute lymphoblastic |
| | leukemia |
| CD19, CD22 | CD19-positive B-cell |
| | precursor acute |
| | lymphoblastic |
| | leukemia |
| CD19, CD22 | CD19-positive B-cell |
| | acute lymphoblastic |
| | leukemia |
| CD19, CD22 | B-cell lymphoma |
| CD19, CD22, CD8 | Chronic lymphocytic |
| | leukemia |
| CD19, CD22, CD8 | Acute lymphoblastic |
| | leukemia |
| CD19, CD22, CD8 | Non-Hodgkin |
| | lymphoma |
| CD19, CD276 | Ewing sarcoma |
| CD19, CD276 | Clear cell sarcoma |
| CD19, CD276 | Retinoblastoma |
| CD19, CD276 | Wilms tumor |
| CD19, CD276 | Neurofibrosarcoma |
| CD19, CD276 | Neuroblastoma |
| CD19, CD276 | sarcoma |
| CD19, CD276 | Synovial sarcoma |
| CD19, CD276 | Rhabdoid tumor |
| CD19, CD276 | Rhabdomyosarcoma |
| CD19, CD276 | Melanoma |
| CD19, CD276 | Hepatoblastoma |
| CD19, CD276 | Recurrent solid tumor |
| CD19, CD276 | Malignant epithelial |
| | tumor |
| CD19, CD276 | Desmoplastic small |
| | round cell tumor |
| CD19, CD3 | Severe combined |
| | immunodeficiency |
| CD19, CD3 | Common variable |
| | immunodeficiency |
| CD19, CD3 | Refractory B-cell |
| | lymphoma |
| CD19, CD3 | Immunodeficiency |
| | syndrome |
| CD19, CD3 | Mendelian |
| | susceptibility to |
| | mycobacterial disease |
| CD19, CD3 | Chronic |
| | granulomatous |
| | disease |
| CD19, CD3 | Lymphoma |
| CD19, CD3 | Relapsed B-cell |
| | lymphoma |
| CD19, CD3 | Wiskott-Aldrich |
| | syndrome |
| CD19, CD3 | Job syndrome |
| CD19, CD3 | B-cell lymphoma |
| CD19, CD4 | Chronic lymphocytic |
| | leukemia |
| CD19, CD4 | Non-Hodgkin |
| | lymphoma |
| CD19, CD7 | Precursor B-cell acute |
| | lymphoblastic |
| | leukemia |
| CD19, CD7 | Acute lymphoblastic |
| | leukemia |
| CD19, CD7 | B-cell leukemia |
| CD19, CD70 | Refractory B-cell |
| | lymphoma |
| CD19, CD70 | Lymphoma |
| CD19, CD70 | Acute myeloid |
| | leukemia |
| CD19, CD70 | Relapsed B-cell |
| | lymphoma |
| CD19, CD70 | Non-Hodgkin |
| | lymphoma |
| CD19, CD70 | B-cell lymphoma |
| CD19, CD79B | Non-Hodgkin |
| | lymphoma |
| CD19, CD8 | Precursor B-cell |
| | lymphoblastic |
| | leukemia lymphoma |
| CD19, CD8 | Chronic lymphocytic |
| | leukemia |
| CD19, CD8 | Non-Hodgkin |
| | lymphoma |
| CD19, CLDN18.2 | Gastric cancer |
| CD19, DR5 | B-cell lymphoma |
| CD19, EBV protein | Acute lymphoblastic |
| | leukemia |
| CD19, EBV protein | B-cell lymphoma |
| CD19, EGFR | Tumor |
| CD19, HPK1 | Acute lymphoblastic |
| | leukemia |
| CD19, HPK1 | B-cell lymphoma |
| CD19, IL15R | Small lymphocytic |
| | lymphoma |
| CD19, IL15R | Mantle cell lymphoma |
| CD19, IL15R | Precursor B-cell acute |
| | lymphoblastic |
| | leukemia |
| CD19, IL15R | Chronic lymphocytic |
| | leukemia |
| CD19, IL15R | Lupus nephritis |
| CD19, IL15R | Macroglobulinemia |
| CD19, IL15R | Large B-cell |
| | lymphoma |
| CD19, IL15R | B-cell malignancy |
| CD19, IL-18 | Acute lymphoblastic |
| | leukemia |
| CD19, IL-18 | B-cell lymphoma |
| CD19, IL18R1 | Refractory B-type |
| | acute lymphoblastic |
| | leukemia |
| CD19, IL18R1 | Chronic lymphocytic |
| | leukemia |
| CD19, IL18R1 | Acute lymphoblastic |
| | leukemia |
| CD19, IL18R1 | Relapsed acute |
| | lymphoblastic |
| | leukemia |
| CD19, IL18R1 | Relapsed and |
| | refractory acute |
| | lymphoblastic |
| | leukemia |
| CD19, IL18R1 | Non-Hodgkin |
| | lymphoma |
| CD19, IL18R1 | CD19 expressing |
| | malignancy |
| CD19, IL-2Rβ | Tumor |
| CD19, IL-2Rβ | Small lymphocytic |
| | lymphoma |
| CD19, IL-2Rβ | Mantle cell lymphoma |
| CD19, IL-2Rβ | Diffuse large B-cell |
| | lymphoma |
| CD19, IL-2Rβ | Chronic lymphocytic |
| | leukemia |
| CD19, IL-2Rβ | Follicular lymphoma |
| CD19, IL-2Rβ | Indolent B-cell non- |
| | Hodgkin lymphoma |
| CD19, IL-2Rβ | Large B-cell |
| | lymphoma |
| CD19, IL-2Rβ | CD19 expressing |
| | malignancy |
| CD19, MUC1 | Tumor |
| CD19, PD-1 | Solid tumor |
| CD19, PD-1 | Refractory cancer |
| CD19, PD-1 | Refractory B-cell |
| | lymphoma |
| CD19, PD-1 | Relapsed non-Hodgkin |
| | lymphoma |
| CD19, PD-1 | Non-Hodgkin |
| | lymphoma |
| CD19, PD-1 | Mesothelin-positive |
| | tumor |
| CD19, PD-1 | CD19 expressing |
| | malignancy |
| CD19, PD-1 | Carney complex |
| CD19, PD-1 | B-cell lymphoma |
| CD19, PD-1, TIGIT | Mediastinal large B- |
| | cell lymphoma |
| CD19, PD-1, TIGIT | Follicular lymphoma |
| CD19, PD-1, TIGIT | High-grade B-cell |
| | lymphoma |
| CD19, PD-1, TIGIT | Large B-cell |
| | lymphoma |
| CD19, STING | Solid tumor |
| CD19, TGF-β2 | Non-Hodgkin |
| | lymphoma |
| CD19, TGF-β2 | CD19-positive diffuse |
| | large B-cell lymphoma |
| CD19, TGF-β2 | CD19-positive B-cell |
| | acute lymphoblastic |
| | leukemia |
| CD1A | Precursor T- |
| | lymphocytic leukemia |
| | lymphoma |
| CD1A | Precursor T- |
| | lymphocytic leukemia |
| | lymphoma |
| CD1A | Lymphoma |
| CD1A | T-cell acute |
| | lymphoblastic |
| | leukemia/lymphoma |
| CD2 | Sezary syndrome |
| CD20 | Mediastinal large B- |
| | cell lymphoma |
| CD20 | Autoimmune disease |
| CD20 | Metastatic melanoma |
| CD20 | Hematologic |
| | malignancy |
| CD20 | Small lymphocytic |
| | lymphoma |
| CD20 | Skin melanoma |
| CD20 | Refractory mantle cell |
| | lymphoma |
| CD20 | Refractory B-cell |
| | lymphoma |
| CD20 | Chronic lymphocytic |
| | leukemia |
| CD20 | Follicular lymphoma |
| CD20 | Waldenstrom's |
| | macroglobulinemia is |
| | refractory |
| CD20 | Melanoma |
| CD20 | Relapsed mantle cell |
| | lymphoma |
| CD20 | Relapsed chronic |
| | lymphocytic leukemia |
| CD20 | Recurrent |
| | macroglobulinemia |
| CD20 | Relapsed B-cell |
| | lymphoma |
| CD20 | Marginal zone B-cell |
| | lymphoma |
| CD20 | CD20-positive B-cell |
| | lymphoma |
| CD20, CD22 | Precursor B-cell |
| | lymphoblastic |
| | leukemia lymphoma |
| CD20, CD22 | Refractory B-cell |
| | lymphoma |
| CD20, CD22 | Non-Hodgkin |
| | lymphoma |
| CD20, CD22, CD38 | Breast cancer |
| CD20, CD23, TLR9 | Hematologic |
| | malignancy |
| CD20, CD79A | Non-Hodgkin |
| | lymphoma |
| CD200R | Immune system |
| | disorder |
| CD22 | Autoimmune disease |
| CD22 | Hairy cell leukemia |
| CD22 | Acute lymphoblastic |
| | leukemia |
| CD22 | Non-Hodgkin |
| | lymphoma |
| CD22 | B-cell leukemia |
| CD22, CD37 | Hematologic |
| | malignancy |
| CD22, PDL1 | sarcoma |
| CD22, PDL1 | Cervical cancer |
| CD22, PDL1 | Non-small cell lung |
| | cancer |
| CD24 | Tumor |
| CD276 | Diffuse midline glioma |
| | with K27M point |
| | mutation in histone H3 |
| CD276 | Pancreatic ductal |
| | adenocarcinoma |
| CD276 | Pancreatic cancer |
| CD276 | Medulloblastoma |
| CD276 | Ependymoma |
| CD276 | Neuroblastoma |
| CD276 | Brain malignant glioma |
| CD276 | Refractory acute |
| | myeloid leukemia |
| CD276 | Diffuse intrinsic |
| | pontine glioma |
| CD276 | Ovarian epithelial |
| | carcinoma |
| CD276 | Colorectal cancer |
| CD276 | Colorectal cancer |
| CD276 | Glioblastoma |
| CD276 | Glioma |
| CD276 | Acute myeloid |
| | leukemia |
| CD276 | Melanoma |
| CD276 | Liver cancer |
| CD276 | Recurrent platinum- |
| | resistant ovarian |
| | cancer |
| CD276 | Lung cancer |
| CD276 | Atypical teratoma |
| CD276 | Recurrent ovarian |
| | tumor of low malignant |
| | potential |
| CD276 | CD276-positive solid |
| | tumor |
| CD276, FGFR4 | Rhabdomyosarcoma |
| CD276, HER2 | Tumor |
| CD276, IL-13Ra2 | Solid tumor |
| CD28, CD80, p53 | Tumor |
| CD28, CSF-2R, | Tumor |
| CTLA4 | |
| CD29, EGFR, | Metastatic non-small |
| LAMA4 | cell lung cancer |
| CD29, EGFR, | Breast cancer |
| LAMA4 | |
| CD29, EGFR, | Glioma |
| LAMA4 | |
| CD3 | Tumor |
| CD3, CD7 | Acute lymphoblastic |
| | leukemia |
| CD3, CD80 | Tumor |
| CD3, CD86, PD-1 | Endometrial cancer |
| CD3, CD86, PD-1 | Bronchogenic |
| | carcinoma |
| CD3, CD86, PD-1 | Pancreatic cancer |
| CD3, CD86, PD-1 | Pharyngeal tumor |
| CD3, CD86, PD-1 | Small cell lung cancer |
| CD3, CD86, PD-1 | Gastrointestinal tumor |
| CD3, CD86, PD-1 | Gastric cancer |
| CD3, CD86, PD-1 | Advanced malignant |
| | solid tumor |
| CD3, CD86, PD-1 | Advanced cancer |
| CD3, CD86, PD-1 | Head and neck tumor |
| CD3, CD86, PD-1 | Fallopian tube cancer |
| CD3, CD86, PD-1 | Esophageal cancer |
| CD3, CD86, PD-1 | Kidney tumor |
| CD3, CD86, PD-1 | Soft tissue sarcoma |
| CD3, CD86, PD-1 | Breast cancer |
| CD3, CD86, PD-1 | Sarcoma |
| CD3, CD86, PD-1 | Female genitor tumor |
| CD3, CD86, PD-1 | Brain cancer |
| CD3, CD86, PD-1 | Ovarian cancer |
| CD3, CD86, PD-1 | Colorectal cancer |
| CD3, CD86, PD-1 | Laryngeal tumor |
| CD3, CD86, PD-1 | Melanoma |
| CD3, CD86, PD-1 | Osteosarcoma |
| CD3, CD86, PD-1 | Bone cancer |
| CD3, CD86, PD-1 | Cervical cancer |
| CD3, CD86, PD-1 | Liver cancer |
| CD3, CD86, PD-1 | Peritoneal cancer |
| CD3, CD86, PD-1 | Recurrent Head and |
| | neck tumor |
| CD3, CD86, PD-1 | Recurrent ovarian |
| | cancer |
| CD3, CD86, PD-1 | Lung cancer |
| CD3, CD86, PD-1 | Non-small cell lung |
| | cancer |
| CD3, CD86, PD-1 | Ear tumor |
| CD3, CD86, PD-1 | Malignant epithelial |
| | tumor |
| CD3, CD86, PD-1 | Nasal tumor |
| CD3, EGFR | Pancreatic |
| | adenocarcinoma |
| CD3, EGFR | Pancreatic cancer |
| CD3, EGFR | Glioblastoma |
| CD3, EGFRVIII, IL-13Rα2 | Glioblastoma |
| CD3, EpCAM | Tumor |
| CD3, EphA2 | Tumor |
| CD3, FAP | Tumor metastasis |
| CD3, FAP | Adenoepithelial tumor |
| CD3, FAP | Malignant epithelial |
| | tumor |
| CD3, gp100 | Uveal melanoma |
| CD3, gp100 | Cutaneous melanoma |
| CD3, gp100 | Residual tumor |
| CD3, GPC3 | Solid tumor |
| CD3, GPRC5D | Tumor |
| CD3, HER2 | Breast cancer |
| CD3, IL-13Rα2, PDL1 | Glioblastoma |
| CD3, IL-13Rα2, PDL1 | Glioma |
| CD3, MUC1 | Advanced pancreatic |
| | adenocarcinoma |
| CD3, MUC1 | Advanced gastric |
| | cancer |
| CD3, MUC1 | Advanced breast |
| | cancer |
| CD3, MUC1 | Kidney tumor |
| CD3, MUC1 | Colorectal cancer |
| CD3, MUC1 | Liver cancer |
| CD3, MUC1 | Lung cancer |
| CD3, PDL1 | Solid tumor |
| CD3, PDL1 | Recurrent cervical |
| | cancer |
| CD3, PRAME | Advanced malignant |
| | solid tumor |
| CD3, PRAME | Cutaneous melanoma |
| CD3, PRAME | Melanoma |
| CD3, SLAMF7 | Refractory plasma cell |
| | myeloma |
| CD3, SLAMF7 | Relapsed Multiple |
| | myeloma |
| CD30 | Peripheral T-cell |
| | lymphoma |
| CD30 | Diffuse large B-cell |
| | lymphoma |
| CD30 | Anaplastic large cell |
| | lymphoma |
| CD30 | Anaplastic large cell |
| | lymphoma |
| CD30 | Hodgkin lymphoma |
| CD30 | T-cell lymphoma |
| CD30 | CD30-positive |
| | lymphoma |
| CD30 | CD30-positive |
| | Hodgkin lymphoma |
| CD30 | CD30-positive diffuse |
| | large B-cell lymphoma |
| CD30 | CD19-positive |
| | lymphoma |
| CD30 | CD19 expressing |
| | malignancy |
| CD30 | B-cell lymphoma |
| CD30 | B-cell leukemia |
| CD33 | Graft-versus-host |
| | disease |
| CD33 | Acute myeloid |
| | leukemia |
| CD33 | Alzheimer's disease |
| CD33, CLL-1 | Hematologic |
| | malignancy |
| CD33, CLL-1 | Chronic myeloid |
| | leukemia |
| CD33, CLL-1 | Chronic |
| | myelomonocytic |
| | leukemia |
| CD33, CLL-1 | Acute myeloid |
| | leukemia |
| CD33, CLL-1 | Acute myeloid |
| | leukemia |
| CD33, CLL-1 | Myelodysplastic |
| | syndrome |
| CD33, FLT3 | Hematologic |
| | malignancy |
| CD33, FLT3 | Refractory acute |
| | myeloblastic leukemia |
| CD33, FLT3 | Myelodysplastic |
| | syndrome |
| CD33, FLT3 | Relapsed acute |
| | myeloblastic leukemia |
| CD33, IL-15Rα, NKG2C | Acute myeloid |
| | leukemia |
| CD33, NKG2A | Acute myeloid |
| | leukemia in adults |
| CD34 | Severe combined |
| | immunodeficiency |
| CD34 | Tumor |
| CD34 | Hematologic |
| | malignancy |
| CD34 | Adenosine deaminase |
| | deficiency |
| CD34 | Myeloid tumor |
| CD34 | Acute leukemia |
| CD34 | Myelodysplastic |
| | syndrome |
| CD34 | Multiple myeloma |
| CD34 | Wiskott-Aldrich |
| | syndrome |
| CD37 | Chronic lymphocytic |
| | leukemia |
| CD37 | T-cell lymphoma |
| CD37 | B-cell lymphoma |
| CD38 | Refractory acute |
| | myeloid leukemia |
| CD38 | Hepatocellular |
| | carcinoma |
| CD38 | Relapsed acute |
| | myeloid leukemia |
| CD38 | Multiple myeloma |
| CD38, CLL-1 | Cancer |
| CD38, DR5 | Multiple myeloma |
| CD38, GPRC5D | Tumor |
| CD39, CD73 | Rheumatoid arthritis |
| CD4 | Hematopoietic stem |
| | cell transplantation |
| CD4 | Graft-versus-host |
| | disease |
| CD4 | Precursor T-cell |
| | lymphocytic |
| | leukemia/lymphoma |
| CD4 | Chronic |
| | myelomonocytic |
| | leukemia |
| CD4 | T-cell lymphoma |
| CD4 | T-cell leukemia |
| CD4 | HIV infection |
| CD4, CD8 | Colorectal cancer |
| CD4, CD8 | HIV infection |
| CD40 | Autoimmune disease |
| CD40 | Tumor metastasis |
| CD40 | Adenoepithelial tumor |
| CD40 | Autoimmune |
| | neurological disorder |
| CD40 | Organ transplant |
| | rejection |
| CD40 | Hypersensitivity |
| | reactions |
| CD40L | Metastatic renal cell |
| | carcinoma |
| CD40L | Tumor |
| CD40L | Chronic lymphocytic |
| | leukemia |
| CD40L | Vaccination |
| CD40L | Glioma |
| CD40L | Melanoma |
| CD40L | Recurrent non-small |
| | cell lung cancer |
| CD40L | Non-muscle invasive |
| | bladder tumor |
| CD40L | Bladder urothelial |
| | bladder |
| CD40L | Bladder cancer |
| CD40L | Stage IV malignant |
| | non-small cell lung |
| | tumor |
| CD40L, CD70, TLR4 | Tumor |
| CD40L, CD70, TLR4 | Melanoma |
| CD40L, CD70, | Stage IV malignant |
| TLR4 | melanoma |
| CD40L, CD70, | Unresectable |
| TLR4 | melanoma |
| CD40L, IFNβ | Tumor |
| CD40L, OX40L | Tumor |
| CD40L, OX40L | Triple-negative breast |
| | cancer |
| CD40L, OX40L | Sarcoma |
| CD40L, OX40L | Melanoma |
| CD40L, OX40L | Non-small cell lung |
| | cancer |
| CD43 | T-cell lymphoma |
| CD43 | T-cell leukemia |
| CD44 | Hematologic |
| | malignancy |
| CD44v6 | Solid tumor |
| CD44v6 | Acute myeloid |
| | leukemia |
| CD44v6 | Multiple myeloma |
| CD45 | Hematologic |
| | malignancy |
| CD45 | Bone marrow tumor |
| CD46 | Rectal cancer |
| CD46 | Head and neck tumor |
| CD46 | Renal cell carcinoma |
| CD46 | Ovarian cancer |
| CD46 | Colorectal cancer |
| CD46 | Non-small cell lung |
| | cancer |
| CD46 | Bladder cancer |
| CD46, PEDF | Geographic atrophy |
| CD47 | Hematologic |
| | malignancy |
| CD47 | Solid tumor |
| CD47 | Sickle cell disease |
| CD47 | Thalassemia |
| CD47, PHD2 | Tumor |
| CD47, PHD2 | Hematologic |
| | malignancy |
| CD47, PHD2 | Solid tumor |
| CD47, SIRPα | Tumor |
| CD47, SIRPα | Advanced malignant |
| | solid tumor |
| CD47, SIRPα | Solid tumor |
| CD47, SIRPα | Refractory non- |
| | Hodgkin lymphoma |
| CD47, SIRPα | Relapsed non-Hodgkin |
| | lymphoma |
| CD47, SIRPα | Atherosclerosis |
| CD49d | Multiple sclerosis |
| CD49d | Duchenne muscular |
| | dystrophy |
| CD5 | Mycosis fungoides |
| CD5 | Hematologic |
| | malignancy |
| CD5 | Peripheral T-cell |
| | lymphoma |
| CD5 | Precursor T-cell |
| | lymphocytic |
| | leukemia/lymphoma |
| CD5 | Precursor B-cell |
| | lymphoblastic |
| | leukemia/lymphoma |
| CD5 | Refractory T-cell acute |
| | lymphoblastic |
| | leukemia |
| CD5 | Refractory B-type |
| | acute lymphoblastic |
| | leukemia |
| CD5 | Lymphoma |
| CD5 | Acute lymphoblastic |
| | leukemia |
| CD5 | Relapsed acute |
| | lymphoblastic |
| | leukemia |
| CD5 | Relapsed T-cell |
| | lymphoma |
| CD5 | Non-Hodgkin |
| | lymphoma |
| CD5 | T-cell lymphoma |
| CD5 | T-cell acute |
| | lymphoblastic |
| | leukemia/lymphoma |
| CD5, IL15R | Hematologic |
| | malignancy |
| CD56 | Extranodal NK-T-cell |
| | lymphoma |
| CD56 | Recurrent |
| | nasopharyngeal |
| | carcinoma |
| CD59 | Age-related macular |
| | degeneration |
| CD59 | Geographic atrophy |
| CD7 | Juvenile idiopathic |
| | arthritis |
| CD7 | Mycosis fungoides |
| CD7 | Peripheral T-cell |
| | lymphoma |
| CD7 | Sezary syndrome |
| CD7 | Precursor T-cell |
| | lymphocytic |
| | leukemia/lymphoma |
| CD7 | Dermatomyositis |
| CD7 | Refractory T-cell |
| | lymphocytic/lymphoma |
| CD7 | Refractory T-cell acute |
| | lymphoblastic |
| | leukemia |
| CD7 | Immunoblastic |
| | lymphadenopathy |
| CD7 | Lymphoblastic |
| | lymphoma |
| CD7 | Ulcerative colitis |
| CD7 | Crohn's disease |
| CD7 | Extranodal NK-T-cell |
| | lymphoma |
| CD7 | Anaplastic large cell |
| | lymphoma |
| CD7 | Acute myeloid |
| | leukemia |
| CD7 | Acute lymphoblastic |
| | leukemia |
| CD7 | Hepatosplenic T-cell |
| | lymphoma |
| CD7 | Relapsed T-cell |
| | lymphoma |
| CD7 | Non-Hodgkin |
| | lymphoma |
| CD7 | Large granular |
| | lymphocytic leukemia |
| CD7 | Adult T-cell |
| | lymphoma/leukemia |
| CD7 | Adult T-cell |
| | leukemia/lymphoma |
| CD7 | Enteropathy- |
| | associated T-cell |
| | lymphoma |
| CD7 | Enteropathy- |
| | associated T-cell |
| | lymphoma |
| CD7 | T-cell lymphocytic |
| | leukemia |
| CD7 | T-cell lymphoma |
| CD7 | T-cell lymphoma |
| CD7 | Recurrent T-cell acute |
| | lymphoblastic |
| | leukemia |
| CD7 | T-cell acute |
| | lymphoblastic |
| | leukemia/lymphoma |
| CD7 | T-cell acute |
| | lymphoblastic |
| | leukemia/lymphoma |
| CD7 | NK-cell lymphoma |
| CD7 | CD7-positive tumor |
| CD7 | CD7-positive acute |
| | myeloid leukemia |
| CD70 | Metastatic clear cell |
| | renal cell carcinoma |
| CD70 | Tumor metastasis |
| CD70 | Pancreatic |
| | adenocarcinoma |
| CD70 | Hematologic |
| | malignancy |
| CD70 | Advanced renal cell |
| | carcinoma |
| CD70 | Advanced solid |
| | malignant tumor |
| CD70 | Advanced cancer |
| CD70 | Esophageal cancer |
| CD70 | Solid tumor |
| CD70 | Solid tumor |
| CD70 | Renal cell carcinoma |
| CD70 | Urologic tumor |
| CD70 | Ovarian cancer |
| CD70 | Lymphoma |
| CD70 | Acute myeloid |
| | leukemia |
| CD70 | Acute myeloid |
| | leukemia |
| CD70 | Cervical cancer |
| CD70 | Non-Hodgkin |
| | lymphoma |
| CD70 | Non-Hodgkin |
| | lymphoma |
| CD70 | Malignant pleural |
| | mesothelioma |
| CD70 | Multiple myeloma |
| CD70 | T-cell lymphoma |
| CD70 | CD70-positive tumor |
| CD70 | CD70-positive female |
| | genitor |
| | tumor |
| CD70, IL-15, TGF-β | Renal cell carcinoma |
| CD70, IL15R | Advanced renal cell |
| | carcinoma |
| CD70, IL15R | Mesothelioma |
| CD70, IL15R | Osteosarcoma |
| CD72 | Acute lymphoblastic |
| | leukemia |
| CD74 | Alzheimer's disease |
| CD79B | Refractory B-cell acute |
| | lymphoblastic |
| | leukemia |
| CD79B | Acute lymphoblastic |
| | leukemia |
| CD79B | B-cell lymphoma |
| CD79B | B-cell lymphoma |
| CD8 | Advanced Head and |
| | neck tumor |
| CD8 | Head and neck tumor |
| CD80, CD86, PDL1 | Non-small cell lung |
| | cancer |
| CD80, GM-CSF | Tumor |
| CD80, IFNα, MIP-1α | Solid tumor |
| CD83 | Tumor |
| CD84 | Adult acute myeloid |
| | leukemia |
| CD86 | Glioma |
| CD86 | Melanoma |
| CD86, HPV E6, | Solid tumor |
| HPV E7, IL-12, IL-2 | |
| CD86, HPV E6, | HPV16-positive solid |
| HPV E7, IL-12, IL-2 | tumor |
| CD8A, NY-ESO-1 | Solid tumor |
| CD94 | Plasmacytic leukemia |
| CD99 | Hematologic |
| | malignancy |
| CD99 | Myeloid leukemia |
| CD99 | Sarcoma |
| CDA | Gastrointestinal tumor |
| CDA | Glioblastoma |
| CDA | High-grade glioma |
| CDA | Recurrent |
| | glioblastoma |
| CDCP1 | Pancreatic ductal |
| | adenocarcinoma |
| CDH17 | Metastatic colorectal |
| | cancer |
| CDH17 | Pancreatic cancer |
| CDH17 | Neuroendocrine tumor |
| CDH17 | Colorectal cancer |
| CDH5 | Diabetic retinopathy |
| CDKL5 | Neurological disorder |
| CDKL5 | X-linked genetic |
| | disease |
| CDKL5 | CDKL5 Deficiency |
| CDKN1A | Non-muscle invasive |
| | bladder tumor |
| CDKs | Tumor |
| CDKs | Sensorineural hearing |
| | loss |
| CEA | Tumor metastasis |
| CEA | Rectal cancer |
| CEA | Pancreatic acinar |
| | carcinoma |
| CEA | Pancreatic cancer |
| CEA | Gastric cancer |
| CEA | Advanced solid |
| | malignant tumor |
| CEA | Esophageal cancer |
| CEA | Oligodendroglioma |
| CEA | Breast cancer |
| CEA | Ovarian cancer |
| CEA | Colorectal cancer |
| CEA | Colorectal cancer |
| CEA | Colon cancer |
| CEA | Colon cancer |
| CEA | Liver metastasis |
| CEA | Liver cancer |
| CEA | Peritoneal cancer |
| CEA | Recurrent cancer |
| CEA | Lung cancer |
| CEA | Cholangiocarcinoma |
| CEA | Biliary tract tumor |
| CEA, IL-15Rα, IL-21R | Colorectal cancer |
| CEACAM5 | Pancreatic acinar |
| | carcinoma |
| CEACAM5 | Pancreatic |
| | adenocarcinoma |
| CEACAM5 | Pancreatic cancer |
| CEACAM5 | Adenocarcinoma |
| CEACAM5 | Gastric cancer |
| CEACAM5 | Gastric cancer |
| CEACAM5 | Breast cancer |
| CEACAM5 | Breast cancer |
| CEACAM5 | Ovarian cancer |
| CEACAM5 | Squamous non-small |
| | cell lung cancer |
| CEACAM5 | Colorectal cancer |
| CEACAM5 | Colorectal cancer |
| CEACAM5 | Liver metastasis |
| CEACAM5 | Liver cancer |
| CEACAM5 | Lung cancer |
| CEACAM5 | Malignant ascites |
| CEACAM5, | Tumor |
| CEACAM6 | |
| CEACAM7 | Pancreatic cancer |
| CEBPA | Hepatitis B |
| CEBPA | Hepatocellular |
| | carcinoma |
| CEBPA | Liver cancer |
| CEBPA | Hepatitis C |
| CEBPB | Breast cancer |
| CEBPB | Melanoma |
| CEBPB | Hepatocellular |
| | carcinoma |
| CEBPB | Bladder cancer |
| Cell adhesion | Mantle cell lymphoma |
| molecules, NF-κB, | |
| elF5A | |
| Cell adhesion | Diffuse large B-cell |
| molecules, NF-κB, | lymphoma |
| elF5A | |
| Cell adhesion | Plasma cell leukemia |
| molecules, NF-κB, | |
| elF5A | |
| Cell adhesion | Relapsed mantle cell |
| molecules, NF-κB, | lymphoma |
| elF5A | |
| Cell adhesion | Relapsed diffuse large |
| molecules, NF-κB, | B-cell lymphoma |
| elF5A | |
| Cell adhesion | Relapsed Multiple |
| molecules, NF-κB, | myeloma |
| elF5A | |
| Cell adhesion | Relapsed B-cell |
| molecules, NF-κB, | lymphoma |
| elF5A | |
| Cell adhesion | Multiple myeloma |
| molecules, NF-κB, | |
| elF5A | |
| Cep290 | Retinal Degenerative |
| | Disease |
| Cep290 | Leber congenital |
| | amaurosis type 10 |
| Cep290, nuclease | Hereditary retinal |
| Cas9 | dystrophy |
| Cep290, nuclease | Eye deformities |
| Cas9 | |
| Cep290, nuclease | Retinal degeneration |
| Cas9 | |
| Cep290, nuclease | Visual impairment |
| Cas9 | |
| CFB | Age-related macular |
| | degeneration |
| CFB | Immune system |
| | disorders |
| CFB | Immunoglobulin A |
| | nephropathy |
| CFB | Geographic atrophy |
| CFH | Macular degeneration |
| CFI | Sagging skin |
| CFI | Age-related macular |
| | degeneration |
| CF | Macular degeneration |
| CF | Dry age-related |
| | macular degeneration |
| CF | Geographic atrophy |
| CFI | Glycogen storage |
| | disease type II |
| CFLAR | Tumor |
| CFTR | Retinal disease |
| CFTR | Cystic fibrosis |
| cGMP-PDE | Retinitis pigmentosa |
| Chk1 | Brain metastases |
| Chk1 | Glioblastoma |
| chloride channel | Cystic fibrosis |
| CHOP | Diabetic nephropathy |
| CHST15 | Ulcerative colitis |
| CHST15 | Crohn's disease |
| chymase | Heart failure |
| CIDEB | Nonalcoholic |
| | steatohepatitis |
| CISH | Gastrointestinal tumor |
| CISH, IL-15 | Hematologic |
| | malignancy |
| CISH, IL-15 | Solid tumor |
| CISH, TGFBR2 | Solid tumor |
| c-Jun | Basal cell carcinoma |
| c-Jun | Melanoma |
| c-Kit | Myocardial ischemia |
| c-Kit | Systemic mastocytosis |
| c-Kit | allergy |
| CLCN7 | Autosomal dominant |
| | osteopetrosis type II |
| CLDN18.2 | Autoimmune disease |
| CLDN18.2 | Pancreatic cancer |
| CLDN18.2 | Gastric |
| | adenocarcinoma |
| CLDN18.2 | Gastroesophageal |
| | junction |
| | adenocarcinoma |
| CLDN18.2 | Gastrointestinal tumor |
| CLDN18.2 | Gastric cancer |
| CLDN18.2 | Advanced solid |
| | malignant tumor |
| CLDN18.2 | Solid tumor |
| CLDN18.2 | Ovarian epithelial |
| | carcinoma |
| CLDN18.2 | Ovarian cancer |
| CLDN18.2 | Advanced gastric |
| | adenocarcinoma |
| CLDN18.2 | CLDN18.2-positive |
| | pancreatic cancer |
| CLDN18.2 | CLDN18.2-positive |
| | gastroesophageal |
| | junction |
| | adenocarcinoma |
| CLDN18.2 | CLDN18.2-positive |
| | gastrointestinal tumor |
| CLDN18.2, CXCR4 | Pancreatic ductal |
| | adenocarcinoma |
| CLDN18.2, GluN2B, | Pancreatic cancer |
| NAMPT, NKG2D, | |
| NMDA receptor | |
| CLDN18.2, GluN2B, | Gastric cancer |
| NAMPT, NKG2D, | |
| NMDA receptor | |
| CLDN18.2, GluN2B, | NKG2DL-positive solid |
| NAMPT, NKG2D, | tumor |
| NMDA receptor | |
| CLDN18.2, GluN2B, | CLDN18.2-positive |
| NAMPT, NKG2D, | solid tumor |
| NMDA receptor | |
| CLDN18.2, IL-15 | Pancreatic cancer |
| CLDN18.2, IL-15 | Melanoma |
| CLDN18.2, PD-1 | Gastric cancer |
| CLDN18.2, PDL1 | CLDN18.2-positive |
| | solid tumor |
| CLDN6 | Solid tumor |
| CLDN6 | Ovarian cancer |
| CLDN6 | Testicular tumor |
| CLEC4K | Langerhans cell |
| | histiocytosis |
| CLL-1 | Chronic lymphocytic |
| | leukemia |
| CLL-1 | Acute myeloid |
| | leukemia |
| CLL-1 | Relapsed acute |
| | myeloid leukemia |
| CLN3 | Neuronal ceroid |
| | lipofuscinosis |
| CLN5 | Neuronal ceroid |
| | lipofuscinosis 5 |
| CLN6 | Neuronal ceroid |
| | lipofuscinosis |
| CLN8 | Neuronal ceroid |
| | lipofuscinosis |
| CLRN1 | Usher syndrome |
| CLU | Metastatic castration- |
| | resistant prostate |
| | cancer |
| CLU | Metastatic non-small |
| | cell lung cancer |
| CLU | Pain |
| CLU | Castration-resistant |
| | prostate cancer |
| CLU | Non-small cell lung |
| | cancer |
| c-Met | Foot ulcer |
| c-Met | Perioperative ischemia |
| c-Met | Solid tumor |
| c-Met | Ulcer |
| c-Met | Cirrhosis |
| c-Met | Atherosclerosis |
| | obliterans |
| c-Met | Hyperlipoproteinemia |
| | type l |
| CMV IE2 | Cytomegalovirus |
| | retinitis |
| CMV IE2 | Cytomegalovirus |
| | infection |
| CMV IE2 | HIV infection |
| CMV pp65, LAMP-1 | Glioblastoma |
| CMV protein | Adenoviridae |
| | Infections |
| CMV protein | Organ transplant |
| | rejection |
| CMV protein | Cytomegalovirus |
| | infection |
| CMV protein | Polyomavirus infection |
| CMV protein | Epstein-Barr virus |
| | infection |
| CMV protein, EBV | Infect |
| protein | |
| c-Myc | Reperfusion injury |
| c-Myc | Primitive |
| | neuroectodermal |
| | tumor |
| c-Myc | Islet cell adenoma |
| c-Myc | Solid tumor |
| C-Myc | Lymphoma |
| c-Myc | Colorectal cancer |
| c-Myc | Huntington's disease |
| c-Myc | Coronary artery |
| | restenosis |
| c-Myc | Hepatocellular |
| | carcinoma |
| c-Myc | Non-small cell lung |
| | cancer |
| c-Myc | Non-Hodgkin |
| | lymphoma |
| c-Myc | Multiple myeloma |
| c-Myc | C-Myc positive solid |
| | tumor |
| CNGA3 | Color vision |
| | impairment |
| CNGA3 | Achromatopsia type 1 |
| CNGB1 | Retinitis pigmentosa |
| CNGB3 | Color vision |
| | impairment |
| CNGB3 | Color vision |
| | impairment |
| CNGB3 | Achromatopsia type 1 |
| CNTF | Retinitis pigmentosa |
| CNTFR | Autism Spectrum |
| | Disorders |
| CNTFR | Autism |
| CNTFR | Peripheral nerve |
| | damage |
| CNTFR | Retinitis pigmentosa |
| CNTFR | Retinal telangiectasia |
| CNTFR | Optic nerve disease |
| CNTFR | Color vision |
| | impairment |
| CNTFR | Glaucoma |
| CNTFR | Parkinson's disease |
| CNTFR | Acute lung injury |
| CNTFR | Amyotrophic lateral |
| | sclerosis |
| CNTFR | Huntington's disease |
| CNTFR | Multiple sclerosis |
| CNTFR | Geographic atrophy |
| CNTFR | Usher syndrome |
| Coagulation factor | Coagulation disorder |
| COL1A1 | Skin aging |
| COL3A1 | Ehlers-Danlos |
| | syndrome |
| COL3A1, elastin | Skin disorders |
| COL5A1 | Ehlers-Danlos |
| | syndrome |
| COL6A3 | Solid tumor |
| COL7A1 | Dystrophic |
| | epidermolysis bullosa |
| COL7A1 | Graft-versus-host |
| | disease |
| COL7A1 | Burn |
| COL7A1 exon 80 | Epidermolysis bullosa |
| Collagen | Dystrophic |
| | epidermolysis bullosa |
| Collagen | Penile edema |
| Collagen | Lower limb ulcers |
| Collagen | Knee Injury |
| Collagen | Tennis elbow |
| Collagen | Cartilage disease |
| Collagen | Tendon injuries |
| Collagen | Tendinopathy |
| Collagen | Osteoarthritis |
| Collagen | Epidermolysis bullosa |
| COP1 | Hepatocellular |
| | carcinoma |
| COPS5, WEE1 | Hepatitis B |
| COX-2 | Prostate cancer |
| COX-2 | Basal cell carcinoma |
| COX-2, PGE2 | Colon cancer |
| COX-2, PGE2 | Liver cancer |
| COX-2, TGF-β1 | Carcinoma in situ |
| COX-2, TGF-β1 | Primary sclerosing |
| | cholangitis |
| COX-2, TGF-β1 | Solid tumor |
| COX-2, TGF-β1 | Breast cancer |
| COX-2, TGF-β1 | Prostate cancer |
| COX-2, TGF-β1 | Hypertrophic scars of |
| | the skin |
| COX-2, TGF-β1 | Squamous cell |
| | carcinoma of the skin |
| COX-2, TGF-β1 | Local obesity |
| COX-2, TGF-β1 | Basal cell carcinoma |
| COX-2, TGF-β1 | Liver metastasis |
| COX-2, TGF-β1 | Cirrhosis |
| COX-2, TGF-β1 | Hepatocellular |
| | carcinoma |
| COX-2, TGF-β1 | Obesity |
| COX-2, TGF-β1 | Cholangiocarcinoma |
| COX-2, TGF-β1 | Keloid |
| CRAF | Tumor |
| CRAF | Pancreatic cancer |
| CRAF | Small cell lung cancer |
| CRAF | Diabetic macular |
| | edema |
| CRAF | Solid tumor |
| CRAF | Renal cell carcinoma |
| CRAF | Breast cancer |
| CRAF | Prostate cancer |
| CRAF | Ovarian cancer |
| CRAF | Colorectal cancer |
| CRAF | Macular degeneration |
| CRAF | Non-small cell lung |
| | cancer |
| CRP | Inflammation |
| CRP | Atrial fibrillation |
| CRP | Kidney disease |
| CRP | Rheumatoid arthritis |
| CRP | Crohn's disease |
| CRX | Cone-rod dystrophy |
| CRX | Retinitis pigmentosa |
| CRX | Retinal disease |
| CRX | Leber congenital |
| | amaurosis |
| CSF-2R | Metastatic solid tumor |
| CSF-2R | Metastatic breast |
| | cancer |
| CSF-2R | Metastatic biliary tract |
| | cancer |
| CSF-2R | Tumor |
| CSF-2R | Central nervous |
| | system tumor |
| CSF-2R | Mucinous |
| | adenocarcinoma |
| CSF-2R | Ewing sarcoma |
| CSF-2R | Signet ring cell |
| | carcinoma |
| CSF-2R | Secondary malignant |
| | tumor of the pancreas |
| CSF-2R | Mycosis fungoides |
| CSF-2R | Eccrine sweat gland |
| | pore carcinoma |
| CSF-2R | Gastric cancer |
| CSF-2R | Microcystic adnexal |
| | carcinoma |
| CSF-2R | Advanced colorectal |
| | adenocarcinoma |
| CSF-2R | Vulvar squamous cell |
| | carcinoma |
| CSF-2R | Head and neck tumor |
| CSF-2R | Retinoblastoma |
| CSF-2R | Esophageal cancer |
| CSF-2R | Solid tumor |
| CSF-2R | Wilms tumor |
| CSF-2R | Neuroblastoma |
| CSF-2R | Sezary syndrome |
| CSF-2R | Extramammary |
| | Paget's disease |
| CSF-2R | Ductal carcinoma |
| CSF-2R | Papillary |
| | adenocarcinoma |
| CSF-2R | Prostate cancer |
| CSF-2R | Sebaceous adenoma |
| CSF-2R | Skin tumor |
| CSF-2R | Squamous cell |
| | carcinoma of the skin |
| CSF-2R | Skin melanoma |
| CSF-2R | Skin adnexal cancer |
| CSF-2R | Brain malignant glioma |
| CSF-2R | Refractory lymphoma |
| CSF-2R | Refractory anaplastic |
| | large cell lymphoma |
| CSF-2R | Refractory T-cell |
| | lymphoma |
| CSF-2R | Merkel cell carcinoma |
| CSF-2R | Helical |
| | adenocarcinoma |
| CSF-2R | Lymphoma |
| CSF-2R | Locally advanced soft |
| | tissue sarcoma |
| CSF-2R | Locally advanced |
| | bladder cancer |
| CSF-2R | Invasive breast cancer |
| CSF-2R | Colorectal cancer liver |
| | metastasis |
| CSF-2R | Colorectal cancer |
| CSF-2R | Keratoacanthoma |
| CSF-2R | Glioblastoma |
| CSF-2R | Mesothelioma |
| CSF-2R | Basal cell carcinoma |
| CSF-2R | Basal squamous cell |
| | carcinoma |
| CSF-2R | Muscle-Invasive |
| | Bladder Cancer |
| CSF-2R | Rhabdomyosarcoma |
| CSF-2R | Melanoma |
| CSF-2R | Sweat gland tumor |
| CSF-2R | Hepatocellular |
| | carcinoma |
| CSF-2R | Liver cancer |
| CSF-2R | Recurrent breast |
| | cancer |
| CSF-2R | Relapsed T-cell |
| | lymphoma |
| CSF-2R | Recurrent HER2- |
| | negative breast cancer |
| CSF-2R | Relapsed peripheral T- |
| | cell lymphoma |
| CSF-2R | Non-muscle invasive |
| | bladder tumor |
| CSF-2R | Malignant cylindrical |
| | tumor |
| CSF-2R | Malignant solid tumor |
| CSF-2R | Ductal carcinoma |
| CSF-2R | Unresectable |
| | melanoma |
| CSF-2R | Bladder metastases |
| CSF-2R | Bladder cancer |
| CSF-2R | NK cell lymphoma |
| CSF-2R, CTLA4 | Tumor |
| CSF-2R, CTLA4, | Microsatellite stable |
| GALV-GP R- | colorectal cancer |
| CSF-2R, CTLA4, | Solid tumor |
| GALV-GP R- | |
| CSF-2R, GALV-GP | Solid tumor |
| R- | |
| CSF-2R, GALV-GP | Triple-negative breast |
| R- | cancer |
| CSF-2R, GALV-GP | Squamous cell |
| R- | carcinoma of the skin |
| CSF-2R, GALV-GP | Skin melanoma |
| R- | |
| CSF-2R, GALV-GP | Melanoma |
| R- | |
| CSF-2R, GALV-GP | Non-small cell lung |
| R- | cancer |
| CSF-2R, GALV-GP | MSI-H cancer |
| R- | |
| CSF-2R, PDL1 | Solid tumor |
| CSF-3R | Neutropenia |
| CSF-3R | Cytokine Release |
| | Syndrome |
| CSF-3R | Knee arthritis |
| CSF-3R | Advanced solid |
| | malignant tumor |
| CSF-3R | Community-acquired |
| | pneumonia |
| CSF-3R | Sepsis |
| CSF-3R | Urinary tract infection |
| CSF-3R | Mesothelioma |
| CSF-3R | Acute cholecystitis |
| CSF-3R | Abdominal infection |
| CSF-3R | Secondary malignant |
| | tumor of peritoneum |
| CSF-3R | Peritoneal cancer |
| CSF-3R | cholangitis |
| CTCFL | Breast cancer |
| CTGF | Head and neck tumor |
| CTGF | Idiopathic pulmonary |
| | fibrosis |
| CTGF | Hypertrophic scars of |
| | the skin |
| CTGF | Ovarian cancer |
| CTGF | Melanoma |
| CTGF | Cirrhosis |
| CTGF | Trauma and Injury |
| CTGF | Scar |
| CTGF, NF-κB | Duchenne muscular |
| | dystrophy |
| CTLA4 | Tumor |
| CTLA4 | Sjogren's syndrome |
| CTLA4, FLT3, IL-12R | Solid tumor |
| CTLA4, IL-12 | Metastatic non-small |
| | cell lung cancer |
| CTLA4, MSLN, PD-1 | Solid tumor |
| CTLA4, PD-1 | Advanced solid |
| | malignant tumor |
| CTLA4, PD-1 | Colon cancer |
| CTLA4, STAT3 | Tumor |
| CTNNB1 | Tumor |
| CTNNB1 | Type 2 diabetes |
| CTNS | Cystinosis |
| CXCR2, GPC3 | Liver cancer |
| CXCR3, IL-12, TGF-β | Tumor |
| CXCR4 | Primary myelofibrosis |
| CXCR4 | Heart failure |
| CXCR4 | Perioperative ischemia |
| CXCR4 | Peripheral arterial |
| | disease |
| CXCR4 | Surgical wound |
| CXCR4 | Myelofibrosis |
| CXCR5, EGFR | Non-small cell lung |
| | cancer |
| CYB5R3 | Tumor |
| CYBB | Chronic |
| | granulomatous |
| | disease |
| Cyclin G1 | Pancreatic cancer |
| Cyclin G1 | Solid tumor |
| Cyclin G1 | Breast cancer |
| Cyclin G1 | Sarcoma |
| Cyclin G1 | Colorectal cancer |
| Cyclin G1 | Osteosarcoma |
| CYP21A2 | Congenital adrenal |
| | hyperplasia |
| CYP27A1 | Cerebrotendinous |
| | xanthomatosis |
| CYP3A4 | Tumor |
| CYP4V2 | Retinal degeneration |
| CYP4V2 | Bietti crystal dystrophy |
| CYPs | Pancreatic cancer |
| CYPs | Colorectal cancer |
| Cytokines | Sarcoma |
| Cytokines | Melanoma |
| Cytokines | Malignant solid tumor |
| C-type lectin | Testicular tumor |
| domain protein | |
| DAF | Macular degeneration |
| DAF, ICAM-1 | Solid tumor |
| DAF, ICAM-1 | Non-small cell lung |
| | cancer |
| DAG1 | Muscular dystrophy |
| DAG1 | Duchenne muscular |
| | dystrophy |
| DAT | Parkinson's disease |
| DDC | Parkinson's disease |
| DDC | Aromatic amino acid |
| | decarboxylase |
| | deficiency |
| DGAT2 | Nonalcoholic |
| | steatohepatitis |
| DGAT2 | Type 2 diabetes |
| DGKK | Fragile X syndrome |
| DKK3 | Mesothelioma |
| DKK3 | Malignant pleural |
| | mesothelioma |
| DLL3 | Recurrence of small |
| | cell lung cancer |
| DLL3 | Small cell lung cancer |
| DLL3 | Large cell |
| | neuroendocrine |
| | carcinoma |
| DMD exon 055 | Muscular dystrophy |
| DMD exon 44 | Muscular dystrophy |
| DMD exon 44 | Duchenne muscular |
| | dystrophy |
| DMD exon 45 | Duchenne muscular |
| | dystrophy |
| DMD exon 45, DMD | Duchenne muscular |
| exon 44 | dystrophy |
| DMD exon 50 | Duchenne muscular |
| | dystrophy |
| DMD exon 51 | Duchenne muscular |
| | dystrophy |
| DMD exon 52 | Muscular dystrophy |
| DMD exon 52 | Duchenne muscular |
| | dystrophy |
| DMD exon 53 | Duchenne muscular |
| | dystrophy |
| DMD exon 53 | Duchenne muscular |
| | dystrophy |
| DMD exon 55 | Duchenne muscular |
| | dystrophy |
| Dm-dNK | Breast cancer |
| Dm-dNK | Colorectal cancer |
| DMPK | Myotonic dystrophy |
| DNA | Pancreatic cancer |
| DNA polymerase, | Cytomegalovirus |
| RT | infection |
| DNA polymerase, RT | Herpes simplex |
| DNA polymerase, | Epstein-Barr virus |
| RT | infection |
| DNA polymerase, | HIV infection |
| RT | |
| DNA polymerase, | Tumor |
| thymidine kinase | |
| DNA-directed DNA | Autoimmune disease |
| polymerase | |
| DNA-directed DNA | Tumor |
| polymerase | |
| DNA-directed DNA | Inflammation |
| polymerase | |
| DNA-directed DNA | Influenza A virus |
| polymerase | infection |
| DNA-directed DNA | Dystonia |
| polymerase | |
| DNAJC15 | Acute kidney injury |
| DNAJC15 | Polycystic kidney |
| | disease |
| DNase l | Pancreatic acinar |
| | carcinoma |
| DNase l | Colorectal cancer |
| DNA repair proteins | Advanced cancer |
| DNA repair proteins | Melanoma |
| DNA-directed RNA | Bacterial infection |
| polymerase | |
| DNM2 | Muscle atrophy |
| DNMT1 | Head and neck tumor |
| DNMT1 | Kidney tumor |
| DNMT1 | Renal cell carcinoma |
| DNMT1 | Acute myeloid |
| | leukemia |
| DNMT1 | Myelodysplastic |
| | syndrome |
| DOK7 | Motor neurone |
| | disease |
| DOK7 | Congenital myasthenic |
| | syndrome |
| DR4, TRAIL | Kidney tumor |
| DR5 | Liver cancer |
| DRDs | Parkinson's disease |
| DSG1, DSG3 | Pemphigus |
| DSG3 | Pemphigus |
| DSG3 | Familial pemphigus |
| | vulgaris |
| DUSP1 | Heart failure |
| DUSP1 | Arrhythmias |
| DUSP1 | Heart failure NYHA |
| | class III |
| DUSP1 | Myocardial ischemia |
| DUSP1 | Diastolic heart failure |
| DUSP1 | Systolic heart failure |
| DUSP1 | Complication |
| DUX4 | Facioscapulohumeral |
| | muscular dystrophy |
| DUX4 | Facioscapulohumeral |
| | muscular dystrophy |
| | type 1a |
| DWORF | Heart failure with |
| | reduced ejection |
| | fraction |
| DWORF | Dilated |
| | cardiomyopathy |
| DYSF | Limb-girdle muscular |
| | dystrophy |
| DYSF | Miyoshi myopathy |
| DYSF | Myofibrillar myopathy |
| DYSF | Dysferlin myopathy |
| DYSF | Limb-Girdle Muscular |
| | Dystrophy Type 2B |
| Dystrophin | Angelman syndrome |
| Dystrophin | Muscular dystrophy |
| Dystrophin | Duchenne muscular |
| | dystrophy |
| E2F1 | End-stage renal |
| | disease |
| E2F1 | Hyperplasia |
| E2F1 | Vascular graft |
| | obstruction |
| E2F1 | Perioperative ischemia |
| E2F1 | Peripheral vascular |
| | disease |
| E2F1 | Coronary artery |
| | restenosis |
| E2F1 | Coronary heart |
| | disease |
| E2F1 | Arterial occlusive |
| | disease |
| E6 | Penile tumor |
| E6 | Vaginal tumor |
| E6 | Vulvar tumor |
| E6 | Papillomavirus |
| | infection |
| E6 | Squamous |
| | intraepithelial lesion |
| E6 | Oropharyngeal tumor |
| E6 | Cervical cancer |
| E6 | Cervical cancer |
| E6 | Anal tumor |
| E6 | Polyomavirus infection |
| E6 | HPV16 infection |
| E6, HPV E7 | Head and neck tumor |
| E6, HPV E7 | Cervical cancer |
| E6, PD-1 | Metastatic cervical |
| | cancer |
| E6, PD-1 | Cervical cancer |
| EBNA1, LMP1, | Hodgkin lymphoma |
| BARF1 proteins | |
| EBNA1, LMP1, | Non-Hodgkin |
| BARF1 proteins | lymphoma |
| EBV proteins | Hematopoietic stem |
| | cell transplantation |
| EBV proteins | Transplant |
| | complications |
| EBV proteins | Hematologic |
| | malignancy |
| EBV proteins | Gastric cancer |
| EBV proteins | Leiomyosarcoma |
| EBV proteins | Nasopharyngeal |
| | carcinoma |
| EBV proteins | Epstein-Barr virus- |
| | positive lymphoma |
| EBV proteins | Epstein-Barr virus- |
| | associated |
| | lymphoproliferative |
| | disease |
| EBV proteins | Epstein-Barr virus- |
| | associated |
| | nasopharyngeal |
| | carcinoma |
| EBV proteins | Ebola virus disease |
| EBV proteins | EBV-related post- |
| | transplant |
| | lymphoproliferative |
| | disorder |
| EGF | HER2-positive breast |
| | cancer |
| EGFR | Metastatic head and |
| | neck squamous cell |
| | carcinoma |
| EGFR | Ewing sarcoma |
| EGFR | Advanced solid |
| | malignant tumor |
| EGFR | Clear cell sarcoma |
| EGFR | Head and neck tumor |
| EGFR | Retinoblastoma |
| EGFR | Solid tumor |
| EGFR | Wilms tumor |
| EGFR | Neurofibrosarcoma |
| EGFR | Neuroblastoma |
| EGFR | Triple-negative breast |
| | cancer |
| EGFR | Squamous cell |
| | carcinoma |
| EGFR | Glioblastoma |
| EGFR | Synovial sarcoma |
| EGFR | Rhabdoid tumor |
| EGFR | Rhabdomyosarcoma |
| EGFR | Hepatocellular |
| | carcinoma |
| EGFR | Hepatoblastoma |
| EGFR | Recurrent solid tumor |
| EGFR | Non-small cell lung |
| | cancer |
| EGFR | Malignant epithelial |
| | tumor |
| EGFR | Desmoplastic small |
| | round cell tumor |
| EGFR | Trauma and Injury |
| EGFR | HER2-positive breast |
| | cancer |
| EGFR | EGFR mutant solid |
| | tumor |
| EGFR | EGFR mutant solid |
| | tumor |
| EGFR, c-Met | Nonalcoholic |
| | steatohepatitis |
| EGFR, EGFRVIII | Glioblastoma |
| EGFR, EGFRVIII, | Recurrent |
| HER2, IL-13Rα2 | glioblastoma |
| EGFR, IL-12 | Colorectal cancer |
| EGFR, IL-13Rα2 | Glioblastoma |
| EGFR, IL-13Rα2 | Recurrent |
| | glioblastoma |
| EGFR, IL-13Rα2 | EGFR-mutated |
| | glioblastoma |
| EGFR, IL-21R | Leukemia |
| EGFR, SSTR | Pancreatic cancer |
| EGFR, SSTR | Head and Neck |
| | Squamous Cell |
| | Carcinoma |
| EGFR, SSTR | Breast cancer |
| EGFR, SSTR | Colorectal cancer |
| EGFR, SSTR | Melanoma |
| EGFR, SSTR | Liver cancer |
| EGFR, TCRDV2 | Pancreatic ductal |
| | adenocarcinoma |
| EGFR, TCRDV2 | Head and Neck |
| | Squamous Cell |
| | Carcinoma |
| EGFR, TCRDV2 | Solid tumor |
| EGFR, TCRDV2 | Colorectal cancer |
| EGFR, TCRDV2 | Non-small cell lung |
| | cancer |
| EGFR, TGF-β | Tumor |
| EGFRvIII | Meningeal tumor |
| EGFRvIII | Glioblastoma |
| EGFRvIII | Liver cancer |
| EGFRvIII | Lung cancer |
| EGFRvIII | Glioblastoma |
| | multiforme |
| EGFRvIII, EphA2, | Glioblastoma |
| HER2, IL-13Rα2 | |
| EGR1 | Tumor |
| EGR1 | Postoperative pain |
| EHMT2 | Prader-Willi syndrome |
| EIF2AK4 | Tumor |
| EIF2AK4 | Immune system |
| | disorders |
| EIF4E | Tumor |
| EIF4E | Advanced cancer |
| EIF4E | Castration-resistant |
| | prostate cancer |
| EIF4E | Prostate cancer |
| EIF4E | Non-small cell lung |
| | cancer |
| ELA2 | Tumor |
| ELA2 | Duchenne muscular |
| | dystrophy |
| elastin | Skin disorders |
| ELAVL1 | Pancreatic cancer |
| ELAVL1 | Ovarian cancer |
| ELF3 | Tuberculosis |
| ELOVL2 | Dry age-related |
| | macular degeneration |
| ELP1, splicing | Familial dysautonomia |
| factor | |
| EMR1 | Leukemia |
| ENaC | Cystic fibrosis |
| enkephalinase | B-cell leukemia |
| eNOS | Myocardial infarction |
| eNOS | Peripheral vascular |
| | disease |
| eNOS | Pulmonary |
| | hypertension |
| env | Chronic hepatitis B |
| EpCAM | Digestive system |
| | tumor |
| EpCAM | Gastric cancer |
| EpCAM | Advanced Gastric |
| | Cancer |
| EpCAM | Solid tumor |
| EpCAM | Liver cancer |
| EphA2 | Pancreatic cancer |
| EphA2 | Solid tumor |
| EphA2 | Ovarian cancer |
| EphA2 | Lymphoma |
| EphA2 | Locally advanced |
| | malignant solid tumor |
| EphA2 | Glioblastoma |
| EphA2 | Glioma |
| EphA2 | Lung cancer |
| EphA2, IL-13Rα2 | EGFR-mutated |
| | glioblastoma |
| EphA3 | Solid tumor |
| EPO receptor | End-stage renal |
| | disease |
| EPO receptor | Renal anemia |
| EPO receptor | Neurodegenerative |
| | disease |
| EPO receptor | Glaucoma |
| EPO receptor | Anemia |
| EPO receptor | Parkinson's disease |
| ERVE-1 | Kidney tumor |
| ERa | Tumor |
| E-sel | Thrombotic occlusive |
| | vasculitis |
| E-sel | Vascular disease |
| E-sel | Peripheral arterial |
| | disease |
| EWS-FLI1 | Ewing sarcoma |
| EZH2 | Solid tumor |
| F10 | Hemophilia A |
| F10, F8 | Hemophilia A |
| F11 | End-stage renal |
| | disease |
| F11 | Thromboembolism |
| F11 | Cardiovascular |
| | disease |
| F11 | Myocardial infarction |
| F11 | Upper extremity deep |
| | vein thrombosis |
| F11 | Coagulation disorders |
| F11 | Stroke |
| F11 | Venous thrombosis |
| F11 | Arterial thrombosis |
| F11R | Androgenic Alopecia |
| F12 | Thromboembolism |
| F12 | Cardiovascular |
| | disease |
| F12 | Metabolic disease |
| F8 | Hemophilia A |
| F8 | Antineutrophil |
| | cytoplasmic antibody- |
| | associated vasculitis |
| F8 | X-linked genetic |
| | disease |
| F8, factor IX | Hemophilia B |
| F8, factor IX | Hemophilia A |
| factor IX | Hemophilia B |
| factor IX | Venous thrombosis |
| factor IX | Acute Coronary |
| | Syndrome |
| factor IX | Acute Coronary |
| | Syndrome |
| factor IX | Coronary heart |
| | disease |
| factor IX | Nonalcoholic |
| | steatohepatitis |
| Factor VII | Thrombosis |
| Factor VII | Factor VII deficiency |
| FANCA | Tyrosinemia |
| FANCA | Fanconi anemia |
| FANCC | Fanconi anemia |
| FAP | Tumor |
| FAP, PDL1 | Solid tumor |
| FasL | Sarcoma |
| FasL | Lymphoma |
| FasL | Malignant epithelial |
| | tumor |
| FASN, MSLN | Fallopian tube cancer |
| FASN, MSLN | Ovarian cancer |
| FASN, MSLN | Peritoneal cancer |
| FasR | Hepatitis |
| FasR | Liver failure |
| FasR, TNFR1 | Metastatic colorectal |
| | cancer |
| FasR, TNFR1 | Colorectal cancer liver |
| | metastasis |
| FasR, TNFR1 | Recurrent platinum- |
| | resistant ovarian |
| | cancer |
| FasR, TNFR1 | Glioblastoma |
| | multiforme |
| FGF19 | Nonalcoholic |
| | steatohepatitis |
| FGF21 | Heart failure |
| FGF21 | Myocardial disease |
| FGF21 | Familial partial lipid |
| | dystrophy |
| FGF21 | obesity |
| FGF21 | Metabolic fatty liver |
| | disease |
| FGF21, FGF21R | Nonalcoholic |
| | steatohepatitis |
| FGF21, TGF-β1 | Heart disease |
| FGF21, TGF-β1 | Senescence |
| FGF21, TGF-β1 | Kidney disease |
| FGF21, TGF-β1 | Metabolic disease |
| FGF4 | Stable angina |
| FGF4 | Refractory angina |
| FGF4 | Coronary heart |
| | disease |
| FGFR3 | Chondrodysplasia |
| FGFR4 | Rhabdomyosarcoma |
| FGFR4 | Hepatocellular |
| | carcinoma |
| FGFR4 | Obesity |
| FGFRs | Hair loss |
| FGFRs | Multiple sclerosis |
| FGL1 | Tumor |
| FKRP | Limb-girdle muscular |
| | dystrophy |
| FKRP | Miyoshi myopathy |
| FLAP | Allergic conjunctivitis |
| FLT3 | Refractory acute |
| | myeloid leukemia |
| FLT3 | Acute myeloid |
| | leukemia |
| FLT3 | Relapsed acute |
| | myeloid leukemia |
| FLT3 | Leukemia |
| FLT3 | FLT3-positive acute |
| | myeloid leukemia |
| FMRP | Fragile X syndrome |
| FOLR1 | Mucinous |
| | cystadenocarcinoma |
| FOLR1 | Primary peritoneal |
| | serous |
| | adenocarcinoma |
| FOLR1 | Undifferentiated |
| | ovarian cancer |
| FOLR1 | Advanced solid tumor |
| FOLR1 | Fallopian tube clear |
| | cell adenocarcinoma |
| FOLR1 | Fallopian tube cancer |
| FOLR1 | Renal cell carcinoma |
| FOLR1 | Urothelial carcinoma |
| FOLR1 | Urothelial carcinoma |
| FOLR1 | Myxoma |
| FOLR1 | Ovarian endometrioid |
| | carcinoma |
| FOLR1 | Ovarian epithelial |
| | carcinoma |
| FOLR1 | Ovarian epithelial |
| | carcinoma |
| FOLR1 | Ovarian mixed |
| | epithelial carcinoma |
| FOLR1 | Ovarian cancer |
| FOLR1 | Serous |
| | cystadenocarcinoma |
| FOLR1 | Osteosarcoma |
| FOLR1 | Peritoneal cancer |
| FOLR1 | Peritoneal cancer |
| FOLR1 | Recurrent primary |
| | peritoneal cancer |
| FOLR1 | Recurrent ovarian |
| | cancer |
| FOLR1 | Non-small cell lung |
| | cancer |
| FOXG1 | Neurological disorders |
| FOXO1 | Solid tumor |
| FSHR | Ovarian cancer |
| FtsZ | Acinetobacter |
| | baumannil infection |
| FUS | Amyotrophic lateral |
| | sclerosis |
| FUS | Amyotrophic lateral |
| | sclerosis |
| FXN | Friedreich's ataxia |
| G6PC1 | Glycogen storage |
| | disease |
| G6PC1 | Glycogen storage |
| | disease type l |
| GABA receptor | Temporal lobe |
| | epilepsy |
| GABA receptor | Drug-resistant |
| | epilepsy |
| GABA receptor | epilepsy |
| GAD | Parkinson's disease |
| GAD2, GDNF, IL-10 | Sciatica |
| GAD2, GDNF, IL-10 | Lumbar radiculopathy |
| GAD2, GDNF, IL-10 | Neuralgia |
| GAG | Mucopolysaccharidosis |
| | type II |
| GAG | HIV infection |
| GALC | Globoid cell |
| | leukodystrophy |
| galectin-3 | Osteosarcoma |
| GALGT2 | Duchenne muscular |
| | dystrophy |
| GALT | Galactosemia |
| GATA3 | asthma |
| GATA3 | Chronic obstructive |
| | pulmonary disease |
| GATA3 | Ulcerative colitis |
| GBA | Parkinson's disease |
| GBA | Gaucher disease |
| GBA | Fabry disease |
| GBA | Gaucher disease type |
| GBA, | Gaucher disease |
| Phosphotransferases | |
| GC-C | Pancreatic cancer |
| GC-C | Colorectal cancer |
| GCDH | Glutaric acidemia |
| GCGR | Type 2 diabetes |
| GCGR | Type 2 diabetes |
| GD2 | Phyllodes |
| GD2 | Small cell lung cancer |
| GD2 | Ependymoma |
| GD2 | Neuroblastoma |
| GD2 | sarcoma |
| GD2 | Uveal melanoma |
| GD2 | Brain cancer |
| GD2 | Refractory |
| | neuroblastoma |
| GD2 | Diffuse intrinsic |
| | pontine glioma |
| GD2 | Glioma |
| GD2 | Rhabdomyosarcoma |
| GD2 | Melanoma |
| GD2 | Osteosarcoma |
| GD2 | High-grade glioma |
| GD2 | Recurrent Ewing |
| | sarcoma |
| GD2 | Recurrent |
| | neuroblastoma |
| GD2 | Recurrent |
| | osteosarcoma |
| GD2 | Glioblastoma |
| | multiforme |
| GD2 | Gaucher disease type II |
| GD2, IL15R | Small cell lung cancer |
| GD2, IL15R | Non-small cell lung |
| | cancer |
| GDNF | Parkinson's disease |
| GDNF | Amyotrophic lateral |
| | sclerosis |
| GFAP | Alexander disease |
| GFRA4 | Medullary thyroid |
| | cancer |
| GHR | Acromegaly |
| GHR | Acromegaly |
| GHRH | Anemia |
| GHRH | Cachexia |
| GITRL | Glioma |
| GJA1 | Arrhythmias |
| GJA1 | Myocardial disease |
| GJA1 | diabetic foot |
| GJA1 | Corneal damage |
| GJB2 | Hearing loss |
| GLA | Fabry disease |
| GLA | Glycogen storage |
| | disease type II |
| GLA | Chagas |
| | cardiomyopathy |
| GlcCer | Neurodegenerative |
| | disease |
| GlcCer | Gaucher disease |
| GlcCer | Gaucher disease type I |
| GLI2 | Tumor |
| GLI2 | Prostate cancer |
| GLIPR1 | Prostate cancer |
| Globo H | Gastric cancer |
| GLP-1R | Weight loss |
| GLP-1R | Obesity |
| GLP-1R | Type 2 diabetes |
| glucokinase | Type 2 diabetes |
| Glucokinase, insulin | Diabetes |
| GluK2, Kainic acid | Temporal lobe |
| receptor | epilepsy |
| GluK2, Kainic acid | Epilepsy |
| receptor | |
| Glycogen | Glycogen storage |
| | disease type II |
| GM2 (ganglioside M2) | Solid tumor |
| GM-CSF | Bladder cancer |
| GM-CSF, decorin | Colorectal cancer |
| GM-CSF, IFNα2, IL- | Advanced solid tumor |
| 12, IL-15 | |
| GM-CSF, IFNα2, IL- | Solid tumor |
| 12, IL-15 | |
| GM-CSF, IFNα2, IL- | Melanoma |
| 12, IL-15 | |
| GM-CSF, RYBP | Tumor |
| GNE | Distal myopathy |
| GNE | Hereditary inclusion |
| | body myopathy |
| GOPC | Tumor |
| GOX | Primary hyperoxaluria |
| | type 1 |
| GOX | Primary hyperoxaluria |
| GOX | Kidney Stone Disease |
| gp100 | Melanoma |
| GPC1 | Pancreatic cancer |
| GPC2 | Small cell lung cancer |
| GPC2 | Neuroblastoma |
| GPC2 | Refractory |
| | neuroblastoma |
| GPC2 | High-risk |
| | neuroblastoma |
| GPC2 | Recurrent |
| | neuroblastoma |
| GPC2 | Bladder cancer |
| GPC3 | Ovarian cancer |
| GPC3 | Squamous cell lung |
| | cancer |
| GPC3 | Hepatocellular |
| | carcinoma |
| GPC3 | Lung cancer |
| GPC3 | Cholangiocarcinoma |
| GPC3, IL-15 | Solid tumor |
| GPC3, IL-15 | Hepatocellular |
| | carcinoma |
| GPC3, IL15R | Liver cancer |
| GPC3, IL-2Rβ | Solid tumor |
| GPC3, NKp46 | Hepatocellular |
| | carcinoma |
| GPCR | Retinitis pigmentosa |
| GPCR, β- | Novel Coronavirus |
| adrenoceptors | Infection |
| GPCR, β- | Heart failure |
| adrenoceptors | |
| GPCR, β- | Chronic fatigue |
| adrenoceptors | syndrome |
| GPCR, β- | Dilated |
| adrenoceptors | cardiomyopathy |
| GPRC5D | Refractory plasma cell |
| | myeloma |
| GPRC5D | Plasma cell leukemia |
| GPRC5D | Plasma cell leukemia |
| GPRC5D | Relapsed Multiple |
| | myeloma |
| GPRC5D | Multiple myeloma |
| GPVI | Vascular disease |
| GR | Cushing syndrome |
| GR | Metabolic disease |
| GR | Type 2 diabetes |
| GRB2 | Endometrial cancer |
| GRB2 | Fallopian tube cancer |
| GRB2 | Ovarian epithelial |
| | carcinoma |
| GRB2 | Peritoneal cancer |
| GRB2 | Recurrent solid tumor |
| GSTP1 | Pancreatic cancer |
| GSTP1 | Colorectal cancer |
| GSTP1 | Non-small cell lung |
| | cancer |
| GSTP1 | KRAS-mutated non- |
| | small cell lung cancer |
| GTPCH1 | Immune system |
| | disorders |
| GUCY2D | Leber congenital |
| | amaurosis type 1 |
| GUCY2D | Leber congenital |
| | amaurosis |
| GYS1 | Glycogen storage |
| | disease type III |
| GYS1 | Lafora disease |
| GYS1 | Glycogen storage |
| | disease type II |
| GYS1 | Glycogen storage |
| | disease type II |
| H1 receptor, Opioid receptors | Upper respiratory tract |
| | allergies |
| H1 receptor, Opioid receptors | Common cold |
| H1 receptor, Opioid receptors | Cough |
| HAAH | Hematologic |
| | malignancy |
| HAAH | Solid tumor |
| HAAH | Glioblastoma |
| harmonin | Usher syndrome |
| HBA1 | Beta thalassemia |
| HBG1 | Sickle cell disease |
| HBG1 | Beta thalassemia |
| HBG1, HBG2 | Sickle cell disease |
| HBG1, HBG2 | Beta thalassemia |
| HBG2 | Sickle cell disease |
| HBG2 | Beta thalassemia |
| HBsAg | Hepatitis B virus- |
| | related hepatocellular |
| | carcinoma |
| HBsAg | Hepatitis B |
| HBsAg | diabetic foot |
| HBsAg | Hepatocellular |
| | carcinoma |
| HBsAg | Hepatitis D |
| HBV pol | Hepatitis B |
| HBV RNA | Hepatitis B |
| HBV-X | Hepatitis B |
| HBV-X | Chronic hepatitis B |
| HBV-X | Hepatitis D |
| HBX | Hepatitis B |
| HCCS1 | Hepatocellular |
| | carcinoma |
| HDAC6 | Tumor |
| HDGFL3 | Tumor |
| hemagglutinin | Niemann-Pick disease |
| | type C2 |
| Hemoglobin HbA | Alpha thalassemia |
| Hemoglobins | Transfusion- |
| | dependent beta- |
| | thalassemia |
| Hemoglobins | Sickle cell disease |
| Hemoglobins | Thalassemia |
| Hemoglobins, | Sickle cell disease |
| KLKB1, TTR | |
| Hemoglobins, | Beta thalassemia |
| KLKB1, TTR | |
| Hepc | Tumor |
| Hepc | End-stage renal |
| | disease |
| Hepc | Inflammation |
| Hepc | Anemia |
| Hepc | endotoxemia |
| Hepc | Chronic |
| Hepc | Anemia with chronic |
| | kidney disease |
| Hepc, IL-10, TGF-β | Kidney disease |
| HER2 | Endometrial cancer |
| HER2 | Endometrial cancer |
| HER2 | Tumor metastasis |
| HER2 | Tumor |
| HER2 | Tumor |
| HER2 | Pancreatic cancer |
| HER2 | Inflammatory breast |
| | tumor |
| HER2 | Small cell lung cancer |
| HER2 | Gastroesophageal |
| | junction cancer |
| HER2 | Gastric cancer |
| HER2 | Head and neck tumor |
| HER2 | Breast cancer |
| HER2 | Prostate cancer |
| HER2 | Urothelial carcinoma |
| HER2 | Brain metastases |
| HER2 | Ovarian epithelial |
| | carcinoma |
| HER2 | Ovarian cancer |
| HER2 | Squamous cell |
| | carcinoma |
| HER2 | Colorectal cancer |
| HER2 | Glioma |
| HER2 | Mesothelioma |
| HER2 | Chemotherapy- |
| | induced nausea and |
| | vomiting |
| HER2 | Osteosarcoma |
| HER2 | Cervical cancer |
| HER2 | Hepatocellular |
| | carcinoma |
| HER2 | Lung cancer |
| HER2 | Obesity |
| HER2 | Non-small cell lung |
| | cancer |
| HER2 | Ductal carcinoma |
| HER2 | Bile duct tumor |
| HER2 | Bladder cancer |
| HER2 | HER2-positive gastric |
| | cancer |
| HER2 | HER2-positive breast |
| | cancer |
| HER2 | Type 2 diabetes |
| HER2, HER3 | Triple-negative breast |
| | cancer |
| HER2, HER3 | Meningeal tumor |
| HER2, HER3 | HER2-positive breast |
| | cancer |
| HER2, HER3 | HER2-negative breast |
| | cancer |
| HER2, IL-13Rα2 | Ovarian cancer |
| HER2, IL15R | Gastric cancer |
| HER2, IL15R | Breast cancer |
| HER2, NKG2D | Solid tumor |
| HER2, PD-1 | Solid tumor |
| HER3 | Tumor |
| HER3 | Breast cancer |
| HER3 | Colon cancer |
| HER3 | Lung cancer |
| hERG | Atrial fibrillation |
| HEXA, HEXB | Tay-Sachs disease |
| HEXA, HEXB | Sandhoff disease |
| HEXA, HEXB | GM2 gangliosidosis |
| HGF | Autoimmune disease |
| HGF | Tumor |
| HGF | Myocardial ischemia |
| HGF | Lower limb ischemia |
| HGF | Peripheral arterial |
| | disease |
| HGF | Diabetic peripheral |
| | neuropathy |
| HGF | Diabetic vascular |
| | disease |
| HGF | Diabetic neuropathy |
| HGF | Diabetic nephropathy |
| HGF | Breast cancer |
| HGF | Ischemia |
| HGF | Chronic limb- |
| | threatening ischemia |
| HGF | Ulcer |
| HGF | Amyotrophic lateral |
| | sclerosis |
| HGF | Coronary heart |
| | disease |
| HGF | Type 1 diabetes |
| HIF-1α | Peripheral arterial |
| | disease |
| HIF-1α | Lymphoma |
| HIF-1α | Hepatocellular |
| | carcinoma |
| HIF-1α | Liver cancer |
| HIF-1α | Liver cancer |
| HIF-1α | Malignant epithelial |
| | tumor |
| HIF-1α, transcription | Tumor |
| factors, and related | |
| regulatory factors | |
| HIF-2α | Renal cell carcinoma |
| HIF-2α | Neuroblastoma |
| Histone H3 | Diffuse midline glioma, |
| | H3 K27M-mutant |
| HIV envelope | AIDS |
| protein gp 120 | |
| HIV gag | AIDS |
| HIV gag | HIV infection |
| HIV-1 integrase | AIDS |
| HIV-1 integrase | HIV infection |
| HIV-1 pol | Opportunistic |
| | infections |
| HIV-1 pol | AIDS |
| HIV-1 pol | Hepatitis C |
| HIV-1 tat | HIV infection |
| HIV RNase H | HIV infection |
| HJV, Hepc | anemia |
| HLA | Head and neck tumor |
| HLA | Hemophagocytic |
| | lymphohistiocytosis |
| HLA | Breast cancer |
| HLA | Prostate cancer |
| HLA | Diffuse large B-cell |
| | lymphoma |
| HLA | Ovarian cancer |
| HLA | Colorectal cancer |
| HLA | Refractory plasma cell |
| | myeloma |
| HLA | Acquired |
| | immunodeficiency |
| | syndrome (AIDS) |
| HLA | Melanoma |
| HLA | Relapsed Multiple |
| | myeloma |
| HLA | Lung cancer |
| HLA | Bladder cancer |
| HLA | Epstein-Barr virus |
| | infection |
| HLA class II | Tumor |
| antigen, | |
| MAGEA3 | |
| HLA class II | Solid tumor |
| antigen, TERT | |
| HLA class I | Myxoid liposarcoma |
| antigen | |
| HLA class I | Synovial sarcoma |
| antigen | |
| HLA class I | Non-small cell lung |
| antigen | cancer (NSCLC) |
| HLA class I | Type 1 diabetes |
| antigen, HLA | |
| class II antigen | |
| HLA class I | Solid tumor |
| antigen, KKLC1 | |
| growth factor β | |
| (TGFß) receptor | |
| HLA, TGFBR2 | Tumor |
| HLA-A2 | Renal transplant |
| | rejection |
| HLA-A2 | Liver transplant |
| | rejection |
| HLA-A2 | Liver failure |
| HLA-A2, NY-ESO-1 | Triple-negative breast |
| | cancer (TNBC) |
| HLA-A2, NY-ESO-1 | Sarcoma |
| HLA-G | Metastatic clear cell |
| | renal cell carcinoma |
| HLA-G | Hematologic |
| | malignancy |
| HLA-G | Solid tumor |
| HLA-G | Renal cell carcinoma |
| HLA-G | Ovarian cancer |
| HLA-G | Locally advanced clear |
| | cell renal cell carcinoma |
| HLA-G | Acute myeloid leukemia |
| | (AML) |
| HMGB1 | Fibrosis |
| HMGB1 | Pain |
| HMGB1 | Sepsis |
| HMGB1 | Pulmonary fibrosis |
| HP-NAP | Breast cancer |
| HPV E6 | Cervical cancer |
| HPV E6, HPV | HPV16-positive solid |
| E7 | tumor |
| HPV E7 | Cervical dysplasia |
| HPV E7 | Tumor metastasis |
| HPV E7 | Vaginal |
| | adenocarcinoma |
| HPV E7 | Novel coronavirus |
| | infection |
| HPV E7 | Vulvar tumor |
| HPV E7 | Vulvar intraepithelial |
| | neoplasia (VIN) |
| HPV E7 | Head and neck tumor |
| HPV E7 | Papillomavirus infection |
| HPV E7 | HPV-associated vulvar |
| | squamous cell |
| | carcinoma |
| HPV E7 | HPV-associated penile |
| | squamous cell |
| | carcinoma |
| HPV E7 | HPV-associated cervical |
| | cancer |
| HPV E7 | Squamous cell tumor |
| HPV E7 | Squamous |
| | intraepithelial lesion |
| | (SIL) |
| HPV E7 | Oropharyngeal tumor |
| HPV E7 | Glioblastoma |
| HPV E7 | Spinal cord injury |
| HPV E7 | Macular degeneration |
| HPV E7 | Laryngeal tumor |
| HPV E7 | Cervical intraepithelial |
| | neoplasia (CIN) |
| HPV E7 | Cervical cancer |
| HPV E7 | Polyomavirus infection |
| HPV E7 | HPV-associated |
| | squamous cell |
| | carcinoma |
| HPV E7 | HPV16-positive solid |
| | tumor |
| HS | Mucopolysaccharidosis |
| | type III (MPS III) |
| Hsp27 | Pancreatic cancer |
| Hsp27 | Prostate cancer |
| Hsp27 | Non-small cell lung |
| | cancer (NSCLC) |
| Hsp27 | Bladder cancer |
| Hsp47 | Fibrosis |
| Hsp47 | Systemic sclerosis |
| Hsp47 | Idiopathic pulmonary |
| | fibrosis |
| HSP70 heat- | Non-small cell lung |
| shock proteins | cancer (NSCLC) |
| HSPA1A, p53 | Tumor |
| HSPA9 | Ovarian cancer |
| HSPGs, MMPs, | Tumor |
| TGF-β1 | |
| HSPGs, MMPs | Trauma and injury |
| TGF-β1 | |
| HTATIP2 | Complement disorder |
| HTT | Hodgkin lymphoma |
| HTT | Huntington's disease |
| Hyaluronic acid | Metastatic pancreatic |
| | adenocarcinoma |
| Hyaluronic acid | Secondary malignant |
| | neoplasm of pancreas |
| Hyaluronic acid | Retinoblastoma |
| Hyaluronic acid | Brain cancer |
| Hyaluronic acid | Melanoma |
| Hypoxia- | Myocardial ischemia |
| inducible factor 1 | |
| Hypoxia- | Peripheral vascular |
| inducible factor 1 | disease |
| Hypoxia- | Peripheral arterial |
| inducible factor 1 | disease |
| Hypoxia- | Intermittent claudication |
| inducible factor 1 | |
| Hypoxia- | Atherosclerosis |
| inducible factor 1 | |
| ICAM-1 | Inflammatory bowel |
| | disease |
| ICAM-1 | Inflammation |
| ICAM-1 | Ulcerative colitis |
| ICAM-1 | Anaplastic thyroid |
| | carcinoma |
| ICAM-1 | Poorly differentiated |
| | thyroid carcinoma |
| ICAM-1 | Recurrent thyroid |
| | cancer |
| ICAM-1 | Non-small cell lung |
| | cancer |
| ICE1, caspase | Neurofibroma |
| ID1 | Tumor |
| IDS | Mucopolysaccharidosis |
| | type II |
| IDUA | Mucopolysaccharidosis |
| | type I |
| IFN α | Head and neck tumor |
| IFN α | Melanoma |
| IFNAR | Pleural effusion |
| IFNAR | Rheumatoid arthritis |
| IFNAR | Colorectal cancer |
| FNAR | Glioblastoma |
| IFNAR | Non-muscle-invasive |
| | bladder tumor |
| IFNAR | Malignant pleural |
| | mesothelioma |
| IFNAR | Pleomorphic |
| | glioblastoma |
| IFNAR | Hepatitis C |
| IFNAR | Bladder cancer |
| IFNAR | Bladder cancer |
| IFNGR | Tumor |
| IFNGR | Heart failure |
| IFNGR | Perioperative ischemia |
| IFNGR | Peripheral vascular |
| | disease |
| FNGR | Neurodegenerative |
| | disease |
| IFNGR | Cutaneous T-cell |
| | lymphoma |
| IFNGR | Candidemia |
| FNGR | Brain injury |
| IFNGR | Basal cell nevus |
| | syndrome |
| IFNGR | Melanoma |
| IFNGR | Lung disease |
| IFNGR | Type 1 diabetes |
| IFNα2 | Melanoma |
| IFNβ | Endometrial cancer |
| IFNβ | Glioma |
| IFNβ | Acute myeloid leukemia |
| IFNβ | Multiple sclerosis |
| IFNβ | Multiple myeloma |
| IFNβ | T-cell lymphoma |
| IFNβ, NIS | Neuroendocrine |
| | carcinoma |
| IFNβ, NIS | Refractory malignant |
| | solid tumor |
| IFNβ, NIS | Colorectal cancer |
| IFNβ, NIS | Acute myeloid leukemia |
| IFNβ, NIS | Liver cancer |
| IFNβ, NIS | Malignant solid tumor |
| IFNβ, NIS | Multiple myeloma |
| IFNβ, NIS | T-cell lymphoma |
| IFNβ, TLR9 | Inflammation |
| IFNγ | Castration-resistant |
| | prostate cancer |
| IFNγ, TERT | Tumor |
| IgE | Immune system disease |
| IgE | Hypersensitivity |
| IgE receptors | Asthma |
| IGF-1 | Tumor |
| IGF-1 | Alzheimer's disease |
| IGF-1 | Type 1 diabete |
| IGF-1R | Psoriasis |
| IGF-1R | Fibrosis |
| IGF-1R | Head and neck tumor |
| IGF-1R | Glaucoma |
| IGF-1R | Prostate cancer |
| IGF-1R | Ovarian cancer |
| IGF-1R | Glioblastoma |
| IGF-1R | Amyotrophic lateral |
| | sclerosis |
| IGF-1R | Graves' ophthalmopathy |
| IGF-1R | Hepatocellular |
| | carcinoma |
| IGF-1R | Laron syndrome |
| IGFBP2, | Breast cancer |
| IGFBP5 | |
| IGHMBP2 | Charcot-Marie-Tooth |
| | disease type 2S |
| IKKy, NF-κB | Tumor |
| IL-10R | Autoimmune hepatitis |
| IL-10R | Inflammation |
| IL-10R | Solid tumor |
| IL-10R | Amyotrophic lateral |
| | sclerosis |
| IL-10R | Musculoskeletal pain |
| IL-10R | Arthralgia |
| IL-10R | Multiple sclerosis |
| IL-10R | Back pain |
| IL-12 | Metastatic melanoma |
| IL-12 | Tumor |
| IL-12 | Pancreatic cancer |
| IL-12 | Adverse drug reaction |
| IL-12 | Novel coronavirus |
| | infection |
| IL-12 | Head and neck tumor |
| IL-12 | Head and neck tumor |
| IL-12 | Triple-negative breast |
| | cancer |
| IL-12 | Breast cancer |
| IL-12 | Prostate cancer |
| IL-12 | Skin tumor |
| IL-12 | Cutaneous T-cell |
| | lymphoma |
| IL-12 | Diffuse intrinsic pontine |
| | glioma |
| IL-12 | Merkel cell carcinoma |
| IL-12 | Ovarian cancer |
| IL-12 | Squamous cell |
| | carcinoma |
| IL-12 | Locally advanced |
| | melanoma |
| IL-12 | Glioblastoma |
| IL-12 | Acute myeloid leukemia |
| IL-12 | Melanoma |
| IL-12 | Lung cancer |
| IL-12 | HPV-associated cancer |
| IL-12, IL-15 | Advanced malignant |
| | solid tumor |
| IL-12, IL-15, PDL1 | Solid tumor |
| IL-12, IL-2 | Melanoma |
| IL-12, MUC16 | Ovarian cancer |
| IL-12, PD-1 | Glioblastoma |
| IL-12, PD-1 | Glioma |
| IL-12, PD-1 | Recurrent glioblastoma |
| IL-12, PD-1 | Glioblastoma multiforme |
| IL-12, thymidine | Astrocytoma |
| kinase | |
| IL-12, thymidine | Recurrent prostate |
| kinase | cancer |
| IL-12R | Metastatic breast |
| | cancer |
| IL-12R | Metastatic colorectal |
| | cancer |
| IL-12R | Primary peritoneal |
| | carcinoma |
| IL-12R | Pediatric cerebellar |
| | astrocytoma |
| IL-12R | Advanced malignant |
| | solid tumor |
| IL-12R | Head and neck tumor |
| IL-12R | Fallopian tube cancer |
| IL-12R | Gliosarcoma |
| IL-12R | Mucolipidosis type II |
| IL-12R | Diffuse intrinsic pontine |
| | glioma |
| IL-12R | Ovarian epithelial carcinoma |
| IL-12R | Ovarian cancer |
| IL-12R | Locally advanced breast |
| | cancer |
| IL-12R | Colorectal cancer with |
| | liver metastasis |
| IL-12R | Colorectal cancer |
| IL-12R | Glioblastoma |
| IL-12R | Glioma |
| IL-12R | Melanoma |
| IL-12R | Secondary malignant |
| | neoplasm of peritoneum |
| IL-12R | Peritoneal cancer |
| IL-12R | Recurrent breast cancer |
| IL-12R | Recurrent glioblastoma |
| IL-12R | Recurrent malignant |
| | glioma |
| IL-12R | Glioblastoma multiforme |
| IL-12R | Metabolic bone disease |
| IL-12R | Unresectable melanoma |
| IL-12R | WHO grade III mixed |
| | glioma |
| IL-12R, decorin | Gastrointestinal tumor |
| IL-12R, decorin | Myeloproliferative |
| | disorder |
| IL-12R, decorin | Multiple myeloma |
| IL-12R, IL15R | Solid tumor |
| IL-12R, IL15R | Renal cell carcinoma |
| IL-12R, IL15R | Breast cancer |
| IL-12R, IL15R | Ovarian cancer |
| IL-12R, IL-15Rα | Hematologic |
| | malignancy |
| IL-12R, IL-15Rα | Solid tumor |
| IL-12R, IL- | Metastatic gastric |
| 15Rα, PDL1 | adenocarcinoma |
| IL-12R, IL- | Metastatic |
| 15Rα, PDL1 | gastroesophageal |
| | adenocarcinoma |
| IL-12R, IL- | Metastatic |
| 15Rα, PDL1 | gastroesophageal |
| | junction |
| | adenocarcinoma |
| IL-12R, IL- | Advanced pancreatic |
| 15Rα, PDL1 | adenocarcinoma |
| IL-12R, IL- | Advanced |
| 15Rα, PDL1 | hepatocellular |
| | carcinoma |
| IL-12R, IL- | Advanced malignant |
| 15Rα, PDL1 | solid tumor |
| IL-12R, IL- | Osteosarcoma |
| 15Rα, PDL1 | |
| IL-12R, IL- | Hepatocellular |
| 15Rα, PDL1 | carcinoma |
| IL-12R, IL- | Intrahepatic |
| 15Ra, PDL1 | cholangiocarcinoma |
| IL-12R, IL- | Liver cancer |
| 15Rα, PDL1 | |
| IL-12R, IL- | Cholangiocarcinoma |
| 15Rα, PDL1 | |
| IL-12R, IL-7Rα | Metastatic solid tumor |
| IL-12R, IL-7Rα | Advanced cancer |
| IL-12R, NY- | Tumor |
| ESO-1 | |
| IL-12R, PD-1 | Endometrial cancer |
| IL-12R, PD-1 | Metastatic colorectal |
| | cancer |
| IL-12R, PD-1 | Advanced malignant |
| | solid tumor |
| IL-12R, PD-1 | Head and neck |
| | squamous cell |
| | carcinoma |
| IL-12R, PD-1 | Esophageal cancer |
| IL-12R, PD-1 | Breast cancer |
| IL-12R, PD-1 | Sarcoma |
| IL-12R, PD-1 | Skin tumor |
| IL-12R, PD-1 | Ovarian cancer |
| IL-12R, PD-1 | Lymphoma |
| L-12R, PD-1 | Colorectal cancer with |
| | liver metastasis |
| IL-12R, PD-1 | Colorectal cancer |
| IL-12R, PD-1 | Melanoma |
| IL-12R, PD-1 | Liver cancer |
| IL-12R, PD-1 | Non-small cell lung |
| | cancer |
| IL-12R, PD-1 | Malignant pleural |
| | mesothelioma |
| IL-12R, PD-1 | Bladder cancer |
| IL-12R, RIG-I | Non-muscle-invasive |
| | bladder tumor |
| IL-12R, STEAP1 | Prostate cancer |
| IL-13R, IL-4Rα | Asthma |
| IL-13R, IL-4Rα | Allergic asthma |
| IL-13R, IL-4Rα | Rhinitis |
| IL-13Rα2 | Brain metastases |
| IL-13Rα2 | Glioblastoma |
| IL-13Rα2 | Glioma |
| IL-13Ra2 | Melanoma |
| IL-13Rα2 | High-grade astrocytoma |
| IL-13Rα2 | Recurrent glioblastoma |
| IL-13Rα2 | Recurrent malignant |
| | glioma |
| IL-13Rα2 | Pleomorphic |
| | glioblastoma |
| IL-15 | Metastatic melanoma |
| IL-15 | Metastatic non-small |
| | cell lung cancer |
| IL-15 | Tumor |
| IL-15 | Gastric cancer |
| IL-15 | Solid tumor |
| IL-15 | Lymphoma |
| IL-15 | Colorectal cancer |
| IL-15 | Secondary malignant |
| | lung tumor |
| IL-15 | Melanoma |
| IL-15 | Non-small cell lung |
| | cancer |
| IL-15, IL-2 | Tumor |
| IL-15, PDL1 | Non-small cell lung |
| | cancer |
| IL-15, PSCA | Pancreatic acinar cell |
| | carcinoma |
| IL-15, PSCA | Gastric cancer |
| IL-15, PSCA | Prostate cancer |
| IL-15, PSCA | Bladder cancer |
| IL15R, MUC16 | Fallopian tube cancer |
| IL15R, MUC16 | Refractory ovarian |
| | cancer |
| IL15R, MUC16 | Ovarian cancer |
| IL 15R, MUC16 | Peritoneal cancer |
| IL15R, MUC16 | Recurrent primary |
| | peritoneal cancer |
| IL15R, MUC16 | Recurrent ovarian |
| | cancer |
| IL15R, MUC16 | Recurrent platinum- |
| | resistant primary |
| | peritoneal cancer |
| IL15R, MUC16 | Platinum-resistant |
| | fallopian tube cancer |
| IL15R, MUC16 | Platinum-resistant |
| | ovarian cancer |
| IL15R, PD-1 | Lung cancer |
| IL15R, PD-1 | Non-small cell lung |
| | cancer |
| IL-15Rα, | Metastatic colorectal |
| NKG2D | cancer |
| IL-15Rα, | Osteosarcoma |
| NKG2D | |
| IL-15Rα, | Hepatocellular |
| NKG2D | carcinoma |
| IL-15Rα, | Intrahepatic |
| NKG2D | cholangiocarcinoma |
| IL-17RA | Psoriasis |
| IL-17RA | Congenital ichthyosis |
| | erythroderma |
| IL-17RA | Hair loss |
| IL-18 | Hematologic |
| | malignancy |
| IL-18 | Solid tumor |
| IL-18 | Renal cell carcinoma |
| IL-18 | Neuralgia |
| IL-1R, MMPs | Liver cirrhosis |
| IL1R1 | Gout |
| IL1R1 | Arthritis |
| IL-1RA | Knee arthritis |
| IL-1RA, PRG4 | Osteoarthritis |
| IL-1α | Osteoarthritis |
| IL-2 | Primary peritoneal |
| | adenocarcinoma |
| IL-2 | Primary peritoneal |
| | cancer |
| IL-2 | Adenocarcinoma |
| IL-2 | Head and neck tumor |
| IL-2 | Fallopian tube cancer |
| IL-2 | Renal cell carcinoma |
| IL-2 | Neuroblastoma |
| IL-2 | Breast cancer |
| IL-2 | Prostate cancer |
| IL-2 | Skin tumor |
| IL-2 | Ovarian |
| | adenocarcinoma |
| IL-2 | Ovarian serous |
| | adenocarcinoma |
| IL-2 | Colon cancer |
| IL-2 | Serous |
| | cystadenocarcinoma |
| IL-2 | Mesothelioma |
| IL-2 | Melanoma |
| IL-2 | Melanoma |
| IL-2 | Hepatocellular |
| | carcinoma |
| IL-2 | Lung cancer |
| IL-2, TNF | Renal cell carcinoma |
| IL-2, TNF | Colon cancer |
| IL-2, TNF | Melanoma |
| IL-2, TNF-α | Metastatic melanoma |
| IL-2, TNF-α | Head and neck |
| | squamous cell |
| | carcinoma |
| IL-2, TNF-α | Solid tumor |
| IL-2, TNF-α | Ovarian cancer |
| IL-2, TNF-α | Melanoma |
| IL-2, TNF-α | Non-small cell lung |
| | cancer |
| IL-21 | Pancreatic cancer |
| IL-21 | Pulmonary arterial |
| | hypertension |
| IL-21R | Poxvirus infection |
| IL-23 | Tumor |
| IL-24 | Pancreatic cancer |
| IL-24 | Head and neck tumor |
| IL-24 | Breast cancer |
| IL-24 | Melanoma |
| IL-24 | Hepatocellular |
| | carcinoma |
| IL-24 | Lung cancer |
| IL-24, THBS1 | Leukemia |
| IL-24, TRAIL | Hepatocellular |
| | carcinoma |
| IL-27 | Tumor |
| IL-2R | Metastatic melanoma |
| IL-2R | Gastrointestinal tumor |
| IL-2R | Renal tumor |
| IL-2R | Renal tumor |
| IL-2R | Renal cell carcinoma |
| IL-2R | Prostate cancer |
| IL-2R | Mesothelioma |
| IL-2R | Melanoma |
| IL-2R | Recurrent prostate |
| | cancer |
| IL-2R | Lung cancer |
| IL-2R, LMP2 | Cancer |
| IL2RA | Tumor |
| IL2RA | Hematologic |
| | malignancy |
| IL-2RY | Immunodeficiency |
| | syndrome |
| IL-2RY | X-linked severe |
| | combined |
| | immunodeficiency |
| IL-3 | CD123-positive acute |
| | myeloid leukemia |
| IL-33R | Asthma |
| IL-4Ra | Asthma |
| IL-6R | Inflammation |
| IL-6R | Arthritis |
| IL-6RA | Rheumatoid arthritis |
| IL-7 | Tumor |
| IL-7 | Melanoma |
| IL-7Rα | Head and neck tumor |
| IL-7Rα | Cervical cancer |
| IL-8 | Pathological |
| | neovascularization |
| influenza virus | Influenza virus infection |
| M1 | |
| influenza virus | Influenza A virus |
| M1 | infection |
| ING4 | Relapsed acute myeloid |
| | leukemia |
| INHBE | Abdominal obesity |
| INSR | Diabetic macular edema |
| INSR | Diabetes |
| INSR | Wet age-related |
| | macular degeneration |
| INSR | Ischemic central retinal |
| | vein occlusion |
| INSR | Choroidal |
| | neovascularization |
| INSR | Corneal transplant |
| | rejection |
| INSR | Macular degeneration |
| INSR | Bladder cancer |
| INSR | Type 1 diabetes |
| Insulin | Type 1 diabetes |
| Integrin | Tumor |
| Integrin | Prostate |
| | adenocarcinoma |
| Integrin | Localized prostate |
| | cancer |
| Ion channels | Idiopathic pulmonary |
| | fibrosis |
| lon channels | Trigeminal neuralgia |
| Ion channels | Partial seizures |
| IRAK1 | Hepatocellular |
| | carcinoma |
| IRAK1 | Trauma and injury |
| IRF4 | Refractory |
| | plasmacytoma |
| IRF4 | Recurrent multiple |
| | myeloma |
| IRF5 | Rheumatoid arthritis |
| ISG15 | Hepatitis C |
| ITGB7 | Multiple myeloma |
| JAK1 | Autoimmune disease |
| JAK1 | Inflammation |
| KHK | Obesity |
| KHK | Type 2 diabetes |
| Kir7.1 | Leber congenital |
| | amaurosis |
| KKLC1 | Pancreatic acinar cell |
| | carcinoma |
| KKLC1 | Gastric cancer |
| KKLC1 | Breast cancer |
| KKLC1 | Cervical cancer |
| KKLC1 | Liver cancer |
| KKLC1 | Lung cancer |
| KKLC1 | Non-small cell lung |
| | cancer |
| KLB | Alzheimer's disease |
| KLK2 | Metastatic castration- |
| | resistant prostate |
| | cancer |
| KLK3 | Prostate cancer |
| KLK5 | Netherton syndrome |
| KLKB1 | Hereditary angioedema |
| KMA | Multiple myeloma |
| KRAS | Tumor |
| KRAS | Pancreatic cancer |
| KRAS | Immune system disease |
| KRAS | Colorectal cancer |
| KRAS | Muscle disorders |
| KRAS | Lung cancer |
| KRAS | Non-small cell lung |
| | cancer |
| KRAS | KRAS-mutant tumor |
| KRAS G12A | Lung cancer |
| KRAS G12C | Tumor |
| KRAS G12C | Lung cancer |
| KRAS G12C | Non-small cell lung |
| | cancer |
| KRAS G12C, | Pancreatic ductal |
| KRAS G12D | adenocarcinoma |
| | KRAS G12C, |
| KRAS G12D | |
| KRAS G12C, | Tumor |
| KRAS G12D, | |
| KRAS G12V | |
| KRAS G12C, | Pancreatic cancer |
| KRAS G12D, | |
| KRAS G12V | |
| KRAS G12C, | Colon cancer |
| KRAS G12D, | |
| KRAS G12V | |
| KRAS G12C, | Lung cancer |
| KRAS G12D, | |
| KRAS G12V | |
| KRAS G12C, | Tumor |
| KRAS G12D, | |
| PI3Kα, p53 | |
| R175H | |
| KRAS G12D | Endometrial cancer |
| KRAS G12D | Metastatic pancreatic |
| | ductal adenocarcinoma |
| KRAS G12D | Rectal cancer |
| KRAS G12D | Pancreatic acinar cell |
| | carcinoma |
| KRAS G12D | Pancreatic ductal |
| | adenocarcinoma |
| KRAS G12D | Pancreatic cancer |
| KRAS G12D | Gastric cancer |
| KRAS G12D | Solid tumor |
| KRAS G12D | Locally advanced |
| | pancreatic |
| | adenocarcinoma |
| KRAS G12D | Colorectal cancer |
| KRAS G12D | Colon cancer |
| KRAS G12D | Lung cancer |
| KRAS G12D | Non-small cell lung |
| | cancer |
| KRAS G12D, | Solid tumor |
| KRAS G12V | |
| KRAS G12V | Metastatic pancreatic |
| | adenocarcinoma |
| KRAS G12V | Metastatic non-small |
| | cell lung cancer |
| KRAS G12V | Tumor |
| KRAS G12V | Pancreatic ductal |
| | adenocarcinoma |
| KRAS G12V | Pancreatic cancer |
| KRAS G12V | Solid tumor |
| KRAS G12V | Colorectal cancer |
| KRAS G12V | Lung cancer |
| KRAS G12V | Non-small cell lung |
| | cancer |
| KRAS G12V | Colonic |
| | adenocarcinoma |
| KRAS G12V | Intestinal tumor |
| KRAS G13D | Pancreatic cancer |
| KRT6A | Congenital |
| | pachyonychia |
| Ku70/80, MRN, | Metastatic solid tumor |
| PARP1 | |
| Ku70/80, MRN, | Breast cancer |
| PARP1 | |
| Ku70/80, MRN, | Prostate cancer |
| PARP1 | |
| Ku70/80, MRN, | Recurrent ovarian |
| PARP1 | cancer |
| Kv7.2 | epilepsy |
| L1CAM | Neuroendocrine |
| | prostate cancer |
| L1CAM | Neuroblastoma |
| L1CAM | leukemia |
| L1CAM | CD22-positive acute |
| | lymphoblastic leukemia |
| L1CAM | CD19 expressing |
| | malignancy |
| LAG3 | Tumor |
| LAGE-1a, NY- | Solid tumor |
| ESO-1 | |
| LAMA1 | Muscular dystrophy |
| LAMP-1, TERT | Acute myeloid leukemia |
| LAMP-2 | Glycogen storage |
| | disease type Ilb |
| LAMP-2 | Diverticular disease |
| LAMP-2 | Huntington's disease |
| LCA5 | Retinal degeneration |
| LCA5 | Leber congenital |
| | amaurosis type 5 |
| LCN2 | Pancreatic cancer |
| LDLR | Hypercholesterolemia |
| LECT2 | Amyloidosis |
| LEKTI | Netherton syndrome |
| | lepB |
| | aeruginosa infection |
| Lewis-Y antigen | Advanced cancer |
| Lewis-Y antigen | Acute myeloid leukemia |
| LGR5 | Metastatic colorectal |
| | cancer |
| LGR5 | Hematologic |
| | malignancy |
| LGR5 | Ovarian cancer |
| LGR5 | Colorectal cancer |
| L-HBsAg | Hepatitis B |
| L-HBsAg | Fibrosis |
| L-HBsAg | Chronic hepatitis B |
| L-HBsAg | Chronic hepatitis D |
| LIGHT | Glioblastoma |
| LILRB4 | Refractory acute |
| | myeloid leukemia |
| LILRB4 | Chronic |
| | myelomonocytic |
| | leukemia |
| LILRB4 | Acute myeloid leukemia |
| LILRB4 | Acute myelomonocytic |
| | leukemia |
| LILRB4 | Acute monocytic |
| | leukemia |
| LILRB4 | Relapsed acute myeloid |
| | leukemia |
| LILRB4 | Multiple myeloma |
| LIN28B | Pancreatic cancer |
| LIPG | Coronary heart disease |
| lipoprotein(a) | Aortic stenosis |
| lipoprotein(a) | Cardiovascular disease |
| lipoprotein(a) | Neurodegenerative |
| | disease |
| lipoprotein(a) | Hyperlipoproteinemia |
| lipoprotein(a) | Liver disease |
| lipoprotein(a) | Atherosclerosis |
| lipoprotein(a) | Hypobetalipoproteinemia |
| LIV-1 | Breast cancer |
| LMNA | Premature aging |
| LMNA | Myocardial disease |
| LMNA | Dilated cardiomyopathy |
| LMP1 | Hematologic |
| | malignancy |
| LMP1 | Nasopharyngeal |
| | carcinoma |
| LMP1, LMP2 | Leiomyosarcoma |
| LMP1, LMP2 | Hodgkin lymphoma |
| LMP1, LMP2 | Non-Hodgkin lymphoma |
| LMP1, LMP2 | Nasopharyngeal |
| | carcinoma |
| LMP1, MAVS | Vaccination |
| LMP1, MAVS | HIV infection |
| LMP2 | Head and neck tumor |
| | metastasis |
| LMP2 | Recurrent |
| | nasopharyngeal |
| | carcinoma |
| LPL | Type V |
| | hyperlipoproteinemia |
| LPL | Hyperlipoproteinemia |
| | type l |
| LptD | Pseudomonas |
| | aeruginosa infection |
| LpxC | Pseudomonas |
| | aeruginosa infection |
| LRRC15 | Tumor |
| LRRK2 | Parkinson's disease |
| L-sel | Inflammation |
| LXR | Nonalcoholic |
| | steatohepatitis |
| LXR | Type II |
| | hyperlipoproteinemia |
| LY86 | Duchenne muscular |
| | dystrophy |
| LZTS1, LZTS2 | Tumor |
| MAFA, PDX1 | Type 1 diabetes |
| MAGEA1 | Tumor |
| MAGEA1 | Head and neck tumor |
| MAGEA1 | Solid tumor |
| MAGEA1 | Triple-negative breast |
| | cancer |
| MAGEA1 | Urothelial carcinoma |
| MAGEA1 | Ovarian cancer |
| MAGEA1 | Melanoma |
| MAGEA1 | Melanoma |
| MAGEA1 | Cervical cancer |
| MAGEA1 | Hepatocellular |
| | carcinoma |
| MAGEA 1 | Liver cancer |
| MAGEA1 | Non-small cell lung |
| | cancer |
| MAGEA1 | HPV-related cancers |
| MAGEA1, | Solid tumor |
| PRAME | |
| MAGEA10 | Head and neck tumor |
| MAGEA10 | Urothelial carcinoma |
| MAGEA 10 | Melanoma |
| MAGEA 10 | Non-small cell lung |
| | cancer |
| MAGEA 10 | Malignant epithelial |
| | tumor |
| MAGEA12, | Metastatic melanoma |
| MAGEA3 | |
| MAGEA 12, | Tumor metastasis |
| MAGEA3 | |
| MAGEA 12, | Kidney tumor |
| MAGEA3 | |
| MAGEA3 | Kidney tumor |
| MAGEA3 | Breast cancer |
| MAGEA3 | Melanoma |
| MAGEA3 | Cervical cancer |
| MAGEA3 | Lung cancer |
| MAGEA3, | Tumor |
| MAGEA6 | |
| MAGEA3, | Solid tumor |
| MAGEA6 | |
| MAGEA4 | Endometrial cancer |
| MAGEA4 | Liposarcoma |
| MAGEA4 | Myxoid liposarcoma |
| MAGEA4 | Gastroesophageal |
| | junction malignant |
| | tumor |
| MAGEA4 | Gastric cancer |
| MAGEA4 | Gastric cancer |
| MAGEA4 | Esophageal cancer |
| MAGEA4 | Solid tumor |
| MAGEA4 | Solid tumor |
| MAGEA4 | Neurofibrosarcoma |
| MAGEA4 | Neuroblastoma |
| MAGEA4 | sarcoma |
| MAGEA4 | Urothelial carcinoma |
| MAGEA4 | Ovarian cancer |
| MAGEA4 | Ovarian cancer |
| MAGEA4 | Synovial sarcoma |
| MAGEA4 | Melanoma |
| MAGEA4 | Osteosarcoma |
| MAGEA4 | Recurrent solid tumor |
| MAGEA4 | Non-small cell lung |
| | cancer |
| MAGEA4 | Bladder cancer |
| MAGEA4, | Solid tumor |
| MAGEA8 | |
| MAGEA4, NY-ESO-1, | Hematologic |
| | malignancy |
| PRAME, SSX2, survivin | |
| MAGEA4, NY-ESO-1, | Acute myeloid leukemia |
| PRAME, SSX2, | |
| survivin | |
| MAGEA4, NY-ESO-1, | Hodgkin lymphoma |
| PRAME, SSX2, | |
| survivin | |
| MAGEA4, NY- | Non-Hodgkin lymphoma |
| ESO-1, | |
| PRAME, SSX2, | |
| survivin | |
| MAGEA4, NY- | Pancreatic cancer |
| ESO-1, | |
| PRAME, SSX2, | |
| WT1, survivin | |
| MAGEA4, NY- | Refractory lymphoma |
| ESO-1, | |
| PRAME, SSX2, | |
| WT1, survivin | |
| MAGEA4, NY- | Refractory non-Hodgkin |
| ESO-1, | lymphoma |
| PRAME, SSX2, | |
| WT1, survivin | |
| MAGEA4, NY- | Hodgkin lymphoma |
| ESO-1, | |
| PRAME, SSX2, | |
| WT1, survivin | |
| MAGEA4, NY- | Relapsed lymphoma |
| ESO-1, | |
| PRAME, SSX2, | |
| WT1, survivin | |
| MAGEA4, NY- | Relapsed non-Hodgkin |
| ESO-1, | lymphoma |
| PRAME, SSX2, | |
| WT1, survivin | |
| MAGEC2 | Head and neck tumor |
| MAGEC2 | Solid tumor |
| MAGEC2 | Ovarian cancer |
| MAGEC2 | Melanoma |
| MAGEC2 | Hepatocellular |
| | carcinoma |
| MAGEC2 | Non-small cell lung |
| | cancer |
| MALAT1 | Tumor |
| MALAT1 | Breast cancer |
| MAX, c-Myc | Tumor |
| MBP | Multiple sclerosis |
| MCM7 | Tumor |
| M-CSF | Tumor |
| MDA5, RIG-I, | Myxoid MFH |
| TLR3 | |
| MDA5, RIG-I, | Head and Neck |
| TLR3 | Squamous Cell |
| | Carcinoma |
| MDA5, RIG-I, | Neurofibrosarcoma |
| TLR3 | |
| MDA5, RIG-I | Breast cancer |
| TLR3 | |
| MDA5, RIG-I, | Sarcoma |
| TLR3 | |
| MDA5, RIG-I, | Dedifferentiated |
| TLR3 | liposarcoma |
| MDA5, RIG-I, | Leiomyosarcoma |
| TLR3 | |
| MDA5, RIG-I, | Synovial sarcoma |
| TLR3 | |
| MDA5, RIG-I, | Rhabdomyosarcoma |
| TLR3 | |
| MDA5, RIG-1, | Hepatoembryosarcoma |
| TLR3 | |
| MDA5, RIG-I, | Retroperitoneal |
| TLR3 | sarcoma |
| MDA5, RIG-1, | Non-small cell lung |
| TLR3 | cancer |
| MDA5, RIG-I, | Malignant fibrous |
| TLR3 | histiocytoma |
| MDH1, MDH2 | Non-small cell lung |
| | cancer |
| MECOM | Ovarian cancer |
| MECOM | Lung cancer |
| MECP2 | Lubbs sex-linked mental |
| | retardation syndrome |
| MECP2 | Spinal muscular atrophy |
| | with respiratory distress |
| | type 1 |
| MECP2 | Charcot-Marie-Tooth |
| | Disease Type 2S |
| MECP2 | Rett syndrome |
| melan-A | Metastatic melanoma |
| melan-A | Melanoma |
| melan-A | Melanoma |
| MerTK | Retinitis pigmentosa |
| MEX3B | Severe asthma |
| MFN2 | Charcot-Marie-Tooth |
| | Disease |
| MFSD8 | Neuronal ceroid |
| | lipofuscinosis |
| MFSD8 | Lysosomal storage |
| | disease |
| MGMT | Glioblastoma |
| MICA | Hepatocellular |
| | carcinoma |
| MICA, MICB | Tumor |
| MICA, MICB, | Solid tumor |
| ULBP1 | |
| micro- | Duchenne muscular |
| dystrophin | dystrophy |
| microRNA let- | Hepatocellular |
| 7i-5p | carcinoma |
| MicroRNAs | Alzheimer's disease |
| Microtubule- | Myocardial infarction |
| associated | |
| proteins | |
| Microtubule- | Nerve damage |
| associated | |
| proteins | |
| Microtubule- | burn |
| associated | |
| proteins | |
| miHAs | Hematopoietic stem cell |
| | transplantation |
| miHAs | Hematologic |
| | malignancy |
| miHAs | Hematologic |
| | malignancy |
| miHAs | Acute myeloid leukemia |
| miHAs | Acute lymphoblastic |
| | leukemia |
| miHAs | Myelodysplastic |
| | syndrome |
| miHAs | leukemia |
| mini-dystrophin | Duchenne muscular |
| | dystrophy |
| MIR103A1, | Nonalcoholic |
| miR-107 | steatohepatitis |
| miR-10b | Pancreatic cancer |
| miR-10b | Small cell lung cancer |
| miR-10b | Advanced solid tumor |
| miR-10b | Breast cancer |
| miR-10b | Ovarian cancer |
| miR-10b | Colon cancer |
| miR-10b | Glioblastoma |
| miR-10b | Osteosarcoma |
| miR-10b, miR-21 | Glioblastoma |
| miR-122 | Hepatitis C |
| miR-126 | Chronic myeloid |
| | leukemia |
| miR-126 | Acute myeloid leukemia |
| | in adults |
| miR-132 | Heart failure |
| miR-132 | Cardiac hypertrophy |
| miR-132 | Heart failure with |
| | preserved ejection |
| | fraction |
| miR-132 | Dilated cardiomyopathy |
| miR-132 | Acute myocardial |
| | infarction |
| MIR135A1 | Depression |
| miR-143 | Colorectal cancer |
| miR-145 | Pulmonary hypertension |
| miR-150 | Rectal cancer |
| miR-150 | Colorectal cancer |
| miR-155 | Wet macular |
| | degeneration |
| miR-155 | Cutaneous T-cell |
| | lymphoma |
| miR-17 | Autosomal dominant |
| | polycystic kidney |
| | disease |
| MIR181A2 | Chondrosarcoma |
| miR-193a-3p | Advanced solid tumor |
| miR-193a-3p | Melanoma |
| miR-193a-3p | Liver cancer |
| miR-195 | Liver cancer |
| miR-195 | Bile duct tumor |
| miR-21 | Post-COVID-19 |
| | Syndrome |
| miR-21 | Nephritis |
| miR-21 | Triple-negative breast |
| | cancer |
| miR-21 | Lung cancer |
| miR-21 | Non-small cell lung |
| | cancer |
| miR-21 | Bladder cancer |
| miR-21, miR- | Triple-negative breast |
| 34a | cancer |
| miR-22 | Fatty liver |
| miR-22 | Optic nerve disease |
| miR-22 | Obesity |
| miR-22 | Nonalcoholic |
| | steatohepatitis |
| miR-22 | Metabolic disease |
| miR-22 | Metabolic fatty liver |
| | disease |
| miR-22 | Type 2 diabetes |
| miR-23b | Osteoarthritis |
| miR-29 | Fibrosis |
| miR-29 | Idiopathic pulmonary |
| | fibrosis |
| miR-328 | Shortsighted |
| miR-33a | Idiopathic pulmonary |
| | fibrosis |
| miR-34a | Small cell carcinoma |
| miR-34a | Renal cell carcinoma |
| miR-34a | Lymphoma |
| miR-34a | Melanoma |
| miR-34a | Liver cancer |
| miR-34a | Non-small cell lung |
| | cancer |
| miR-34a | Multiple myeloma |
| MIR449A | Breathing disorders |
| MIR92A1 | Heart failure |
| miR-96 | Diabetic nephropathy |
| MIRN122 | Parkinson's disease |
| microRNA | |
| MIRN122 | Amyotrophic lateral |
| microRNA | sclerosis |
| MIRN122 | Cholestasis |
| microRNA | |
| MIRN122 | Hepatitis C |
| microRNA | |
| MIRN122 | Alzheimer's disease |
| microRNA | |
| Mitochondrial | sarcoma |
| proteins | |
| Mitochondrial | Skin tumor |
| proteins | |
| Mitochondrial | Basal cell carcinoma |
| proteins | |
| Mitochondrial | Melanoma |
| proteins | |
| MKI67 | Bladder cancer |
| MLC | White matter disease |
| MMP1 | Tumor |
| MMP1 | Systemic scleroderma |
| MMP1 | Skin laxity |
| MMP1 | Facial wrinkle |
| MMP1 | aging |
| MMP1 | Trauma and Injury |
| MMP2 | Prostate cancer |
| MMP2 | Colorectal cancer |
| MMP2 | Glioblastoma |
| MMP2 | Melanoma |
| MMP2 | MMP2-positive |
| | glioblastoma |
| MMP-7 | Idiopathic pulmonary |
| | fibrosis |
| MNK1 | Non-small cell lung |
| | cancer |
| MnSOD | Colorectal cancer |
| MnSOD, TRAIL | Non-small cell lung |
| | cancer |
| MnSOD, | Motor neurone disease |
| transcription | |
| factors, and | |
| related | |
| regulatory | |
| factors | |
| MnSOD, | Traumatic brain injury |
| transcription | |
| factors, and | |
| related | |
| regulatory | |
| factors | |
| MnSOD, | Alzheimer's disease |
| transcription | |
| factors, and | |
| related | |
| regulatory | |
| factors | |
| MSH3 | Spinocerebellar ataxia |
| MSH3 | Myotonic dystrophy |
| MSH3 | Huntington's disease |
| MSLN | Mediastinal large B-cell |
| | lymphoma |
| MSLN | Metastatic non-small |
| | cell lung cancer |
| MSLN | Tumor |
| MSLN | Primary peritoneal |
| | cancer |
| MSLN | Pancreatic acinar cell |
| | carcinoma |
| MSLN | Pancreatic |
| | adenocarcinoma |
| MSLN | Pancreatic cancer |
| MSLN | Adenocarcinoma |
| MSLN | Gastric cancer |
| MSLN | Advanced cancer |
| MSLN | Fallopian tube cancer |
| MSLN | Solid tumor |
| MSLN | Triple-negative breast |
| | cancer |
| MSLN | Breast cancer |
| MSLN | Refractory B-cell |
| | lymphoma |
| MSLN | Follicular lymphoma |
| MSLN | Ovarian |
| | adenocarcinoma |
| MSLN | Ovarian epithelial |
| | cancer |
| MSLN | Ovarian epithelial |
| | cancer |
| MSLN | Ovarian serous |
| | adenocarcinoma |
| MSLN | High-grade serous |
| | ovarian |
| | adenocarcinoma |
| MSLN | Ovarian cancer |
| MSLN | Ovarian cancer |
| MSLN | Colorectal cancer |
| MSLN | Mesothelioma |
| MSLN | Mesothelioma |
| MSLN | Malignant peritoneal |
| | mesothelioma |
| MSLN | Lung cancer |
| MSLN | Non-small cell lung |
| | cancer |
| MSLN | Malignant pleural |
| | mesothelioma |
| MSLN | Cholangiocarcinoma |
| MSLN | Cholangiocarcinoma |
| MSLN | Diffuse large B-cell |
| | lymphoma |
| MSLN | Mesothelin-expressing |
| | tumor |
| MSLN | Mesothelin-expressing |
| | solid tumor |
| MSLN, MUC16 | Pancreatic acinar cell |
| | carcinoma |
| MSLN, MUC16 | Ovarian epithelial |
| | cancer |
| MSLN, PD-1 | Advanced malignant |
| | solid tumor |
| MSLN, PD-1 | Breast cancer |
| MSLN, PD-1 | Mesothelioma |
| MSLN, PD-1 | Mesothelioma |
| MSLN, PD-1 | Mesothelin-expressing |
| | solid tumor |
| MSLN, PDL1 | Ovarian cancer |
| MSP-1 | Malaria |
| MSTN | Muscle atrophy |
| MSTN | Duchenne muscular |
| | dystrophy |
| MSTN | Inclusion body myositis |
| MTM1 | Congenital myopathy |
| MTM1 | Rheumatoid arthritis |
| MTM1 | Muscle disorders |
| MT-ND1 | Leber Hereditary Optic |
| | Neuropathy, ND1 |
| | mutation |
| MT-ND4 | Leber Hereditary Optic |
| | Neuropathy, ND4 |
| | mutation |
| mTOR | Tumor |
| mTOR | Hemophilia A |
| mTOR | Diabetic retinopathy |
| mTOR | Retinal disease |
| mTOR | Methylmalonic acidemia |
| MUC1 | Metastatic solid tumor |
| MUC1 | Metastatic breast |
| | cancer |
| MUC1 | Pancreatic acinar cell |
| | carcinoma |
| MUC1 | Pancreatic ductal |
| | carcinoma |
| MUC1 | Pancreatic cancer |
| MUC1 | Gastric cancer |
| MUC1 | Advanced gastric |
| | cancer |
| MUC1 | Advanced malignant |
| | solid tumor |
| MUC1 | Head and neck |
| | squamous cell |
| | carcinoma |
| MUC1 | Fallopian tube cancer |
| MUC1 | Esophageal cancer |
| MUC1 | Renal cell carcinoma |
| MUC1 | Breast cancer |
| MUC1 | Prostate cancer |
| MUC1 | Brain malignant glioma |
| MUC1 | Ovarian epithelial |
| | carcinoma |
| MUC1 | Ovarian cancer |
| MUC1 | Colorectal cancer |
| MUC1 | Glioma |
| MUC1 | Hepatocellular |
| | carcinoma |
| MUC1 | Intrahepatic |
| | cholangiocarcinoma |
| MUC1 | Lung cancer |
| MUC1 | Non-small cell lung |
| | cancer |
| MUC1 | Non-small cell lung |
| | cancer |
| MUC1 | Multiple myeloma |
| MUC1 | Nasopharyngeal tumor |
| MUC1 | MUC1-expressing solid |
| | tumor |
| MUC16 | Breast cancer |
| MUC16 | Ovarian epithelial |
| | carcinoma |
| Mucin | Tumor |
| Mucin | Asthma |
| Mucin | Chronic obstructive |
| | pulmonary disease |
| MUSK | Myasthenia gravis |
| MUT | Methylmalonic acidemia |
| MUT, mTOR | Mesothelioma |
| MYB | Hematologic |
| | malignancy |
| MYB | Chronic myeloid |
| | leukemia |
| MYB | Leukemia |
| MYBPC3 | Hypertrophic |
| | cardiomyopathy |
| MYO6 | Hearing impairment |
| MYO7A | Usher syndrome |
| MYO7A | Usher syndrome type |
| | 1B |
| NaCT | Epilepsy |
| NAGA | Fabry disease |
| NAGLU | Lower urinary tract |
| | symptom |
| NAGLU | Mucopolysaccharidosis |
| | type III |
| Nav1.1 | Mucopolysaccharidosis |
| | type l |
| Nav1.1 | Rett syndrome |
| Nav1.1 | Dravet syndrome |
| Nav1.1 | CDKL5 Deficiency |
| Nav1.2 | Genetic disease |
| Nav1.2 | Precocious puberty |
| Nav1.2 | Epileptic |
| | encephalopathy |
| Nav1.2 | SCN2A-related |
| | encephalopathy |
| Nav1.6 | Developmental and |
| | epileptic |
| | encephalopathy |
| Nav1.6 | CDKL5 deficiency |
| Nav1.7 | Small fiber neuropathy |
| Nav1.7 | Pain |
| Nav1.7 | Neuralgia |
| Nav1.7 | Erythromelalgia |
| Nav1.7, Nav1.8 | Pain |
| Nav1.7, | Neuralgia |
| Voltage-gated | |
| sodium | |
| channels | |
| Nav1.8 | Pain |
| Nav1.8, TRKA | Neurological disorders |
| NCALD | Spinal muscular atrophy |
| NCF1 | Chronic granulomatous |
| | disease |
| NCL | Tumor |
| NCL | Tumor |
| NCL | Triple-negative breast |
| | cancer |
| NCL | Myelodysplastic |
| | syndrome |
| NCOR2 | Tumor |
| NCR2, NKG2D | Advanced solid tumor |
| NCR3LG1 | Tumor |
| NCR3LG1 | Solid tumor |
| NECTIN2 | Tumor |
| Nectin-4 | Tumor |
| NEGF2 | Tumor |
| NEK2 | Pancreatic cancer |
| NEK2 | Cholangiocarcinoma |
| NF-κB | Atopic dermatitis |
| NF-κB | Sepsis |
| NF-κB | Acute kidney injury |
| NF-κB | Obstetric delivery |
| | (premature birth) |
| NGF | Hearing loss |
| NGF | Retinitis pigmentosa |
| NGF | Retinal disease |
| NGF | Neuralgia |
| NGF | Glaucoma |
| NGF | Parkinson's disease |
| NGF | Stroke |
| NGF | Keratitis |
| NGF | Spinal cord injury |
| NGF | Macular degeneration |
| NGF | Huntington's disease |
| NGF | Osteoporosis |
| NGF | Osteoarthritis |
| NGF | Alzheimer's disease |
| NGLY1 | Genetic disease |
| NGLY1 | NGLY1 deficiency |
| Nicotine | Tobacco dependence |
| NIS | Pancreatic ductal |
| | adenocarcinoma |
| NIS | Fallopian tube cancer |
| NIS | Breast cancer |
| NIS | Prostate cancer |
| NIS | Ovarian cancer |
| NIS | Squamous cell |
| | carcinoma |
| NIS | Mesothelioma |
| NIS | Rhabdomyosarcoma |
| NIS | Peritoneal cancer |
| NIS | Multiple myeloma |
| NIS | Bladder cancer |
| NK1 | Hypertonia |
| NK2R, mGluRs | Major depressive |
| | disorder |
| NKG2D | Metastatic solid tumor |
| NKG2D | Rectal cancer |
| NKG2D | Hematologic |
| | malignancy |
| NKG2D | Novel Coronavirus |
| | Infection |
| NKG2D | Medulloblastoma |
| NKG2D | Medulloblastoma |
| NKG2D | Refractory acute |
| | myeloid leukemia |
| NKG2D | Colorectal cancer |
| NKG2D | Colon cancer liver |
| | metastasis |
| NKG2D | Colon cancer |
| NKG2D | Glioblastoma |
| NKG2D | Glioblastoma |
| NKG2D | Acute myeloid leukemia |
| NKG2D | Acute myeloid leukemia |
| NKG2D | Myelodysplastic |
| | syndrome |
| NKG2D | Hepatocellular |
| | carcinoma |
| NKG2D | Relapsed acute myeloid |
| | leukemia |
| NKG2D | Unresectable colorectal |
| | cancer |
| NKG2D | NKG2DL-positive solid |
| | tumor |
| NLGase | Gaucher disease |
| NLRP1, NLRP3 | Neurodegenerative |
| | disease |
| NLRP3 | Inflammatory bowel |
| | disease |
| NLRP3 | Inflammation |
| NMNAT1 | Leber congenital |
| | amaurosis |
| N-Myc | Small cell lung cancer |
| N-Myc | Neuroblastoma |
| N-Myc | Soft tissue sarcoma |
| NOD2 | Crohn's disease |
| NOS | Head and neck tumor |
| NOS | Prostate cancer |
| NOS | Liver cancer |
| NOS | Pulmonary hypertension |
| | due to lung disease |
| NOS | Pulmonary arterial |
| | hypertension |
| NOS2 | Vascular restenosis |
| NOS2 | Pulmonary arterial |
| | hypertension |
| NOX | Chronic granulomatous |
| | disease |
| NOX1 | Chronic granulomatous |
| | disease |
| NPC1 | Niemann-Pick disease |
| | type C |
| NPRA | Tumor |
| NPRA | Immune system |
| | disorder |
| NPY receptor | Epilepsy |
| NR2E3 | Retinitis pigmentosa |
| NR2E3 | Leber congenital |
| | amaurosis |
| NR2F6 | Tumor |
| NR2F6 | Solid tumor |
| NR2F6 | Myelodysplastic |
| | syndrome |
| NR2F6 | Leukemia |
| NRARP | Wet age-related |
| | macular degeneration |
| NRARP | Wet macular |
| | degeneration |
| NRARP | Choroidal |
| | neovascularization |
| NRARP | Macular degeneration |
| NRAS | Tumor |
| NRAS | Hematologic |
| | malignancy |
| NRAS | Solid tumor |
| Nrf2 | Nonalcoholic |
| | steatohepatitis |
| Nrf2 | Arteriosclerosis |
| NRP-1 | Tumor |
| NRP-2 | Metastatic melanoma |
| NRP-2 | Tumor metastasis |
| NRP-2 | Tumor |
| NRP-2 | Kidney tumor |
| NRTN | Ocular disease |
| NRTN | Parkinson's disease |
| NRTN | Huntington's disease |
| NT-4 | Motor neurone disease |
| NT-4 | Hearing impairment |
| NT-4 | Diabetic retinopathy |
| NT-4 | Glaucoma |
| NT-4 | Uveitis |
| NT-4 | Parkinson's disease |
| NT-4 | Huntington's disease |
| NTCP | Liver disease |
| NTCP | Cholestasis |
| NTF3 | Charcot-Marie-Tooth |
| | Disease |
| Nucleic acids | Duchenne muscular |
| | dystrophy |
| NXNL1 | Cone-rod dystrophy |
| NXNL1 | Retinitis pigmentosa |
| NY-ESO-1 | Metastatic solid tumor |
| NY-ESO-1 | Tumor |
| NY-ESO-1 | Myxoid liposarcoma |
| NY-ESO-1 | Advanced solid |
| | malignant tumor |
| NY-ESO-1 | Esophageal squamous |
| | cell carcinoma |
| NY-ESO-1 | Esophageal cancer |
| NY-ESO-1 | Esophageal cancer |
| NY-ESO-1 | Solid tumor |
| NY-ESO-1 | Solid tumor |
| NY-ESO-1 | Neuroblastoma |
| NY-ESO-1 | Triple-negative breast |
| | cancer |
| NY-ESO-1 | Soft tissue sarcoma |
| NY-ESO-1 | Breast cancer |
| NY-ESO-1 | Breast cancer |
| NY-ESO-1 | Sarcoma |
| NY-ESO-1 | Meningioma |
| NY-ESO-1 | Refractory ovarian |
| | cancer |
| NY-ESO-1 | Epithelial ovarian |
| | carcinoma |
| NY-ESO-1 | Ovarian cancer |
| NY-ESO-1 | Synovial sarcoma |
| NY-ESO-1 | Synovial sarcoma |
| NY-ESO-1 | Melanoma |
| NY-ESO-1 | Hepatocellular |
| | carcinoma |
| NY-ESO-1 | Liver cancer |
| NY-ESO-1 | Recurrent primary |
| | peritoneal cancer |
| NY-ESO-1 | Lung cancer |
| NY-ESO-1 | Non-small cell lung |
| | cancer |
| NY-ESO-1 | Multiple myeloma |
| NY-ESO-1 | Platinum-resistant |
| | fallopian tube cancer |
| NY-ESO-1 | Platinum-resistant |
| | ovarian cancer |
| NY-ESO-1 | Platinum-sensitive |
| | primary peritoneal |
| | carcinoma |
| NY-ESO-1 | Platinum-sensitive |
| | fallopian tube cancer |
| NY-ESO-1 | Platinum-resistant |
| | primary peritoneal |
| | cancer |
| NY-ESO-1 | Urothelial bladder |
| | cancer |
| NY-ESO-1 | Bladder cancer |
| NY-ESO-1 | FGF 19-positive solid |
| | tumor |
| NY-ESO-1, PD-1 | Myxoid liposarcoma |
| NY-ESO-1, PD-1 | Synovial sarcoma |
| NY-ESO-1, PD-1 | Melanoma |
| NY-ESO-1, PD-1 | Multiple myeloma |
| NY-ESO-1, | Acute myeloid leukemia |
| PRAME, WT1 | |
| NY-ESO-1, | Acute lymphoblastic |
| PRAME, WT1 | leukemia |
| NY-ESO-1, | Solid tumor |
| TCR | |
| NY-ESO-1, | Myxoid liposarcoma |
| TGF-ß | |
| NY-ESO-1, | Synovial sarcoma |
| TGF-B | |
| OPA1 | Optic atrophy |
| OPA1 | Autosomal dominant |
| | optic atrophy |
| OPCML | Ovarian cancer |
| OTCase | Urea cycle disorder |
| OTCase | Ornithine |
| | transcarbamylase |
| | deficiency |
| OTOF | Hearing loss |
| OTOF | Deafness |
| OX40 | Metastatic colorectal |
| | cancer |
| OX40 | Gastrointestinal stromal |
| | tumor |
| OX40 | Gastric cancer |
| OX40 | Kidney tumor |
| OX40 | Breast cancer |
| OX40 | Sarcoma |
| OX40 | Squamous cell |
| | carcinoma |
| OX40 | Colorectal cancer liver |
| | metastasis |
| OX40 | Melanoma |
| OX40 | Liver metastasis |
| OX40 | Abdominal tumor |
| OX40L | Lymphoma |
| p24 antigen | HIV infection |
| p53 | Delayed recovery of |
| | graft function |
| p53 | Transplant complication |
| p53 | Pancreatic cancer |
| p53 | Small lymphocytic |
| | lymphoma |
| p53 | Advanced solid |
| | malignant tumor |
| p53 | Advanced bile duct |
| | cancer |
| p53 | Head and neck tumor |
| p53 | Fallopian tube cancer |
| p53 | Leukoplakia |
| p53 | Chronic lymphocytic |
| | leukemia |
| p53 | Ovarian cancer |
| p53 | Glioblastoma |
| p53 | Acute myeloid leukemia |
| p53 | Acute kidney injury |
| p53 | Myelodysplastic |
| | syndrome |
| p53 | Liver cancer |
| p53 | Peritoneal cancer |
| p53 | Non-small cell lung |
| | cancer |
| p53 | Malignant ascites |
| p53 | Nasopharyngeal |
| | carcinoma |
| p53 | Bladder cancer |
| p53 | Vitiligo |
| p53 R175H | Metastatic solid tumor |
| p53 R175H | Pancreatic |
| | adenocarcinoma |
| p53 R175H | Head and Neck |
| | Squamous Cell |
| | Carcinoma |
| p53 R175H | Breast cancer |
| p53 R175H | Colorectal cancer |
| p53 R175H | Non-small cell lung |
| | cancer |
| p75NTR | Alzheimer's disease |
| PA protein, | Influenza virus infection |
| influenza virus | |
| M1 | |
| PABPN1 | Oculopharyngeal |
| | muscular dystrophy |
| PABPN1 | Swallowing disorders |
| PAH | Phenylketonuria |
| PAI-1, tPA, | Thrombotic |
| VWF | microangiopathy |
| PAI-1, tPA, | Novel Coronavirus |
| VWF | Infection |
| PAI-1, tPA, | Veno-occlusive disease |
| VWF | |
| PAI-1, tPA, | High-risk neuroblastoma |
| VWF | |
| PAI-1, tPA, | Hepatic veno-occlusive |
| VWF | disease |
| PAK4 | Non-muscle invasive |
| | bladder tumor |
| PAK4 | Bladder cancer |
| PARP | Tumor |
| PARP | Solid tumor |
| PARP1 | Tumor |
| PAX4 | Type 1 diabetes |
| PBGD | Acute intermittent |
| | porphyria |
| PCSK9 | Lipid metabolism |
| | disorder |
| PCSK9 | Heterozygous familial |
| | hypercholesterolemia |
| PCSK9 | Primary |
| | hypercholesterolemia |
| PCSK9 | Hereditary |
| | Leiomyomatosis and |
| | Renal Cell Carcinoma |
| PCSK9 | Peripheral arterial |
| | disease |
| PCSK9 | Stroke |
| PCSK9 | Familial combined |
| | hyperlipidemia |
| PCSK9 | Coronary artery disease |
| PCSK9 | Hyperlipidemia |
| PCSK9 | hypertension |
| PCSK9 | Hypercholesterolemia |
| PCSK9 | Liver disease |
| PCSK9 | Atherosclerosis |
| PCSK9 | Homozygous familial |
| | hypercholesterolemia |
| PCSK9 | Type lla |
| | hyperlipoproteinemia |
| PD-1 | Metastatic melanoma |
| PD-1 | Metastatic non-small |
| | cell lung cancer |
| PD-1 | Advanced non-small cell |
| | lung cancer |
| PD-1 | Advanced solid |
| | malignant tumor |
| PD-1 | Esophageal cancer |
| PD-1 | Triple-negative breast |
| | cancer |
| PD-1 | Skin tumor |
| PD-1 | Squamous cell |
| | carcinoma of the skin |
| PD-1 | Skin melanoma |
| PD-1 | Brain metastases |
| PD-1 | Refractory cancer |
| PD-1 | Urinary system tumor |
| PD-1 | Merkel cell carcinoma |
| PD-1 | Ovarian cancer |
| PD-1 | Colorectal cancer |
| PD-1 | Glioma |
| PD-1 | Mesothelioma |
| PD-1 | Melanoma |
| PD-1 | Hepatocellular |
| | carcinoma |
| PD-1 | Non-small cell lung |
| | cancer stage III |
| PD-1 | Non-small cell lung |
| | cancer |
| PD-1 | Glioblastoma multiforme |
| PD-1 | Unresectable melanoma |
| PD-1 | Mesothelin-positive |
| | tumor |
| PD-1 | Mesothelin-positive |
| | solid tumor |
| PD-1 | IV Malignant Non-Small |
| | Cell Lung Tumor |
| PD-1 | Stage III colon cancer |
| PD-1 | Stage IIA non-small cell |
| | lung cancer |
| PD-1 | Carney complex |
| PD-1, PSCA, | Prostate cancer |
| PSMA | |
| PDCD5 | Leukemia |
| PDE4B, | Chronic obstructive |
| PDE4D, PDE7A | pulmonary disease |
| PDGFB | diabetic foot |
| PDGFB, | Macular degeneration |
| VEGFR | |
| PDGFR | Tumor |
| PDGFR | Ocular disease |
| PDGFR | Fibrosis |
| PDGFR | Hippel-Lindau syndrome |
| PDGFR | Retinoblastoma |
| PDGFR | Wet age-related |
| | macular degeneration |
| PDGFR | Wet macular |
| | degeneration |
| PDGFR | Macular degeneration |
| PDGFR, | Wet age-related |
| VEGFR1 | macular Degeneration |
| PDGFRα | Glioma |
| PDGFRβ | Cirrhosis |
| PDHK1 | Allergic conjunctivitis |
| PDL1 | Autoimmune hepatitis |
| PDL1 | Metastatic head and |
| | neck squamous cell |
| | carcinoma |
| PDL1 | Tumor metastasis |
| PDL1 | Tumor |
| PDL1 | Hepatitis B |
| PDL1 | Graft-versus-host |
| | disease |
| PDL1 | Pancreatic ductal |
| | adenocarcinoma |
| PDL1 | Pancreatic cancer |
| PDL1 | Gastric cancer |
| PDL1 | Triple-negative breast |
| | cancer |
| PDL1 | Ovarian epithelial |
| | carcinoma |
| PDL1 | Locally advanced |
| | malignant solid tumor |
| PDL1 | Glioblastoma |
| PDL1 | Hodgkin lymphoma |
| PDL1 | Melanoma |
| PDL1 | Peritoneal cancer |
| PDL1 | Recurrent squamous |
| | cell carcinoma of the |
| | head and neck |
| PDL1, TLR9 | Tumor |
| PDL1, VEGFR1 | Tumor |
| PDL1, VEGFR1 | Malignant ascites |
| PECAM1 | Solid tumor |
| PECAM1 | Lung damage |
| PEDF | Retinitis pigmentosa |
| PfCSP | Plasmodium falciparum |
| PGF, VEGF-A, | Diabetic macular edema |
| VEGF-B, | |
| VEGF-C | |
| PGF, VEGF-A, | Wet age-related |
| VEGF-B, | macular degeneration |
| VEGF-C | |
| P-gp | Breast cancer |
| P-gp | Non-Hodgkin lymphoma |
| P-gp | Bladder cancer |
| PGRN | Frontotemporal |
| | dementia |
| PhD2 | Anemia |
| PhD2 | Acute kidney injury |
| Phosphotransfe | Restenosis |
| rases | |
| Phosphotransfe | Tumor |
| rases, p38α | |
| Phosphotransfe | Restenosis |
| rases, p38α | |
| PI3K family, | Solid tumor |
| mTORC1, | |
| mTORC2 | |
| PI3Kα | Tumor |
| PI3Kα H1047R | Tumor |
| PI3Kα H1047R | Advanced breast cancer |
| PI3Kα H1047R | Advanced solid |
| | malignant tumor |
| PI3Kα H1047R | Advanced cancer |
| PI3Kα H1047R | Breast cancer |
| PIGF, VEGF-A, | Wet macular |
| VEGF-B | degeneration |
| PIKFYVE | Amyotrophic lateral |
| | sclerosis |
| PIKFYVE | Frontotemporal |
| | dementia |
| PIWIL1 | Pancreatic cancer |
| PIWIL1 | Gastrointestinal tumor |
| PIWIL1 | Colorectal cancer |
| PKA | Tumor |
| PKA | Solid tumor |
| PKA | Breast cancer |
| PKA | Ovarian cancer |
| PKA | Colon cancer |
| PKA | Lung cancer |
| PKCa | Lung cancer |
| PKCa | Non-small cell lung |
| | cancer |
| PKLR | Erythrocyte pyruvate |
| | kinase deficiency |
| PKN3 | Pancreatic cancer |
| PKN3 | Advanced solid |
| | malignant tumor |
| PKN3 | Head and neck tumor |
| PLA2 | Tumor |
| PLA2G6 | Neuroaxonal dystrophy |
| PLA2R | Membranous |
| | Glomerulonephritis |
| plakophilin-2 | Arrhythmic right |
| | ventricular dysplasia |
| plakophilin-2 | Myocardial disease |
| PLK1 | Advanced |
| | hepatocellular |
| | carcinoma |
| PLK1 | Advanced solid |
| | malignant tumor |
| PLK1 | Adrenal cortical |
| | carcinoma |
| PLK1 | Neuroendocrine tumor |
| PLK1 | Lymphoma |
| PLK1 | Hepatocellular |
| | carcinoma |
| PLK4 | Solid tumor |
| PLK4 | Breast cancer |
| PLP1 | Pelizaeus-Merzbacher |
| | disease |
| PMP22 | Charcot-Marie-Tooth |
| | Disease Type la |
| PMVK | Pancreatic cancer |
| PMVK | Non-small cell lung |
| | cancer |
| PNP | Pharyngeal tumor |
| PNP | Advanced Head and |
| | neck tumor |
| PNP | Head and neck tumor |
| PNP | Prostate cancer |
| PNP | Squamous cell |
| | carcinoma |
| PNPLA3 | Fatty liver |
| PNPLA3 | Cardiovascular disease |
| PNPLA3 | Fibrosis |
| PNPLA3 | hepatitis |
| PNPLA3 | Nonalcoholic |
| | steatohepatitis |
| PNPLA3 | Metabolic disease |
| PNPLA3 | Metabolic fatty liver |
| | disease |
| POSTN | Diabetic retinopathy |
| Potassium | Urge incontinence |
| channel | |
| Potassium | Overactive bladder |
| channel | |
| PPAR | Acne vulgaris |
| PPT1 | Neuronal ceroid |
| | lipofuscinosis |
| PR1 | Acute myeloid leukemia |
| | in adults |
| PRAME | Endometrial cancer |
| PRAME | Tumor |
| PRAME | Head and neck tumor |
| PRAME | Solid tumor |
| PRAME | Uveal melanoma |
| PRAME | Skin melanoma |
| PRAME | Ovarian cancer |
| PRAME | Acute myeloid leukemia |
| PRAME | Synovial sarcoma |
| PRAME | Melanoma |
| PRAME | Myelodysplastic |
| | syndrome |
| PRAME | Myelodysplastic |
| | syndrome |
| PRAME | Non-small cell lung |
| | cancer |
| PRAME, WT1 | Prostate cancer |
| PRAME, WT1, | Acute myeloid leukemia |
| cyclin A1 | |
| PRAME, WT1, | Myelodysplastic |
| cyclin A1 | syndrome |
| PRDM2 | Prostate cancer |
| PRDM2 | Liver cancer |
| PRDM2 | Non-small cell lung |
| | cancer |
| PRG4 | Osteoarthritis |
| PRNP | Prion disease |
| Protein kinases | Tumor |
| PRPF31 | Retinal degenerative |
| | disease associated with |
| | biallelic RPE65 |
| | mutations |
| PRPF31 | Retinitis pigmentosa |
| | type 11 |
| PSCA | Esophageal |
| | adenocarcinoma |
| PSCA | Bladder cancer |
| P-sel | Sickle cell disease |
| PSMA | Autoimmune disease |
| PSMA | Metastatic castration- |
| | resistant prostate |
| | cancer |
| PSMA | Tumor |
| PSMA | Mucoepidermoid |
| | carcinoma |
| PSMA | Adenoid cystic |
| | carcinoma |
| PSMA | Acinar cell carcinoma |
| PSMA | Salivary gland cancer |
| PSMA | Salivary gland tumor |
| PSMA | Renal cell carcinoma |
| PSMA | Breast cancer |
| PSMA | Castration-resistant |
| | prostate cancer |
| PSMA | Castration-resistant |
| | prostate cancer |
| PSMA | Prostate cancer |
| PSMA | Prostate cancer |
| PSMA | Colorectal cancer |
| PSMA | Non-small cell lung |
| | cancer |
| PSMA | Multiple myeloma |
| PSMA | Ductal carcinoma |
| PSMA | PSMA-positive tumor |
| PSMA, | Metastatic castration- |
| TCRDV2 | resistant prostate |
| | cancer |
| PSMA, TGF-β | Castration-resistant |
| | prostate cancer |
| PSMA, TGF-β | Prostate cancer |
| PSMA, Trop-2 | Prostate cancer |
| PTEN | Severe Acute |
| | Respiratory Syndrome |
| PTEN | Spinal cord injury |
| PTEN | Acute spinal cord injury |
| PTH | Postmenopausal |
| | osteoporosis |
| PTK7 | Colorectal cancer |
| PTP1B | obesity |
| PTP1B | Type 2 diabetes |
| PYK2 | Ovarian cancer |
| PYK2 | Colorectal cancer |
| PYK2 | Thyroid cancer |
| Raf kinase | Head and neck tumor |
| Raf kinase | Breast cancer |
| RAG-1 | Glioblastoma |
| RANGRF | Brugada syndrome |
| Ras | Tumor |
| Ras | Tumor |
| Ras | Pancreatic acinar |
| | carcinoma |
| Ras | Secondary malignant |
| | tumor of the pancreas |
| Ras | Advanced colorectal |
| | adenocarcinoma |
| Ras | Solid tumor |
| Ras | Breast cancer |
| Ras | Locally advanced |
| | pancreatic |
| | adenocarcinoma |
| Ras | Colorectal cancer |
| Ras | Recurrent colorectal |
| | cancer |
| Ras | Non-small cell lung |
| | cancer |
| RB1 | Pancreatic cancer |
| RB1 | Head and neck tumor |
| RB1 | Mesothelioma |
| RB1 | Lung cancer |
| RDH12 | Leber congenital |
| | amaurosis |
| RdRp | Novel Coronavirus |
| | Infection |
| Redd1 | Diabetic macular edema |
| Redd1 | Macular degeneration |
| regnase-1 | Fibrosis |
| regnase-1 | Adult respiratory |
| | distress syndrome |
| RELA | Rheumatoid arthritis |
| RELA | Osteoarthritis |
| renin receptor | Diabetic retinopathy |
| renin receptor | Uveitis |
| renin receptor | Macular degeneration |
| REP1 | Choroideremia |
| REP1 | Retinal dysplasia |
| REP1 | X-linked retinitis |
| | pigmentosa |
| REV | HIV infection |
| RHO | Retinitis pigmentosa |
| | type 4 |
| RHO | Retinitis pigmentosa |
| RHO | Color vision impairment |
| RHO | Macular degeneration |
| RHO | Geographic atrophy |
| RHOK | Retinal disease |
| RIG-I | Tumor |
| RIG-I | Melanoma |
| RIG-I | Coronavirus infection |
| RIG-I, TLR3 | Viral infection |
| RLBP1 | Retinitis pigmentosa |
| RNAP | Hepatitis B |
| RNA binding | Metastatic breast |
| proteins | cancer |
| RNR | Metastatic solid tumor |
| RNR | Tumor |
| RNR | Rectal cancer |
| RNR | Primitive |
| | neuroectodermal tumor |
| RNR | Solid tumor |
| RNR | Kidney tumor |
| RNR | Renal cell carcinoma |
| RNR | Breast cancer |
| RNR | Prostate cancer |
| RNR | Prostate cancer |
| RNR | Refractory |
| | undifferentiated acute |
| | leukemia |
| RNR | Supratentorial tumor |
| RNR | Chronic myeloid |
| | leukemia |
| RNR | Lymphoma |
| RNR | Locally advanced non- |
| | small cell lung cancer |
| RNR | Colorectal cancer |
| RNR | Glioblastoma |
| RNR | Secondary |
| | myelodysplastic |
| | syndrome |
| RNR | Chronic myeloid |
| | leukemia in acute |
| | transformation |
| RNR | Acute promyelocytic |
| | leukemia |
| RNR | Acute leukemia |
| RNR | Myelodysplastic |
| | syndrome |
| RNR | Recurrent prostate |
| | cancer |
| RNR | Recurrent colon cancer |
| RNR | Relapsed acute myeloid |
| | leukemia |
| RNR | Recurrent non-small cell |
| | lung cancer |
| RNR | Relapsed adult acute |
| | lymphoblastic leukemia |
| RNR | Non-small cell lung |
| | cancer stage IIIB |
| RNR | Non-small cell lung |
| | cancer stage IIIA |
| RNR | Non-small cell lung |
| | cancer |
| RNR | Acute myeloid leukemia |
| | in adults |
| RNR | Adult acute |
| | lymphoblastic leukemia |
| RNR | Bladder cancer |
| RNR | Acute myeloid leukemia |
| | with 11q23 |
| | abnormalities |
| RNR | IV malignant non-small |
| | cell lung tumor |
| ROBO1 | Tumor |
| ROBO1 | Pancreatic cancer |
| ROBO1 | Solid tumor |
| ROCK | Tumor |
| ROCK | Retinitis pigmentosa |
| ROCK | Corneal edema |
| ROCK | Corneal endotheliitis |
| ROCK | Bullous keratopathy |
| ROCK | Fuchs endothelial |
| | dystrophy |
| ROR1 | Metastatic non-small |
| | cell lung cancer |
| ROR1 | Hematologic |
| | malignancy |
| ROR1 | Advanced breast cancer |
| ROR1 | Mantle cell lymphoma |
| ROR1 | Solid tumor |
| ROR1 | Triple-negative breast |
| | cancer |
| ROR1 | Breast cancer |
| ROR1 | Breast cancer |
| ROR1 | Refractory malignant |
| | solid tumor |
| ROR1 | Diffuse large B-cell |
| | lymphoma |
| ROR1 | Chronic lymphocytic |
| | leukemia |
| ROR1 | Ovarian cancer |
| ROR1 | Acute lymphoblastic |
| | leukemia |
| ROR1 | Recurrent breast cancer |
| ROR1 | Recurrent non-small cell |
| | lung cancer |
| ROR1 | Non-Hodgkin lymphoma |
| ROR1 | Leukemia |
| ROR1 | B-cell malignancy |
| ROR2 | Pancreatic cancer |
| ROR2 | Gastric cancer |
| ROR2 | Kidney tumor |
| ROR2 | Renal cell carcinoma |
| ROR2 | Sarcoma |
| ROR2 | Recurrent solid tumor |
| ROR2 | Bladder cancer |
| RORG | Immune system |
| | disorders |
| RORα | Stargardt disease |
| RORα | Retinitis pigmentosa |
| | type 19 |
| RORα | Dry age-related macular |
| | degeneration |
| RORα | Geographic atrophy |
| RORα | Cone-rod dystrophy |
| | type 3 |
| RPE65 | Leber congenital |
| | amaurosis |
| RPE65 | Leber's hereditary optic |
| | atrophy |
| RPGR | Choroideremia |
| RPGR | Retinoschisis |
| RPGR | X-linked retinitis |
| | pigmentosa |
| RPN2 | Breast cancer |
| RRM2 | Tumor |
| RRM2 | Solid tumor |
| RT | Lymphoma |
| RT | HIV infection |
| RTK | Ovarian cancer |
| RUNX3 | Tumor |
| RYR2 | Catecholaminergic |
| | polymorphic ventricular |
| | tachycardia |
| SANS | Usher syndrome |
| SARM1 | Peripheral nervous |
| | system disease |
| SARS-COV-2 | Novel Coronavirus |
| 3CLpro | Infection |
| SARS-COV-2 N | Novel Coronavirus |
| protein, SARS- | Infection |
| CoV-2 ORF1Ab | |
| SARS-COV-2 S | Novel Coronavirus |
| protein | Infection |
| SARS-COV-2 S | Coronavirus infection |
| protein | |
| SATB1 | Prostate cancer |
| SCAP | Dyslipidemia |
| SCAP | Metabolic fatty liver |
| | disease |
| SDF-1 | Hematopoietic stem cell |
| | transplantation |
| SDF-1 | Pancreatic cancer |
| SDF-1 | Heart damage |
| SDF-1 | Diabetic retinopathy |
| SDF-1 | Chronic lymphocytic |
| | leukemia |
| SDF-1 | Colorectal cancer |
| SDF-1 | Relapsed chronic |
| | lymphocytic leukemia |
| SDF-1 | Multiple myeloma |
| Selectins | Inflammation |
| Selectins | Colon cancer |
| Selectins | Melanoma |
| SEMA4A | Multiple myeloma |
| SERCA2 | Heart failure |
| SERCA2 | Diastolic heart failure |
| SERCA2 | Heart failure with |
| | preserved ejection |
| | fraction |
| SERCA2 | Ischemic |
| | cardiomyopathy |
| SERCA2 | Urinary incontinence |
| SERCA2 | Dilated cardiomyopathy |
| SERCA2 | Pulmonary hypertension |
| SERCA2 | Nonischemic |
| | cardiomyopathy |
| SERCA2 | Duchenne muscular |
| | dystrophy |
| SERCA2 | DMD-related dilated |
| | cardiomyopathy |
| SGCA | Limb-girdle muscular |
| | dystrophy |
| SGCA | Sarcoglycanosis |
| SGCB | Limb-girdle muscular |
| | dystrophy |
| SGCB | 2e type of muscular |
| | dystrophy |
| SGCG | Limb-girdle muscular |
| | dystrophy R5 |
| SGCG | Limb-girdle muscular |
| | dystrophy type 2C |
| SGLT2 | Type 2 diabetes |
| SGSH | Mucopolysaccharidosis |
| | type III |
| SGSH, SUMF1 | Mucopolysaccharidosis |
| | type III |
| SHANK3 | Autism Spectrum |
| | Disorders |
| SHANK3 | Phelan-McDermid |
| | syndrome |
| SIRPα | Tumor |
| SIRT6 | Nonalcoholic |
| | steatohepatitis |
| SLAMF7 | Hematologic |
| | malignancy |
| SLAMF7 | Multiple myeloma |
| SLC6A8 | Duchenne muscular |
| | dystrophy |
| SMAC | Pancreatic cancer |
| SMAC, TRAIL | Hepatocellular |
| | carcinoma |
| SMAD2, | Choroidal |
| TGFBR2, | neovascularization |
| VEGF-A | |
| SMAD4 | Colorectal cancer |
| SMAD7 | Crohn's disease |
| SMAD7 | Cachexia |
| SMAD7 | Duchenne muscular |
| | dystrophy |
| SMAD7 | Inclusion body myositis |
| SMN1 | Spinal muscular atrophy |
| SMN1 | Muscular dystrophy |
| SMN1 | Spinal muscular atrophy |
| | type l |
| SMN2 | Infantile-onset |
| | spinocerebellar ataxia |
| SMN2 | Spinal muscular atrophy |
| SMN2 exon 7 | Spinal muscular atrophy |
| SMPD1 | Niemann-Pick disease |
| SNAP25 | Primary axillary |
| | hyperhidrosis |
| SOCS1 | Advanced solid |
| | malignant tumor |
| SOCS1 | Head and neck tumor |
| SOCS1 | Head and Neck |
| | Squamous Cell |
| | Carcinoma |
| SOCS1 | Melanoma |
| SOCS1 | Lung cancer |
| SOCS1 | Non-small cell lung |
| | cancer |
| SOCS3 | Hepatocellular |
| | carcinoma |
| SOD1 | Motor neurone disease |
| SOD1 | Motor neurone disease |
| SOD1 | Amyotrophic lateral |
| | sclerosis |
| SOD1 | Amyotrophic lateral |
| | sclerosis type 1 |
| SOS1 | Lung cancer |
| SOST | Tumor |
| SOST | Triple-negative breast |
| | cancer |
| SOST | Familial |
| | hypophosphatemic |
| | rickets |
| SOST | Osteoporosis |
| SOST | Osteogenesis |
| | imperfecta |
| SOST | Type 2 diabetes |
| SPAG9 | Prostate cancer |
| SPAT | Primary hyperoxaluria |
| SPHK1 | Hepatocellular |
| | carcinoma |
| SPSB1 | Non-small cell lung |
| | cancer |
| SRY | Parkinson's disease |
| SSTR, | Ovarian cancer |
| thymidine | |
| kinase | |
| SSTR, | Recurrent ovarian |
| thymidine | cancer |
| kinase | |
| SSTR2 | Melanoma |
| ST13 | Colorectal cancer |
| ST13, TRAIL | Pancreatic ductal |
| | carcinoma |
| STAT1 | Asthma |
| STAT3 | Metastatic head and |
| | neck squamous cell |
| | carcinoma |
| STAT3 | Hereditary nonpolyposis |
| | colorectal cancer type 1 |
| STAT3 | Pancreatic acinar |
| | carcinoma |
| STAT3 | Advanced colorectal |
| | adenocarcinoma |
| STAT3 | Advanced non-small cell |
| | lung cancer |
| STAT3 | Advanced solid |
| | malignant tumor |
| STAT3 | Head and neck tumor |
| STAT3 | Kidney tumor |
| STAT3 | Adrenoleukodystrophy |
| STAT3 | Breast cancer |
| STAT3 | Prostate cancer |
| | metastasis |
| STAT3 | Refractory colorectal |
| | cancer |
| STAT3 | Refractory lung cancer |
| STAT3 | Ovarian cysts |
| STAT3 | Glioma |
| STAT3 | Acute myeloid leukemia |
| STAT3 | Myelodysplastic |
| | syndrome |
| STAT3 | Hepatocellular |
| | carcinoma |
| STAT3 | Recurrent squamous |
| | cell carcinoma of the |
| | head and neck |
| STAT3 | Non-small cell lung |
| | cancer |
| STAT3 | Bladder cancer |
| STAT3 | MSI-H Colorectal |
| | Cancer |
| STAT3, mTOR | Pancreatic cancer |
| STAT3, mTOR | Bladder cancer |
| STAT3, TLR9 | Tumor |
| STAT3, TLR9 | Acute myeloid leukemia |
| STAT3, TLR9 | B-cell lymphoma |
| STAT5 | Breast cancer |
| transcription | |
| factor | |
| STAT6 | Pancreatic acinar |
| | carcinoma |
| STAT6 | Gastric cancer |
| STAT6 | Advanced |
| | hepatocellular |
| | carcinoma |
| STAT6 | Breast cancer |
| STAT6 | Uveal melanoma |
| STAT6 | Ovarian epithelial |
| | carcinoma |
| STAT6 | Colorectal cancer |
| STAT6 | Glioma |
| STAT6 | Thyroid cancer |
| STAT6 | Liver metastasis |
| STAT6 | Liver cancer |
| STAT6 | Lung cancer |
| STEAP1 | Metastatic castration- |
| | resistant prostate |
| | cancer |
| STEAP1 | Prostate cancer |
| | metastasis |
| STEAP2, | Prostate cancer |
| TGFBR2 | metastasis |
| STEAP2, | Prostate cancer |
| TGFBR2 | |
| STING | Liver cancer |
| STMN1 | Solid tumor |
| STMN2 | Amyotrophic lateral |
| | sclerosis |
| STMN2 | Frontotemporal |
| | dementia |
| STRC | Hearing impairment |
| STRIP1 | Solid tumor |
| STRIP1 | Glioma |
| STXBP1 | STXBP1-related |
| | epileptic |
| | encephalopathy |
| SULF2, TGF-β1 | Pancreatic acinar |
| | carcinoma |
| SULF2, TGF-β1 | Liver cancer |
| SULF2, TGF-β1 | Lung cancer |
| SUMO1 | Heart failure |
| SURF1 | Leigh disease |
| Survivin | Metastatic non-small |
| | cell lung cancer |
| Survivin | Tumor |
| Survivin | Advanced |
| | hepatocellular |
| | carcinoma |
| Survivin | Solid tumor |
| Survivin | Castration-resistant |
| | prostate cancer |
| Survivin | Precursor T-lymphocytic |
| | leukemia lymphoma |
| Survivin | Prostate cancer |
| Survivin | Lymphoma |
| Survivin | Locally advanced non- |
| | small cell lung cancer |
| Survivin | Glioblastoma |
| Survivin | Acute myeloid leukemia |
| Survivin | Acute lymphoblastic |
| | leukemia |
| Survivin | Hepatocellular |
| | carcinoma |
| Survivin | Relapsed adult acute |
| | lymphoblastic leukemia |
| Survivin | Non-small cell lung |
| | cancer |
| SYF2 | Amyotrophic lateral |
| | sclerosis |
| SYF2 | Frontotemporal |
| | dementia |
| Syk | Inflammation |
| Syk | Asthma |
| SYT11 | Gastric cancer |
| SYT11 | Colorectal cancer |
| SYT11 | Lung cancer |
| TAG-72 | Ovarian cancer |
| TAG-72 | Platinum-resistant |
| | Ovarian epithelial |
| | carcinoma |
| TAG-72 | T-cell lymphoma |
| TAU | Mild cognitive |
| | impairment |
| TAU | Pick's disease dementia |
| TAU | Progressive |
| | supranuclear palsy |
| TAU | Dementia due to |
| | Alzheimer's disease |
| TAU | Alzheimer's disease |
| TAU, α- | Neurodegenerative |
| synuclein | disease |
| TBX21 | Psoriasis |
| TBX21 | Chronic obstructive |
| | pulmonary disease |
| TBXA2R | Tumor |
| TBXA2R | Cardiovascular disease |
| TCF7L2 | Tumor |
| TCR | B-cell lymphoma |
| TCR, VCAM1 | Neuroblastoma |
| TDP43 | Amyotrophic lateral |
| | sclerosis |
| TDP43 | Frontotemporal |
| | dementia |
| TECPR2 | Spastic paraplegia |
| Telomerase | Gastroesophageal |
| | junction |
| | adenocarcinoma |
| Telomerase | Gastric cancer |
| Telomerase | Perioperative ischemia |
| Telomerase | Esophageal cancer |
| Telomerase | Glioblastoma |
| Telomerase | Melanoma |
| Telomerase | Liver cancer |
| Telomerase | Non-small cell lung |
| | cancer |
| TERC | Neutropenia |
| TERC | Juvenile |
| | myelomonocytic |
| | leukemia |
| TERC | Transfusion-dependent |
| | anemia |
| TERC | Acute myeloid leukemia |
| TERC | Myelodysplastic |
| | syndrome |
| TERC | Myelofibrosis |
| TERT | Advanced non-small cell |
| | lung cancer |
| TERT | Head and neck tumor |
| TERT | Glioblastoma |
| TERT | Melanoma |
| TERT | Hepatocellular |
| | carcinoma |
| TERT | Non-small cell lung |
| | cancer |
| TERT, c-Myc | Tumor |
| TERT, αvβ3, αvβ5 | Tumor |
| TERT, ανβ5 | Tumor |
| TFDP3 | Tumor |
| TFPI | Hemophilia B |
| TFPI | Hemophilia A |
| TFPI | hemophilia |
| TfR1 | Neuromuscular |
| | disorders |
| TfR1 | Duchenne muscular |
| | dystrophy |
| TG | Brain metastases |
| TGFBI | Hereditary corneal |
| | dystrophy |
| TGFBR2 | Tumor |
| TGFBR2 | Pulmonary fibrosis |
| TGF-β | Heart failure |
| TGF-β | Gastric cancer |
| TGF-β | Solid tumor |
| TGF-β | Chondrosarcoma |
| TGF-β | Relapsed plasma cell |
| | myeloma |
| TGF-β | Multiple myeloma |
| TGF-β | Infectious |
| | mononucleosis |
| TGF-β | Nasopharyngeal |
| | carcinoma |
| TGF-β | Niemann-Pick disease |
| | type C |
| TGF-β1 | Novel Coronavirus |
| | Infection |
| TGF-β1 | Idiopathic pulmonary |
| | fibrosis |
| TGF-β1 | Corneal disease |
| TGF-β1 | Basal cell carcinoma |
| TGF-β1 | Osteoarthritis |
| TGF-β1 | Liver cancer |
| TGF-β1, | Tumor |
| VEGFR2 | |
| TGF-β1, α-Klotho | Joint disease |
| TGF-β2 | Metastatic solid tumor |
| TGF-β2 | Metastatic non-small |
| | cell lung cancer |
| TGF-β2 | Tumor |
| TGF-β2 | Bronchogenic |
| | carcinoma |
| TGF-β2 | Pancreatic cancer |
| TGF-β2 | Advanced non-small cell |
| | lung cancer |
| TGF-β2 | Diabetic macular edema |
| TGF-β2 | Solid tumor |
| TGF-β2 | Wet macular |
| | degeneration |
| TGF-β2 | Renal cell carcinoma |
| TGF-β2 | Diffuse midline glioma |
| TGF-β2 | Diffuse intrinsic pontine |
| | glioma |
| TGF-β2 | Glioblastoma |
| TGF-β2 | Glioma |
| TGF-β2 | Melanoma |
| TGF-β2 | Non-small cell lung |
| | cancer |
| TGF-β2 | Malignant pleural |
| | mesothelioma |
| TGM1 | Autosomal recessive |
| | ichthyosis |
| TGM2 | Tumor |
| TGM2 | Immune system |
| | disorders |
| THBS1 | Pancreatic cancer |
| THBS1 | Lung cancer |
| THPO | Thrombocytopenia |
| thrombin | Thrombosis |
| thrombin | heart disease |
| Thymidine | Tumor |
| kinase | |
| Thymidine | Graft-versus-host |
| kinase | disease |
| Thymidine | Pancreatic |
| kinase | adenocarcinoma |
| Thymidine | Hematologic |
| kinase | malignancy |
| Thymidine | Gastric cancer |
| kinase | |
| Thymidine | Fallopian tube cancer |
| kinase | |
| Thymidine | Ovarian cancer |
| kinase | |
| Thymidine | Localized prostate |
| kinase | cancer |
| Thymidine | Colorectal cancer |
| kinase | |
| Thymidine | Glioma |
| kinase | |
| Thymidine | Melanoma |
| kinase | |
| Thymidine | Liver cancer |
| kinase | |
| Thymidine | Platinum-sensitive |
| kinase | primary peritoneal |
| | carcinoma |
| Thymidine | Leukemia |
| kinase | |
| Tie-2 | Diabetic retinopathy |
| TIGIT | Tumor |
| TLR | Hepatocellular |
| | carcinoma |
| TLR2 | Parkinson's disease |
| TLR3 | Breast cancer |
| TLR3 | Immune system |
| | disorders |
| TLR3 | Ovarian cancer |
| TLR4 | Stroke |
| TLR4 | Lymphatic |
| | malformations |
| TLR4 | Infect |
| TLR4 | Non-muscle invasive |
| | bladder tumor |
| TLR7 | Idiopathic pulmonary |
| | fibrosis |
| TLR7 | Non-small cell lung |
| | cancer |
| TLR7, TLR8 | Tumor |
| TLR7, TLR8 | Infect |
| TLR7, TLR8, | psoriasis |
| TLR9 | |
| TLR7, TLR8, | Dermatomyositis |
| TLR9 | |
| TLR7, TLR8, | Refractory diffuse large |
| TLR9 | B-cell lymphoma |
| TLR7, TLR8, | Diffuse large B-cell |
| TLR9 | lymphoma |
| TLR7, TLR8, | Macroglobulinemia |
| TLR9 | |
| TLR7, TLR8, | Muscular dystrophy |
| TLR9 | |
| TLR7, TLR8, | Waldenstrom's |
| TLR9 | macroglobulinemia is |
| | refractory |
| TLR7, TLR8, | Relapsed diffuse large |
| TLR9 | B-cell lymphoma |
| TLR7, TLR8, | Plaque psoriasis |
| TLR9 | |
| TLR7. TLR9 | Tumor |
| TLR7, TLR9 | Inflammatory bowel |
| | disease |
| TLR7, TLR9 | Systemic lupus |
| | erythematosus |
| TLR7, TLR9 | Rheumatoid arthritis |
| TLR7, TLR9 | Hyperlipidemia |
| TLR7, TLR9 | Multiple sclerosis |
| TLR7. TLR9 | Plaque psoriasis |
| TLR9 | Autoimmune hepatitis |
| TLR9 | Metastatic head and |
| | neck squamous cell |
| | carcinoma |
| TLR9 | Metastatic breast |
| | cancer |
| TLR9 | Metastatic melanoma |
| TLR9 | Metastatic non-small |
| | cell lung cancer |
| TLR9 | Myasthenia gravis |
| TLR9 | Primary malignant liver |
| | tumor |
| TLR9 | Psoriasis |
| TLR9 | Pancreatic ductal |
| | adenocarcinoma |
| TLR9 | Pancreatic cancer |
| TLR9 | Mycosis fungoides |
| TLR9 | asthma |
| TLR9 | Small lymphocytic |
| | lymphoma |
| TLR9 | Advanced pancreatic |
| | ductal adenocarcinoma |
| TLR9 | Advanced non-small cell |
| | lung cancer |
| TLR9 | Advanced non-small cell |
| | lung cancer |
| TLR9 | Head and neck tumor |
| TLR9 | Head and Neck |
| | Squamous Cell |
| | Carcinoma |
| TLR9 | Eosinophilic asthma |
| TLR9 | Renal cell carcinoma |
| TLR9 | Ductal carcinoma |
| TLR9 | Breast cancer |
| TLR9 | Uveal melanoma |
| TLR9 | Skin melanoma |
| TLR9 | Cutaneous T-cell |
| | lymphoma |
| TLR9 | Refractory non-Hodgkin |
| | lymphoma |
| TLR9 | Refractory malignant |
| | solid tumor |
| TLR9 | Diffuse large B-cell |
| | lymphoma |
| TLR9 | Merkel cell carcinoma |
| TLR9 | Chronic obstructive |
| | pulmonary disease |
| TLR9 | Chronic lymphocytic |
| | leukemia |
| TLR9 | Chronic Hepatitis C |
| | Genotype 1 |
| TLR9 | Follicular lymphoma |
| TLR9 | Influenza virus infection |
| TLR9 | Squamous cell |
| | carcinoma |
| TLR9 | Ulcerative colitis |
| TLR9 | Macroglobulinemia |
| TLR9 | Locally advanced |
| | pancreatic |
| | adenocarcinoma |
| TLR9 | Locally advanced breast |
| | cancer |
| TLR9 | Colorectal cancer liver |
| | metastasis |
| TLR9 | Colorectal cancer |
| TLR9 | Conjunctival tumor |
| TLR9 | Hepatocellular |
| | carcinoma |
| TLR9 | Intrahepatic bile duct |
| | carcinoma |
| TLR9 | Relapsed mantle cell |
| | lymphoma |
| TLR9 | Relapsed marginal zone |
| | lymphoma |
| TLR9 | Relapsed grade 3a |
| | follicular lymphoma |
| TLR9 | Non-small cell lung |
| | cancer |
| TLR9 | Nonalcoholic |
| | steatohepatitis |
| TLR9 | Indolent B-cell non- |
| | Hodgkin lymphoma |
| TLR9 | Multiple sclerosis |
| TLR9 | Hepatitis C |
| TLR9 | T-cell lymphoma |
| TLR9 | EGFR-positive non- |
| | small cell lung cancer |
| TLR9 | CD20-positive non- |
| | Hodgkin lymphoma |
| TM4SF1 | Digestive system tumor |
| TMC1 | Hearing loss |
| TMPRSS6 | Polycythemia vera |
| TMPRSS6 | High-risk |
| | myelodysplastic |
| | syndrome |
| TMPRSS6 | Thalassemia |
| TNF | psoriasis |
| TNF | Renal cell carcinoma |
| TNF | Prostate cancer |
| TNF | Brain cancer |
| TNF | Huntington's disease |
| TNF | Melanoma |
| TNF | Alzheimer's disease |
| TNF-α | Tumor |
| TNF-α | Rectal cancer |
| TNF-α | Psoriasis |
| TNF-α | Pancreatic cancer |
| TNF-α | Inflammation |
| TNF-α | Periodontal disease |
| TNF-α | Retinitis pigmentosa |
| TNF-α | Esophageal cancer |
| TNF-α | Sarcoma |
| TNF-α | Prostate cancer |
| TNF-α | Rheumatoid arthritis |
| TNF-α | Crohn's disease |
| TNF-α | Locally advanced |
| | prostate cancer |
| TNF-α | Colorectal cancer |
| TNF-α | Melanoma |
| TNF-α | Melanoma |
| TNF-α | Osteosarcoma |
| TNSALP | Hypoalkaline |
| | phosphatase |
| TOP1 | Tumor |
| TP63 | Colorectal cancer |
| TPP1 | Neuronal ceroid |
| | lipofuscinosis |
| TPP1 | Neuronal ceroid |
| | lipofuscinosis 2 |
| TRAIL | Metastatic non- |
| | squamous non-small |
| | cell lung cancer |
| TRAIL | Pancreatic cancer |
| TRAIL | Hepatocellular |
| | carcinoma |
| TRAIL | Lung adenocarcinoma |
| TRBC1 | Immunoblastic |
| | lymphadenopathy |
| TRBC1 | Relapsed T-cell |
| | lymphoma |
| TRBC1 | TRBC1-positive T-cell |
| | lymphoma |
| TRBC2 | Peripheral T-cell |
| | lymphoma |
| TRKA | pain |
| Trop-2 | Breast cancer |
| Trop-2 | Colon cancer |
| Trop-2 | Lung cancer |
| Trop-2 | Malignant epithelial |
| | tumor |
| TRPV1 | Eye pain |
| TRPV1 | Dry eye syndrome |
| TRPV1 | Complex Regional Pain |
| | Syndrome |
| Trypsin | Cystic fibrosis |
| Trypsin | Chronic obstructive |
| | pulmonary disease |
| Trypsin | Respiratory syncytial |
| | virus infection |
| Trypsin | Emphysema |
| Trypsin | Alpha-1 antitrypsin |
| | deficiency |
| TSHR | Thyroid cancer |
| TSLPR | B-cell leukemia |
| TTK | Corneal disease |
| TTR | Transthyretin |
| | Amyloidosis |
| TTR | Transthyretin cardiac |
| | amyloidosis |
| TTR | Transthyretin- |
| | associated familial |
| | amyloid polyneuropathy |
| TTR | Hematopoietic stem cell |
| | transplantation |
| TTR | Stargardt disease |
| TTR | Senile cardiac |
| | amyloidosis |
| TTR | Transthyretin amyloid |
| | cardiomyopathy |
| TTR | Familial amyloid |
| | neuropathy |
| TTR | Polyneuropathy |
| TTR | Amyloidosis |
| TUBB4A | Type 6 myelin-reduced |
| | leukodystrophy |
| TUSC2 | Breast cancer |
| TUSC2 | Lung cancer |
| TUSC2 | Non-small cell lung |
| | cancer |
| TYK2 | Autoimmune disease |
| TYK2 | Novel Coronavirus |
| | Infection |
| TYMP | Mitochondrial |
| | encephalomyopathy |
| TYMS | Pancreatic cancer |
| TYMS | Gastric cancer |
| TYMS | Prostate cancer |
| TYMS | Ovarian cancer |
| TYMS | Glioblastoma |
| TYMS | Bone cancer |
| TYR | Hyperpigmentation |
| TYR | Melanoma |
| UBE3A | Angelman syndrome |
| UCN2 | Heart failure |
| UCN2 | Abdominal obesity and |
| | metabolic syndrome |
| UCN2 | Obesity |
| UCN2 | Nonalcoholic |
| | steatohepatitis |
| UCN2 | Type 2 diabetes |
| UGT1A1 | Nonalcoholic |
| | steatohepatitis |
| UGT1A1 | Crigler-Najjar syndrome |
| ULBP1 | Hematologic |
| | malignancy |
| ULBP1 | Advanced solid tumor |
| ULBP1 | Advanced cancer |
| ULBP1 | Refractory cancer |
| UNC13A | Amyotrophic lateral |
| | sclerosis |
| UNC13A | Frontotemporal |
| | dementia |
| UPF1 | Amyotrophic lateral |
| | sclerosis |
| USH2A | Usher syndrome |
| UTRN | Duchenne muscular |
| | dystrophy |
| VAMP1+VAMP2 | Nasopharyngeal tumor |
| VCAM1 | Inflammatory bowel |
| | disease |
| VDR | Multiple sclerosis |
| VEGF | Diabetic retinopathy |
| VEGF | Wet age-related |
| | macular degeneration |
| VEGF | Macular degeneration |
| VEGF-A | Myocardial ischemia |
| VEGF-A | Peripheral arterial |
| | disease |
| VEGF-A | Diabetic macular edema |
| VEGF-A | Retinal vein occlusion |
| VEGF-A | Wet age-related |
| | macular degeneration |
| VEGF-A | Breast cancer |
| VEGF-A | Colorectal cancer |
| VEGF-A | Macular degeneration |
| VEGF-A | Liver cancer |
| VEGF-A | Malignant ascites |
| VEGF-A, | Angina |
| VEGFR | |
| VEGF-A, | Ischemia |
| VEGFR | |
| VEGF-A, | Coronary heart disease |
| VEGFR | |
| VEGF-C | Lymphedema |
| VEGF-D | Peripheral vascular |
| | disease |
| VEGFR | Tumor |
| VEGFR | Head and Neck |
| | Squamous Cell |
| | Carcinoma |
| VEGFR | Retinal disease |
| VEGFR | Macular degeneration |
| VEGFR | Nasopharyngeal |
| | carcinoma |
| VEGFR1 | Preeclampsia |
| VEGFR1 | Age-related macular |
| | degeneration |
| VEGFR1 | Choroidal |
| | neovascularization |
| VEGFR1 | Macular degeneration |
| VEGFR1, | Mesothelioma |
| VEGFR2, | |
| VEGFR3 | |
| VEGFR2 | Tumor |
| VEGFR2 | Wet age-related |
| | macular degeneration |
| VEGFR2 | Hepatocellular |
| | carcinoma |
| Viral proteins | Hepatitis B |
| Viral proteins | Chronic hepatitis B |
| Viral proteins | Marburg virus disease |
| VP24 | Marburg virus disease |
| VP24, Viral | Ebola virus disease |
| RNA | |
| VP35, Viral | Ebola virus disease |
| RNA | |
| VSTM1 | Tumor |
| VWF | Hemophilia A |
| VWF | Thrombotic |
| | thrombocytopeni |
| | purpura |
| VWF | Thrombotic |
| | microangiopathy |
| VWF | Thrombosis |
| VWF | Hemolytic uremic |
| | syndrome |
| VWF | Intracranial embolism |
| VWF | Carotid artery stenosis |
| VWF | Acute ischemic stroke |
| VWF | Acquired thrombotic |
| | thrombocytopenia |
| | purpura |
| VWF | Coronary artery |
| | thrombosis |
| VWF | Von Willebrand disease |
| VWF | Von Willebrand disease |
| | type 2 |
| WASP | Angina |
| WASP | Wiskott-Aldrich |
| | syndrome |
| WT1 | Acute myeloid leukemia |
| WT1 | Myelodysplastic |
| | syndrome |
| WT1 | Multiple myeloma |
| XIAP | Pancreatic cancer |
| XIAP | Breast cancer |
| XIAP | Chronic lymphocytic |
| | leukemia |
| XIAP | Acute myeloid leukemia |
| XIAP | Acute myelomonocytic |
| | leukemia |
| XIAP | Hepatocellular |
| | carcinoma |
| XIAP | Non-small cell lung |
| | cancer |
| XIAP | Malignant epithelial |
| | tumor |
| XIAP | Multiple sclerosis |
| XIAP | B-cell lymphoma |
| XKR8 | Pancreatic cancer |
| XKR8 | Colon cancer |
| XLRS1 | Retinoschisis |
| XO | Gout |
| YAP1 | Advanced solid tumor |
| ZSCAN4 | Dyskeratosis congenita |
| ZSCAN4 | Pancytopenia |
| ZSCAN4 | Bone marrow failure |
| | disease |
| α-glucosidase | Glycogen storage |
| | disease type II |
| α-Klotho | Amyotrophic lateral |
| | sclerosis |
| α-synuclein | Parkinson's disease |
| α-synuclein | Parkinson's disease |
| α-synuclein | Multiple System Atrophy |
| αvß3 | Breast cancer |
| αvß3 | Melanoma |
| αvß6 | Tumor |
| αvß6 | Pancreatic cancer |
| ανβ6 | Amyotrophic lateral |
| | sclerosis |
| β2-adrenergic | Glaucoma |
| receptor | |
| β2-adrenergic | Ocular hypertension |
| receptor | |
| β-galactosidase | Gangliosidosis |
| β-galactosidase | GM 1 gangliosidosis |
| β-globin | Sickle cell anemia |
| β-globin | Beta thalassemia |
| Amiloride- | Cystic fibrosis |
| sensitive | |
| sodium channel | |
| Amiloride- | Chronic bronchitis |
| sensitive | |
| sodium channel | |
| Intercellular | Inflammation |
| adhesion | |
| molecule | |
| Viral RNA | Pseudohyperkalemia |
| | Cardiff type |
| Viral RNA | Hepatitis C |
| Viral Protein | Merkel cell carcinoma |
| Viral Protein | Resectable Merkel cell |
| | carcinoma |
| Collagen I | Achilles tendinitis |
| Immunoglobulin | Lymphoma |
| light chain | |
| kappa | |
| Immunoglobulin | Multiple myeloma |
| light chain | |
| kappa | |
| Immunoglobulin | leukemia |
| light chain | |
| kappa | |
| Endotoxin A | Prostate cancer |
| Coagulation | Hemophilia A |
| Factor VIII | |
| Human | Solid tumor |
| endogenous | |
| retroviral | |
| elements | |
| Growth factor | Neurodegenerative |
| receptors | disease |
| Cell adhesion | Inflammation |
| molecules | |
| Cell adhesion | Septic shock |
| molecules | |
| SARS-COV-2 | Novel Coronavirus |
| Antigen | Infection |
| Folate receptor | Tumor |
| Apolipoprotein | Alzheimer's disease |
| Tumor antigen | Liver cancer |
| MG7 | |
| Transforming | Tumor |
| growth factor β | |
| receptor | |
| Transcription | Larsen syndrome |
| factors and | |
| related | |
| regulatory | |
| factors | |

What is claimed is:

1. A deaminase, wherein the deaminase comprises an amino acid sequence having at least 80% sequence identity to a sequence set forth in SEQ ID NO: 6 or SEQ ID NO:169, and the deaminase is non-naturally occurring or engineered.

2. The deaminase according to claim 1, wherein the deaminase deaminates at least one nucleotide in a polynucleotide.

3. The deaminase according to claim 1, wherein the deaminase comprises an amino acid sequence having at least 80% sequence identity to the sequence set forth in SEQ ID NO: 169.

4. The deaminase according to claim 1, wherein the deaminase comprises a mutation at a position corresponding to amino acid residue 122, 114, 2, 176, and/or 68 of a reference sequence set forth in SEQ ID NO: 169.

5. The deaminase according to claim 1, wherein
(a) the deaminase has a mutation to H at the position corresponding to the amino acid residue 122 of the reference sequence set forth in SEQ ID NO: 169,
(b) the deaminase has a mutation to R at the position corresponding to the amino acid residue 191 of the reference sequence set forth in SEQ ID NO: 169,
(c) the deaminase has a mutation to T at the position corresponding to the amino acid residue 114 of the reference sequence set forth in SEQ ID NO: 169,
(d) the deaminase has a mutation to M at the position corresponding to the amino acid residue 2 of the reference sequence set forth in SEQ ID NO: 169,
(e) the deaminase has a mutation to D at the position corresponding to the amino acid residue 89 of the reference sequence set forth in SEQ ID NO: 169,
(f) the deaminase has a mutation to F at the position corresponding to the amino acid residue 176 of the reference sequence set forth in SEQ ID NO: 169,
(g) the deaminase has a mutation to I at the position corresponding to amino acid residue 68 of the reference sequence set forth in SEQ ID NO: 169; or
(h) the deaminase has a mutation to I at the position corresponding to the amino acid residue 68 and a mutation to T at the position corresponding to the amino acid residue 114 of the reference sequence set forth in SEQ ID NO: 169.

6. A fusion protein, wherein the fusion protein comprises the deaminase according to claim 1.

7. The fusion protein according to claim 6, wherein the fusion protein comprises: a) a nucleic acid-binding polypeptide that binds to a target polynucleotide; and b) the deaminase.

8. The fusion protein according to claim 7, wherein the nucleic acid-binding polypeptide is a TALEN nuclease, a zinc finger nuclease (ZFN), an RNA-guided nuclease, or a meganuclease.

9. The fusion protein according to claim 7, wherein the nucleic acid-binding polypeptide is a variant of an RNA-guided nuclease with partially inactivated nuclease activity compared to a wild-type form of the RNA-guided nuclease.

10. The fusion protein according to claim 7, wherein the nucleic acid-binding polypeptide is an RNA-guided nuclease, the fusion protein is capable of forming a complex with a guide RNA, and the guide RNA guides the complex to bind to the target polynucleotide and deaminate a base in the target polynucleotide.

11. The fusion protein according to claim 7, wherein
(a) the nucleic acid-binding polypeptide is selected from Cas9, Cas12, Cas13, TnpB, IscB, IsrB, a Fancor nuclease, or a fragment of any one of the Cas9, the Cas12, the Cas13, the TnpB, the IscB, the IsrB, and the Fancor nuclease;
(b) the fusion protein comprises one or more Uracil Glycosylase Inhibitors (UGIs); and/or
(c) the fusion protein comprises one or more Nuclear Localization Signals (NLSs).

12. An isolated nucleic acid, wherein the nucleic acid encodes the fusion protein according to claim 6.

13. A pharmaceutical composition, wherein
the pharmaceutical composition comprises the deaminase according to claim 1, a fusion protein comprising the deaminase, or an isolated nucleic acid encoding the deaminase, and a pharmaceutically acceptable delivery carrier.

14. A system for modifying a target polynucleotide, wherein the system comprises:
a) one or more guide RNAs (gRNAs) or one or more nucleic acids encoding the one or more gRNAs, the one or more gRNAs being capable of hybridizing with the target polynucleotide; and
b) the deaminase according to claim 1, or a nucleic acid encoding the deaminase; and a nucleic acid-binding polypeptide, or a nucleic acid encoding the nucleic acid-binding polypeptide;
wherein the deaminase, the nucleic acid-binding polypeptide, and the one or more gRNAs form a complex to deaminate a base in the target polynucleotide, or the deaminase and the nucleic acid-binding polypeptide are guided to the target polynucleotide by the one or more gRNAs to deaminate the base in the target polynucleotide.

15. A method for modifying a target polynucleotide, wherein the method comprises contacting the target polynucleotide with the deaminase according to claim 1, a fusion protein, an isolated nucleic acid, a pharmaceutical composition, or a system for modifying the target polynucleotide, the fusion protein comprises the deaminase,
the isolated nucleic acid encodes the fusion protein,
the pharmaceutical composition comprises the deaminase, the fusion protein, or the isolated nucleic acid, and a pharmaceutically acceptable delivery carrier, and
the system for modifying the target polynucleotide comprises:
a) one or more guide RNAs (gRNAs) or one or more nucleic acids encoding the one or more gRNAs, the one or more gRNAs being capable of hybridizing with the target polynucleotide; and
b) the deaminase, or the nucleic acid encoding the deaminase; and a nucleic acid-binding polypeptide, or a nucleic acid encoding the nucleic acid-binding polypeptide;
wherein the deaminase, the nucleic acid-binding polypeptide, and the one or more gRNAs form a complex to deaminate a base in the target polynucleotide, or the deaminase and the nucleic acid-binding polypeptide are guided to the target polynucleotide by the one or more gRNAs to deaminate the base in the target polynucleotide.
